SEQUENCE LISTING (117 sequences)

SEQ ID NO: 1 - DNA, 661 bp, Homo sapiens
acacagtgtgcacacacgtgcagggacatacccccttccccaactgcctggcctgcacacttggcatttccagtatttctaggaagtgatggctctgtgcatcctgagccaatccagctccgagcctccaaggcatcctggtgatgggcagctggaagctctgcctctgaggccttcacacacccaccttcggtcaaacttgcttctgctgaggaacttggtgtgtcttccttctgggcaggaggtcacatttgagagcacaggagcagtgcctgccccccgggaatgtggctctgggtagaattgcaggctcaggggttttgggcaggagagcaccaaccgtgccacacccacacagacacggtcactggggccctgcagcagggacgaccgcacttcccaaagggctgggaagccatgtccagaggaggccatgctctagctcccttgggcagggctggctgcaaggagggtgaagttgggcatcttgaacccagagaagtagaggactcagcaccagcacaaccagctcggcgcattaatacacattcctctcccacttctccccaagcctgaaaaaacctcaaaccagcctctttgcagctccctgaggtcatgactcacgaaccatgctcggggcaggggaaaagaaaagcatccg

SEQ ID NO: 2 - DNA, 627 bp, Homo sapiens
ctaaaggaggaatagatgtctttaagaagaaatgaaaaaataaagtaaatgtgaaaatttcccttacttatttccaaacaagtgctcctccaaaaaaatgcaaataattaagtttctgaaatggtgaacatatcagattagtagacatatggcaggagcagcaaatgagcagatcaagttgaagtcctagtattaccaatctgttaatgttgacaggaagactcattttgactgttccttttatatcaataaatgagtggatttcaactactctaaataggaatgctaaaagcagcactgctaaaagtgcatatcaaaccaataattttctgatgctgttttggtatatcctacaaacatttgtaggacaacaactcagaagggaaaaaaatatcttatgcctttgaggtctgtactgaatgctaatgcatttgtatatgatgggtttaatacagaactgagaataaattactttcagcagctgcactctagacctataaatcgctctgagtactacaaaatccatacaaaggaagaacagctggataatttacaccaccagtatttgtcaaaaaaaaaaaaaaaaagctgaaaatacagaacctgattttgtccctttttcgagta

SEQ ID NO: 3 - DNA, 297 bp, Homo sapiens
aggggccctccagcactggtcttgaaggggtgacagggtctggggtctgactcccacctccaccacttcccacctgagggccctggaatgaatcctttcctggatctgagctgccacatcatcagtgaaaatgacacctatatgggacttcagtgagaacacaaatgcaacgttcctgccacggaacaacccatgtactcactgggagcattgagagtagatccacactgattgacacagggactccaggcctgacccatgatatgtactggatacatggccatgagtgctccacag

SEQ ID NO: 4 - DNA, 649 bp, Homo sapiens
acaccaaatactgctttgctgccttaggcttcagcacattagcatggcttcctcccttggcatggtaactgtagctgaacttggagggtttgtattacccattataattattacttatttcacatggaaaacaagaaaatctttatgggaattccaagttccccctaggaataccaaagagaggaaaaaggctttgaggatggtcctgatgtgtgaagtggtgttcattgtgtgtttcactccttaccacctcaacttcccattctttatgatggtgaaggaacatgtctttttgaactgctcttttataaagatcattctctgtttccacattatttccctgtgtcttgcaaatctgaattgttgtcttgatccagttgtatattattttatgacctcaaaatttcatgatcaattttcagatcatggcagcttggttcttcagtcatgtatgagatgtaataacagtaccttagaaattcatcagaggaagggaggatcttcaaactatctctcttgaatgtttgaaagattccaagacaatataatcaaataattaactagaaaaatcgatatgctctattagtgtatctatgtcactttgaagatttttcttttttttttttctttttttttattatactttaag

SEQ ID NO: 5 - DNA, 626 bp, Homo sapiens
aatgctactgctcctgcatataatagctgctttgaagtgtttgtctgctatgtccaaaacatagactctttcaaaagcactttctgttgtctcctttttctcttgcataggagtcacatttttctgtctcttcacatatttcatatttatttttgttgaaaaccagacattttagataatgtgttgtagcagtccagatactgattctctcccccaggagctgttgtctttcttacttgtatatgtgtttagtgacttggctggactattttaataatgtgtatttccctgtagtatataccatcttttatactaatgttacttttccgatagtgcagccttgggcatggacagagttatcctgggatgacagtaacttttaatagggctctctatgactatctctttccctgatgtccctgttaagctatctgcatctcttggtatcacacctagcctttgacttccactaattgtttgatcattgcctcactgtttttggcagtgccctaaggcataaagtgttccacagtctgatataattaaattcagattcttacaagagtggtctttgaggccagtccttgaggtttgtgctgactctgggagggctcaaatgtttccct

SEQ ID NO: 6 - DNA, 400 bp, Homo sapiens
cctcacatccccttcccctcaaaccctggcaaccccaaactgttcttgacagcctcctttggcatttcctcattttggtgtcagatctcacagcagaatttcttacctattatataccagtgcctcagtgtgaagttccggtttaacttcttgttaccacgagcccactatcttgccccaataataccctcccccaattcacaaacacacaagcattccctcctacagctttgggcctcctatctgagtccttcaggaaagaagtgctgtgtaactcccttggcagtgagtgtagacttggtccaaggaagatgagcaccagtcagggcagctgggccctcttctctccctggccatcagcaaatcagcactgcccatcgatgcccaggcaatgggagcg

SEQ ID NO: 7 - DNA, 556 bp, Homo sapiens
attactatttttcaacctcttttactccagggacttctatgcaccctctccctcaactccccctcaatttgttctcataatccccatgacccccagttttataacaccactgtcaggagcccaaagctgccattcattcacttccattagcatgactcttcatgtactttggggtcttcagtctctccccttctcctaatttccagggttccattctgcttctgctggcttccctacaaagcctgcaacatcataagccatttcaggaaagagcttgatcatcttttgatgaaccctgcattcatgactcactgccttacctgtctttggctctgcatgtcccccagtttccgtttctttctctggaaagagagattgcccaagagtcctgcacatcagcattactagaaatgcatgcagaccagcttcagctgcttgccaactctttaaaaaatgagtaaacaattttctaaaggggaaaaaatctcttcacctcctcacaccaactatttgcataattcagtgaccttttataaaccgtgccattgtataagca

SEQ ID NO: 8 - DNA, 670 bp, Homo sapiens
caccgtcctcatcatgatcgtcttcgtcatctgctgctgggggccctactgcttcctggtgctgctggccgccgcccggcaggcccagaccatgcaggccccctcgctcctcagcgtggtggccgtctggctgacctgggccaatggggccatcaaccctgtcatctacgccatccgcaatcccaacatttcgatgctcctagggcgcaaccgcgaggagggctaccggactaggaatgtggacgctttcctgcccagccagggcccgggtctgcaagccagaagccgcagtcgccttcgaaaccgctatgccaaccggctgggggcctgcaacaggatgtcctcttccaacccggccagcggagtggcaggggacgtggccatgtgggcccgcaaaaatccagttgtacttttctgccgagagggaccaccagagccggtgacggcagtgaccaaacagcctaaatccgaagctggggataccagcctctaagacggttggaatggccagcttatgaaggcaaatttccactcgcattatttaatgatggaagattctgggggagagttgtggatttcataaagccaaacatttaaagctagagacgggggaggcttaccactttccccaaacaacataaaagacaatgtcccttctttcaaaaagtgc

SEQ ID NO: 9 - DNA, 1860 bp, Homo sapiens
cgctcctgcgtaaacacgcggttccctcggcaacgctggaacccacgtcaaaggctccgccaggtccccagcgaccgccacccctccggccgagcccagctccccgcggcggccgctagcccccggccccgagccaccactccgacctagcggccgccgcccccggtgcgggatgaggagatccgcggccgccactgggccccatggaggagccgcagccgccccgcccaccagcgagcatggccttactgggcagccagcactccggcgccccctccgcggccggcccacctggcgggacttcctccgcggccacggcggccgtgctctccttcagcaccgtggcgaccgcggcgctggggaacctgagcgacgcaagcggaggcggcacagctgccgctcccggtggcggcggccttggcgggtccggggcagcgcgggaggcgggggcggcggtgaggcggccgctagcgacggaggcggcgccgctgctgtcgcacggagctgcagtggcggcccaggcgctcgtcctcctgctcatcttcctgctgtctagccttggcaactgcgcggtgatgggggtgattgtgaagcaccggcagctccgcaccgtcaccaacgccttcatcctgtcgctgtccctatcggatctgctcacggcgctgctctgcctgcccgccgccttcctggacctcttcactccgcccgggggttcggcgcctgccgccgccgcggggccctggcgcggcttctgcgccgccagccgcttcttcagctcgtgcttcggcatcgtgtccacgctcagcgtggcgctcatctcgttggaccgttactgcgctatcgtgcggccgccgcgggagaagatcggccgccgccgcgcgctgcagctgctggcgggcgcctggctgacggccctgggcttctccttgccctgggagctgctcggggcgccccgggaactcgcggcggcgcagagcttccacggctgcctctaccggacctccccggaccccgcgcagctgggcgcggccttcagcgtggggctggtggtggcctgctacctgctgcccttcctgctcatgtgcttctgccactaccacatctgcaagacggtgcgcctgtcggacgtgcgcgtgcggccggtgaacacctacgcgcgcgtgctgcgcttcttcagcgaggtgcgcacggccaccaccgtcctcatcatgatcgtcttcgtcatctgctgctgggggccctactgcttcctggtgctgctggccgccgcccggcaggcccagaccatgcaggccccctcgctcctcagcgtggtggccgtctggctgacctgggccaatggggccatcaaccctgtcatctacgccatccgcaatcccaacatttcgatgctcctagggcgcaaccgcgaggagggctaccggactaggaatgtggacgctttcctgcccagccagggcccgggtctgcaagccagaagccgcagtcgccttcgaaaccgctatgccaaccggctgggggcctgcaacaggatgtcctcttccaacccggccagcggagtggcaggggacgtggccatgtgggcccgcaaaaatccagttgtacttttctgccgagagggaccaccagagccggtgacggcagtgaccaaacagcctaaatccgaagctggggataccagcctctaagacggttggaatggccagcttatgaaggcaaatttccactcgcattatttaatgatggaagattctgggggagagttgtggatttcataaagccaaacatttaaagctagagacgggggaggcttaccactttccccaaacaacataaaagacaatgtcccttcttcaaaag

SEQ ID NO: 10 - DNA, 630 bp, Homo sapiens
tctcaaaaaataaataaaaaccactgtacatcaacaaggcccttgggggacagctggggcataagtaggtgtcagccatacatcagagcagtgtgcctgccctgagctgcttggggttgaccagcctggtgtccagaaatgcctgctggagggagtcgtggtacaggaaaccttgtgctcttagaaggtctcctgagaggccctgcaaagccagagtccctcttagcagctcagatcagtgctatcaaagtatagctcggggattgctgccagcatacaaacttttactggtctgcagcgagataagtacagaaattgaaagtaagcatttagaaacttttataacaattttacaaggtcttgtcaaatgttattaaaacaaagctgaggctggaatttcacctttttcatttcgtttttttcaatttaaacaaattgtagtaaaatataggtaatataaatgtaccattttagccatttttgagcgtacaatttagtagcagtaagtgctttcacaatattgtgtaaccactagtattatatagtatatatttttaaaattttacagaagtattaagttagcagcagattaaacatttttttcttaaattgagcttgagaagcgctggc

SEQ ID NO: 11 - DNA, 683 bp, Homo sapiens
agagagcagattgccctgtgtaggtcaggtctgggttctttctagtccagagtagggaagaaacaggaaagagggctggtgttgaaggaccttcagccacgagaagggctgtgtaccatgtagccctctggggaggcacaaaaaggctcaccattttctgaaaatgactagactgcaggatccacgtgagtgtgactattgcattcatgaccttatccacagggcctcacaaggtgcctgacatgcagtaggctccagatgcatatttattataaagtgaatagtccttaagctgcagggtcccttctatttgcattctaagaaatagtcacttttatgcctaattttgtatttgcagttttataagttttataagagggtctcccaaatagtataaacttcaagccccacaaaacctatgtttgcctcccataggcatgcaataaatgttcgtggatctaatgagtaacaagaaaaagaaggaacaaaaccctaacccctcccctacccaaaccagtggcaaccggggaggatcaaattcaaccttgatcagtcagaggcagcattcctaaattattcccaagcagcaatagacaatgatttacctcaattaattcagccagttaaaagcttagttcttacttgccaaccgaaggcttgaaggcaaaatgtgtttaagcctc

SEQ ID NO: 12 - DNA, 481 bp, Homo sapiens
ttcaggcagatgtcagttaaaaacttacctctgcacactgcaaaaactgtatagccctgaacagatacttttcttgagcatagttcctttgtctctaaagcaggcataattgccaatgtggggatgatatttagaaatctgaactgatgtttattctctaggggtcttctcatttgagctgggattggagatgtctagtgtctcagagcagcaataagaaaacagaaacctcttccagcttctgacatccaaatgtcaagctcttaggagaagaatggaaagtcctcaagaaatgcaaatagctttggcagaatagctgatgaagaccacctctcccccctccagaaaggcattggttccccattcatggaaaagggaatgtagagagagattagataatagtacatccataaggttcctggaatctgcatctgaggaagaggggcgtcagagaccccagctgttatctataatccctcct

SEQ ID NO: 13 - DNA, 693 bp, Homo sapiens
gcactagggcaaagtcaagacatacgggtgtccagttctagctttgcaactaactggttatatatttttaagttacagtcactctgcgccagtttcctcatttttaatagagtgggttagaactagataaatactttcattttgtcaagctctaaattctgacttcaggaaaaaaccataaggcactggaggtttattcataggtttttctgctgaccccgtccctctctgtttcttcaaccaccacaagacaatcaacttccctgattggagattggaacaggtgtgttctaattctaaatgcatcacttaactattagttccaactctctggggcttccttcaaataggggaattagactggtctccaatctctttgtacagatgagtaactttatttacccaaagatttagtattaacagtcgggagcaggagggagaatacttatgagacaacagccatttccacagtggagaggaatggtttgttcccaatagaagttaccagatttcagtcccattgccaaatagatattatgagcaaggaagaaatctatagtagtaacttaagaccaccagaaagatcaaagcccagagggtgagggtatggcaataaacattagacatatctctaaccctcttttgtttgaaatactcattaccctgtggtactgggaatacctgtgcctacaa

SEQ ID NO: 14 - DNA, 733 bp, Homo sapiens
ttctgccatcgcaaggggagggaagagcacctaaagggcttatgagaggtttgactgaccaagggagggaacagaacacattcctttccattggctaggactcggtcacatggctttccctcattattagtgaggcttggagaattcatctatttgtgagcccaggaagaagagaaaacaaattgtggtaaacatttagcagtctctatgacaatagtctgtatgttgactgcaaaggtggatgaacaaaaccaagcctcctttaaagcaatacaatctggcagagtccctgggttatcattctgaacatagatgcttattgttcaagagttaagaaaattagcatgactgcattccagttctataaatttaatctttattcagcatattgtcatccacatgtcttaaaaaataaaataaaaaacaaaaaacctagtaactacgttttatatagcaaggaacactcatatatatcacttcattgtatccttacaacaatcctgtgcagtatatgttttactccctttcttctatgttttgtatataaagaaatgagccccagggagttgaatggcttgccccaactagtgaagctaaaactccaatccaggtctttttatttccaaatccataatctacaaccatctgtagagagttataattaagagatatgaatggtcaggggcctttccatttcagtgcaagtctgcccagctccaactaccagcatctg

SEQ ID NO: 15 - DNA, 694 bp, Homo sapiens
aatcctgcatttcccatgctctggggtgagaaggaattagctgggagccaattagcaatcttgtcagaagcaaatgaattttgaataaactggatacttaaactgaaatgagaccattgaaaccagaagagcctgagatccatcagttagaggaaataaaagaagtggcattttccttgccatctgggtgcagtgtgagtgatttttataatcctaccacatttttatccctgcttcccttaaactgtaggacccaaggaacctggctgttttgttcaacatgatgtgaccccatacctaaccaggccaggcacaaaattggcctccaataagtagtggatcaaagtatgaatggataaactgaatgaatgaagccaaacttgaatttctccatagcttatccaaatgggaatggtaaaaatcataagcttttgagaagagaacttattaagaagccctacatcagtcatgactggcatcattggttagtttacccaattttctccttcccttcatcttcctaatgcaactctggtttgggctgcagtatacccagttagaatacgccctccccagagtctcatccagctggagatgatcaagtcaccagttctagctaataagttgccagccaaagtattcaggatgagacttccagaaaaagcattgttttcctgataacaatggacagacc

SEQ ID NO: 16 - DNA, 674 bp, Homo sapiens
gactgttattactgaaagtcattgcttttagactcttccaactacagagcaaggaagtttatgtgtatatagtaatctgtgaatatacacatacatacacatatttctatatgtaatcatccatatttaaattaagtagaatatgagttcatactgatatctccaatcctaatcagttaccacagggattattccggcctttttcccttggaagtttgcaactcctgcttcaacagttagaaatctggcttccatattcatttgcttaattgttcaattccagtacacataaatggtggcttcagaattaataacttatacctccatgggaaataactttattaactaaagtacagcacttatgtatagtactttttgaatttttagacttagagattcctcttcttttccaaagttacttaggtcagaaccattttccattcttcagtgaagttgtcttatgtatttgtaatacagttagattgttctgtcatatggtgcattccatcctgggatttcctatctctttttttaacatttgcatatattaagtttcattcttttgtgctgtatcattctatgggtttcaattaatgcatagtgtcatgaatctgccaccataggagcatcatacagagtagtttcaccaacttaaaaaattccctatgttttac

SEQ ID NO: 17 - DNA, 645 bp, Homo sapiens
cgttccttcctctgtgtcataatggacatgatgatagttggctcactcagtaaacattctgtgtctggaaggatttgatttgtccttttcttgaggcaacaattttgaggtgatttgaaaaatctttcttgaaaaattaaaaaatttttctaattaaaaataatgcaagctcattagaaaaaaattgaaaaataaataaaagcacaatttttcttaaccacttaaagatgaccattgttagtttttttttttttttggtgcttttttccgtttccaatctcttttctattaaaacttctgaaatgtgattgtagcaatgacgcataaggggcccttgacacattgagaaatttataaatacgctggcttcttgtcttgcttttgtccccagcttaactgggaactctttttctatatctttgaaactccaaatcctagataattcttcaaggtcaagctccaatgtcttgctggattcttctcagcaggaattgatctattttctctgtatttttgtgccacaggatctatgactctcttatggcaactaccaccttctgccttatattacgatttttgaatcttccaacaaagtctaattttttttttttcaaatgaagtctcgctatattgcccaagctggag

SEQ ID NO: 18 - DNA, 707 bp, Homo sapiens
atgtcatgggaaatgcaagaatatgtgtccagcatggaagggaatcagtatggaagtcttttgataaattgtggcatttatcactaacattgcctcaaaactttagactacctgccatatacaaattagaggtgaaaattacttccatgtaatatacaagccaacacaaagaatcctatcccagtttcttggatggataggcaagaatctgggtaaggtttattgtgcaataatcctcttctctcttctataggccaggatttaagtttacctcaaaaatggaaaattttggctgggaaaattacatgtgggaagacatcttcagtggagattttagtaattacagtttcagctatgaccctaccccttttctactagattctgccccatgttggccagaatccctagaaatcaattatgttttgatcatcatctatgccctgatgtttctactgaacgtgatgtgaaactccctgccgatgctggtcatcttattcagctgagtcagccactgtcaccgatgtctacctgctgaccctggccttggccgacctgttcttttccctgacattgcccatcttggctgcctccaagaatgaatggctgggatttttggcacaatctgtgccaggtggtctagctcctgaaggaagtcaacttctacggggggtattctactactggcctgccgcagcatgggactgtta

SEQ ID NO: 19 - DNA, 680 bp, Homo sapiens
tatgatatttcagccatggtgctgaacatttccaaacagcataaatgcaccatgtgtgtatgttttcctttgggatgctgtgcttagagggtagcagacagggtgcaaagtgagaaggacctggctctgcacccaacactgccagtattgaatcctgactccatcatctgggagctgtgcaacctacgcaaggtacttggcctcagtttcctcatcatccccatggcatttttgtgagaattaaatgagctgaaaccttgaaaccccttcaaacagcagctggcacagaggaagcacacaatcaatgtcagctgtactcttcctggcagtgtggagatcccagctctgcccctagctagtcacttctcttcttggaatctcagttccttcatctgggaaatgggagcagatgtgaaaaggggcagggtgagaatacatatgaaagtgctggctcctggtgcatagcaggcacttaataatgatacacttttccatcttctgccttccccagggatgcattgtgccatgtaagagagagagcctccagggttggcgagagtttttgatccaggctttttcaggtgtcaaagatgagctgggtgattctccatagattttcctttctaacaggtgacagttctgtttcagaaatactgtggatgttcaggtttacagcacat

SEQ ID NO: 20 - DNA, 479 bp, Homo sapiens
tttatgctcatgtagttctttccaagaagagaaattacagagtcaaattgtagaaatatttaaaaatctttggcacacataaacagtatccatataatttataccatcttttagatgagttttaacaccaaatgatagaaatctcagtttcatacagatttggtgggctggaaccaaatacttgcctgataggctgtcccctcgtctttcctagctgttctgggaaaggcagttcctggtaagaactctccctacggcccctttcatctcactgttcctcagggcatagataagtgggttgagcagtggggttcccaatgtgtacaccagtgagatgaactgatcttgcttggggttgtagctggagctggggcacaggtacatgaaggcacagcagccatactgcagcagcaccacagtgaggtgggaagagcaggtggagaaagcccggtggcggccagcagccgagtggatcttga

SEQ ID NO: 21 - DNA, 709 bp, Homo sapiens
tttaagccacccagtctgtggtgttctgttatggcagcccaagccagctactacagggtgggacgaggggaggagcatggcctctgctggaagtgcaggcaaatgatcccccaggaacaatgatgggagcttctgattgctctcattatctctgcaaagtaggaagaaagattcatcagctgagcatgaggatggtagaaaacatctttgggaaatttcagaagtgaaggaaggcataaaatagtcatctaaaaaaagcaggaaagggaaaagacagagaaatccagtatgagtcccaggactccaggaagcatcaggacccacttgaaattgccaatgctgaatttaaaatgaggccagtctgtacagaagcacttctggaatttgctaacagctaaatagagtagaatcaatactttagagaatacgagtaaccaaaggaataaaattaactgatcaacttttgtggtttttactattaatattttcttcagtgtaaatcatagctgcctgaattcctgaacccctcttatataaatctaaaaagctctggtttatcatggttgaaaattcatggctaacttatcaggcaaactgtccctaaagcattttttgaatagctttagtatcaagatggtactgagtgtacatttcatttccctgcttaaaggaaggcttagttattttaaaccaagtcttatttttatag

SEQ ID NO: 22 - DNA, 517 bp, Homo sapiens
atttctggatttatgcctcccctgacccattccaggatttaccccaaaccttccacactctcttctaacagggaaagttctgttatgacacaatagtacttattaagacagatttaccttctaagtctcaggacagcatttcacaaccagaaataactggtcacatgaagaaccaggagtctggtagtagtgaaattcattttccttcttgaaaaagtggatcaaaggattcaaacagcaagtggtgaatcaatgaaaagtggtaaaatggtgaggaaaaaatgttactaaaagatgacctcaagattactggtgcatatgaattgcttttttatataggaaaatactggataatttcttattgtcatagtataattagaagcaatttcatgtgttcattttgccacatgagtttaaatggaatagatttggttccctctctaacatgagttcagtgtctgaacttgggcaaatttctaaacaattctgagcttcactacctctgcttgaaagtgag

SEQ ID NO: 23 - DNA, 695 bp, Homo sapiens
ctttggaattttattctaagcatcaatcaagaggtatagtacgagaaaggtagaacatgtaattataaattcaggattcaggaagtttatttttctcttctttttaattctctcaaaatgatcttgattcctgcaaagtgttagtatatctggtaagtaagagtctatttcttttaaacttcatctgtattaaccagctttatatgaccaaaatgtcccccaaatttaaatctttgcacagtaaggccttatatgtacacctggcctcatttcaaaagactaaagcagttgttctcaaattcagctgcacattaatataaactggaaaactgtttaagctcctgatgacaaagccacatgtgagactaatttatgctgaatcactgggccaaggacccaggtatcagcattttttaaaactatagaggaataaccagggttgagaaccactgcacaaaatggtaaatgcaacttttatttaagttattttttttaaataaataatggttgaattgatactgatcttagtaccaagtcatggcaattttttcagacttagagaattcatcctggcattgagattattaaagaacctagaaatccaagtgtttttgtttatatttttcctgtaaatattagagtatgctagtgctcatccttatttgataattttggaaaaatatattaaaacattt

SEQ ID NO: 24 - DNA, 677 bp, Homo sapiens
attatcatttgaatgttgatattacatcatatacaaattgattgcaacatagttatttaatgtaacattctattttaaaagataaatttatcagaatcatacattgctacagtagtctcccttatccacaggttcattttctatggtttcagttacctactgtcaacaaggatccaacaatattacatgggaaaatcacagaaataaacagtttgtaagttttaatttgtgcgctgttctgggcaacgtgataaaatctcatgctgttcctctctatcttgcctgaacatgaattatcctttgtccagtatatccacactacatatgctaccttcccattcatcatttagtagctgttttgattatctgatagaaaaaacacacagtatatatagagttttttatggggcaagggaaaactttctctttgtcctctgaagattcactgaaaactcaactcacaagggcagactaataggaatgaaggtaaaaaaaaaaatatattaacatcaatggagataactacagagtgattattccattgccatcaatggactacagtggcttaaatatcgttttgaggttacaaaaagagtggaagtcttgggatcttggcaaaacaggttatgggaagaagagaagagaaaccctggttagcaaaggtcatcttgtgatgc

SEQ ID NO: 25 - DNA, 361 bp, Homo sapiens
ttttttccccctgagtgtttctctcatgctttcctccaaatggagatggagaggtttcacctcacttttctctaactctccctagttttttggtttcttttcctccacatctaaaagtgtgcagaatgtccctttagcacatagaaaatcttttcttgaccctgccacctacttaactaaaatcccacacttttcttcttcttttaagatttcctttataatggtgtgtgtcaatggccacatccaccttatccattccttcttaaagttccagaaaaacggttttgtttcctgttactttaatggaattatttttccaaagatcaacaggactttccctcaagcccaatccagtcggtag

SEQ ID NO: 26 - DNA, 459 bp, Homo sapiens
atacatgataaggtacatggatccaggggaaggatgaagggcagtgtgggattgcttttgaatttctccaaactcgcccataaaagcagacaggacaaactaagataactaaacaaaaaaacccacagacaaaactattacaaaccccaaaagaagtgtggtgggaacaaacatctgatagaatcagacacattactggtgaccggacataagccctgttaatgagaagcttacatttaggagagtcaattaagtacacgctatacacaacctaaagtggtaaatgctaccttggttattcaacttcactgttacatgccttgaagtgtggggtgcactggcctgaaccattctggttgtgtttgattccttaggatgccaccaacaaataacattgagaaatacccagctacttttcattgttctccaatggcagcaaagtacaaatgatctctatga

SEQ ID NO: 27 - DNA, 625 bp, Homo sapiens
tccaggcagtattctccatgacaatgaggaaggtaagtctgcaacagaagaacaatggcagaaattttaagaaaagtttaccgcctgggactatgactcaccttttgggagaaaatgtgactaaccctttgtaagagcttgttgagagctcactttcctgggaggagtcgggagaaggggagcatcagctgacgaagaggtgaaggaggtacccaacaagaaaagcgtagaaggaccagggatttggggtcgggtcttcctcctgattccaagggatggcataagatattgccaagtgaaggaagcgaagtagagccagcaaaggaaggtgaactgttgtttcattagaaataatatgttgtgataattatacaaagtactaattagtaaatttctttccaacctcgacactccaaaaatccctgtacttatatcccgaaggcctcttcttccccaagctggaagacacggtcactcattagtcacccactgtcacaggagtaacagagactacaaatattggacaggacataagtgagggtcaagcatctggatgcagatgcatgacaggatgcaagtcttcccagctctcatggactttgcgacagatgcacagagtgaggta

SEQ ID NO: 28 - DNA, 601 bp, Homo sapiens
attaaatgcctgaactcccttcagctctgaaactctgtggtgtattctctgaggacattacctctctaagggacccaaattaaacagctcacaccatccatcatttttctgtctgaggttttatttccctaatcagatttgggtaaattttcagcctctctctgtctcttacttccaatccaataaaacctgtatggatttgttttgtatttcatctaatgtcattattcattctaagagtcactgcctgaccatttccctgcctatagcataattagctattaaaaagctacactggcatggttttcaaacttgcatcctctttttctgaggtggattgattctaaactgattaaaatatctcagaatttccaatacaatttttaaaatgcaacagattttcaagactgcctcatgactctgccaagccaagggagttagctgccaactctctctgactgccaaggaagccaaataaataatcctgatggtggttttaaaatgagaggcaagtgcccatttcttaggttgacagtgccaccctacacattgacttctccagggtttgtaagacaccaagggtgatgtttcagattttccc

SEQ ID NO: 29 - DNA, 597 bp, Homo sapiens
tccacattctttctaaagttctgagcttttccatgggcttccatggtagggaaagcacatggcctgggtgtgggtagagcaggtgcggccatttatatgtatggttctttgcaagtctggcatttgtaaaatgggtgatgcttgtattgtgtttatttattcaatcatgtaatagaagatgcacataagattattttgaaaagtatgccttccattttcatgctgagaataatgcaggaagttcagtgtaatgcagttataataaaatagtagcaaaacaatattttgctttaaatcatggaattagcaagtaaagactaattggaagccaatcttttgcaaattttttaaatgtaagtttatttggaggatatgacttgttggcccagagtacatataaagaacaaaagagtataattaacaacagtttcaaatatggacttaccaggcatcttgataaaatcagtattgacatgtatgtgaatgccaacattgtgtttttccaattcaatactatgttatgccataaaactggtagcagttatgaaaattagaattggttaaaaactgttgaaatctttaaatttttcctgttta

SEQ ID NO: 30 - DNA, 618 bp, Homo sapiens
tacgtgggtttccctatcgtcctcttcatgagttctttgtgaaaacagaaagactgagtctgccaataaccagcaagagaacaagataaaataaataaaattaaccataagactttaacatatgacaaacaactggtaaggattttcaaaatcttttggtcaactttgatggtatttttccatacaatgaactctaaaatatgaaaaacgtacatccatattttagatataaaagtctcttgcacaggccagaaaatgaaactttaatttaagcaataaaattcccctttgtagactgcaaatggagaacatgctatctagcttcatttttcttcaacttacataaaaatgaaacaatggttaatgttctggcggcatctctaaacatattcagtgaaacaaaatttccttacaaatgtcaacagcttacaacaaataacattttatcctgtttaattatttagaaacaaaatcagttatgctgagatatgtttgcatgggatttatatactctgatcatagaaacaaattattgacatctgaatctgaaagctgcaaaacatgataaaagacataataaaatcacagatttgttattctctcaggaactttttctag

SEQ ID NO: 31 - DNA, 655 bp, Homo sapiens
ggtgcccatgcttggtggtaggatgtatgaagctccttgctcctccagctgggcatcctgccacttgctgagccaaataaggaatgtggggaagcagcaggccaccagccagatgagggccactgccttccaggcagccccatgggacatgaaggagaggtagcgcagtggatggatgactgccaggtaggtgtgcagcacaatggcggtgaaggacaggatggtgctggtgcaggcggcgaagacagcatcagtgagaatgccacaggccatgcggcccagctcccagccacccaggctgctggaggagatgagcatgtggaggagaatgtaggccaggtctgagagcaggatgttagccgggagcaggtagtggggctcctgtcgcagccgttggttccgcaggatggtcaccagcagcagggggctgacagccagtgtggctgcagccagcaggcttgagggaaggaaaagccagtacagcatggagctgggcaccctgaggtcccccaggcccaaggaagtgttgctggctgcttggggcatgcagggtgtcttgctgatgagctggatcagggccggccaagctgtagtgcccacagggcaaggtgccagctcatcccccatgcttcctggcagggatggctggctttgt

SEQ ID NO: 32 - DNA, 697 bp, Homo sapiens
aacactgacttctctgaagcagttgtctaaaagaacctacaccatttttatttagcaaaaaggcttttgttaaaagcaggggatagcagaaagagctttgtaaaaaatatgtcatggattttaggagtttctaagagcaagaaaacgtttcttaaatagaggaatgaagcaattagagttccataaaaatcacctaatgggccttccaaaaggcaaatgctaaagccccagaaatcatcactgaggaagtctgaagtaggaagagaccttgttctagaaagccgacaaggtagaaattaaaatggaacaggcccaacttgaaattccgagaccaaaagaggagctgatgacattggtgggagacaggtgtgggaataaagaatgttggtagattctagagacattccagcgataacacagacaggactttgtgactgactgtatggggcagctgcaggggtaggagaggaggaacgattaagacatgatgaactgggctatgagttggcagctccatttactccagagaacacaggaggtgaaaatcatgggagacttgatgaaaacactttgagaggcaccatggggataaaagccagaaataaggtgggaaatggtggaagctattcattctagaaaagagggtgggaggatgagcataagttaacaggaaacaagttaattttttaaaagtgct

SEQ ID NO: 33 - DNA, 611 bp, Homo sapiens
tttcccagataaattgtatgcacagtaactggtgttgcagtataccatagcatatatacatccatttggcacactgcaggtgccagtgggacaacataccagagtgtaagtcttcctgatcattttcatgatgtcctcagttatttaccttgtaataagcttgtaaacgtctatgattgtttttgagtcatcccaatgcagtcatgtaataacaacatgtattttaaatgaaacttggggattttcctccatacctgaatctctagtattcacataaatgaaaaatcaaaattaggataagttagtgtcaaacattaatggatttttacaatgctaattggtgttcctttttaaattattgctgcctacagacacatagctatagttccatgcactttcaaccaccaatgctgccaggctagtaaagcagttaatgtatatttggggttaattatcagaatcaccagaaacaatttttttaatttttaaaatattttatttttccacagattattggggtacagatgctgtttgattacataagttctttactggtgatttgagagatttgggtgcacccaacatccgagcagtatacattattccctatg

SEQ ID NO: 34 - DNA, 602 bp, Homo sapiens
cgggttatttagagaaccacttgaaataccacctccttggtaacaccagctccctccaccccctgagctcacggtctcttccctgtgagatgcagcaccaggtaaggtcattaacaaccaggtttagagtaaacagtgctgggctgtatttctgatcctgcctttccctaactgggtgctctttggcaagttattaagttacttcatctgtacaatgggttacacttatgccttttacatatggttgttgcgaagattgagtgatatgcataccaaaaatgctgagcagaacaccttgtccatatctttcctctctgttattaaatggaggcctttaaggttaagtaatttgttattgttgtggttaattttagtcctctgaattttaatctagtacaaattgtgctgcatttggcacatggtacatgttcatgaatattgagtgttgtataaaggaatgaaaaatcaattacatgaaaagaaattccaaatcttacattttacaaacacagacacaaagaatactaagatttaactcaggggcaaaagttaagatttggccaccagcacgtggtgagcttccttgaaagtttgtttctggc

SEQ ID NO: 35 - DNA, 428 bp, Homo sapiens
attctaatggcattaatccatagcattatctccatctctgttttaaatatcatgcatcctcattctatgattcaatttaaaggaatccttcaaaaggatcaatctaaataaataacaagttagctttcaggcaaacaaataaatttgctttgttttatattcaccataaatatttcacttaattactgaggtaccttgttcaggaaacacaaaacaacattataaattaattagcactgtccctgctgacgttttagtcctgtggaatgcaaaagctaaaagtaaaaacaggccatgaagcccaaccagagcacacatcgtatgcaaatgataaagcccacaaacatcatgggatcattcctgggacattctgaatcaccaaaattttgtctttaatcaagtattgccctatttattttcaaattcaa

SEQ ID NO: 36 - DNA, 643 bp, Homo sapiens
tttgggtctagaatccccttggtttgggaagcatccagaaggagcttccatccccatccatttcttgctcacttcctcctctctagctttgtttacatgtctctcgatacctagacagaggcagaggcatggacttctggttctaatcattcaagccttacacgtccttcaaggctccattcagaattatctttcctcggggagtctgcttctcctatttcaggatttactgactattctcttatctcttgtaacatttaatatcccactccttagcattaacttttaaacttgctttctaatcctgaggtttgtgttcctttgccttgtaaaattctttgtaaatggccagcccagtacgtagcccagtcccaagcaccacgtaggcaattgaaggagctgggacaaaaagagttctttgtttgaatttcttttactgctctgagtttactctgtatttgcacatgagttttaatgttttggggccattgaactatttgagaatctagaagataatacacctcttttcagaaaaacacatatgaatacacacacacacatgccacctacacacacaattttgcatgtaattttaaggattcattaaccttagcttaccagactgtaagttcctttgcatatt

SEQ ID NO: 37 - DNA, 567 bp, Homo sapiens
gcaagttatctgtatttatccccctacaaacacacactcctaacatacagtggtgagagaggaacaacataactgcagaggaagtaagtgagagacacaaagcagtcattggttcattgctataatgaaattctcctagacaaatgctgccaggatctcttccctggggataaggtctagttatcttcctggaagtggtttccagctcactattctctactgtataattacagtgactccctcatccatcctcttgtcttctcagatcttaactttatcctctagactccaggctcctcctctgagatgttctcacttttctgcaacaaaagctgagtctatttctcaatctgtttgctgtccatagaaaatggaaggttcagaggcttttattcaattttctcagtctctttattgcaagctgggtcccatttacttatataactcttttaaaaagtttttgtgggctttctatgtatcagataatagaccacttcatttgataaaaagccacattctttgttttccagacaagctttctatattttggacaagtaaggccactta

SEQ ID NO: 38 - DNA, 594 bp, Homo sapiens
ttatggtgtttgtaagatcttattgcccaaagagtctgttctgtccatcttatgatatctgttttaacattaatgatgctcagttgtgtctagaccctaaaagaagaagtttgtatgactttccatgctgttatggtcaggaatttagttttaagcttttttggggcctctaagccacaaggggatctgttcagtcagttcagtagagggcttaggatttatcatctttaattcacattcccccattttggtcaaaatatgccaaaagtagcatcaatagccaagctcttatttcattccatattattaccaggtggtgtggctatctatctcagatatattctgttcttcaatgggacccatatagccaagggacttatagccaaaagacttacagccaattaaacattctaggacaaaagggaatggaggtgggaaggcattcattattccttaaaaaccttttgagcaatataagagccacaaaccaaaagccaaaaagtaagcttacaaaaccgatttatctataagttctatgtgttgggccatcggctcttaggcatctgtgagcccatcttttttggaggatctgaa

SEQ ID NO: 39 - DNA, 282 bp, Homo sapiens
aaggcagagggggccagcagggcgggttacagaaccatgatgtgtttttaactggactcacttctgccagtatctgcctgactcttcagcccatgtctcttttccttgttgtaatactaatgggggcattaaggagccagagaaggggcctccgacgccactgcttgtacctctggagttacatttagcggcatttatattttgtcatgtgaaattcgaaatcctcatccaaaatgcaactgtgggggaactctcataggaatttcagccaattctggctcc

SEQ ID NO: 40 - DNA, 626 bp, Homo sapiens
cccttcctccccagccataccgtgacccacccataagctggcccccttagctctggctcacctggctcagacttagaggtggcaggattcctgctgctcaggaaataaggactgctcttgagctcctcacaggccccaggaatcccaacaaaagccaaccaaggctaccttcaggccttccagaagggggtggtagtgtcctcatcaggttccccaagtttagggagagggcagctgggcccagggcccttctccttgtggctcaggatttagccccacttaccatggtgcagccccagccttccagccaacccagcattagaggcagtggctcctcttaatgccaggccctagttggctcaggcataatccagccaggaaacctctcacctttccacagcaatggccaccagtgtgaaaacggaagccgacacagacatgccctgcaccaagccgctcatcttgcatgtggcattgtcgaagggccaccctgcaatgacagaggcccccacagagtgagagatgcccacgcatcaagagccagagactgaaagccctccaagccaggtcccctctgagcttggatctttcctccatgacctgctaggtgttatctggtctctgct

SEQ ID NO: 41 - DNA, 685 bp, Homo sapiens
aaactcccaaacgatagtaacttaaataacttaggtctttaatactctcttcagtaaaagaattctagtagttggagagtccaccatccctaggaatgtagttcttgtcctcatggttcaatatagctgctgatgctccagccattacagccacattccagacagcaaaatatggaaagagaatgaagagaagaagagcgtgcctaggagtcccatgtattatttccatatatatttgggcagaacctagtcacagggccactccatacgtatctgttagctattgctacatagcaaccacaaaatttccatgtcatacaacacatatctgcaggttggctagggttcagttcctccatgctggtctcagacaggcagttctgcttcgggttacagtggctgagctgattccatttctcactgcaggtctgtgtttcagttgagtgactgtcccatgtgcctttcatcttccttgggttgatgaaaggaagccacatctttcaacagggctagccacatctgttcctcatggcccaaagagacaccaaagagcagatagaaataggtgagacctcttaaggtctagactcaaaactggcacactgccacgtctgttcacaagctattagccaaagcatagatgcattaccaagccccaagtcaagggcaagaagtacaatccacc

SEQ ID NO: 42 - DNA, 566 bp, Homo sapiens
acaataatttgttgtatattccaaaatagctagtagtgtaatgtttccaatacaaagaaaagataaatgtttgtggtgatgcatatttcaagtaccctgatctgataattgcacattgtatacatctatcaaaatatcagcagtacctccaaaatatgctcaattattgtataagtacaaaaaaatttaaacaattataatgtattatttatttctaaatggtttattagatttaaaattttcttggtgtttaattttttcatatattaccttataccctttaacttcctaaaatatattaggtcttcatattttagagtaaaattctgaaaatcctttgagtatctgattttacaatcttttcttccactgattttcccttagcaatggcctgtttaaagtgttgtgatgatgttactgagaaatgggctggctacctgatgcacatagaagccaatactatggcagtggttttctagaaaagaaaaggctttactgtgagtctactggcaaggagacaggtggcaacactcaaatctgtctccctgaactgaggatggtggggt

SEQ ID NO: 43 - DNA, 578 bp, Homo sapiens
cctcttacttgggccccgttcactagtccttcagccaaactgcctcacatgctattcccagtatgaaaatcttgccattccctttatcttttttctcttctctcatttacagccctgtgctagtttcttcattcccttcaagttctggccaaactttatttacctcttgactgaccactccatctaaaatagtactcatcactgtgtatcccctcaacacactttataggtcatggccatcacctgataatgtgttatgtattttttggtttacttgttgtgttagttcattcttgcattgctgtaaagaaattcctgagactgggtaatttataaagaaaagaggtttaattgactcacagttctgcaggctgtatgggaagcatgttgctggcatctgcttggcttctggggaggactcaggaaacttacaatcatggggaaggtgacgggggagcaggcacatctgacatagcaggagcagcaagtgagcaaagggggacgtgccacacacttctaagtaaccagacctcatgagaactcactatcatgagaacagtaccaggggatggtgctag

SEQ ID NO: 44 - DNA, 684 bp, Homo sapiens
agtataacaattcactgctttacatctctatattttgcttatctcaagtatccactttgtctggtatagtgtgctcattccacagtttttggctgtcctgggaacaacaatctagtgcaactccagcaatgtgagttatagtgcaaatgtcaaaccagagcagcatcaccatctagaggtcaaaatgataactgcaaactttctcacctttatgagccttccgtattctgtatacatagcagtttatgtgaatgtacagaaaataatgtttgctattgttttctctccagttgggtttccagaaagagatcatggcataaagcaggaaccacctgtatttacagatggcatagggaagcatacatcgcagagccatatatcagcagcactacagcatgtttcaaccaaagatgagcctcccacatgtcagacaaaccacctacattgggaccacagcagtgacagtgttttttagcacattcctgataatgaaatctatgttgaactcaacatgaatggcttttcctttctcttggcagtcaacagcctacaccattctgcatttgactgtttagtttattctcccctctggaaaggcatgactatggaaacagagtagaggatattttggggatttatgaaactattaatataatttactctcattgctgtgctttctacaaa

SEQ ID NO: 45 - DNA, 693 bp, Homo sapiens
tcagcttatctggtcaatagcttttcgctctgttgcataccttgagcatatgcatcagctacaatgtttataggtagctgtatggtgtttgacacagcacatggcgtacctttaaaacaattatagcactgggatttggatctgaatttatgttgccttgtcaaagtttcctctttgtaacatggtagccttttaaatattaggcagctacctgcaacactgggcattcagactaacccatcaggcttatggcatcttgctcttctcgttccctctctgtgtgttggtacatcatgttaggtttatgcagtagacgtagataggaagcaagccaattggctacagggtattgaaagtcaattgctgagaatgataaaagacaaggatagccttctctgcaaagaagtgctaagaagattctaaacgtatacaaggatctcaagagaaacagtcccagatagcaacactattcagtcttagactatggctgatactatacacttctccagctcctctgctcctcagagcagaaaacagaagattttgaaatgagcaccaccccagctcctgaatacaatggtacctttcatctatttctggtgacttttattttcttttgttgctggatcccctacataattgtaagcatatcgcaggcaagcacaatggtaaacagtgggtggacgcttcctc

SEQ ID NO: 46 - DNA, 677 bp, Homo sapiens
atagaacttgattatattggtatttttatttcaaattttcaatttttggaatggcagaatgttgctattgaaaagtgtcttaaaggtcaccactgtaaccccttcattgtgcttgagacctgctcagctcctaaatttaacaggggacggatctgagaaactgactccaagttgtaacctcttgcttagttttctttctagggagatatccgtctctccaaacctgtcgaaatctaaatttattacctcttacctaatacttggtcccctgtggacttcacttcactgtttgtgctaatagccttttcatcaccatcttgactttggattctagagcatcacctacttccccattttctgtgacccttacattcctcctgtcagtcactatgtctgatttattgttctcccctatcttttgccctttgcaaatcctcaagccctcattctggttcagacctttaaaaggctgagttactggagtatagtgttacccaaagtgagttgttccataaaaaattagtaagttggaaaaaaaaacaaaaaacaaaaaaataccctacccataaagttggtaaatgttcctgtaaaaagggttccttggccaggtacatgttagaatagctggttaagtttctttgcagaaagacttctcctggccttcatttgtgactgtg

SEQ ID NO: 47 - DNA, 729 bp, Homo sapiens
gcaattaagttttgtactgtatggacagtgtgaaaaacattatggaaaaacaacttgaaagaaaatgtgacagaatttctcctaacaatgtcattgcttcaaccagctacaaatttccaacctagtttctttcttttgctgtttcttcttttgtctttgatacaatcatacagcctctcttccttgaagagataataaaagactaacagttaaaagatctggaagactcatattcttttctttttcactggctacggttttgaaaagaggctgttggcttttgattttttcttttgggttctttacatcgcccaattcaaacaggtctgctctcaaagaaaacaaatcaaaattgtcaagacctgtgaagcatgaaaaataaattgctttttccaactccaaaaagcaccagaaaagcattaattttgatcttttataaacctctatcccctatcctctaatctatagatttcacagaatgtttatatattcttctgtataatacaggagatcaaaccttattatgaataaattgaattgaacctgtaatacaactaatatttaaactagtgttattttggagttcaactagacacatataaaacatttcaagtgagatgacacaaattcctggggctgccagtataaaataaacagtccagtaagctgcatctaccatgccgttaagggactctgtccttttagctggtgggagcacaggcttcataa

SEQ ID NO: 48 - DNA, 595 bp, Homo sapiens
tcctgagaagacctgcagcacagggtaaaatatgcaagggagggccatataacttttatctttacttaatttattttaatttactaatttttaagtattaacctattttgtttttattaaatctctgtggttgcacagaattcaaattgcagcaaaaatcattcagggctaaacactggaaaaatctcttaattctaaggtacatgacacaatggactcaaaaacagttgctgagtccctttcactggagaaatttaaagaaagggtatagaaaagttttgaccaattccacccaatcctgcatccccaattccaatctcaaggaccagtttccatctgatctctctccacctacagatggtggtcctgaatctccaaatcaacaaaccaaaaactgaatccatcatcttctcacacctggtttttccttccaactccctcatttctgtgacctgccccataaccttaccaggaatccagcccccaaagcagggtggactcctccctctgcaatggacaccagggattcaggtcctgttgctggctccaaaatgcccacaatgccctgttctcccaaatcagcacattcaacagt

SEQ ID NO: 49 - DNA, 710 bp, Homo sapiens
tttgacttaaccctttgtagcccaggtaataaatccaaactcagcaagtatgggctggaccccagtagctctgtggttgccactttttggcccatattgaaccgacgtccccttggcatctaccagggactcctcagggagagtgtgggaatgatgggggaagactcgtcactcttttgtagagcgtggggcagatgatagcagagaccttccagggcccagggctggggtcttgtcttccttggatgtggtctagcgttgctccagatggtgggtttgtggcaggtggggcagaagcagatgatgcagttgaggcgggtctctggtagagagtgatgtcaaagatgagcactccttttatcccctgactcttctgaggatggctgcctccttggtgagccacttggaggtctcaggccgatcatgcgggatggtggcccagatgaggaaggggatccaaggcggtggccttcccagatgcactgggccccagcccttcttcctagcttcggctgattactgtgggcttcagcaaccagggcctacctgtaggtctccacatttgtaagcaccacagaacccagtgcatctttgtgcactcaaatcgcccttgatccaggggtattttctcattcagaacacactttgaaaggcggccattccccttctggagaaagcctggagaatctacagtgcccttaattacagtg

SEQ ID NO: 50 - DNA, 550 bp, Homo sapiens
agatgcccagacaccttcacttcagcagacaaggggcagagtcctggaaaatctaggcagggaagacttgcgcctctaagagtaaaaggcctcccagagaggacatggatgaaaggaggaccaccttccaatgccactctccaaagcaggaaacatccaaataaaggatgttgattttcaggaccccatcccttcatgagtgcttacacaactggtatatcctctcccgtctcttcctctggtagccaagaccttataccagtttgagtatcctttatccaaaatgcttggggtcagaagtgttttgaatttcagatatttttaaattttggaatatttatatcatacctcttggttgaaccttccagatacaaaaatctggagtccagtgagtatttcctttgagtgtcatgtcagtgctcaaaaagttttagattttggagcgtttcagatttcaggtttttgaaattggaatactcaacctgtactctctgtccttgttctacctctaccagaccctcccccacaggaatgaatttagatctgaaaa

SEQ ID NO: 51 - DNA, 747 bp, Homo sapiens
tcatcctccgctgtctattttgagctgtgagtttatccacaaaggaacagagctgaaatgaaacaatttcaccacagtaacttgttaatcgggcatcctttaagtatgctggatttaacactggaagttcttttgaagactctgaaagttttctttaatcgtcatgagatttttccaaactaagttcatgatatggatttttttcactgtatctagcttaagtcacatttcaattcaaatctaaacctaaactgatggagctggagctagtgacttcaggcaattggcatcttttcgctgaatacaaacatcctatttaaaagaccaaacacatgactccattcaaaaattaaaacagtcatgtgtagtgaaacagcaagaacacggtctgagaaacgtgtccttgcacacacagcgtgaatgcactcacgcaagcctagacggtgcggctgccgcacaccaggccctgtggtacagcctgtcaattccaggccccaagcctgcataccatgttgctgtgcgggacgctgccggcggctgtagcacaatgctaagtatctgtgtatctcaacacagaagaggtagagtaaagtacagtattatgatcgtacgggacgcgtgttgtacacacagtctatcattgatggaagcatcgttatatggcacattactgcactgtaaaaagacaccaaacttcggccggcgcagtggctcatgcctgtaatcccagcactttgggaggctgag

SEQ ID NO: 52 - DNA, 695 bp, Homo sapiens
ttcttccttttcctttcattatcattttctttttgtctcaaaataatgaaaaatgcataagggtctgtagagagaagaaaatgtccttgcccatgaacttcttgcaggtatttatcttgcttctttatcttactaaaaatagaattgaaagtttttcattttttgtttttcaattttagaggatacaatggagattcaggaacgaatagaaaatagttttaagtctttactagaccagttaaaaggtaagttttcctactgtagattcctgtatttgtatctggttgtatggcaatagcttcgaagttctttcccctattcccaagcccaatcacccagagataagtaagtagttttaacactttggagtcaatactcctagatgccacctaaacacatatgtgtgtgaatgaaaatacagataaaaagtaatctttaaacataggaaatggtgtaatccatgcttttttgactttaatttttttgttattttggatacctttccatgtcagttatatataccccatttattttcaagactgcgtaatattctatagtattgtattaacattttttatgttatcgcaattggtgacatattatgtatatgagttatttcttctactgatgctgaaatgaatatcttgggacaaattgttaggggtattatttgagtccttccttgggattaaatt

SEQ ID NO: 53 - DNA, 735 bp, Homo sapiens
cttttgaggattaaaaattcctgcttactgtcgttataacacggggattaataagcaccttactggaatctctcacctaccataattttagtatgctatgtgagggaatgaacagtctcacacatttaataatgactactcatataatgcttttaattggtaatgacctatatgaaacatgatatagaaaacacattacagcttctcaaatgacccctataagttaaccaattgcttaggtttctgacaaatttgaatctggccccatgcacctttgctgggccccacaaaacaaggaggtagattatttatgaaggtcaaccactctggcaatatcaccattaaatatcaagctcatctgccccatagctcctccatcttcaggtccaggactctggattggaatgacctacctccacattcagttctgtaagtcattaggcatcatccaagatggtagatgatgaataaatggacaatgacttaagctttttttactctctcatccattccaatgctttcttccctggtctttgctcattatttccatgttatttaatatatatttggaagaattcatggcagtgataacaataatggctacaattttttattacctatgtatgccaggcattgtgctaagtgcttcaggtataagatcttgtaagggattggttacattttacagatggtaagactgggattcagatgttagttgcctgtttaagtcaataa

SEQ ID NO: 54 - DNA, 427 bp, Homo sapiens
ctcttctccctaggtggtttgctggcaatctttggcattccttagcttgtggaagtatcactccatctctgtcctgatttctacatggtgttcttcctgtgtgcatgtctgtctccaaatttccccattttataaggacacagtcatactggattcgggctcattctaaagacctcatttaatttaattccataaagaccctatctccaaataatgtcacattctgtggtactgggggttatgacttaaacatataaattttagggagacaaatttgaacctctaacagtactgaacatccaggatggaagaacatggtattaggttgagccaaacacagttgcttacgttttggttttcctcaccaggacaagaaacccccagtgcaggaaaattggagacatggaaaacagggcttaagtaaaca

SEQ ID NO: 55 - DNA, 713 bp, Homo sapiens
ttattatactcaacactgctaggaaagaatcagtgatgttgaagatatatatatatatatttgcttgtgtatttgtgtgtgagagacacacatagaaaaaaagagagagagaaatatattggttgacactggcttctttgaaaaaaggcagtttagtaacaatggcctttactagacagacatgttagaaggcagcaggagaaagggaatgtggtatcagatattttctgtaaaaggtttgttattaatattcatgtggcaaattgtagctgatgtcaaagtagttataaagcaaggggaacacaattcttttacagcaatgttgaggtctaagaaacataaaacaaatacctggtaagtaccatgcatatatacatacataaacaatcaataactcacaaaacattcacatatttgcaacactgcttttcagtttatgcagtttattttttgttctttttaagctttttattatagtgaatgtcttatatttcattaaaagttttgatattatatgtgaaacaacagttctgataaagcaatatctagataaaggctattacttacctttctcaaattgatagattttctccttgtaacaagctctgatataaaatatgataatttgttgaaaacttttacacattcaaaactaaattatcatatatttaatgagactttgggtgtgtatgtgtgagtgtgtgtctgtgtgt

SEQ ID NO: 56 - DNA, 607 bp, Homo sapiens
aaaacttggttttttaaagcaaacacagaaacaatgtaatataggtcttattacatatgtaggaaataaaaataatatgtatgacgacaacagtagtctaaaattcaggagacagagaatggaagtacattgttgcaaggttttctaatacacatgtacaaagtggtataatgttacttgaaagataactgtgataagttaaagacgtaatcaatgacactatatcaaccactaaaataatacaacaaaggatatacgaaatatttttaaaagtataattaacccaaaagaaagcatagaggaaaaagggaacaaagaataatagatggaataaacagaaaaaactagccagctggtaaatttaaaaccgatcatatacatattcacattaaatacaaaaagtttaaacacttcaaagtcaagtcagaggtgtcatattggataaaaagaaagactcaactatatgttacctataaggaatgcactttaaatatacaaacatattaaaataaaaagatgaaaagttatatacaatgttaatactcatcaaaataaagctaatgaggctatattcatattaaaaagtaggttttaaagcaaagattac

SEQ ID NO: 57 - DNA, 746 bp, Homo sapiens
tcaagtccatgcttttacggaaagaccccagttcctgcctcttctatatatttatctaccttgtggtgaagagcatgtgtgtgcaacacctttgcctgaaatggtatggtttggcattaatgaattgtgggtccattgaaaagaaatctcctcttgtttctcgtgttatggacagttcaaggtttgccttagaactaacttcaaggaaaagtagcagaatcgtaggaagggacaatcttgccttcagtcccaccctctgttccgggcaggtctgggtggctatcttctttcgggggcttttccttgcagaagaacttcttcagcatgtcctggatttccttcttaatggtcttgtgcatgtagccatagacataggggtggatgcagcactgcaggaagaaaagccagatgattatggtgatcacccactggggtacctgggtttcgacatccacccacacggccaggactgctaaaaagcagtagggccccagggatagcacataggagaaaatgatgatgaagatcactttagcagctttgcactggtagcacctgggcagaggagggttgctgttgctgttacgacgactgggtgggaggctctccgggatgttcactgcctcgacgtcatcctcactgaaattgatgtcgtcttcaccaaactccatgtcatcttcacccaagtcaatgctgcactggttgacctctgtgcgacccttgtctgccttcatgctgttctcctc

SEQ ID NO: 58 - DNA, 638 bp, Homo sapiens
agtggaaagaccacacctaggaaccgactctagctcttaccaccctgtaagcctgaggctcagttgctgtccctggagaacagaaaacataatcatggctattctgagggtcaggggcaagtgctttgcaagtgggattgtggtgggcagtgggagggattctggggttcactgtcatgctagttgtgtaactgggcaatgcaaccgtgtaagtgtcaggaaaccctcaataagactgagccagaggccaataagaagccagcatttacatgatgttcttttcctttttgtaactaggaaatttcgatttgcacactgatttggcccaccattcctggagagatctcgtgggatgtctcttttgttactttgaacttcttggtgccaggactggtcattgtgatcagttactccaaaattttacaggtatgttttctgcaagtgctgccactgaacttcacccaggcttggggttatttctgctagaatcttagaatttggggtcggagaacacctaagagttcacgccagctcaatcttgattcactgcccaggtctacaacactgaggaaggagaggatttttttagaagttatatctttgtgattatgttttttgctcatcactaaagtaatact

SEQ ID NO: 59 - protein, 216 aa, Homo sapiens [sequence omitted]

SEQ ID NO: 60 - protein, 204 aa, Homo sapiens [sequence omitted]

SEQ ID NO: 61 - protein, 96 aa, Homo sapiens [sequence omitted]

SEQ ID NO: 62 - protein, 213 aa, Homo sapiens
HQILLCCLRLQHISMASSLGMVTVAELGGFVLPIIIITYFTWKTRKSLWEFQVPPRNTKERKKALRMVLMCEVVFIVCFTPYHLNFPFFMMVKEHVFLNCSFIKIILCFHIISLCLANLNCCLDPVVYYFMTSKFHDQFSDHGSLVLQSCMRCNNSTLEIHQRKGGSSNYLSMFERFQDNIIKLTRKIDMLYCIYVTLKIFLFFFSFFLLYFK

SEQ ID NO: 63 - protein, 197 aa, Homo sapiens [sequence omitted]

SEQ ID NO: 64 - protein, 132 aa, Homo sapiens [sequence omitted]

SEQ ID NO: 65 - protein, 202 aa, Homo sapiens
ITIFQPLLLQGLLCTLSLNSPSICSHNPHDPQFYNTTVRSPKLPFIHFHITIFQPLLLQGLLCTLSLNSHDSSCTLGSSVSPLLLISRVPFCFCWLPYKACNIISHFRKELDHLLMNPAFMTHCLTCLWLCMSPSFRFFLWKERLPKSPAHQHYKCMQTSFSCLPTLKMSKQFSKGEKISSPPHTNYLHNSVTFYKPCHCIS

SEQ ID NO: 66 - protein, 221 aa, Homo sapiens
TVLIMIVFVICCWGPYCFLVLLAAARQAQTMQAPSLLSVVAVWLTWANGAINPVIYAIRNPNISMLLGRNREEGYRTRNVDAFLPSQGPGLQARSRSRLRNRYANRLGACNRMSSSNPASGVAGDVAMWARKNPVVLFCREGPPEPVTAVTKQPKSEAGDTSLDGWNGQLMKANFHSHYLMMEDSGGELWISSQTFKARDGGGLPLSPNNIKDNVPSFKKC

SEQ ID NO: 67 - protein, 595 aa, Homo sapiens
LEPTSKAPPGPQRPPPLRPSPAPRGGRPPAPSHHSDLAAAAPGAGGDPRPPLGPMEEPQPPRPPASMALLGSQHSGAPSAAGPPGGTSSAATAAVLSFSTVATAALGNLSDASGGGTAAAPGGGGLGGSGAAREAGAAVRRPLATEAAPLLSHGAAVAAQALVLLLIFLLSSLGNCAVMGVIVKHRQLRTVTNAFILSLSLSDLLTALLCLPAAFLDLFTPPGGSAPAAAAGPWRGFCAASRFFSSCGIVSTLSVALISLDRYCAIVRPPREKIGRRRALQLLAGAWLTALGFSLPWELLGAPRELAAAQSFHGCLYRTSPDPAQLGAAFSVGLVVACYLLPFLLMCFCHYHICKTVRLSDVRVRPVNTYARVLRFFSEVRTATTVLIMIVFVICCWGPYCFLVLLAAARQAQTMQAPSLLSVVAVWLTWANGAINPVIYAIRNPNISMLLGRNREEGYRTRNVDAFLPSQGPGLQARSRSRLRNRYANRLGACNRMSSSNPASGVAGDVAMWARKNPVVLFCREGPPEPVTAVTKQPKSEAGDTSLDGWNGQLMKANFHSHYLMMEDSGGELWISSQTFKARDGGGLPLSPNNI

SEQ ID NO: 68 - protein, 201 aa, Homo sapiens [sequence omitted]

SEQ ID NO: 69 - protein, 217 aa, Homo sapiens [sequence omitted]

SEQ ID NO: 70 - protein, 156 aa, Homo sapiens [sequence omitted]

SEQ ID NO: 71 - protein, 221 aa, Homo sapiens [sequence omitted]

SEQ ID NO: 72 - protein, 237 aa, Homo sapiens [sequence omitted]

SEQ ID NO: 73 - protein, 224 aa, Homo sapiens [sequence omitted]

SEQ ID NO: 74 - protein, 216 aa, Homo sapiens [sequence omitted]

SEQ ID NO: 75 - protein, 204 aa, Homo sapiens [sequence omitted]

SEQ ID NO: 76 - protein, 228 aa, Homo sapiens [sequence omitted]

SEQ ID NO: 77 - protein, 220 aa, Homo sapiens [sequence omitted]

SEQ ID NO: 78 - protein, 157 aa, Homo sapiens
KIHSAAGRHRAFSTCSSHLTVVLLQYGCCAFMYLCPSSSYNPKQDQFISLVYTLGTPLLNPLIYALRNSEMKGAVGRVLTRNCLSQNSERRGDSLSGKYLVPAHQICMKLRFLSFGVKTHLKDGINYMDTVYVCQRFLNISTILCNFSSWKELHEHK

SEQ ID NO: 79 - protein, 227 aa, Homo sapiens [sequence omitted]

SEQ ID NO: 80 - protein, 164 aa, Homo sapiens [sequence omitted]

SEQ ID NO: 81 - protein, 221 aa, Homo sapiens [sequence omitted]

SEQ ID NO: 82 - protein, 216 aa, Homo sapiens [sequence omitted]

SEQ ID NO: 83 - protein, 118 aa, Homo sapiens [sequence omitted]

SEQ ID NO: 84 - protein, 147 aa, Homo sapiens [sequence omitted]

SEQ ID NO: 85 - protein, 202 aa, Homo sapiens [sequence omitted]

SEQ ID NO: 86 - protein, 189 aa, Homo sapiens [sequence omitted]

SEQ ID NO: 87 - protein, 191 aa, Homo sapiens [sequence omitted]

SEQ ID NO: 88 - protein, 194 aa, Homo sapiens [sequence omitted]

SEQ ID NO: 89 - protein, 218 aa, Homo sapiens
QSQPSLPGSMGDELAPCPVGTTAWPALIQLISKTPCMPQAASNTSLGLGDLRVPSSMLYWLFLPSSLLAAATLAVSPLLLVTILRNQRLRQEPHYLLPANILLSDLAYILLHMLISSSSLGGWELGRMACGILTDAVFAACTSTILSFTAIVLHTYLAVIHPLRYLSFMSHGAAWKAVALIWLVACCFPTFLIWLSKWQDAQLEEQGASYILPPSMGT

SEQ ID NO: 90 - protein, 223 aa, Homo sapiens [sequence omitted]

SEQ ID NO: 91 - protein, 193 aa, Homo sapiens [sequence omitted]

SEQ ID NO: 92 - protein, 191 aa, Homo sapiens [sequence omitted]

SEQ ID NO: 93 - protein, 133 aa, Homo sapiens [sequence omitted]

SEQ ID NO: 94 - protein, 202 aa, Homo sapiens [sequence omitted]

SEQ ID NO: 95 - protein, 178 aa, Homo sapiens [sequence omitted]

SEQ ID NO: 96 - protein, 191 aa, Homo sapiens [sequence omitted]

SEQ ID NO: 97 - protein, 91 aa, Homo sapiens
GRGGQQGGLQNHDVFLTGLTSASICLTLQPMSLFLVVILMGALRSQRRGLRRHCLYLWSYIRHLYFVMNSKSSSKMQLWGNSHRNFSQFWL

SEQ ID NO: 98 - protein, 201 aa, Homo sapiens [sequence omitted]

SEQ ID NO: 99 - protein, 218 aa, Homo sapiens [sequence omitted]

SEQ ID NO: 100 - protein, 175 aa, Homo sapiens [sequence omitted]

SEQ ID NO: 101 - protein, 184 aa, Homo sapiens [sequence omitted]

SEQ ID NO: 102 - protein, 212 aa, Homo sapiens [sequence omitted]

SEQ ID NO: 103 - protein, 219 aa, Homo sapiens [sequence omitted]

SEQ ID NO: 104 - protein, 208 aa, Homo sapiens [sequence omitted]

SEQ ID NO: 105 - protein, 231 aa, Homo sapiens [sequence omitted]

SEQ ID NO: 106 - protein, 188 aa, Homo sapiens [sequence omitted]

SEQ ID NO: 107 - protein, 230 aa, Homo sapiens [sequence omitted]

SEQ ID NO: 108 - protein, 178 aa, Homo sapiens [sequence omitted]

SEQ ID NO: 109 - protein, 237 aa, Homo sapiens [sequence omitted]

SEQ ID NO: 110 - protein, 221 aa, Homo sapiens [sequence omitted]

SEQ ID NO: 111 - protein, 235 aa, Homo sapiens [sequence omitted]

SEQ ID NO: 112 - protein, 134 aa, Homo sapiens [sequence omitted]

SEQ ID NO: 113 - protein, 229 aa, Homo sapiens [sequence omitted]

SEQ ID NO: 114 - protein, 189 aa, Homo sapiens [sequence omitted]

SEQ ID NO: 115 - protein, 242 aa, Homo sapiens [sequence omitted]

SEQ ID NO: 116 - protein, 206 aa, Homo sapiens [sequence omitted]

SEQ ID NO: 117 - protein, 9 aa, Artificial Sequence
  misc_feature: Peptide substrate
APRTPGGRR

(19) United States
(12) Patent Application Publication
(10) Pub. No.: US 2002/0015998 A1
(43) Pub. Date: Feb. 7, 2002

(54) Novel G protein-coupled receptors

(76) Inventor: Gabriel Vogeli, Seattle, WA (US)

Correspondence Address: WOODCOCK WASHBURN KURTZ MACKIEWICZ & NORRIS LLP, ATTENTION: SUZANNE E. MILLER ESQ., ONE LIBERTY PLACE, 46TH FLOOR, PHILADELPHIA, PA, 19103, US

(21) Appl. No.: 09/828,644

(22) Filed: Apr. 6, 2001

Related U.S. Application Data
(60) Provisional application No. 60/195,150, filed on Apr. 6, 2000.
(60) Provisional application No. 60/195,099, filed on Apr. 6, 2000.
(60) Provisional application No. 60/195,151, filed on Apr. 6, 2000.
(60) Provisional application No. 60/195,148, filed on Apr. 6, 2000.
(60) Provisional application No. 60/195,093, filed on Apr. 6, 2000.
(60) Provisional application No. 60/195,098, filed on Apr. 6, 2000.
(60) Provisional application No. 60/230,149, filed on Sep. 5, 2000.

(51) Int. Cl. C12N 15/861; C07H 21/02; C07H 21/04; C12P 21/02; C12N 5/06

(52) U.S. Cl. 435/320.1; 536/23.1; 435/325; 435/69.1; 435/456

(57) ABSTRACT

The present invention provides a gene encoding a G protein-coupled receptor termed nGPCR-x; constructs and recombinant host cells incorporating the genes; the nGPCR-x polypeptides encoded by the gene; antibodies to the nGPCR-x polypeptides; and methods of making and using all of the foregoing.

### CROSS-REFERENCE TO RELATED APPLICATIONS

The present application claims priority of application Ser. No. 60/195,150, filed Apr. 6, 2000; application Ser. No. 60/195,099 filed Apr. 6, 2000; application Ser. No. 60/195,151 filed Apr. 6, 2000; application Ser. No. 60/195,148 filed Apr. 6, 2000; application Ser. No. 60/195,093 filed Apr. 6, 2000; application Ser. No. 60/195,098 filed Apr. 6, 2000; and application Ser. No. 60/230,149 filed Sep. 5, 2000; each of which is hereby incorporated by reference in its entirety.

### FIELD OF THE INVENTION

The present invention relates generally to the fields of genetics and cellular and molecular biology. More particularly, the invention relates to novel G protein coupled receptors, to polynucleotides that encode such novel receptors, to reagents such as antibodies, probes, primers and kits comprising such antibodies, probes, primers related to the same, and to methods which use the novel G protein coupled receptors, polynucleotides or reagents.

### BACKGROUND OF THE INVENTION

The G protein-coupled receptors (GPCRs) form a vast superfamily of cell surface receptors which are characterized by an amino-terminal extracellular domain, a carboxyl-terminal intracellular domain, and a serpentine structure that passes through the cell membrane seven times. Hence, such receptors are sometimes also referred to as seven transmembrane (7TM) receptors. These seven transmembrane domains define three extracellular loops and three intracellular loops, in addition to the amino- and carboxy- terminal domains. The extracellular portions of the receptor have a role in recognizing and binding one or more extracellular binding partners (e.g., ligands), whereas the intracellular portions have a role in recognizing and communicating with downstream molecules in the signal transduction cascade.

The G protein-coupled receptors bind a variety of ligands including calcium ions, hormones, chemokines, neuropeptides, neurotransmitters, nucleotides, lipids, odorants, and even photons, and are important in the normal (and sometimes the aberrant) function of many cell types. [See generally Strosberg, Eur. J. Biochem. 196:1-10 (1991) and Bohm et al., Biochem J. 322:1-18 (1997).] When a specific ligand binds to its corresponding receptor, the ligand typically stimulates the receptor to activate a specific heterotrimeric guanine-nucleotide-binding regulatory protein (G-protein) that is coupled to the intracellular portion of the receptor. The G protein in turn transmits a signal to an effector molecule within the cell, by either stimulating or inhibiting the activity of that effector molecule. These effector molecules include adenylate cyclase, phospholipases and ion channels. Adenylate cyclase and phospholipases are enzymes that are involved in the production of the second messenger molecules cAMP, inositol triphosphate and diacyglycerol. It is through this sequence of events that an extracellular ligand stimuli exerts intracellular changes through a G protein-coupled receptor. Each such receptor has its own characteristic primary structure, expression pattern, ligand-binding profile, and intracellular effector system.

Because of the vital role of G protein-coupled receptors in the communication between cells and their environment, such receptors are attractive targets for therapeutic intervention, for example by activating or antagonizing such receptors. For receptors having a known ligand, the identification of agonists or antagonists may be sought specifically to enhance or inhibit the action of the ligand. Some G protein-coupled receptors have roles in disease pathogenesis (e.g., certain chemokine receptors that act as HIV co-receptors may have a role in AIDS pathogenesis), and are attractive targets for therapeutic intervention even in the absence of knowledge of the natural ligand of the receptor. Other receptors are attractive targets for therapeutic intervention by virtue of their expression pattern in tissues or cell types that are themselves attractive targets for therapeutic intervention. Examples of this latter category of receptors include receptors expressed in immune cells, which can be targeted to either inhibit autoimmune responses or to enhance immune responses to fight pathogens or cancer; and receptors expressed in the brain or other neural organs and tissues, which are likely targets in the treatment of mental disorder, depression, bipolar disease, or other neurological disorders. This latter category of receptor is also useful as a marker for identifying and/or purifying (e.g., via fluorescence-activated cell sorting) cellular subtypes that express the receptor. Unfortunately, only a limited number of G protein receptors from the central nervous system (CNS) are known. Thus, a need exists for G protein-coupled receptors that have been identified and show promise as targets for therapeutic intervention in a variety of animals, including humans.

### SUMMARY OF THE INVENTION

The present invention relates to an isolated nucleic acid molecule that comprises a nucleotide sequence that encodes a polypeptide comprising an amino acid sequence homologous to sequences selected from the group consisting of SEQ ID NO:59 to SEQ ID NO: 116, or a fragment thereof. The nucleic acid molecule encodes at least a portion of nGPCR-x. In some embodiments, the nucleic acid molecule comprises a sequence that encodes a polypeptide comprising a sequence selected from the group consisting of SEQ ID NO:59 to SEQ ID NO: 116, or a fragment thereof. In some embodiments, the nucleic acid molecule comprises a sequence homologous to a sequence selected from the group consisting of SEQ ID NO: 1 to SEQ ID NO:58, or a fragment thereof. In some embodiments, the nucleic acid molecule comprises a sequence selected from the group consisting of SEQ ID NO: 1 to SEQ ID NO:58, and fragments thereof.

According to some embodiments, the present invention provides vectors which comprise the nucleic acid molecule of the invention. In some embodiments, the vector is an expression vector.

According to some embodiments, the present invention provides host cells which comprise the vectors of the invention. In some embodiments, the host cells comprise expression vectors.

The present invention provides an isolated nucleic acid molecule comprising a nucleotide sequence complementary to at least a portion of a sequence selected from the group consisting of SEQ ID NO: 1 to SEQ ID NO:58, said portion comprising at least 10 nucleotides.

The present invention provides a method of producing a polypeptide comprising a sequence selected from the group consisting of SEQ ID NO:59 to SEQ ID NO:1 16, or a homolog or fragment thereof. The method comprising the steps of introducing a recombinant expression vector that includes a nucleotide sequence that encodes the polypeptide into a compatible host cell, growing the host cell under conditions for expression of the polypeptide and recovering the polypeptide.

The present invention provides an isolated antibody which binds to an epitope on a polypeptide comprising a sequence selected from the group consisting of SEQ ID NO:59 to SEQ ID NO: 116, or a homolog or fragment thereof.

The present invention provides an method of inducing an immune response in a mammal against a polypeptide comprising a sequence selected from the group consisting of SEQ ID NO:59 to SEQ ID NO:116, or a homolog or fragment thereof. The method comprises administering to a mammal an amount of the polypeptide sufficient to induce said immune response.

The present invention provides a method for identifying a compound which binds nGPCR-x. The method comprises the steps of contacting nGPCR-x with a compound and determining whether the compound binds nGPCR-x.

The present invention provides a method for identifying a compound which binds a nucleic acid molecule encoding nGPCR-x. The method comprises the steps of contacting said nucleic acid molecule encoding nGPCR-x with a compound and determining whether said compound binds said nucleic acid molecule.

The present invention provides a method for identifying a compound which modulates the activity of nGPCR-x. The method comprises the steps of contacting nGPCR-x with a compound and determining whether nGPCR-x activity has been modulated.

The present invention provides a method of identifying an animal homolog of nGPCR-x. The method comprises the steps screening a nucleic acid database of the animal with a sequence selected from the group consisting of SEQ ID NO: 1 to SEQ ID NO:58, or a portion thereof and determining whether a portion of said library or database is homologous to said sequence selected from the group consisting of SEQ ID NO: 1 to SEQ ID NO:58, or portion thereof.

The present invention provides a method of identifying an animal homolog of nGPCR-x. The methods comprises the steps screening a nucleic acid library of the animal with a nucleic acid molecule having a sequence selected from the group consisting of SEQ ID NO: 1 to SEQ ID NO:58, or a portion thereof; and determining whether a portion of said library or database is homologous to said sequence selected from the group consisting of SEQ ID NO: 1 to SEQ ID NO:58, or a portion thereof.

Another aspect of the present invention relates to methods of screening a human subject to diagnose a disorder affecting the brain or genetic predisposition therefor. The methods comprise the steps of assaying nucleic acid of a human subject to determine a presence or an absence of a mutation altering an amino acid sequence, expression, or biological activity of at least one nGPCR-x that is expressed in the brain. The nGPCR-x comprise an amino acid sequence selected from the group consisting of SEQ ID NO:59 to SEQ ID NO: 116, and allelic variants thereof. A diagnosis of the disorder or predisposition is made from the presence or absence of the mutation. The presence of a mutation altering the amino acid sequence, expression, or biological activity of the nGPCR-x in the nucleic acid correlates with an increased risk of developing the disorder.

The present invention further relates to methods of screening for a nGPCR-x hereditary mental disorder genotype in a human patient. The methods comprise the steps of providing a biological sample comprising nucleic acid from the patient, in which the nucleic acid includes sequences corresponding to alleles of nGPCR-x. The presence of one or more mutations in the nGPCR-x allele is indicative of a hereditary mental disorder genotype.

The present invention provides kits for screening a human subject to diagnose mental disorder or a genetic predisposition therefor. The kits include an oligonucleotide useful as a probe for identifying polymorphisms in a human nGPCR-x gene. The oligonucleotide comprises 6-50 nucleotides in a sequence that is identical or complementary to a sequence of a wild type human nGPCR-x gene sequence or nGPCR-x coding sequence, except for one sequence difference selected from the group consisting of a nucleotide addition, a nucleotide deletion, or nucleotide substitution. The kit also includes a media packaged with the oligonucleotide. The media contains information for identifying polymorphisms that correlate with mental disorder or a genetic predisposition therefor, the polymorphisms being identifiable using the oligonucleotide as a probe.

The present invention further relates to methods of identifying nGPCR-x allelic variants that correlates with mental disorders. The methods comprise the steps of providing biological samples that comprise nucleic acid from a human patient diagnosed with a mental disorder, or from the patient's genetic progenitors or progeny, and detecting in the nucleic acid the presence of one or more mutations in an nGPCR-x that is expressed in the brain. The nGPCR-x comprises an amino acid sequence selected from the group consisting of SEQ ID NO:59 to SEQ ID NO: 116, and allelic variants thereof. The nucleic acid includes sequences corresponding to the gene or genes encoding nGPCR-x. The one or more mutations detected indicate an allelic variant that correlates with a mental disorder.

The present invention further relates to purified polynucleotides comprising nucleotide sequences encoding alleles of nGPCR-x from a human with mental disorder. The polynucleotide hybridizes to the complement of a sequence selected from the group consisting of SEQ ID NO:1 to SEQ ID NO:58 under the following hybridization conditions: (a) hybridization for 16 hours at 42° C in a hybridization solution comprising 50% formamide, 1% SDS, 1 M NaCl, 10% dextran sulfate and (b) washing 2 times for 30 minutes at 60° C in a wash solution comprising 0.1g×SSC and 1% SDS. The polynucleotide that encodes nGPCR-x amino acid sequence of the human differs from a sequence selected from the group consisting of SEQ ID NO:59 to SEQ ID NO:116 by at least one residue.

The present invention also provides methods for identifying a modulator of biological activity of nGPCR-x comprising the steps of contacting a cell that expresses nGPCR-x in the presence and in the absence of a putative modulator compound and measuring nGPCR-x biological activity in the cell. The decreased or increased nGPCR-x biological activity in the presence versus absence of the putative modulator is indicative of a modulator of biological activity.

The present invention further provides methods to identify compounds useful for the treatment of mental disorders. The methods comprise the steps of contacting a composition comprising nGPCR-x with a compound suspected of binding nGPCR-x. The binding between nGPCR-x and the compound suspected of binding nGPCR-x is detected. Compounds identified as binding nGPCR-x are candidate compounds useful for the treatment of mental disorder. Compounds identified as binding nGPCR-x may be further tested in other assays including, but not limited to, in vivo models, in order to confirm or quantitate their activity.

The present invention further provides methods for identifying a compound useful as a modulator of binding between nGPCR-x and a binding partner of nGPCR-x. The methods comprise the steps of contacting the binding partner and a composition comprising nGPCR-x in the presence and in the absence of a putative modulator compound and detecting binding between the binding partner and nGPCR-x. Decreased or increased binding between the binding partner and nGPCR-x in the presence of the putative modulator, as compared to binding in the absence of the putative modulator is indicative a modulator compound useful for the treatment of a related disease or disorder. Compounds identified as modulating binding between nGPCR-x and a nGPCR-x binding partner may be further tested in other assays including, but not limited to, in vivo models, in order to confirm or quantitate their activity as modulators.

Another aspect of the present invention relates to methods of purifying a G protein from a sample containing a G protein. The methods comprise the steps of contacting the sample with an nGPCR-x for a time sufficient to allow the G protein to form a complex with the nGPCR-x; isolating the complex from remaining components of the sample; maintaining the complex under conditions which result in dissociation of the G protein from the nGPCR-x; and isolating said G protein from the nGPCR-x.

### DETAILED DESCRIPTION OF PREFERRED EMBODIMENTS

Definitions

Various definitions are made throughout this document. Most words have the meaning that would be attributed to those words by one skilled in the art. Words specifically defined either below or elsewhere in this document have the meaning provided in the context of the present invention as a whole and as are typically understood by those skilled in the art.

“Synthesized” as used herein and understood in the art, refers to polynucleotides produced by purely chemical, as opposed to enzymatic, methods. “Wholly” synthesized DNA sequences are therefore produced entirely by chemical means, and “partially” synthesized DNAs embrace those wherein only portions of the resulting DNA were produced by chemical means.

By the term “region” is meant a physically contiguous portion of the primary structure of a biomolecule. In the case of proteins, a region is defined by a contiguous portion of the amino acid sequence of that protein.

The term “domain” is herein defined as referring to a structural part of a biomolecule that contributes to a known or suspected function of the biomolecule. Domains may be co-extensive with regions or portions thereof; domains may also incorporate a portion of a biomolecule that is distinct from a particular region, in addition to all or part of that region. Examples of GPCR protein domains include, but are not limited to, the extracellular (i.e., N-terminal), transmembrane and cytoplasmic (i.e., C-terminal) domains, which are co-extensive with like-named regions of GPCRs; each of the seven transmembrane segments of a GPCR; and each of the loop segments (both extracellular and intracellular loops) connecting adjacent transmembrane segments.

As used herein, the term “activity” refers to a variety of measurable indicia suggesting or revealing binding, either direct or indirect; affecting a response, i.e. having a measurable affect in response to some exposure or stimulus, including, for example, the affinity of a compound for directly binding a polypeptide or polynucleotide of the invention, or, for example, measurement of amounts of upstream or downstream proteins or other similar functions after some stimulus or event.

Unless indicated otherwise, as used herein, the abbreviation in lower case (gpcr) refers to a gene, cDNA, RNA or nucleic acid sequence, while the upper case version (GPCR) refers to a protein, polypeptide, peptide, oligopeptide, or amino acid sequence. The term “nGPCR-x” refers to any of the nGPCRs taught herein, while specific reference to a nGPCR (for example nGPCR-2073) refers only to that specific nGPCR.

As used herein, the term “antibody” is meant to refer to complete, intact antibodies, and Fab, Fab′, F(ab)2, and other fragments thereof. Complete, intact antibodies include monoclonal antibodies such as murine monoclonal antibodies, chimeric antibodies and humanized antibodies.

As used herein, the term “binding” means the physical or chemical interaction between two proteins or compounds or associated proteins or compounds or combinations thereof. Binding includes ionic, non-ionic, Hydrogen bonds, Van der Waals, hydrophobic interactions, etc. The physical interaction, the binding, can be either direct or indirect, indirect being through or due to the effects of another protein or compound. Direct binding refers to interactions that do not take place through or due to the effect of another protein or compound but instead are without other substantial chemical intermediates. Binding may be detected in many different manners. As a non-limiting example, the physical binding interaction between a nGPCR-x of the invention and a compound can be detected using a labeled compound. Alternatively, functional evidence of binding can be detected using, for example, a cell transfected with and expressing a nGPCR-x of the invention. Binding of the transfected cell to a ligand of the nGPCR-x that was transfected into the cell provides functional evidence of binding. Other methods of detecting binding are well known to those of skill in the art.

As used herein, the term “compound” means any identifiable chemical or molecule, including, but not limited to, small molecule, peptide, protein, sugar, nucleotide, or nucleic acid, and such compound can be natural or synthetic.

As used herein, the term “complementary” refers to Watson-Crick basepairing between nucleotide units of a nucleic acid molecule.

As used herein, the term “contacting” means bringing together, either directly or indirectly, a compound into physical proximity to a polypeptide or polynucleotide of the invention. The polypeptide or polynucleotide can be in any number of buffers, salts, solutions etc. Contacting includes, for example, placing the compound into a beaker, microtiter plate, cell culture flask, or a microarray, such as a gene chip, or the like, which contains the nucleic acid molecule, or polypeptide encoding the nGPCR or fragment thereof.

As used herein, the phrase “homologous nucleotide sequence,” or “homologous amino acid sequence,” or variations thereof, refers to sequences characterized by a homology, at the nucleotide level or amino acid level, of at least the specified percentage. Homologous nucleotide sequences include those sequences coding for isoforms of proteins. Such isoforms can be expressed in different tissues of the same organism as a result of, for example, alternative splicing of RNA. Alternatively, isoforms can be encoded by different genes. Homologous nucleotide sequences include nucleotide sequences encoding for a protein of a species other than humans, including, but not limited to, mammals. Homologous nucleotide sequences also include, but are not limited to, naturally occurring allelic variations and mutations of the nucleotide sequences set forth herein. A homologous nucleotide sequence does not, however, include the nucleotide sequence encoding other known GPCRs. Homologous amino acid sequences include those amino acid sequences which contain conservative amino acid substitutions and which polypeptides have the same binding and/or activity. A homologous amino acid sequence does not, however, include the amino acid sequence encoding other known GPCRs. Percent homology can be determined by, for example, the Gap program (Wisconsin Sequence Analysis Package, Version 8 for Unix, Genetics Computer Group, University Research Park, Madison Wis.), using the default settings, which uses the algorithm of Smith and Waterman (Adv. Appl. Math., 1981, 2, 482-489, which is incorporated herein by reference in its entirety).

As used herein, the term “isolated” nucleic acid molecule refers to a nucleic acid molecule (DNA or RNA) that has been removed from its native environment. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules.

As used herein, the terms “modulates” or “modifies” means an increase or decrease in the amount, quality, or effect of a particular activity or protein.

As used herein, the term “oligonucleotide” refers to a series of linked nucleotide residues which has a sufficient number of bases to be used in a polymerase chain reaction (PCR). This short sequence is based on (or designed from) a genomic or cDNA sequence and is used to amplify, confirm, or reveal the presence of an identical, similar or complementary DNA or RNA in a particular cell or tissue. Oligonucleotides comprise portions of a DNA sequence having at least about 10 nucleotides and as many as about 50 nucleotides, preferably about 15 to 30 nucleotides. They are chemically synthesized and may be used as probes.

As used herein, the term “probe” refers to nucleic acid sequences of variable length, preferably between at least about 10 and as many as about 6,000 nucleotides, depending on use. They are used in the detection of identical, similar, or complementary nucleic acid sequences. Longer length probes are usually obtained from a natural or recombinant source, are highly specific and much slower to hybridize than oligomers. They may be single- or double-stranded and carefully designed to have specificity in PCR, hybridization membrane-based, or ELISA-like technologies.

The term “preventing” refers to decreasing the probability that an organism contracts or develops an abnormal condition.

The term “treating” refers to having a therapeutic effect and at least partially alleviating or abrogating an abnormal condition in the organism.

The term “therapeutic effect” refers to the inhibition or activation factors causing or contributing to the abnormal condition. A therapeutic effect relieves to some extent one or more of the symptoms of the abnormal condition. In reference to the treatment of abnormal conditions, a therapeutic effect can refer to one or more of the following: (a) an increase in the proliferation, growth, and/or differentiation of cells; (b) inhibition (i.e., slowing or stopping) of cell death; (c) inhibition of degeneration; (d) relieving to some extent one or more of the symptoms associated with the abnormal condition; and (e) enhancing the function of the affected population of cells. Compounds demonstrating efficacy against abnormal conditions can be identified as described herein.

The term “abnormal condition” refers to a function in the cells or tissues of an organism that deviates from their normal functions in that organism. An abnormal condition can relate to cell proliferation, cell differentiation, cell signaling, or cell survival. An abnormal condition may also include obesity, diabetic complications such as retinal degeneration, and irregularities in glucose uptake and metabolism, and fatty acid uptake and metabolism.

Abnormal cell proliferative conditions include cancers such as fibrotic and mesangial disorders, abnormal angiogenesis and vasculogenesis, wound healing, psoriasis, diabetes mellitus, and inflammation.

Abnormal differentiation conditions include, but are not limited to, neurodegenerative disorders, slow wound healing rates, and slow tissue grafting healing rates. Abnormal cell signaling conditions include, but are not limited to, psychiatric disorders involving excess neurotransmitter activity.

Abnormal cell survival conditions may also relate to conditions in which programmed cell death (apoptosis) pathways are activated or abrogated. A number of protein kinases are associated with the apoptosis pathways. Aberrations in the function of any one of the protein kinases could lead to cell immortality or premature cell death.

The term “administering” relates to a method of incorporating a compound into cells or tissues of an organism. The abnormal condition can be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. For cells outside of the organism, multiple techniques exist in the art to administer the compounds, including (but not limited to) cell microinjection techniques, transformation techniques and carrier techniques.

The abnormal condition can also be prevented or treated by administering a compound to a group of cells having an aberration in a signal transduction pathway to an organism. The effect of administering a compound on organism function can then be monitored. The organism is preferably a mouse, rat, rabbit, guinea pig or goat, more preferably a monkey or ape, and most preferably a human.

By “amplification” it is meant increased numbers of DNA or RNA in a cell compared with normal cells. “Amplification” as it refers to RNA can be the detectable presence of RNA in cells, since in some normal cells there is no basal expression of RNA. In other normal cells, a basal level of expression exists, therefore in these cases amplification is the detection of at least 1 to 2-fold, and preferably more, compared to the basal level.

As used herein, the phrase “stringent hybridization conditions” or “stringent conditions” refers to conditions under which a probe, primer, or oligonucleotide will hybridize to its target sequence, but to no other sequences. Stringent conditions are sequence-dependent and will be different in different circumstances. Longer sequences hybridize specifically at higher temperatures. Generally, stringent conditions are selected to be about 5° C lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present in excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C for short probes, primers or oligonucleotides (e.g. 10 to 50 nucleotides) and at least about 60° C for longer probes, primers or oligonucleotides. Stringent conditions may also be achieved with the addition of destablizing agents, such as formamide.

The amino acid sequences are presented in the amino to carboxy direction, from left to right. The amino and carboxy groups are not presented in the sequence. The nucleotide sequences are presented by single strand only, in the 5′ to 3′ direction, from left to right. Nucleotides and amino acids are represented in the manner recommended by the IUPAC-ruB Biochemical Nomenclature Commission or (for amino acids) by three letters code.

Polynucleotides

The present invention provides purified and isolated polynucleotides (e.g., DNA sequences and RNA transcripts, both sense and complementary antisense strands, both single- and double-stranded, including splice variants thereof) that encode unknown G protein-coupled receptors heretofore termed novel GPCRs, or nGPCRs. These genes are described herein and designated herein collectively as nGPCR-x (where x is 86-93, 2588, 2589, 2591, 2592, 2593, 2594, 2595, 2596, 2598, 2600, 2601, 2602, 2603, 2604, 2606, 2607, 2608, 2609, 2610, 2611, 2613, 2614, 2615, 2616, 2617, 2618, 2619, 2621, 2624, 2625, 2626, 2627, 2628, 2629, 2630, 2631, 2632, 2633, 2634, 2635, 2636, 2637, 2639, 2640, 2641, 2642, 2643, 2644, and 2645). Table 1 below identifies the novel gene sequence nGPCR-x designation, the SEQ ID NO: of the gene sequence, the SEQ ID NO: of the polypeptide encoded thereby, and the U.S. Provisional Application in which the gene sequence has been disclosed.

TABLE 1

| | Nucleotide | Amino acid | | | Nucleotide | Amino acid | |
|---|---|---|---|---|---|---|---|
| | Sequence | Sequence | | | Sequence | Sequence | |
| | (SEQ ID | (SEQ ID | Griginally | | (SEQ ID | (SEQ ID | Griginally |
| nGPCR | NO:) | NO:) | filed in: | nGPCR | NO:) | NO:) | filed in: |
| 86 | 1 | 59 | A | 2613 | 30 | 88 | D |
| 87 | 2 | 60 | A | 2614 | 31 | 89 | D |
| 88 | 3 | 61 | A | 2615 | 32 | 90 | D |
| 89 | 4 | 62 | A | 2616 | 33 | 91 | D |
| 90 | 5 | 63 | A | 2617 | 34 | 92 | D |
| 91 | 6 | 64 | A | 2618 | 35 | 93 | D |
| 92 | 7 | 65 | A | 2619 | 36 | 94 | D |
| 93 | 8 | 66 | A | 2621 | 37 | 95 | D |
| 93 | 9 | 67 | G | 2624 | 38 | 96 | D |
| 2588 | 10 | 68 | B | 2625 | 39 | 97 | D |
| 2589 | 11 | 69 | B | 2626 | 40 | 98 | E |
| 2591 | 12 | 70 | B | 2627 | 41 | 99 | E |
| 2592 | 13 | 71 | B | 2628 | 42 | 100 | E |
| 2593 | 14 | 72 | B | 2629 | 43 | 101 | E |
| 2594 | 15 | 73 | B | 2630 | 44 | 102 | E |
| 2595 | 16 | 74 | B | 2631 | 45 | 103 | E |
| 2596 | 17 | 75 | B | 2632 | 46 | 104 | E |
| 2598 | 18 | 76 | B | 2633 | 47 | 105 | E |
| 2600 | 19 | 77 | B | 2634 | 48 | 106 | E |
| 2601 | 20 | 78 | C | 2635 | 49 | 107 | B |
| 2602 | 21 | 79 | C | 2636 | 50 | 108 | F |
| 2603 | 22 | 80 | C | 2637 | 51 | 109 | F |
| 2604 | 23 | 81 | C | 2639 | 52 | 110 | F |
| 2606 | 24 | 82 | C | 2640 | 53 | 111 | F |
| 2607 | 25 | 83 | C | 2641 | 54 | 112 | F |
| 2608 | 26 | 84 | C | 2642 | 55 | 113 | F |
| 2609 | 27 | 85 | C | 2643 | 56 | 114 | F |
| 2610 | 28 | 86 | C | 2644 | 57 | 115 | F |
| 2611 | 29 | 87 | C | 2645 | 58 | 116 | F |

Legend
A = Ser. No. 60/195,150
B = Ser. No. 60/195,099
C = Ser. No. 60/195,151
D = 5cr. No. 60/195,148
E = Ser. No. 60/195,093
F = Ser. No. 60/195,098
G = Ser. No. 60/230,149

When a specific nGPCR is identified (for example nGPCR-261 1), it is understood that only that speci nGPCR is being referred to.

It is well known that GPCRs are expressed in many different tissues, including the brain. Accordingly, the nGPCR-x of the present invention may be useful, inter alia, for treating and/or diagnosing mental disorders. Following the techniques described in Example 5, below, those skilled in the art could readily ascertain if nGPCR-x is expressed in a particular tissue or region.

The invention provides purified and isolated polynucleotides (e.g., cDNA, genomic DNA, synthetic DNA, RNA, or combinations thereof, whether single- or double-stranded) that comprise a nucleotide sequence encoding the amnino acid sequence of the po1lypepti des of the invention. Such polynucleotides are useful for recombinantly expressing the receptor and also for detecting expression of the receptor in cells (e.g., using Northern hybridization and in situ hybridization assays). Such polynucleotides also are useful in the design of antisense and other molecules for the suppression of the expression of nGPCR-x in a cultured cell, a tissue, or an animal; for therapeutic purposes; or to provide a model for diseases or conditions characterized by aberrant nGPCR-x expression. Specifically excluded from the definition of polynucleoti des of the invention are entire isolated, non-recombinant native chromosomes of host cells. A preferred polynucleotide has a sequence selected from the group consisting of SEQ if) NO: I to SEQ ID NO:58, which correspond to naturally occurring nGPCR-x sequences. It will be appreciated that numerous other polynucleotide sequences exist that also encode nGPCR-x having the sequence selected from the group consisting of SEQ ID NO:59 to SEQ ID NO: 116, due to the well-known degeneracy of the universal genetic code.

The invention also provides a purified and isolated polynucleotide comprising a nucleotide sequence that encodes a mammalian polypeptide, wherein the polynucleotide hybridizes to a polynucleotide having the sequence set forth in sequences selected from the group consisting of SEQ ID NO:1 to SEQ ID NO:58, or the non-coding strand complementary thereto, under the following hybridization conditions:

(a) hybridization for 16 hours at 42° C. in a hybridization solution comprising 50% formamide, 1% SDS, 1 M NaCl, 10% dextran sulfate; and

(b) washing 2 times for 30 minutes each at 60° C. in a wash solution comprising 0.1% SSC, 1% SDS. Polynucleotides that encode a human allelic variant are highly preferred.

The present invention relates to molecules which comprise the gene sequences that encode the nGPCRs; constructs and recombinant host cells incorporating the gene sequences; the novel GPCR polypeptides encoded by the gene sequences; antibodies to the polypeptides and homologs; kits employing the polynucleotides and polypeptides, and methods of making and using all of the foregoing. In addition, the present invention relates to homologs of the gene sequences and of the polypeptides and methods of making and using the same.

Genomic DNA of the invention comprises the protein-coding region for a polypeptide of the invention and is also intended to include allelic variants thereof. It is widely understood that, for many genes, genomic DNA is transcribed into RNA transcripts that undergo one or more splicing events wherein intron (i.e., non-coding regions) of the transcripts are removed, or “spliced out.” RNA transcripts that can be spliced by alternative mechanisms, and therefore be subject to removal of different RNA sequences but still encode a nGPCR-x polypeptide, are referred to in the art as splice variants which are embraced by the invention. Splice variants comprehended by the invention therefore are encoded by the same original genomic DNA sequences but arise from distinct mRNA transcripts. Allelic variants are modified forms of a wild-type gene sequence, the modification resulting from recombination during chromosomal segregation or exposure to conditions which give rise to genetic mutation. Allelic variants, like wild type genes, are naturally occurring sequences (as opposed to non-naturally occurring variants that arise from in vitro manipulation).

The invention also comprehends cDNA that is obtained through reverse transcription of an RNA polynucleotide encoding nGPCR-x (conventionally followed by second strand synthesis of a complementary strand to provide a double-stranded DNA).

Preferred DNA sequences encoding human nGPCR-x polypeptides are selected from the group consisting of SEQ ID NO: 1 to SEQ ID NO:58. A preferred DNA of the invention comprises a double stranded molecule along with the complementary molecule (the “non-coding strand” or “complement”) having a sequence unambiguously deducible from the coding strand according to Watson-Crick base-pairing rules for DNA. Also preferred are other polynucleotides encoding the nGPCR-x polypeptide selected from the group consisting of SEQ ID NO:59 to SEQ ID NO:116, which differ in sequence from the polynucleotides selected from the group consisting of SEQ ID NO: 1 to SEQ ID NO:58, by virtue of the well-known degeneracy of the universal nuclear genetic code.

The invention further embraces other species, preferably mammalian, homologs of the human nGPCR-x DNA. Species homologs, sometimes referred to as “orthologs,” in general, share at least 35%, at least 40%, at least 45%, at least 50%, at least 60%, at least 65%, at least 70%, at least 75%, at least 80%, at least 85%, at least 90%, at least 95%, at least 98%, or at least 99% homology with human DNA of the invention. Generally, percent sequence “homology” with respect to polynucleotides of the invention may be calculated as the percentage of nucleotide bases in the candidate sequence that are identical to nucleotides in the nGPCR-x sequence set forth in sequences selected from the group consisting of SEQ ID NO: 1 to SEQ ID NO:58, after aligning the sequences and introducing gaps, if necessary, to achieve the maximum percent sequence identity.

Polynucleotides of the invention permit identification and isolation of polynucleotides encoding related nGPCR-x polypeptides, such as human allelic variants and species homologs, by well-known techniques including Southern and/or Northern hybridization, and polymerase chain reaction (PCR). Examples of related polynucleotides include human and non-human genomic sequences, including allelic variants, as well as polynucleotides encoding polypeptides homologous to nGPCR-x and structurally related polypeptides sharing one or more biological, immunological, and/or physical properties of nGPCR-x. Non-human species genes encoding proteins homologous to nGPCR-x can also be identified by Southern and/or PCR analysis and are useful in animal models for nGPCR-x disorders. Knowledge of the sequence of a human nGPCR-x DNA also makes possible through use of Southern hybridization or polymerase chain reaction (PCR) the identification of genomic DNA sequences encoding nGPCR-x expression control regulatory sequences such as promoters, operators, enhancers, repressors, and the like. Polynucleotides of the invention are also useful in hybridization assays to detect the capacity of cells to express nGPCR-x. Polynucleotides of the invention may also provide a basis for diagnostic methods useful for identifying a genetic alteration(s) in a nGPCR-x locus that underlies a disease state or states, which information is useful both for diagnosis and for selection of therapeutic strategies.

According to the present invention, the nGPCR-x nucleotide sequences disclosed herein may be used to identify homologs of the nGPCR-x, in other animals, including but not limited to humans and other mammals, and invertebrates. Any of the nucleotide sequences disclosed herein, or any portion thereof, can be used, for example, as probes to screen databases or nucleic acid libraries, such as, for example, genomic or cDNA libraries, to identify homologs, using screening procedures well known to those skilled in the art. Accordingly, homologs having at least 50%, more preferably at least 60%, more preferably at least 70%, more preferably at least 80%, more preferably at least 90%, more preferably at least 95%, and most preferably at least 100% homology with nGPCR-x sequences can be identified.

The disclosure herein of full-length polynucleotides encoding nGPCR-x polypeptides makes readily available to the worker of ordinary skill in the art every possible fragment of the full-length polynucleotide.

One preferred embodiment of the present invention provides an isolated nucleic acid molecule comprising a sequence homologous sequences selected from the group consisting of SEQ ID NO: 1 to SEQ ID NO:58, and fragments thereof. Another preferred embodiment provides an isolated nucleic acid molecule comprising a sequence selected from the group consisting of SEQ ID NO:1 to SEQ ID NO:58, and fragments thereof.

As used in the present invention, fragments of nGPCR-x-encoding polynucleotides comprise at least 10, and preferably at least 12, 14, 16, 18, 20, 25, 50, or 75 consecutive nucleotides of a polynucleotide encoding nGPCR-x. Preferably, fragment polynucleotides of the invention comprise sequences unique to the nGPCR-x-encoding polynucleotide sequence, and therefore hybridize under highly stringent or moderately stringent conditions only (i.e., “specifically”) to polynucleotides encoding nGPCR-x (or fragments thereof). Polynucleotide fragments of genomic sequences of the invention comprise not only sequences unique to the coding region, but also include fragments of the full-length sequence derived from introns, regulatory regions, and/or other non-translated sequences. Sequences unique to polynucleotides of the invention are recognizable through sequence comparison to other known polynucleotides, and can be identified through use of alignment programs routinely utilized in the art, e.g., those made available in public sequence databases. Such sequences also are recognizable from Southern hybridization analyses to determine the number of fragments of genornic DNA to which a polynucleotide will hybridize. Polynucleotides of the invention can be labeled in a manner that permits their detection, including radioactive, fluorescent, and enzymatic labeling.

Fragment polynucleotides are particularly useful as probes for detection of full-length or fragments of nGPCR-x polynucleotides. One or more polynucleotides can be included in kits that are used to detect the presence of a polynucleotide encoding nGPCR-x, or used to detect variations in a polynucleotide sequence encoding nGPCR-x.

The invention also embraces DNAs encoding nGPCR-x polypeptides that hybridize under moderately stringent or high stringency conditions to the non-coding strand, or complement, of the polynucleotides set forth in sequences selected from the group consisting of SEQ ID NO:1 to SEQ ID NO:58.

Exemplary highly stringent hybridization conditions are as follows: hybridization at 42° C. in a hybridization solution comprising 50% formamide, 1% SDS, 1 M NaCl, 10% Dextran sulfate, and washing twice for 30 minutes at 60° C. in a wash solution comprising 0.1× SSC and 1% SDS. It is understood in the art that conditions of equivalent stringency can be achieved through variation of temperature and buffer, or salt concentration as described Ausubel et al. (Eds.), Protocols in Molecular Biology, John Wiley & Sons (1994), pp.6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

With the knowledge of the nucleotide sequence information disclosed in the present invention, one skilled in the art can identify and obtain nucleotide sequences which encode nGPCR-x from different sources (i.e., different tissues or different organisms) through a variety of means well known to the skilled artisan and as disclosed by, for example, Sambrook et al., “Molecular cloning: a laboratory manual”, Second Edition, Cold Spring Harbor Press, Cold Spring Harbor, N.Y. (1989), which is incorporated herein by reference in its entirety.

For example, DNA that encodes nGPCR-x may be obtained by screening of mRNA, cDNA, or genomic DNA with oligonucleotide probes generated from the nGPCR-x gene sequence information provided herein. Probes may be labeled with a detectable group, such as a fluorescent group, a radioactive atom or a cherniluminescent group in accordance with procedures known to the skilled artisan and used in conventional hybridization assays, as described by, for example, Sambrook et al.

A nucleic acid molecule comprising any of the nGPCR-x nucleotide sequences described above can alternatively be synthesized by use of the polymerase chain reaction (PCR) procedure, with the PCR oligonucleotide primers produced from the nucleotide sequences provided herein. See U.S. Pat. Nos. 4,683,195 to Mullis et al. and 4,683,202 to Mullis. The PCR reaction provides a method for selectively increasing the concentration of a particular nucleic acid sequence even when that sequence has not been previously purified and is present only in a single copy in a particular sample. The method can be used to amplify either single- or double-stranded DNA. The essence of the method involves the use of two oligonucleotide probes to serve as primers for the template-dependent, polymerase mediated replication of a desired nucleic acid molecule.

A wide variety of alternative cloning and in vitro amplification methodologies are well known to those skilled in the art. Examples of these techniques are found in, for example, Berger et al., Guide to Molecular Cloning Techniques, Methods in Enzymology 152, Academic Press, Inc., San Diego, CA (Berger), which is incorporated herein by reference in its entirety.

Automated sequencing methods can be used to obtain or verify the nucleotide sequence of nGPCR-x. The nGPCR-x nucleotide sequences of the present invention are believed to be 100% accurate. However, as is known in the art, nucleotide sequence obtained by automated methods may contain some errors. Nucleotide sequences determined by automation are typically at least about 90%, more typically at least about 95% to at least about 99.9% identical to the actual nucleotide sequence of a given nucleic acid molecule. The actual sequence may be more precisely determined using manual sequencing methods, which are well known in the art. An error in a sequence which results in an insertion or deletion of one or more nucleotides may result in a frame shift in translation such that the predicted amino acid sequence will differ from that which would be predicted from the actual nucleotide sequence of the nucleic acid molecule, starting at the point of the mutation.

The nucleic acid molecules of the present invention, and fragments derived therefrom, are useful for screening for restriction fragment length polymorphism (RFLP) associated with certain disorders, as well as for genetic mapping.

The polynucleotide sequence information provided by the invention makes possible large-scale expression of the encoded polypeptide by techniques well known and routinely practiced in the art.

Vectors

Another aspect of the present invention is directed to vectors, or recombinant expression vectors, comprising any of the nucleic acid molecules described above. Vectors are used herein either to amplify DNA or RNA encoding nGPCR-x and/or to express DNA which encodes nGPCR-x. Preferred vectors include, but are not limited to, plasmids, phages, cosmids, episomes, viral particles or viruses, and integratable DNA fragments (i.e., fragments integratable into the host genome by homologous recombination). Preferred viral particles include, but are not limited to, adenoviruses, baculoviruses, parvoviruses, herpesviruses, poxviruses, adeno-associated viruses, Semliki Forest viruses, vaccinia viruses, and retroviruses. Preferred expression vectors include, but are not limited to, pcDNA3 (Invitrogen) and pSVL (Pharmacia Biotech). Other expression vectors include, but are not limited to, pSPORT™ vectors, pGEM™ vectors (Promega), pPROEXvectors™ (LTI, Bethesda, Md.), Bluescript™ vectors (Stratagene), pQE™ vectors (Qiagen), pSE42O™ (Invitrogen), and pYES2™(Invitrogen).

Expression constructs preferably comprise GPCR-x-encoding polynucleotides operatively linked to an endogenous or exogenous expression control DNA sequence and a transcription terminator. Expression control DNA sequences include promoters, enhancers, operators, and regulatory element binding sites generally, and are typically selected based on the expression systems in which the expression construct is to be utilized. Preferred promoter and enhancer sequences are generally selected for the ability to increase gene expression, while operator sequences are generally selected for the ability to regulate gene expression. Expression constructs of the invention may also include sequences encoding one or more selectable markers that permit identification of host cells bearing the construct. Expression constructs may also include sequences that facilitate, and preferably promote, homologous recombination in a host cell. Preferred constructs of the invention also include sequences necessary for replication in a host cell.

Expression constructs are preferably utilized for production of an encoded protein, but may also be utilized simply to amplify a nGPCR-x-encoding polynucleotide sequence. In preferred embodiments, the vector is an expression vector wherein the polynucleotide of the invention is operatively linked to a polynucleotide comprising an expression control sequence. Autonomously replicating recombinant expression constructs such as plasmid and viral DNA vectors incorporating polynucleotides of the invention are also provided. Preferred expression vectors are replicable DNA constructs in which a DNA sequence encoding nGPCR-x is operably linked or connected to suitable control sequences capable of effecting the expression of the nGPCR-x in a suitable host. DNA regions are operably linked or connected when they are functionally related to each other. For example, a promoter is operably linked or connected to a coding sequence if it controls the transcription of the sequence. Amplification vectors do not require expression control domains, but rather need only the ability to replicate in a host, usually conferred by an origin of replication, and a selection gene to facilitate recognition of transformants. The need for control sequences in the expression vector will vary depending upon the host selected and the transformation method chosen. Generally, control sequences include a transcriptional promoter, an optional operator sequence to control transcription, a sequence encoding suitable MRNA ribosomal binding and sequences which control the termination of transcription and translation.

Preferred vectors preferably contain a promoter that is recognized by the host organism. The promoter sequences of the present invention may be prokaryotic, eukaryotic or viral. Examples of suitable prokaryotic sequences include the P_{R} and P_{L} promoters of bacteriophage lambda (The bacteriophage Lambda, Hershey, A. D., Ed., Cold Spring Harbor Press, Cold Spring Harbor, N.Y. (1973), which is incorporated herein by reference in its entirety; Lambda II, Hendrix, R. W., Ed., Cold Spring Harbor Press, Cold Spring Harbor, N.Y. (1980), which is incorporated herein by reference in its entirety); the trp, recA, heat shock, and lacZ promoters of E. coli and the SV40 early promoter (Benoist et al. Nature, 1981, 290, 304-310, which is incorporated herein by reference in its entirety). Additional promoters include, but are not limited to, mouse mammary tumor virus, long terminal repeat of human immunodeficiency virus, maloney virus, cytomegalovirus immediate early promoter, Epstein Barr virus, Rous sarcoma virus, human actin, human myosin, human hemoglobin, human muscle creatine, and human metalothionein.

Additional regulatory sequences can also be included in preferred vectors. Preferred examples of suitable regulatory sequences are represented by the Shine-Dalgamo of the replicase gene of the phage MS-2 and of the gene clI of bacteriophage lambda. The Shine-Dalgarno sequence may be directly followed by DNA encoding nGPCR-x and result in the expression of the mature nGPCR-x protein.

Moreover, suitable expression vectors can include an appropriate marker that allows the screening of the transformed host cells. The transformation of the selected host is carried out using any one of the various techniques well known to the expert in the art and described in Sambrook et al., supra.

An origin of replication can also be provided either by construction of the vector to include an exogenous origin or may be provided by the host cell chromosomal replication mechanism. If the vector is integrated into the host cell chromosome, the latter may be sufficient. Alternatively, rather than using vectors which contain viral origins of replication, one skilled in the art can transform mammalian cells by the method of co-transformation with a selectable marker and nGPCR-x DNA. An example of a suitable marker is dihydrofolate reductase (DBFR) or thymidine kinase (see, U.S. Pat. No. 4,399,216).

Nucleotide sequences encoding GPCR-x may be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesiderable joining, and ligation with appropriate ligases. Techniques for such manipulation are disclosed by Sambrook et al., supra and are well known in the art. Methods for construction of mammalian expression vectors are disclosed in, for example, Okayama et al., Mol. Cell. Biol., 1983, 3, 280, Cosman et al., Mol. Immunol., 1986, 23, 935, Cosman et al., Nature, 1984, 312, 768, EP-A-0367566, and WO 91/18982, each of which is incorporated herein by reference in its entirety.

Host cells

According to another aspect of the invention, host cells are provided, including prokaryotic and eukaryotic cells, comprising a polynucleotide of the invention (or vector of the invention) in a manner that permits expression of the encoded nGPCR-x polypeptide. Polynucleotides of the invention may be introduced into the host cell as part of a circular plasmid, or as linear DNA comprising an isolated protein coding region or a viral vector. Methods for introducing DNA into the host cell that are well known and routinely practiced in the art include transformation, transfection, electroporation, nuclear injection, or fusion with carriers such as liposomes, micelles, ghost cells, and protoplasts. Expression systems of the invention include bacterial, yeast, fungal, plant, insect, invertebrate, vertebrate, and mammalian cells systems.

The invention provides host cells that are transformed or transfected (stably or transiently) with polynucleotides of the invention or vectors of the invention. As stated above, such host cells are useful for amplifying the polynucleotides and also for expressing the nGPCR-x polypeptide or fragment thereof encoded by the polynucleotide.

In still another related embodiment, the invention provides a method for producing a nGPCR-x polypeptide (or fragment thereof) comprising the steps of growing a host cell of the invention in a nutrient medium and isolating the polypeptide or variant thereof from the cell or the medium. Because nGPCR-x is a seven transmembrane receptor, it will be appreciated that, for some applications, such as certain activity assays, the preferable isolation may involve isolation of cell membranes containing the polypeptide embedded therein, whereas for other applications a more complete isolation may be preferable.

According to some aspects of the present invention, transformed host cells having an expression vector comprising any of the nucleic acid molecules described above are provided.

Expression of the nucleotide sequence occurs when the expression vector is introduced into an appropriate host cell. Suitable host cells for expression of the polypeptides of the invention include, but are not limited to, prokaryotes, yeast, and eukaryotes. If a prokaryotic expression vector is employed, then the appropriate host cell would be any prokaryotic cell capable of expressing the cloned sequences. Suitable prokaryotic cells include, but are not limited to, bacteria of the genera Escherichia, Bacillus, Salmonella, Pseudomonas, Streptomyces, and Staphylococcus.

If an eukaryotic expression vector is employed, then the appropriate host cell would be any eukaryotic cell capable of expressing the cloned sequence. Preferably, eukaryotic cells are cells of higher eukaryotes. Suitable eukaryotic cells include, but are not limited to, non-human mammalian tissue culture cells and human tissue culture cells. Preferred host cells include, but are not limited to, insect cells, HeLa cells, Chinese hamster ovary cells (CHO cells), African green monkey kidney cells (COS cells), human BEK-293 cells, and murine 3T3 fibroblasts. Propagation of such cells in cell culture has become a routine procedure (see, Tissue Culture, Academic Press, Kruse and Patterson, eds. (1973), which is incorporated herein by reference in its entirety).

In addition, a yeast host may be employed as a host cell. Preferred yeast cells include, but are not limited to, the genera Saccharomyces, Pichia, and Kluveromyces. Preferred yeast hosts are S. cerevisiae and P. pastoris. Preferred yeast vectors can contain an origin of replication sequence from a 2T yeast plasmid, an autonomously replication sequence (ARS), a promoter region, sequences for polyadenylation, sequences for transcription termination, and a selectable marker gene. Shuttle vectors for replication in both yeast and E. coli are also included herein.

Alternatively, insect cells may be used as host cells. In a preferred embodiment, the polypeptides of the invention are expressed using a baculovirus expression system (see, Luckow et al., Bio/Technology, 1988, 6, 47, Baculovirus Expression Vectors: A Laboratory Manual, O'Rielly et al. (Eds.), W.H. Freeman and Company, New York, 1992, and U.S. Pat. No. 4,879,236, each of which is incorporated herein by reference in its entirety). In addition, the MAXBACT complete baculovirus expression system (Invitrogen) can, for example, be used for production in insect cells.

Host cells of the invention are a valuable source of immunogen for development of antibodies specifically immunoreactive with nGPCR-x. Host cells of the invention are also useful in methods for the large-scale production of nGPCR-x polypeptides wherein the cells are grown in a suitable culture medium and the desired polypeptide products are isolated from the cells, or from the medium in which the cells are grown, by purification methods known in the art, e.g., conventional chromatographic methods including immunoaffinity chromatography, receptor affinity chromatography, hydrophobic interaction chromatography, lectin affinity chromatography, size exclusion filtration, cation or anion exchange chromatography, high pressure liquid chromatography (HPLC), reverse phase HPLC, and the like. Still other methods of purification include those methods wherein the desired protein is expressed and purified as a fusion protein having a specific tag, label, or chelating moiety that is recognized by a specific binding partner or agent. The purified protein can be cleaved to yield the desired protein, or can be left as an intact fusion protein. Cleavage of the fusion component may produce a form of the desired protein having additional amino acid residues as a result of the cleavage process.

Knowledge of nGPCR-x DNA sequences allows for modification of cells to permit, or increase, expression of endogenous nGPCR-x. Cells can be modified (e.g., by homologous recombination) to provide increased expression by replacing, in whole or in part, the naturally occurring nGPCR-x promoter with all or part of a heterologous promoter so that the cells express nGPCR-x at higher levels. The heterologous promoter is inserted in such a manner that it is operatively linked to endogenous nGPCR-x encoding sequences. (See, for example, PCT International Publication No. WO 94/12650, PCT International Publication No. WO 92/20808, and PCT International Publication No. WO 91/09955.) It is also contemplated that, in addition to heterologous promoter DNA, amplifiable marker DNA (e.g., ada, dhfr, and the multifunctional CAD gene which encodes carbamoyl phosphate synthase, aspartate transcarbamylase, and dihydroorotase) and/or intron DNA may be inserted along with the heterologous promoter DNA. If linked to the nGPCR-x coding sequence, amplification of the marker DNA by standard selection methods results in co-amplification of the nGPCR-x coding sequences in the cells.

Knock-outs

The DNA sequence information provided by the present invention also makes possible the development (e.g., by homologous recombination or “knock-out” strategies; see Capecchi, Science 244:1288-1292 (1989), which is incorporated herein by reference) of animals that fail to express functional nGPCR-x or that express a variant of nGPCR-x. Such animals (especially small laboratory animals such as rats, rabbits, and mice) are useful as models for studying the in vivo activities of nGPCR-x and modulators of nGPCR-x.

Antisense

Also made available by the invention are anti-sense polynucleotides that recognize and hybridize to polynucleotides encoding nGPCR-x. Full-length and fragment anti-sense polynucleotides are provided. Fragment antisense molecules of the invention include (i) those that specifically recognize and hybridize to nGPCR-x RNA (as determined by sequence comparison of DNA encoding nGPCR-x to DNA encoding other known molecules). Identification of sequences unique to nGPCR-x encoding polynucleotides can be deduced through use of any publicly available sequence database, and/or through use of commercially available sequence comparison programs. After identification of the desired sequences, isolation through restriction digestion or amplification using any of the various polymerase chain reaction techniques well known in the art can be performed. Anti-sense polynucleotides are particularly relevant to regulating expression of nGPCR-x by those cells expressing nGPCR-x mRNA.

Antisense nucleic acids (preferably 10 to 30 base-pair oligonucleotides) capable of specifically binding to nGPCR-x expression control sequences or nGPCR-x RNA are introduced into cells (e.g., by a viral vector or colloidal dispersion system such as a liposome). The antisense nucleic acid binds to the nGPCR-x target nucleotide sequence in the cell and prevents transcription and/or translation of the target sequence. Phosphorothioate and methylphosphonate antisense oligonucleotides are specifically contemplated for therapeutic use by the invention. The antisense oligonucleotides may be further modified by adding poly-L-lysine, transferrin polylysine, or cholesterol moieties at their 5′ end. Suppression of nGPCR-x expression at either the transcriptional or translational level is useful to generate cellular or animal models for diseases/conditions characterized by aberrant nGPCR-x expression.

Antisense oligonucleotides, or fragments of sequences selected from the group consisting of SEQ ID NO: 1 to SEQ ID NO:58, or sequences complementary or homologous thereto, derived from the nucleotide sequences of the present invention encoding nGPCR-x are useful as diagnostic tools for probing gene expression in various tissues. For example, tissue can be probed in situ with oligonucleotide probes carrying detectable groups by conventional autoradiography techniques to investigate native expression of this enzyme or pathological conditions relating thereto. Antisense oligonucleotides are preferably directed to regulatory regions of sequences selected from the group consisting of SEQ ID NO: 1 to SEQ ID NO:58, or mRNA corresponding thereto, including, but not limited to, the initiation codon, TATA box, enhancer sequences, and the like.

Transcription factors

The nGPCR-x sequences taught in the present invention facilitate the design of novel transcription factors for modulating nGPCR-x expression in native cells and animals, and cells transformed or transfected with nGPCR-x polynucleotides. For example, the Cys₂-His₂ zinc finger proteins, which bind DNA via their zinc finger domains, have been shown to be amenable to structural changes that lead to the recognition of different target sequences. These artificial zinc finger proteins recognize specific target sites with high affinity and low dissociation constants, and are able to act as gene switches to modulate gene expression. Knowledge of the particular nGPCR-x target sequence of the present invention facilitates the engineering of zinc finger proteins specific for the target sequence using known methods such as a combination of structure-based modeling and screening of phage display libraries (Segal et al., Proc. Natl. Acad. Sci. (USA) 96:2758-2763 (1999); Liu et al., Proc. Natl. Acad. Sci. (USA) 94:5525-5530 (1997); Greisman et al., Science 275:657-661 (1997); Choo et al., J. Mol. Biol. 273:525-532 (1997)). Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence (Segal et al.) The artificial zinc finger repeats, designed based on nGPCR-x sequences, are fused to activation or repression domains to promote or suppress nGPCR-x expression (Liu et al.) Alternatively, the zinc finger domains can be fused to the TATA box-binding factor (TBP) with varying lengths of linker region between the zinc finger peptide and the TBP to create either transcriptional activators or repressors (Kim et al., Proc. Nati. Acad. Sci. (USA) 94:3616-3620 (1997). Such proteins and polynucleotides that encode them, have utility for modulating nGPCR-x expression in vivo in both native cells, animals and humans; and/or cells transfected with nGPCR-x-encoding sequences. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to antisense or catalytic RNA methods (McColl et al., Proc. Natl. Acad. Sci. (USA) 96:9521-9526 (1997); Wu et al., Proc. Natl. Acad. Sci. (USA) 92:344-348 (1995)). The present invention contemplates methods of designing such transcription factors based on the gene sequence of the invention, as well as customized zinc finger proteins, that are useful to modulate nGPCR-x expression in cells (native or transformed) whose genetic complement includes these sequences. Polypeptides

The invention also provides purified and isolated mammalian nGPCR-x polypeptides encoded by a polynucleotide of the invention. Presently preferred is a human nGPCR-x polypeptide comprising the amino acid sequence set out in sequences selected from the group consisting of SEQ ID NO:59 to SEQ ID NO: 116, or fragments thereof comprising an epitope specific to the polypeptide. By “epitope specific to” is meant a portion of the nGPCR receptor that is recognizable by an antibody that is specific for the nGPCR, as defined in detail below.

Although the sequences provided are particular human sequences, the invention is intended to include within its scope other human allelic variants; non-human mammalian forms of nGPCR-x, and other vertebrate forms of nGPCR-x.

It will be appreciated that extracellular epitopes are particularly useful for generating and screening for antibodies and other binding compounds that bind to receptors such as nGPCR-x. Thus, in another preferred embodiment, the invention provides a purified and isolated polypeptide comprising at least one extracellular domain (e.g., the N-terminal extracellular domain or one of the three extracellular loops) of nGPCR-x. Purified and isolated polypeptides comprising the N-terminal extracellular domain of nGPCR-x are highly preferred. Also preferred is a purified and isolated polypeptide comprising a nGPCR-x fragment selected from the group consisting of the N-terninal extracellular domain of nGPCR-x, transmembrane domains of nGPCR-x, an extracellular loop connecting transmembrane domains of nGPCR-x, an intracellular loop connecting transmembrane domains of nGPCR-x, the C-terminal cytoplasmic region of nGPCR-x, and fusions thereof. Such fragments may be continuous portions of the native receptor. However, it will also be appreciated that knowledge of the nGPCR-x gene and protein sequences as provided herein permits recombining of various domains that are not contiguous in the native protein. Using a FORTRAN computer program called “tmtrest.all” [Parodi et al., Comput. Appl. Biosci. 5:527-535 (1994)], nGPCR-x was shown to contain transmembrane-spanning domains.

The invention also embraces polypeptides that have at least 99%, at least 95%, at least 90%, at least 85%, at least 80%, at least 75%, at least 70%, at least 65%, at least 60%, at least 55% or at least 50% identity and/or homology to the preferred polypeptide of the invention. Percent amino acid sequence “identity” with respect to the preferred polypeptide of the invention is defined herein as the percentage of amino acid residues in the candidate sequence that are identical with the residues in the nGPCR-x sequence after aligning both sequences and introducing gaps, if necessary, to achieve the maximum percent sequence identity, and not considering any conservative substitutions as part of the sequence identity. Percent sequence “homology” with respect to the preferred polypeptide of the invention is defined herein as the percentage of amino acid residues in the candidate sequence that are identical with the residues in the nGPCR-x sequence after aligning the sequences and introducing gaps, if necessary, to achieve the maximum percent sequence identity, and also considering any conservative substitutions as part of the sequence identity.

In one aspect, percent homology is calculated as the percentage of amino acid residues in the smaller of two sequences which align with identical amino acid residue in the sequence being compared, when four gaps in a length of 100 amino acids may be introduced to maximize alignment (Dayhoff, in Atlas of Protein Sequence and Structure, Vol. 5, p. 124, National Biochemical Research Foundation, Washington, D.C. (1972), incorporated herein by reference).

Polypeptides of the invention may be isolated from natural cell sources or may be chemically synthesized, but are preferably produced by recombinant procedures involving host cells of the invention. Use of mammalian host cells is expected to provide for such post-translational modifications (e.g., glycosylation, truncation, lipidation, and phosphorylation) as may be needed to confer optimal biological activity on recombinant expression products of the invention. Glycosylated and non-glycosylated forms of nGPCR-x polypeptides are embraced by the invention.

The invention also embraces variant (or analog) nGPCR-x polypeptides. In one example, insertion variants are provided wherein one or more amino acid residues supplement a nGPCR-x amino acid sequence. Insertions may be located at either or both termini of the protein, or may be positioned within internal regions of the nGPCR-x amino acid sequence. Insertional variants with additional residues at either or both termini can include, for example, fusion proteins and proteins including amino acid tags or labels.

Insertion variants include nGPCR-x polypeptides wherein one or more amino acid residues are added to a nGPCR-x acid sequence or to a biologically active fragment thereof.

Variant products of the invention also include mature nGPCR-x products, i.e., nGPCR-x products wherein leader or signal sequences are removed, with additional amino terminal residues. The additional amino terminal residues may be derived from another protein, or may include one or more residues that are not identifiable as being derived from specific proteins. nGPCR-x products with an additional methionine residue at position-1 (Met⁻¹nGPCR-x) are contemplated, as are variants with additional methionine and lysine residues at positions -2 and -1 (Met⁻²-Lys⁻¹-nGPCR-x). Variants of nGPCR-x with additional Met, Met-Lys, Lys residues (or one or more basic residues in general) are particularly useful for enhanced recombinant protein production in bacterial host cells.

The invention also embraces nGPCR-x variants having additional amino acid residues that result from use of specific expression systems. For example, use of commercially available vectors that express a desired polypeptide as part of a glutathione-S-transferase (GST) fusion product provides the desired polypeptide having an additional glycine residue at position-1 after cleavage of the GST component from the desired polypeptide. Variants that result from expression in other vector systems are also contemplated.

Insertional variants also include fusion proteins wherein the amino terminus and/or the carboxy terminus of nGPCR-x is/are fused to another polypeptide.

In another aspect, the invention provides deletion variants wherein one or more amino acid residues in a nGPCR-x polypeptide are removed. Deletions can be effected at one or both termini of the nGPCR-x polypeptide, or with removal of one or more non-terminal amino acid residues of nGPCR-x. Deletion variants, therefore, include all fragments of a nGPCR-x polypeptide.

The invention also embraces polypeptide fragments of sequences selected from the group consisting of SEQ ID NO:59 to SEQ ID NO:116, wherein the fragments maintain biological (e.g., ligand binding and/or intracellular signaling) immunological properties of a nGPCR-x polypeptide.

In one preferred embodiment of the invention, an isolated nucleic acid molecule comprises a nucleotide sequence that encodes a polypeptide comprising an amino acid sequence homologous to sequences selected from the group consisting of SEQ ID NO:59 to SEQ ID NO: 116, and fragments thereof, wherein the nucleic acid molecule encoding at least a portion of nGPCR-x. In a more preferred embodiment, the isolated nucleic acid molecule comprises a sequence that encodes a polypeptide comprising sequences selected from the group consisting of SEQ ID NO:59 to SEQ ID NO: 116, and fragments thereof.

As used in the present invention, polypeptide fragments comprise at least 5, 10, 15, 20, 25, 30, 35, or 40 consecutive amino acids of sequences selected from the group consisting of SEQ ID NO:59 to SEQ ID NO: 116. Preferred polypeptide fragments display antigenic properties unique to, or specific for, human nGPCR-x and its allelic and species homologs. Fragments of the invention having the desired biological and immunological properties can be prepared by any of the methods well known and routinely practiced in the art.

In still another aspect, the invention provides substitution variants of nGPCR-x polypeptides. Substitution variants include those polypeptides wherein one or more amino acid residues of a nGPCR-x polypeptide are removed and replaced with alternative residues. In one aspect, the substitutions are conservative in nature; however, the invention embraces substitutions that are also non-conservative. Conservative substitutions for this purpose may be defined as set out in Tables 2, 3, or 4 below.

Variant polypeptides include those wherein conservative substitutions have been introduced by modification of polynucleotides encoding polypeptides of the invention. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table 2 (from WO 97/09433, page 10, published Mar. 13, 1997 (PCT/GB96/02197, filed Sep. 6, 1996), immediately below.

TABLE 2
Conservative Substitutions I

| SIDE CHAIN | |
|---|---|
| CHARACTERISTIC | AMINO ACID |
| Aliphatic | |
| Non-polar | G A P |
| | I L V |
| Polar - uncharged | C S T M |
| | N Q |
| Polar - charged | D E |
| | K R |
| Aromatic | H F W Y |
| Other | N Q D E |

Alternatively, conservative amino acids can be grouped as described in Lehninger, [Biochernistry, Second Edition; Worth Publishers, Inc. NY, N.Y. (1975), pp.71-77] as set out in Table 3, below.

TABLE 3
Conservative Substitutions II

| SIDE CHAIN | |
|---|---|
| CHARACTERISTIC | AMINO ACID |
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

As still another alternative, exemplary conservative substitutions are set out in Table 4, below.

TABLE 4
Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

It should be understood that the definition of polypeptides of the invention is intended to include polypeptides bearing modifications other than insertion, deletion, or substitution of amino acid residues. By way of example, the modifications may be covalent in nature, and include for example, chemical bonding with polymers, lipids, other organic, and inorganic moieties. Such derivatives may be prepared to increase circulating half-life of a polypeptide, or may be designed to improve the targeting capacity of the polypeptide for desired cells, tissues, or organs. Similarly, the invention further embraces nGPCR-x polypeptides that have been covalently modified to include one or more water-soluble polymer attachments such as polyethylene glycol, polyoxyethylene glycol, or polypropylene glycol. Variants that display ligand binding properties of native nGPCR-x and are expressed at higher levels, as well as variants that provide for constitutively active receptors, are particularly useful in assays of the invention; the variants are also useful in providing cellular, tissue and animal models of diseases/conditions characterized by aberrant nGPCR-x activity.

In a related embodiment, the present invention provides compositions comprising purified polypeptides of the invention. Preferred compositions comprise, in addition to the polypeptide of the invention, a pharmaceutically acceptable (i.e., sterile and non-toxic) liquid, semisolid, or solid diluent that serves as a pharmaceutical vehicle, excipient, or medium. Any diluent known in the art may be used. Exemplary diluents include, but are not limited to, water, saline solutions, polyoxyethylene sorbitan monolaurate, magnesium stearate, methyl- and propylhydroxybenzoate, talc, alginates, starches, lactose, sucrose, dextrose, sorbitol, mannitol, glycerol, calcium phosphate, mineral oil, and cocoa butter.

Variants that display ligand binding properties of native nGPCR-x and are expressed at higher levels, as well as variants that provide for constitutively active receptors, are particularly useful in assays of the invention; the variants are also useful in assays of the invention and in providing cellular, tissue and animal models of diseases/conditions characterized by aberrant nGPCR-x activity.

The G protein-coupled receptor functions through a specific heterotrimeric guanine-nucleotide-binding regulatory protein (G-protein) coupled to the intracellular portion of the G protein-coupled receptor molecule. Accordingly, the G protein-coupled receptor has a specific affinity to G protein. G proteins specifically bind to guanine nucleotides. Isolation of G proteins provides a means to isolate guanine nucleotides. G proteins may be isolated using commercially available anti-G protein antibodies or isolated G protein-coupled receptors. Similarly, G proteins may be detected in a sample isolated using commercially available detectable anti-G protein antibodies or isolated G protein-coupled receptors.

According to the present invention, the isolated nGPCR-x proteins of the present invention are useful to isolate and purify G proteins from samples such as cell lysates. Example 15 below sets forth an example of isolation of G proteins using isolated nGPCR-x proteins. Such methodology may be used in place of the use of commercially available anti-G protein antibodies which are used to isolate G proteins. Moreover, G proteins may be detected using n-GPCR-x proteins in place of commercially available detectable anti-G protein antibodies. Since nGPCR-x proteins specifically bind to G proteins, they can be employed in any specific use where G protein specific affinity is required such as those uses where commercially available anti-G protein antibodies are employed.

Antibodies

Also comprehended by the present invention are antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for nGPCR-x or fragments thereof. Preferred antibodies of the invention are human antibodies that are produced and identified according to methods described in WO93/11236, published Jun. 20, 1993, which is incorporated herein by reference in its entirety. Antibody fragments, including Fab, Fab′, F(ab′)₂, and Fᵥ, are also provided by the invention. The term “specific for,” when used to describe antibodies of the invention, indicates that the variable regions of the antibodies of the invention recognize and bind nGPCR-x polypeptides exclusively (i.e., are able to distinguish nGPCR-x polypeptides from other known GPCR polypeptides by virtue of measurable differences in binding affinity, despite the possible existence of localized sequence identity, homology, or similarity between nGPCR-x and such polypeptides). It will be understood that specific antibodies may also interact with other proteins (for example, S. aureus protein A or other antibodies in ELISA techniques) through interactions with sequences outside the variable region of the antibodies, and, in particular, in the constant region of the molecule. Screening assays to determine binding specificity of an antibody of the invention are well known and routinely practiced in the art. For a comprehensive discussion of such assays, see Harlow et al. (Eds.), Antibodies A Laboratory Manual; Cold Spring Harbor Laboratory; Cold Spring Harbor, N.Y. (1988), Chapter 6. Antibodies that recognize and bind fragments of the nGPCR-x polypeptides of the invention are also contemplated, provided that the antibodies are specific for nGPCR-x polypeptides. Antibodies of the invention can be produced using any method well known and routinely practiced in the art.

The invention provides an antibody that is specific for the nGPCR-x of the invention. Antibody specificity is described in greater detail below. However, it should be emphasized that antibodies that can be generated from polypeptides that have previously been described in the literature and that are capable of fortuitously cross-reacting with nGPCR-x (e.g., due to the fortuitous existence of a similar epitope in both polypeptides) are considered “cross-reactive” antibodies. Such cross-reactive antibodies are not antibodies that are “specific” for nGPCR-x. The determination of whether an antibody is specific for nGPCR-x or is cross-reactive with another known receptor is made using any of several assays, such as Western blotting assays, that are well known in the art. For identifying cells that express nGPCR-x and also for modulating nGPCR-x-ligand binding activity, antibodies that specifically bind to an extracellular epitope of the nGPCR-x are preferred.

In one preferred variation, the invention provides monoclonal antibodies. Hybridomas that produce such antibodies also are intended as aspects of the invention. In yet another variation, the invention provides a humanized antibody. Humanized antibodies are useful for in vivo therapeutic indications.

In another variation, the invention provides a cell-free composition comprising polyclonal antibodies, wherein at least one of the antibodies is an antibody of the invention specific for nGPCR-x. Antisera isolated from an animal is an exemplary composition, as is a composition comprising an antibody fraction of an antisera that has been resuspended in water or in another diluent, excipient, or carrier.

In still another related embodiment, the invention provides an anti-idiotypic antibody specific for an antibody that is specific for nGPCR-x.

It is well known that antibodies contain relatively small antigen binding domains that can be isolated chemically or by recombinant techniques. Such domains are useful nGPCR-x binding molecules themselves, and also may be reintroduced into human antibodies, or fused to toxins or other polypeptides. Thus, in still another embodiment, the invention provides a polypeptide comprising a fragment of a nGPCR-x-specific antibody, wherein the fragment and the polypeptide bind to the nGPCR-x. By way of non-limiting example, the invention provides polypeptides that are single chain antibodies and CDR-grafted antibodies.

Non-human antibodies may be humanized by any of the methods known in the art. In one method, the non-human CDRs are inserted into a human antibody or consensus antibody framework sequence. Further changes can then be introduced into the antibody framework to modulate affinity or immunogenicity.

Antibodies of the invention are useful for, e.g., therapeutic purposes (by modulating activity of nGPCR-x), diagnostic purposes to detect or quantitate nGPCR-x, and purification of nGPCR-x. Kits comprising an antibody of the invention for any of the purposes described herein are also comprehended. In general, a kit of the invention also includes a control antigen for which the antibody is immunospecific.

Compositions

Mutations in the nGPCR-x gene that result in loss of normal function of the nGPCR-x gene product underlie nGPCR-x-related human disease states. The invention comprehends gene therapy to restore nGPCR-x activity to treat those disease states. Delivery of a functional nGPCR-x gene to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, no. 6679, pp.25-20 (1998). For additional reviews of gene therapy technology see Friedmann, Science, 244: 1275-1281 (1989); Verma, Scientific American: 68-84 (1990); and Miller, Nature, 357: 455-460 (1992). Alternatively, it is contemplated that in other human disease states, preventing the expression of, or inhibiting the activity of, nGPCR-x will be useful in treating disease states. It is contemplated that antisense therapy or gene therapy could be applied to negatively regulate the expression of nGPCR-x.

Another aspect of the present invention is directed to compositions, including pharmaceutical compositions, comprising any of the nucleic acid molecules or recombinant expression vectors described above and an acceptable carrier or diluent. Preferably, the carrier or diluent is pharmaceutically acceptable. Suitable carriers are described in the most recent edition of Remington's Pharmaceutical Sciences, A. Osol, a standard reference text in this field, which is incorporated herein by reference in its entirety. Preferred examples of such carriers or diluents include, but are not limited to, water, saline, Ringer's solution, dextrose solution, and 5% human serum albumin. Liposomes and nonaqueous vehicles such as fixed oils may also be used. The formulations are sterilized by commonly used techniques.

Also within the scope of the invention are compositions comprising polypeptides, polynucleotides, or antibodies of the invention that have been formulated with, e.g., a pharmaceutically acceptable carrier.

The invention also provides methods of using antibodies of the invention. For example, the invention provides a method for modulating ligand binding of a nGPCR-x comprising the step of contacting the nGPCR-x with an antibody specific for the nGPCR-x, under conditions wherein the antibody binds the receptor.

As discussed above, it is well known that GPCRs are expressed in many different tissues and regions, including in the brain. GPCRs that may be expressed in the brain, such as nGPCR-x, provide an indication that aberrant nGPCR-x signaling activity may correlate with one or more neurological or psychological disorders. The invention also provides a method for treating a neurological or psychiatric disorder comprising the step of administering to a mammal in need of such treatment an amount of an antibody-like polypeptide of the invention that is sufficient to modulate ligand binding to a nGPCR-x in neurons of the mammal. nGPCR-x may also be expressed in other tissues, including but not limited to, peripheral blood lymphocytes, pancreas, ovary, uterus, testis, salivary gland, thyroid gland, kidney, adrenal gland, liver, bone marrow, prostate, fetal liver, colon, muscle, and fetal brain, and may be found in many other tissues. Within the brain, nGPCR-x mRNA transcripts may be found in many tissues, including, but not limited to, frontal lobe, hypothalamus, pons, cerebellum, caudate nucleus, and medulla.

Kits

The present invention is also directed to kits, including pharmaceutical kits. The kits can comprise any of the nucleic acid molecules described above, any of the polypeptides described above, or any antibody which binds to a polypeptide of the invention as described above, as well as a negative control. The kit preferably comprises additional components, such as, for example, instructions, solid support, reagents helpful for quantification, and the like.

In another aspect, the invention features methods for detection of a polypeptide in a sample as a diagnostic tool for diseases or disorders, wherein the method comprises the steps of: (a) contacting the sample with a nucleic acid probe which hybridizes under hybridization assay conditions to a nucleic acid target region of a polypeptide having sequences selected from the group consisting of SEQ ID NO:59 to SEQ ID NO: 116, said probe comprising the nucleic acid sequence encoding the polypeptide, fragments thereof, and the complements of the sequences and fragments; and (b) detecting the presence or amount of the probe:target region hybrid as an indication of the disease.

In preferred embodiments of the invention, the disease is selected from the group consisting of thyroid disorders (e.g. thyreotoxicosis, myxoedema); renal failure; inflammatory conditions (e.g., Crohn′ s disease); diseases related to cell differentiation and homeostasis; rheumatoid arthritis; autoimmune disorders; movement disorders; CNS disorders (e.g., pain including migraine; stroke; psychotic and neurological disorders, including anxiety, mental disorder, manic depression, anxiety, generalized anxiety disorder, post-traumatic-stress disorder, depression, bipolar disorder, delirium, dementia, severe mental retardation; dyskinesias, such as Huntington's disease or Tourette's Syndrome; attention disorders including ADD and ADHD, and degenerative disorders such as Parkinson's, Alzheimer's; movement disorders, including ataxias, supranuclear palsy, etc.); infections, such as viral infections caused by HIV-1 or HIV-2; metabolic and cardiovascular diseases and disorders (e.g., type 2 diabetes, impaired glucose tolerance, dyslipidemia, obesity, anorexia, hypotension, hypertension, thrombosis, myocardial infarction, cardiomyopathies, atherosclerosis, etc.); proliferative diseases and cancers (e.g., different cancers such as breast, colon, lung, etc., and hyperproliferative disorders such as psoriasis, prostate hyperplasia, etc.); hormonal disorders (e.g., male/female hormonal replacement, polycystic ovarian syndrome, alopecia, etc.); and sexual dysfunction, among others.

Kits may be designed to detect either expression of polynucleotides encoding nGPCR-x expressed in the brain or the nGPCR-x proteins themselves in order to identify tissue as being neurological. For example, oligonucleotide hybridization kits can be provided which include a container having an oligonucleotide probe specific for the nGPCR-x-specific DNA and optionally, containers with positive and negative controls and/or instructions. Similarly, PCR kits can be provided which include a container having primers specific for the nGPCR-x-specific sequences, DNA and optionally, containers with size markers, positive and negative controls and/or instructions.

Hybridization conditions should be such that hybridization occurs only with the genes in the presence of other nucleic acid molecules. Under stringent hybridization conditions only highly complementary nucleic acid sequences hybridize. Preferably, such conditions prevent hybridization of nucleic acids having 1 or 2 mismatches out of 20 contiguous nucleotides. Such conditions are defined supra.

The diseases for which detection of genes in a sample could be diagnostic include diseases in which nucleic acid (DNA and/or RNA) is amplified in comparison to normal cells. By “amplification” is meant increased numbers of DNA or RNA in a cell compared with normal cells.

The diseases that could be diagnosed by detection of nucleic acid in a sample preferably include central nervous system and metabolic diseases. The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

Alternatively, immunoassay kits can be provided which have containers container having antibodies specific for the nGPCR-x-protein and optionally, containers with positive and negative controls and/or instructions.

Kits may also be provided useful in the identification of GPCR binding partners such as natural ligands or modulators (agonists or antagonists). Substances useful for treatment of disorders or diseases preferably show positive results in one or more in vitro assays for an activity corresponding to treatment of the disease or disorder in question. Substances that modulate the activity of the polypeptides preferably include, but are not limited to, antisense oligonucleotides, agonists and antagonists, and inhibitors of protein kinases.

Methods of inducing immune response

Another aspect of the present invention is directed to methods of inducing an immune response in a mammal against a polypeptide of the invention by administering to the mammal an amount of the polypeptide sufficient to induce an immune response. The amount will be dependent on the animal species, size of the animal, and the like but can be determined by those skilled in the art.

Methods of identifying ligands

The invention also provides assays to identify compounds that bind nGPCR-x. One such assay comprises the steps of: (a) contacting a composition comprising a nGPCR-x with a compound suspected of binding nGPCR-x; and (b) measuring binding between the compound and nGPCR-x. In one variation, the composition comprises a cell expressing nGPCR-x on its surface. In another variation, isolated nGPCR-x or cell membranes comprising nGPCR-x are employed. The binding may be measured directly, e.g., by using a labeled compound, or may be measured indirectly by several techniques, including measuring intracellular signaling of nGPCR-x induced by the compound (or measuring changes in the level of nGPCR-x signaling). Following steps (a) and (b), compounds identified as binding nGPCR-x may be tested in other assays including, but not limited to, in vivo models, to confirm or quantitate binding to nGPCR-x.

Specific binding molecules, including natural ligands and synthetic compounds, can be identified or developed using isolated or recombinant nGPCR-x products, nGPCR-x variants, or preferably, cells expressing such products. Binding partners are useful for purifying nGPCR-x products and detection or quantification of nGPCR-x products in fluid and tissue samples using known immunological procedures. Binding molecules are also manifestly useful in modulating (i.e., blocking, inhibiting or stimulating) biological activities of nGPCR-x, especially those activities involved in signal transduction.

The DNA and amino acid sequence information provided by the present invention also makes possible identification of binding partner compounds with which a nGPCR-x polypeptide or polynucleotide will interact. Methods to identify binding partner compounds include solution assays, in vitro assays wherein nGPCR-x polypeptides are immobilized, and cell-based assays. Identification of binding partner compounds of nGPCR-x polypeptides provides candidates for therapeutic or prophylactic intervention in pathologies associated with nGPCR-x normal and aberrant biological activity.

The invention includes several assay systems for identifying nGPCR-x binding partners. In solution assays, methods of the invention comprise the steps of (a) contacting a nGPCR-x polypeptide with one or more candidate binding partner compounds and (b) identifying the compounds that bind to the nGPCR-x polypeptide. Identification of the compounds that bind the nGPCR-x polypeptide can be achieved by isolating the nGPCR-x polypeptidetbinding partner complex, and separating the binding partner compound from the nGPCR-x polypeptide. An additional step of characterizing the physical, biological, and/or biochemical properties of the binding partner compound is also comprehended in another embodiment of the invention, wherein compounds identified as binding nGPCR-x may be tested in other assays including, but not limited to, in vivo models, to confirm or quantitate binding to nGPCR-x. In one aspect, the nGPCR-x polypeptidelbinding partner complex is isolated using an antibody immunospecific for either the nGPCR-x polypeptide or the candidate binding partner compound.

In still other embodiments, either the nGPCR-x polypeptide or the candidate binding partner compound comprises a label or tag that facilitates its isolation, and methods of the invention to identify binding partner compounds include a step of isolating the nGPCR-x polypeptidelbinding partner complex through interaction with the label or tag. An exemplary tag of this type is a poly-histidine sequence, generally around six histidine residues, that permits isolation of a compound so labeled using nickel chelation. Other labels and tags, such as the FLAG® tag (Eastman Kodak, Rochester, N.Y.), well known and routinely used in the art, are embraced by the invention.

In one variation of an in vitro assay, the invention provides a method comprising the steps of (a) contacting an immobilized nGPCR-x polypeptide with a candidate binding partner compound and (b) detecting binding of the candidate compound to the nGPCR-x polypeptide. In an alternative embodiment, the candidate binding partner compound is immobilized and binding of nGPCR-x is detected. Immobilization is accomplished using any of the methods well known in the art, including covalent bonding to a support, a bead, or a chromatographic resin, as well as non-covalent, high affinity interactions such as antibody binding, or use of streptavidin/biotin binding wherein the immobilized compound includes a biotin moiety. Detection of binding can be accomplished (i) using a radioactive label on the compound that is not immobilized, (ii) using of a fluorescent label on the non-immobilized compound, (iii) using an antibody immunospecific for the non-immobilized compound, (iv) using a label on the non-immobilized compound that excites a fluorescent support to which the immobilized compound is attached, as well as other techniques well known and routinely practiced in the art.

The invention also provides cell-based assays to identify binding partner compounds of a nGPCR-x polypeptide. In one embodiment, the invention provides a method comprising the steps of contacting a nGPCR-x polypeptide expressed on the surface of a cell with a candidate binding partner compound and detecting binding of the candidate binding partner compound to the nGPCR-x polypeptide. In a preferred embodiment, the detection comprises detecting a calcium flux or other physiological event in the cell caused by the binding of the molecule.

Another aspect of the present invention is directed to methods of identifying compounds that bind to either nGPCR-x or nucleic acid molecules encoding nGPCR-x, comprising contacting nGPCR-x, or a nucleic acid molecule encoding the same, with a compound, and determining whether the compound binds nGPCR-x or a nucleic acid molecule encoding the same. Binding can be determined by binding assays which are well known to the skilled artisan, including, but not limited to, gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like, which are described in, for example, Current Protocols in Molecular Biology, 1999, John Wiley & Sons, NY, which is incorporated herein by reference in its entirety. The compounds to be screened include (which may include compounds which are suspected to bind nGPCR-x, or a nucleic acid molecule encoding the same), but are not limited to, extracellular, intracellular, biologic or chemical origin. The methods of the invention also embrace ligands, especially neuropeptides, that are attached to a label, such as a radiolabel (e.g., ¹²⁵I, ³⁵S, ³²P, ³³P, ³H), a fluorescence label, a chemiluminescent label, an enzymic label and an immunogenic label. Modulators falling within the scope of the invention include, but are not limited to, non-peptide molecules such as non-peptide mimetics, non-peptide allosteric effectors, and peptides. The nGPCR-x polypeptide or polynucleotide employed in such a test may either be free in solution, attached to a solid support, borne on a cell surface or located intracellularly or associated with a portion of a cell. One skilled in the art can, for example, measure the formation of complexes between nGPCR-x and the compound being tested. Alternatively, one skilled in the art can examine the diminution in complex formation between nGPCR-x and its substrate caused by the compound being tested.

In another embodiment of the invention, high throughput screening for compounds having suitable binding affinity to nGPCR-x is employed. Briefly, large numbers of different test compounds are synthesized on a solid substrate. The peptide test compounds are contacted with nGPCR-x and washed. Bound nGPCR-x is then detected by methods well known in the art. Purified polypeptides of the invention can also be coated directly onto plates for use in the aforementioned drug screening techniques. In addition, non-neutralizing antibodies can be used to capture the protein and immobilize it on the solid support.

Generally, an expressed nGPCR-x can be used for HTS binding assays in conjunction with its defined ligand, in this case the corresponding neuropeptide that activates it. The identified peptide is labeled with a suitable radioisotope, including, but not limited to, ¹²⁵I, ³H, ³⁵S or ³²P, by methods that are well known to those skilled in the art. Alternatively, the peptides may be labeled by well-known methods with a suitable fluorescent derivative (Baindur et al., Drug Dev. Res., 1994, 33, 373-398; Rogers, Drug Discovery Today, 1997, 2, 156-160). Radioactive ligand specifically bound to the receptor in membrane preparations made from the cell line expressing the recombinant protein can be detected in HTS assays in one of several standard ways, including filtration of the receptor-ligand complex to separate bound ligand from unbound ligand (Williams, Med. Res. Rev., 1991, 11, 147-184; Sweetnam et al., J. Natural Products, 1993, 56, 441-455). Alternative methods include a scintillation proximity assay (SPA) or a FlashPlate format in which such separation is unnecessary (Nakayama, Cur. Opinion Drug Disc. Dev., 1998, 1, 85-91 Bossé et al., J. Biomolecular Screening, 1998, 3, 285-292.). Binding of fluorescent ligands can be detected in various ways, including fluorescence energy transfer (FRET), direct spectrophotofluorometric analysis of bound ligand, or fluorescence polarization (Rogers, Drug Discovery Today, 1997, 2, 156-160; Hill, Cur. Opinion Drug Disc. Dev., 1998, 1, 92-97).

Other assays may be used to identify specific ligands of a nGPCR-x receptor, including assays that identify ligands of the target protein through measuring direct binding of test ligands to the target protein, as well as assays that identify ligands of target proteins through affinity ultrafiltration with ion spray mass spectroscopy/BPLC methods or other physical and analytical methods. Alternatively, such binding interactions are evaluated indirectly using the yeast two-hybrid system described in Fields et al., Nature, 340:245-246 (1989), and Fields et al., Trends in Genetics, 10:286-292 (1994), both of which are incorporated herein by reference. The two-hybrid system is a genetic assay for detecting interactions between two proteins or polypeptides. It can be used to identify proteins that bind to a known protein of interest, or to delineate domains or residues critical for an interaction. Variations on this methodology have been developed to clone genes that encode DNA binding proteins, to identify peptides that bind to a protein, and to screen for drugs. The two-hybrid system exploits the ability of a pair of interacting proteins to bring a transcription activation domain into close proximity with a DNA binding domain that binds to an upstream activation sequence (UAS) of a reporter gene, and is generally performed in yeast. The assay requires the construction of two hybrid genes encoding (1) a DNA-binding domain that is fused to a first protein and (2) an activation domain fused to a second protein. The DNA-binding domain targets the first hybrid protein to the UAS of the reporter gene; however, because most proteins lack an activation domain, this DNA-binding hybrid protein does not activate transcription of the reporter gene. The second hybrid protein, which contains the activation domain, cannot by itself activate expression of the reporter gene because it does not bind the UAS. However, when both hybrid proteins are present, the noncovalent interaction of the first and second proteins tethers the activation domain to the UAS, activating transcription of the reporter gene. For example, when the first protein is a GPCR gene product, or fragment thereof, that is known to interact with another protein or nucleic acid, this assay can be used to detect agents that interfere with the binding interaction. Expression of the reporter gene is monitored as different test agents are added to the system. The presence of an inhibitory agent results in lack of a reporter signal.

The yeast two-hybrid assay can also be used to identify proteins that bind to the gene product. In an assay to identify proteins that bind to a nGPCR-x receptor, or fragment thereof, a fusion polynucleotide encoding both a nGPCR-x receptor (or fragment) and a UAS binding domain (i.e., a first protein) may be used. In addition, a large number of hybrid genes each encoding a different second protein fused to an activation domain are produced and screened in the assay. Typically, the second protein is encoded by one or more members of a total cDNA or genomic DNA fusion library, with each second protein-coding region being fused to the activation domain. This system is applicable to a wide variety of proteins, and it is not even necessary to know the identity or function of the second binding protein. The system is highly sensitive and can detect interactions not revealed by other methods; even transient interactions may trigger transcription to produce a stable mRNA that can be repeatedly translated to yield the reporter protein.

Other assays may be used to search for agents that bind to the target protein. One such screening method to identify direct binding of test ligands to a target protein is described in U.S. Pat. No. 5,585,277, incorporated herein by reference. This method relies on the principle that proteins generally exist as a mixture of folded and unfolded states, and continually alternate between the two states. When a test ligand binds to the folded form of a target protein (i.e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method that distinguishes between the folded and unfolded states of the target protein. The function of the target protein need not be known in order for this assay to be performed. Virtually any agent can be assessed by this method as a test ligand, including, but not limited to, metals, polypeptides, proteins, lipids, polysaccharides, polynucleotides and small organic molecules.

Another method for identifying ligands of a target protein is described in Wieboldt et al., Anal. Chem., 69:1683-1691 (1997), incorporated herein by reference. This technique screens combinatorial libraries of 20-30 agents at a time in solution phase for binding to the target protein. Agents that bind to the target protein are separated from other library components by simple membrane washing. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

Other embodiments of the invention comprise using competitive screening assays in which neutralizing antibodies capable of binding a polypeptide of the invention specifically compete with a test compound for binding to the polypeptide. In this manner, the antibodies can be used to detect the presence of any peptide that shares one or more antigenic determinants with nGPCR-x. Radiolabeled competitive binding studies are described in A.H. Lin et al. Antimicrobial Agents and Chemotherapy, 1997, vol. 41, no. 10. pp. 2127-2131, the disclosure of which is incorporated herein by reference in its entirety.

Identification of modulating agents

The invention also provides methods for identifying a modulator of binding between a nGPCR-x and a nGPCR-x binding partner, comprising the steps of: (a) contacting a nGPCR-x binding partner and a composition comprising a nGPCR-x in the presence and in the absence of a putative modulator compound; (b) detecting binding between the binding partner and the nGPCR-x; and (c) identifying a putative modulator compound or a modulator compound in view of decreased or increased binding between the binding partner and the nGPCR-x in the presence of the putative modulator, as compared to binding in the absence of the putative modulator. Following steps (a) and (b), compounds identified as modulating binding between nGPCR-x and a nGPCR-x binding partner may be tested in other assays including, but not limited to, in vivo models, to confirm or quantitate modulation of binding to nGPCR-x.

nGPCR-x binding partners that stimulate nGPCR-x activity are useful as agonists in disease states or conditions characterized by insufficient nGPCR-x signaling (e.g., as a result of insufficient activity of a nGPCR-x ligand). nGPCR-x binding partners that block ligand-mediated nGPCR-x signaling are useful as nGPCR-x antagonists to treat disease states or conditions characterized by excessive nGPCR-x signaling. In addition nGPCR-x modulators in general, as well as nGPCR-x polynucleotides and polypeptides, are useful in diagnostic assays for such diseases or conditions.

In another aspect, the invention provides methods for treating a disease or abnormal condition by administering to a patient in need of such treatment a substance that modulates the activity or expression of a polypeptide having sequences selected from the group consisting of SEQ ID NO:59 to SEQ ID NO:116.

Agents that modulate (i.e., increase, decrease, or block) nGPCR-x activity or expression may be identified by incubating a putative modulator with a cell containing a nGPCR-x polypeptide or polynucleotide and determining the effect of the putative modulator on nGPCR-x activity or expression. The selectivity of a compound that modulates the activity of nGPCR-x can be evaluated by comparing its effects on nGPCR-x to its effect on other GPCR compounds. Following identification of compounds that modulate nGPCR-x activity or expression, such compounds may be further tested in other assays including, but not limited to, in vivo models, in order to confirm or quantitate their activity. Selective modulators may include, for example, antibodies and other proteins, peptides, or organic molecules that specifically bind to a nGPCR-x polypeptide or a nGPCR-x-encoding nucleic acid. Modulators of nGPCR-x activity will be therapeutically useful in treatment of diseases and physiological conditions in which normal or aberrant nGPCR-x activity is involved. nGPCR-x polynucleotides, polypeptides, and modulators may be used in the treatment of such diseases and conditions as infections, such as viral infections caused by HIV-1 or HIV-2; pain; cancers; metabolic and cardiovascular diseases and disorders (e.g., type 2 diabetes, impaired glucose tolerance, dyslipidemia, obesity, anorexia, hypotension, hypertension, thrombosis, myocardial infarction, cardiomyopathies, atherosclerosis, etc.); Parkinson's disease; and psychotic and neurological disorders, including schizophrenia, migraine, ADHH, major depression, anxiety, mental disorder, manic depression, delirium, dementia, severe mental retardation and dyskinesias, such as Huntington's disease or Tourette's Syndrome, among others. nGPCR-x polynucleotides and polypeptides, as well as nGPCR-x modulators, may also be used in diagnostic assays for such diseases or conditions.

Methods of the invention to identify modulators include variations on any of the methods described above to identify binding partner compounds, the variations including techniques wherein a binding partner compound has been identified and the binding assay is carried out in the presence and absence of a candidate modulator. A modulator is identified in those instances where binding between the nGPCR-x polypeptide and the binding partner compound changes in the presence of the candidate modulator compared to binding in the absence of the candidate modulator compound. A modulator that increases binding between the nGPCR-x polypeptide and the binding partner compound is described as an enhancer or activator, and a modulator that decreases binding between the nGPCR-x polypeptide and the binding partner compound is described as an inhibitor. Following identification of modulators, such compounds may be further tested in other assays including, but not limited to, in vivo models, in order to confirm or quantitate their activity as modulators.

The invention also comprehends high-throughput screening (HTS) assays to identify compounds that interact with or inhibit biological activity (i.e., affect enzymatic activity, binding activity, etc.) of a nGPCR-x polypeptide. HTS assays permit screening of large numbers of compounds in an efficient manner. Cell-based HTS systems are contemplated to investigate nGPCR-x receptor-ligand interaction. HTS assays are designed to identify “hits” or “lead compounds” having the desired property, from which modifications can be designed to improve the desired property. Chemical modification of the “hit” or “lead compound” is often based on an identifiable structure/activity relationship between the “hit” and the nGPCR-x polypeptide.

Another aspect of the present invention is directed to methods of identifying compounds which modulate (i.e., increase or decrease) an activity of nGPCR-x comprising contacting nGPCR-x with a compound, and determining whether the compound modifies activity of nGPCR-x. The activity in the presence of the test compared is measured to the activity in the absence of the test compound. Where the activity of the sample containing the test compound is higher than the activity in the sample lacking the test compound, the compound will have increased activity. Similarly, where the activity of the sample containing the test compound is lower than the activity in the sample lacking the test compound, the compound will have inhibited activity. Following the identification of compounds that modulate an activity of nGPCR-x, such compounds can be further tested in other assays including, but not limited to, in vivo models, in order to confirm or quantitate their activity.

The present invention is particularly useful for screening compounds by using nGPCR-x in any of a variety of drug screening techniques. The compounds to be screened include (which may include compounds which are suspected to modulate nGPCR-x activity), but are not limited to, extracellular, intracellular, biologic or chemical origin. The nGPCR-x polypeptide employed in such a test may be in any form, preferably, free in solution, attached to a solid support, borne on a cell surface or located intracellularly. One skilled in the art can, for example, measure the formation of complexes between nGPCR-x and the compound being tested. Alternatively, one skilled in the art can examine the diminution in complex formation between nGPCR-x and its substrate caused by the compound being tested.

The activity of nGPCR-x polypeptides of the invention can be determined by, for example, examining the ability to bind or be activated by chemically synthesized peptide ligands. Alternatively, the activity of nGPCR-x polypeptides can be assayed by examining their ability to bind calcium ions, hormones, chemokines, neuropeptides, neurotransmitters, nucleotides, lipids, odorants, and photons. Alternatively, the activity of the nGPCR-x polypeptides can be determined by examining the activity of effector molecules including, but not limited to, adenylate cyclase, phospholipases and ion channels. Thus, modulators of nGPCR-x polypeptide activity may alter a GPCR receptor function, such as a binding property of a receptor or an activity such as G protein-mediated signal transduction or membrane localization. In various embodiments of the method, the assay may take the form of an ion flux assay, a yeast growth assay, a non-hydrolyzable GTP assay such as a [³⁵S]-GTP γS assay, a cAMP assay, an inositol triphosphate assay, a diacylglycerol assay, an Aequorin assay, a Luciferase assay, a FLIPR assay for intracellular Ca²⁺ concentration, a mitogenesis assay, a MAP Kinase activity assay, an arachidonic acid release assay (e.g., using [³H]-arachidonic acid), and an assay for extracellular acidification rates, as well as other binding or function-based assays of nGPCR-x activity that are generally known in the art. In several of these embodiments, the invention comprehends the inclusion of any of the G proteins known in the art, such as G ₁₆, G ₁₅, or chimeric G_{qd5}, G_{qs5}, G_{qos}, G_{q25}, and the like. nGPCR-x activity can be determined by methodologies that are used to assay for FaRP activity, which is well known to those skilled in the art. Biological activities of nGPCR-x receptors according to the invention include, but are not limited to, the binding of a natural or an unnatural ligand, as well as any one of the functional activities of GPCRs known in the art. Non-limiting examples of GPCR activities include transmembrane signaling of various forms, which may involve G protein association and/or the exertion of an influence over G protein binding of various guanidylate nucleotides; another exemplary activity of GPCRs is the binding of accessory proteins or polypeptides that differ from known G proteins.

The modulators of the invention exhibit a variety of chemical structures, which can be generally grouped into non-peptide mimetics of natural GPCR receptor ligands, peptide and non-peptide allosteric effectors of GPCR receptors, and peptides that may function as activators or inhibitors (competitive, uncompetitive and non-competitive) (e.g., antibody products) of GPCR receptors. The invention does not restrict the sources for suitable modulators, which may be obtained from natural sources such as plant, animal or mineral extracts, or non-natural sources such as small molecule libraries, including the products of combinatorial chemical approaches to library construction, and peptide libraries. Examples of peptide modulators of GPCR receptors exhibit the following primary structures: GLGPRPLRFamide, GNSFLRFamide, GGPQGPLRFamide, GPSGPLRFamide, PDVDHVFLRFamide, and pyro-EDVDHVFLRFamide.

Other assays can be used to examine enzymatic activity including, but not limited to, photometric, radiometric, HPLC, electrochemical, and the like, which are described in, for example, Enzyme Assays: A Practical Approach, eds. R. Eisenthal and M. J. Danson, 1992, Oxford University Press, which is incorporated herein by reference in its entirety.

The use of cDNAs encoding GPCRs in drug discovery programs is well-known; assays capable of testing thousands of unknown compounds per day in high-throughput screens (HTSs) are thoroughly documented. The literature is replete with examples of the use of radiolabeled ligands in HTS binding assays for drug discovery (see Williams, Medicinal Research Reviews, 1991, 11, 147-184.; Sweetnam, et al., J. Natural Products, 1993, 56, 441-455 for review). Recombinant receptors are preferred for binding assay HTS because they allow for better specificity (higher relative purity), provide the ability to generate large amounts of receptor material, and can be used in a broad variety of formats (see Hodgson, Bio/Technology, 1992, 10, 973-980; each of which is incorporated herein by reference in its entirety).

A variety of heterologous systems is available for functional expression of recombinant receptors that are well known to those skilled in the art. Such systems include bacteria (Strosberg, et al., Trends in Pharmacological Sciences, 1992, 13, 95-98), yeast (Pausch, Trends in Biotechnology, 1997, 15, 487-494), several kinds of insect cells (Vanden Broeck, Int. Rev. Cytology, 1996, 164, 189-268), amphibian cells (Jayawickreme et al., Current Opinion in Biotechnology, 1997, 8, 629-634) and several mammalian cell lines (CHO, HEK-293, COS, etc.; see Gerhardt, et al., Eur. J. Pharmacology, 1997, 334, 1-23). These examples do not preclude the use of other possible cell expression systems, including cell lines obtained from nematodes (PCT application WO 98/37177).

In preferred embodiments of the invention, methods of screening for compounds that modulate nGPCR-x activity comprise contacting test compounds with nGPCR-x and assaying for the presence of a complex between the compound and nGPCR-x. In such assays, the ligand is typically labeled. After suitable incubation, free ligand is separated from that present in bound form, and the amount of free or uncomplexed label is a measure of the ability of the particular compound to bind to nGPCR-x.

It is well known that activation of heterologous receptors expressed in recombinant systems results in a variety of biological responses, which are mediated by G proteins expressed in the host cells. Occupation of a GPCR by an agonist results in exchange of bound GDP for GTP at a binding site on the Gc, subunit; one can use a radioactive, non-hydrolyzable derivative of GTP, GTPγ[³⁵S], to measure binding of an agonist to the receptor (Sim et al., Neuroreport, 1996, 7, 729-733). One can also use this binding to measure the ability of antagonists to bind to the receptor by decreasing binding of GTPγ[³⁵S] in the presence of a known agonist. One could therefore construct a HTS based on GTPγ[³⁵S] binding, though this is not the preferred method.

The G proteins required for functional expression of heterologous GPCRs can be native constituents of the host cell or can be introduced through well-known recombinant technology. The G proteins can be intact or chimeric. Often, a nearly universally competent G protein (e.g., G_{α16}) is used to couple any given receptor to a detectable response pathway. G protein activation results in the stimulation or inhibition of other native proteins, events that can be linked to a measurable response.

Examples of such biological responses include, but are not limited to, the following: the ability to survive in the absence of a limiting nutrient in specifically engineered yeast cells (Pausch, Trends in Biotechnology, 1997, 15, 487-494); changes in intracellular Ca2, concentration as measured by fluorescent dyes (Murphy, et al., Cur. Opinion Drug Disc. Dev., 1998, 1, 192-199). Fluorescence changes can also be used to monitor ligand-induced changes in membrane potential or intracellular pH; an automated system suitable for HTS has been described for these purposes (Schroeder, et al., J. Biomolecular Screening, 1996, 1, 75-80). Melanophores prepared from Xenopus laevis show a ligand-dependent change in pigment organization in response to heterologous GPCR activation; this response is adaptable to HTS formats (Jayawickreme et al., Cur. Opinion Biotechnology, 1997, 8, 629-634). Assays are also available for the measurement of common second messengers, including cAMP, phosphoinositides and arachidonic acid, but these are not generally preferred for HTS.

Preferred methods of HTS employing these receptors include permanently transfected CHO cells, in which agonists and antagonists can be identified by the ability to specifically alter the binding of GTPγ[³⁵S] in membranes prepared from these cells. In another embodiment of the invention, permanently transfected CHO cells could be used for the preparation of membranes which contain significant amounts of the recombinant receptor proteins; these membrane preparations would then be used in receptor binding assays, employing the radiolabeled ligand specific for the particular receptor. Alternatively, a functional assay, such as fluorescent monitoring of ligand-induced changes in internal Ca²⁺ concentration or membrane potential in permanently transfected CHO cells containing each of these receptors individually or in combination would be preferred for HTS. Equally preferred would be an alternative type of mammalian cell, such as HEK-293 or COS cells, in similar formats. More preferred would be permanently transfected insect cell lines, such as Drosophila S2 cells. Even more preferred would be recombinant yeast cells expressing the Drosophila melanogaster receptors in HTS formats well known to those skilled in the art (e.g., Pausch, Trends in Biotechnology, 1997, 15, 487-494).

The invention contemplates a multitude of assays to screen and identify inhibitors of ligand binding to nGPCR-x receptors. In one example, the nGPCR-x receptor is immobilized and interaction with a binding partner is assessed in the presence and absence of a candidate modulator such as an inhibitor compound. In another example, interaction between the nGPCR-x receptor and its binding partner is assessed in a solution assay, both in the presence and absence of a candidate inhibitor compound. In either assay, an inhibitor is identified as a compound that decreases binding between the nGPCR-x receptor and its binding partner. Following the identification of compounds which inhibit ligand binding to nGPCR-x receptors, such compounds may be further tested in other assays including, but not limited to, in vivo models, in order to confirm or quantitate their activity. Another contemplated assay involves a variation of the dihybrid assay wherein an inhibitor of protein/protein interactions is identified by detection of a positive signal in a transformed or transfected host cell, as described in PCT publication number WO 95/20652, published Aug. 3, 1995.

Candidate modulators contemplated by the invention include compounds selected from libraries of either potential activators or potential inhibitors. There are a number of different libraries used for the identification of small molecule modulators, including: (1) chemical libraries, (2) natural product libraries, and (3) combinatorial libraries comprised of random peptides, oligonucleotides or organic molecules. Chemical libraries consist of random chemical structures, some of which are analogs of known compounds or analogs of compounds that have been identified as “hits” or “leads” in other drug discovery screens, some of which are derived from natural products, and some of which arise from non-directed synthetic organic chemistry. Natural product libraries are collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282:63-68 (1998). Combinatorial libraries are composed of large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of modulators through use of the various libraries described herein permits modification of the candidate “hit” (or “lead”) to optimize the capacity of the “hit” to modulate activity.

Still other candidate inhibitors contemplated by the invention can be designed and include soluble forms of binding partners, as well as such binding partners as chimeric, or fusion, proteins. A “binding partner” as used herein broadly encompasses non-peptide modulators, as well as such peptide modulators as neuropeptides other than natural ligands, antibodies, antibody fragments, and modified compounds comprising antibody domains that are immunospecific for the expression product of the identified nGPCR-x gene.

The polypeptides of the invention are employed as a research tool for identification, characterization and purification of interacting, regulatory proteins. Appropriate labels are incorporated into the polypeptides of the invention by various methods known in the art and the polypeptides are used to capture interacting molecules. For example, molecules are incubated with the labeled polypeptides, washed to remove unbound polypeptides, and the polypeptide complex is quantified. Data obtained using different concentrations of polypeptide are used to calculate values for the number, affinity, and association of polypeptide with the protein complex.

Labeled polypeptides are also useful as reagents for the purification of molecules with which the polypeptide interacts including, but not limited to, inhibitors. In one embodiment of affinity purification, a polypeptide is covalently coupled to a chromatography column. Cells and their membranes are extracted, and various cellular subcomponents are passed over the column. Molecules bind to the-column by virtue of their affinity to the polypeptide. The polypeptide-complex is recovered from the column, dissociated and the recovered molecule is subjected to protein sequencing. This amino acid sequence is then used to identify the captured molecule or to design degenerate oligonucleotides for cloning the corresponding gene from an appropriate cDNA library.

Alternatively, compounds may be identified which exhibit similar properties to the ligand for the nGPCR-x of the invention, but which are smaller and exhibit a longer half time than the endogenous ligand in a human or animal body. When an organic compound is designed, a molecule according to the invention is used as a “lead” compound. The design of mimetics to known pharmaceutically active compounds is a well-known approach in the development of pharmaceuticals based on such “lead” compounds. Mimetic design, synthesis and testing are generally used to avoid randomly screening a large number of molecules for a target property. Furthermore, structural data deriving from the analysis of the deduced amino acid sequences encoded by the DNAs of the present invention are useful to design new drugs, more specific and therefore with a higher pharmacological potency.

Comparison of the protein sequence of the present invention with the sequences present in all the available databases showed a significant homology with the transmembrane portion of G protein coupled receptors. Accordingly, computer modeling can be used to develop a putative tertiary structure of the proteins of the invention based on the available information of the transmembrane domain of other proteins. Thus, novel ligands based on the predicted structure of nGPCR-x can be designed.

In a particular embodiment, the novel molecules identified by the screening methods according to the invention are low molecular weight organic molecules, in which case a composition or pharmaceutical composition can be prepared thereof for oral intake, such as in tablets. The compositions, or pharmaceutical compositions, comprising the nucleic acid molecules, vectors, polypeptides, antibodies and compounds identified by the screening methods described herein, can be prepared for any route of administration including, but not limited to, oral, intravenous, cutaneous, subcutaneous, nasal, intramuscular or intraperitoneal. The nature of the carrier or other ingredients will depend on the specific route of administration and particular embodiment of the invention to be administered. Examples of techniques and protocols that are useful in this context are, inter alia, found in Remington's Pharmaceutical Sciences, 16^{th} edition, Osol, A (ed.), 1980, which is incorporated herein by reference in its entirety.

The dosage of these low molecular weight compounds will depend on the disease state or condition to be treated and other clinical factors such as weight and condition of the human or animal and the route of administration of the compound. For treating human or animals, between approximately 0.5 mg/kg of body weight to 500 mg/kg of body weight of the compound can be administered. Therapy is typically administered at lower dosages and is continued until the desired therapeutic outcome is observed.

The present compounds and methods, including nucleic acid molecules, polypeptides, antibodies, compounds identified by the screening methods described herein, have a variety of pharmaceutical applications and may be used, for example, to treat or prevent unregulated cellular growth, such as cancer cell and tumor growth. In a particular embodiment, the present molecules are used in gene therapy. For a review of gene therapy procedures, see e.g. Anderson, Science, 1992, 256, 808-813, which is incorporated herein by reference in its entirety.

The present invention also encompasses a method of agonizing (stimulating) or antagonizing a nGPCR-x natural binding partner associated activity in a mammal comprising administering to said mammal an agonist or antagonist to one of the above disclosed polypeptides in an amount sufficient to effect said agonism or antagonism. One embodiment of the present invention, then, is a method of treating diseases in a mammal with an agonist or antagonist of the protein of the present invention comprises administering the agonist or antagonist to a mammal in an amount sufficient to agonize or antagonize nGPCR-x-associated functions.

In an effort to discover novel treatments for diseases, biomedical researchers and chemists have designed, synthesized, and tested molecules that modulate the function of G protein coupled receptors. Some small organic molecules form a class of compounds that modulate the function of G protein coupled receptors.

Exemplary diseases and conditions amenable to treatment based on the present invention include, but are not limited to, thyroid disorders (e.g. thyreotoxicosis, myxoedema); renal failure; inflammatory conditions (e.g., Chron's disease); diseases related to cell differentiation and homeostasis; rheumatoid arthritis; autoimmune disorders; movement disorders; CNS disorders (e.g., pain including migraine; stroke; psychotic and neurological disorders, including anxiety, mental disorder, manic depression, anxiety, generalized anxiety disorder, post-traumatic-stress disorder, depression, bipolar disorder, delirium, dementia, severe mental retardation; dyskinesias, such as Huntington's disease or Tourette's Syndrome; attention disorders including ADD and ADHD, and degenerative disorders such as Parkinson's, Alzheimer's; movement disorders, including ataxias, supranuclear palsy, etc.); infections, such as viral infections caused by HIV-1 or HIV-2; metabolic and cardiovascular diseases and disorders (e.g., type 2 diabetes, impaired glucose tolerance, dyslipidemia, obesity, anorexia, hypotension, hypertension, thrombosis, myocardial infarction, cardiomyopathies, atherosclerosis, etc.); proliferative diseases and cancers (e.g., different cancers such as breast, colon, lung, etc., and hyperproliferative disorders such as psoriasis, prostate hyperplasia, etc.); hormonal disorders (e.g., male/female hormonal replacement, polycystic ovarian syndrome, alopecia, etc.); sexual dysfunction, among others.

Methods of determining the dosages of compounds to be administered to a patient and modes of administering compounds to an organism are disclosed in U.S. application Ser. No. 08/702,282, filed Aug. 23, 1996 and International patent publication number WO 96/22976, published Aug. 1, 1996, both of which are incorporated herein by reference in their entirety, including any drawings, figures or tables. Those skilled in the art will appreciate that such descriptions are applicable to the present invention and can be easily adapted to it.

The proper dosage depends on various factors such as the type of disease being treated, the particular composition being used and the size and physiological condition of the patient. Therapeutically effective doses for the compounds described herein can be estimated initially from cell culture and animal models. For example, a dose can be formulated in animal models to achieve a circulating concentration range that initially takes into account the IC₅₀ as determined in cell culture assays. The animal model data can be used to more accurately determine useful doses in humans.

Plasma half-life and biodistribution of the drug and metabolites in the plasma, tumors and major organs can also be determined to facilitate the selection of drugs most appropriate to inhibit a disorder. Such measurements can be carried out. For example, HPLC analysis can be performed on the plasma of animals treated with the drug and the location of radiolabeled compounds can be determined using detection methods such as X-ray, CAT scan and MRI. Compounds that show potent inhibitory activity in the screening assays, but have poor pharmacokinetic characteristics, can be optimized by altering the chemical structure and retesting. In this regard, compounds displaying good pharmacokinetic characteristics can be used as a model.

Toxicity studies can also be carried out by measuring the blood cell composition. For example, toxicity studies can be carried out in a suitable animal model as follows: 1) the compound is administered to mice (an untreated control mouse should also be used); 2) blood samples are periodically obtained via the tail vein from one mouse in each treatment group; and 3) the samples are analyzed for red and white blood cell counts, blood cell composition and the percent of lymphocytes versus polymorphonuclear cells. A comparison of results for each dosing regime with the controls indicates if toxicity is present.

At the termination of each toxicity study, further studies can be carried out by sacrificing the animals (preferably, in accordance with the American Veterinary Medical Association guidelines Report of the American Veterinary Medical Assoc. Panel on Euthanasia, Journal of American Veterinary Medical Assoc., 202:229-249, 1993). Representative animals from each treatment group can then be examined by gross necropsy for immediate evidence of metastasis, unusual illness or toxicity. Gross abnormalities in tissue are noted and tissues are examined histologically. Compounds causing a reduction in body weight or blood components are less preferred, as are compounds having an adverse effect on major organs. In general, the greater the adverse effect the less preferred the compound.

For the treatment of many diseases, the expected daily dose of a hydrophobic pharmaceutical agent is between 1 to 500 mg/day, preferably 1 to 250 mg/day, and most preferably 1 to 50 mg/day. Drugs can be delivered less frequently provided plasma levels of the active moiety are sufficient to maintain therapeutic effectiveness. Plasma levels should reflect the potency of the drug. Generally, the more potent the compound the lower the plasma levels necessary to achieve efficacy.

As discussed above, it is well known that GPCRs are expressed in many different tissues and regions, including in the brain. nGPCR-x MRNA transcripts may found in many other tissues, including, but not limited to peripheral blood lymphocytes, pancreas, ovary, uterus, testis, salivary gland, kidney, adrenal gland, liver, bone marrow, prostate, fetal liver, colon, muscle, and fetal brain, and may be found in many other tissues. Within the brain, nGPCR-x mRNA transcripts may be found in many tissues, including, but not limited to, frontal lobe, hypothalamus, pons, cerebellum, cerebrum, caudate nucleus, and medulla.

Sequences selected from the group consisting of SEQ ID NO: 1 to SEQ ID NO:58 will, as detailed above, enable screening the endogenous neurotransmitterslhormones/ligands which activate, agonize, or antagonize nGPCR-x and for compounds with potential utility in treating disorders including, but not limited to, thyroid disorders (e.g. thyreotoxicosis, myxoedema); renal failure; inflammatory conditions (e.g., Chron's disease); diseases related to cell differentiation and homeostasis; rheumatoid arthritis; autoimmune disorders; movement disorders; CNS disorders (e.g., pain including schizophrenia, migraine; stroke; psychotic and neurological disorders, including anxiety, mental disorder, manic depression, anxiety, generalized anxiety disorder, post-traumatic-stress disorder, depression, bipolar disorder, delirium, dementia, severe mental retardation; dyskinesias, such as Huntington's disease or Tourette's Syndrome; attention disorders including ADD and ADHD, and degenerative disorders such as Parkinson's, Alzheimer's; movement disorders, including ataxias, supranuclear palsy, etc.); infections, such as viral infections caused by HIV-1 or HIV-2; metabolic and cardiovascular diseases and disorders (e.g., type 2 diabetes, impaired glucose tolerance, dyslipidemia, obesity, anorexia, hypotension, hypertension, thrombosis, myocardial infarction, cardiomyopathies, atherosclerosis, etc.); proliferative diseases and cancers (e.g., different cancers such as breast, colon, lung, etc., and hyperproliferative disorders such as psoriasis, prostate hyperplasia, etc.); hormonal disorders (e.g., male/female hormonal replacement, polycystic ovarian syndrome, alopecia, etc.); sexual dysfunction, among others.

For example, nGPCR-x may be useful in the treatment of respiratory ailments such as asthma, where T cells are implicated by the disease. Contraction of airway smooth muscle is stimulated by thrombin. Cicala et al (1999) Br J Pharmacol 126:478-484. Additionally, in bronchiolitis obliterans, it has been noted that activation of thrombin receptors may be deleterious. Hauck et al. (1999) Am J Physiol 277:L22-L29. Furthermore, mast cells have also been shown to have thrombin receptors. Cirino et al (1996) J Exp Med 183:821-827. nGPCR-x may also be useful in remodeling of airway structure s in chronic pulmonary inflammation via stimulation of fibroblast procollagen synthesis. See, e.g., Chambers et al. (1998) Biochem J 333:121-127; Trejo et al. (1996) J Biol Chem 271:21536-21541.

In another example, increased release of sCD40L and expression of CD40L by T cells after activation of thrombin receptors suggests that nGPCR-x may be useful in the treatment of unstable angina due to the role of T cells and inflammation. See Aukrust et al. (1999) Circulation 100:614-620.

A further example is the treatment of inflammatory diseases, such as psoriasis, inflammatory bowel disease, multiple sclerosis, rheumatoid arthritis, and thyroiditis. Due to the tissue expression profile of nGPCR-x, inhibition of thrombin receptors may be beneficial for these diseases. See, e.g., Morris et al. (1996) Ann Rheum Dis 55:841-843. In addition to T cells, NK cells and monocytes are also critical cell types which contribute to the pathogenesis of these diseases. See, e.g., Naldini & Carney (1996) Cell Immunol 172:35-42; Hoffman & Cooper (1995) Blood Cells Mol Dis 21:156-167; Colotta et al. (1994) Am J Pathol 144:975-985.

Expression of nGPCR-x in bone marrow and spleen may suggest that it may play a role in the proliferation of hematopoietic progenitor cells. See DiCuccio et al. (1996) Exp Hematol 24:914-918.

As another example, nGPCR-x may be useful in the treatment of acute and/or traumatic brain injury. Astrocytes have been demonstrated to express thrombin receptors. Activation of thrombin receptors may be involved in astrogliosis following brain injury. Therefore, inhibition of receptor activity may be beneficial for limiting neuroinflammation. Scar formation mediated by astrocytes may also be limited by inhibiting thrombin receptors. See, e.g, Pindon et al. (1998) Eur J Biochem 255:766-774; Ubl & Reiser. (1997) Glia 21:361-369; Grabham & Cunningham (1995) J Neurochem 64:583-591.

nGPCR-x receptor activation may mediate neuronal and astrocyte apoptosis and prevention of neurite outgrowth. Inhibition would be beneficial in both chronic and acute brain injury. See, e.g., Donovan et al. (1997) J Neurosci 17:5316-5326; Turgeon et al (1998) J Neurosci 18:6882-6891; Smith-Swintosky et al. (1997) J Neurochem 69:1890-1896; Gill et al. (1998) Brain Res 797:321-327; Suidan et al. (1996) Semin Thromb Hemost 22:125-133.

The attached Sequence Listing contains the sequences of the polynucleotides and polypeptides of the invention and is incorporated herein by reference in its entirety.

Methods of Screening Human Subjects

Thus in yet another embodiment, the invention provides genetic screening procedures that entail analyzing a person's genome—particular their alleles for the nGPCR-x of the invention—to determine whether the individual possesses a genetic characteristic found in other individuals that are considered to be afflicted with, or at risk for, developing a mental disorder or disease of the brain that is suspected of having a hereditary component. For example, in one embodiment, the invention provides a method for determining a potential for developing a disorder affecting the brain in a human subject comprising the steps of analyzing the coding sequence of one or more nGPCR-x genes from the human subject; and determining development potential for the disorder in said human subject from the analyzing step.

More particularly, the invention provides a method of screening a human subject to diagnose a disorder affecting the brain or genetic predisposition therefor, comprising the steps of: (a) assaying nucleic acid of a human subject to determine a presence or an absence of a mutation altering the amino acid sequence, expression, or biological activity of at least one seven transmembrane receptor that is expressed in the brain, wherein the seven transmembrane receptor comprises an amino acid sequence selected from the group consisting of SEQ ID NO: 1 to SEQ ID NO:58, or an allelic variant thereof, and wherein the nucleic acid corresponds to the gene encoding the seven transmembrane receptor; and (b) diagnosing the disorder or predisposition from the presence or absence of said mutation, wherein the presence of a mutation altering the amino acid sequence, expression, or biological activity of allele in the nucleic acid correlates with an increased risk of developing the disorder.

By “human subject” is meant any human being, human embryo, or human fetus. It will be apparent that methods of the present invention will be of particular interest to individuals that have themselves been diagnosed with a disorder affecting the brain or have relatives that have been diagnosed with a disorder affecting the brain.

By “screening for an increased risk” is meant determination of whether a genetic variation exists in the human subject that correlates with a greater likelihood of developing a disorder affecting the brain than exists for the human population as a whole, or for a relevant racial or ethnic human sub-population to which the individual belongs. Both positive and negative determinations (i.e., determinations that a genetic predisposition marker is present or is absent) are intended to fall within the scope of screening methods of the invention. In preferred embodiments, the presence of a mutation altering the sequence or expression of at least one nGPCR-x seven transmembrane receptor allele in the nucleic acid is correlated with an increased risk of developing mental disorder, whereas the absence of such a mutation is reported as a negative determination.

The “assaying” step of the invention may involve any techniques available for analyzing nucleic acid to determine its characteristics, including but not limited to well-known techniques such as single-strand conformation polymorphism analysis (SSCP) [Orita et al., Proc Natl. Acad. Sci. USA, 86: 2766-2770 (1989)]; heteroduplex analysis [White et al., Genomics, 12: 301-306 (1992)]; denaturing gradient gel electrophoresis analysis [Fischer et al., Proc. Natl. Acad. Sci. USA, 80: 1579-1583 (1983); and Riesner et al., Electrophoresis, 10: 377-389 (1989)]; DNA sequencing; RNase cleavage [Myers et al., Science, 230: 1242-1246 (1985)]; chemical cleavage of mismatch techniques [Rowley et al., Genomics, 30: 574-582 (1995); and Roberts et al., Nucl. Acids Res., 25: 3377-3378 (1997)]; restriction fragment length polymorphism analysis; single nucleotide primer extension analysis [Shumaker et al., Hum. Mutat., 7: 346-354 (1996); and Pastinen et al., Genome Res., 7: 606-614 (1997)]; 5′ nuclease assays [Pease et al., Proc. Natl. Acad. Sci. USA, 91:5022-5026 (1994)]; DNA Microchip analysis [Ramsay, G., Nature Biotechnology, 16: 40-48 (1999); and Chee et al., U.S. Pat. No. 5,837,832]; and ligase chain reaction [Whiteley et al., U.S. Pat. No. 5,521,065]. [See generally, Schafer and Hawkins, Nature Biotechnology, 16: 33-39 (1998).] All of the foregoing documents are hereby incorporated by reference in their entirety.

Thus, in one preferred embodiment involving screening nGPCR-x sequences, for example, the assaying step comprises at least one procedure selected from the group consisting of: (a) determining a nucleotide sequence of at least one codon of at least one nGPCR-x allele of the human subject; (b) performing a hybridization assay to determine whether nucleic acid from the human subject has a nucleotide sequence identical to or different from one or more reference sequences; (c) performing a polynucleotide migration assay to determine whether nucleic acid from the human subject has a nucleotide sequence identical to or different from one or more reference sequences; and (d) performing a restriction endonuclease digestion to determine whether nucleic acid from the human subject has a nucleotide sequence identical to or different from one or more reference sequences.

In a highly preferred embodiment, the assaying involves sequencing of nucleic acid to determine nucleotide sequence thereof, using any available sequencing technique. [See, e.g., Sanger et al., Proc. Natl. Acad. Sci. (USA), 74: 5463-5467 (1977) (dideoxy chain termination method); Mirzabekov, TIBTECH, 12: 27-32 (1994) (sequencing by hybridization); Drmanac et al., Nature Biotechnology, 16: 54-58 (1998); U.S. Pat. No. 5,202,231; and Science, 260: 1649-1652 (1993) (sequencing by hybridization); Kieleczawa et al., Science, 258: 1787-1791 (1992) (sequencing by primer walking); (Douglas et al., Biotechniques, 14: 824-828 (1993) (Direct sequencing of PCR products); and Akane et al., Biotechniques 16: 238-241 (1994); Maxam and Gilbert, Meth. Enzymol., 65: 499-560 (1977) (chemical termination sequencing), all incorporated herein by reference.] The analysis may entail sequencing of the entire nGPCR gene genomic DNA sequence, or portions thereof; or sequencing of the entire seven transmembrane receptor coding sequence or portions thereof. In some circumstances, the analysis may involve a determination of whether an individual possesses a particular allelic variant, in which case sequencing of only a small portion of nucleic acid—enough to determine the sequence of a particular codon characterizing the allelic variant—is sufficient. This approach is appropriate, for example, when assaying to determine whether one family member inherited the same allelic variant that has been previously characterized for another family member, or, more generally, whether a person's genome contains an allelic variant that has been previously characterized and correlated with a mental disorder having a heritable component.

In another highly preferred embodiment, the assaying step comprises performing a hybridization assay to determine whether nucleic acid from the human subject has a nucleotide sequence identical to or different from one or more reference sequences. In a preferred embodiment, the hybridization involves a determination of whether nucleic acid derived from the human subject will hybridize with one or more oligonucleotides, wherein the oligonucleotides have nucleotide sequences that correspond identically to a portion of the nGPCR-x gene sequence taught herein, or that correspond identically except for one mismatch. The hybridization conditions are selected to differentiate between perfect sequence complementarity and imperfect matches differing by one or more bases. Such hybridization experiments thereby can provide single nucleotide polymorphism sequence information about the nucleic acid from the human subject, by virtue of knowing the sequences of the oligonucleotides used in the experiments.

Several of the techniques outlined above involve an analysis wherein one performs a polynucleotide migration assay, e.g., on a polyacrylamide electrophoresis gel (or in a capillary electrophoresis system), under denaturing or non-denaturing conditions. Nucleic acid derived from the human subject is subjected to gel electrophoresis, usually adjacent to (or co-loaded with) one or more reference nucleic acids, such as reference GPCR-x encoding sequences having a coding sequence identical to all or a portion of SEQ ID NO: 1 to SEQ ID NO:58 (or identical except for one known polymorphism). The nucleic acid from the human subject and the reference sequence(s) are subjected to similar chemical or enzymatic treatments and then electrophoresed under conditions whereby the polynucleotides will show a differential migration pattern, unless they contain identical sequences. [See generally Ausubel et al. (eds.), Current Protocols in Molecular Biology, New York: John Wiley & Sons, Inc. (1987-1999); and Sambrook et al., (eds.), Molecular Cloning, A Laboratory Manual, Cold Spring Harbor, New York: Cold Spring Harbor Laboratory Press (1989), both incorporated herein by reference in their entirety.]

In the context of assaying, the term “nucleic acid of a human subject” is intended to include nucleic acid obtained directly from the human subject (e.g., DNA or RNA obtained from a biological sample such as a blood, tissue, or other cell or fluid sample); and also nucleic acid derived from nucleic acid obtained directly from the human subject. By way of non-limiting examples, well known procedures exist for creating cDNA that is complementary to RNA derived from a biological sample from a human subject, and for amplifying (e.g., via polymerase chain reaction (PCR)) DNA or RNA derived from a biological sample obtained from a human subject. Any such derived polynucleotide which retains relevant nucleotide sequence information of the human subject's own DNA/RNA is intended to fall within the definition of “nucleic acid of a human subject” for the purposes of the present invention.

In the context of assaying, the term “mutation” includes addition, deletion, and/or substitution of one or more nucleotides in the GPCR gene sequence (e.g., as compared to the seven transmembrane receptor-encoding sequences set forth of SEQ ID NO: 1 to SEQ ID NO:58, and other polymorphisms that occur in introns (where introns exist) and that are identifiable via sequencing, restriction fragment length polymorphism, or other techniques. The various activity examples provided herein permit determination of whether a mutation modulates activity of the relevant receptor in the presence or absence of various test substances.

In a related embodiment, the invention provides methods of screening a person's genotype with respect to the nGPCR-x of the invention, and correlating such genotypes with diagnoses for disease or with predisposition for disease (for genetic counseling). For example, the invention provides a method of screening for an nGPCR-x hereditary mental disorder genotype in a human patient, comprising the steps of: (a) providing a biological sample comprising nucleic acid from the patient, the nucleic acid including sequences corresponding to said patient's nGPCR-x alleles; (b) analyzing the nucleic acid for the presence of a mutation or mutations; (c) determining a nGPCR-x genotype from the analyzing step; and (d) correlating the presence of a mutation in an nGPCR-x allele with a hereditary mental disorder genotype. In a preferred embodiment, the biological sample is a cell sample containing human cells that contain genomic DNA of the human subject. The analyzing can be performed analogously to the assaying described in preceding paragraphs. For example, the analyzing comprises sequencing a portion of the nucleic acid (e.g., DNA or RNA), the portion comprising at least one codon of the nGPCR-x alleles.

Although more time consuming and expensive than methods involving nucleic acid analysis, the invention also may be practiced by assaying one or more proteins of a human subject to determine the presence or absence of an amino acid sequence variation in GPCR protein from the human subject. Such protein analyses may be performed, e.g., by fragmenting GPCR protein via chemical or enzymatic methods and sequencing the resultant peptides; or by Western analyses using an antibody having specificity for a particular allelic variant of the GPCR.

The invention also provides materials that are useful for performing methods of the invention. For example, the present invention provides oligonucleotides useful as probes in the many analyzing techniques described above. In general, such oligonucleotide probes comprise 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides that have a sequence that is identical, or exactly complementary, to a portion of a human GPCR gene sequence taught herein (or allelic variant thereof), or that is identical or exactly complementary except for one nucleotide substitution. In a preferred embodiment, the oligonucleotides have a sequence that corresponds in the foregoing manner to a human GPCR coding sequence taught herein, and in particular, the coding sequences set forth in SEQ ID NO: 1 to SEQ ID NO:58. In one variation, an oligonucleotide probe of the invention is purified and isolated. In another variation, the oligonucleotide probe is labeled, e.g., with a radioisotope, chromophore, or fluorophore. In yet another variation, the probe is covalently attached to a solid support. [See generally Ausubel et al. and Sambrook et al., supra.]

In a related embodiment, the invention provides kits comprising reagents that are useful for practicing methods of the invention. For example, the invention provides a kit for screening a human subject to diagnose a mental disorder or a genetic predisposition therefor, comprising, in association: (a) an oligonucleotide useful as a probe for identifying polymorphisms in a human nGPCR-x seven transmembrane receptor gene, the oligonucleotide comprising 6-50 nucleotides that have a sequence that is identical or exactly complementary to a portion of a human nGPCR-x gene sequence or nGPCR-x coding sequence, except for one sequence difference selected from the group consisting of a nucleotide addition, a nucleotide deletion, or nucleotide substitution; and (b) a media packaged with the oligonucleotide containing information identifying polymorphisms identifiable with the probe that correlate with mental disorder or a genetic predisposition therefor. Exemplary information-containing media include printed paper package inserts or packaging labels; and magnetic and optical storage media that are readable by computers or machines used by practitioners who perform genetic screening and counseling services. The practitioner uses the information provided in the media to correlate the results of the analysis with the oligonucleotide with a diagnosis. In a preferred variation, the oligonucleotide is labeled.

In still another embodiment, the invention provides methods of identifying those allelic variants of GPCRs of the invention that correlate with mental disorders. For example, the invention provides a method of identifying a seven transmembrane allelic variant that correlates with a mental disorder, comprising steps of: (a) providing a biological sample comprising nucleic acid from a human patient diagnosed with a mental disorder, or from the patient's genetic progenitors or progeny; (b) analyzing the nucleic acid for the presence of a mutation or mutations in at least one seven transmembrane receptor that is expressed in the brain, wherein the at least one seven transmembrane receptor comprises an amino acid sequence selected from the group consisting of SEQ ID NO: 1 to SEQ ID NO:58 or an allelic variant thereof, and wherein the nucleic acid includes sequence corresponding to the gene or genes encoding the at least one seven transmembrane receptor; (c) determining a genotype for the patient for the at least one seven transmembrane receptor from said analyzing step; and (d) identifying an allelic variant that correlates with the mental disorder from the determining step. To expedite this process, it may be desirable to perform linkage studies in the patients (and possibly their families) to correlate chromosomal markers with disease states. The chromosomal localization data provided herein facilitates identifying an involved nGPCR with a chromosomal marker.

The foregoing method can be performed to correlate the nGPCR-x of the invention to a number of disorders having hereditary components that are causative or that predispose persons to the disorder. For example, in one preferred variation, the disorder is a mental disorder.

Also contemplated as part of the invention are polynucleotides that comprise the allelic variant sequences identified by such methods, and polypeptides encoded by the allelic variant sequences, and oligonucleotide and oligopeptide fragments thereof that embody the mutations that have been identified. Such materials are useful in in vitro cell-free and cell-based assays for identifying lead compounds and therapeutics for treatment of the disorders. For example, the variants are used in activity assays, binding assays, and assays to screen for activity modulators described herein. In one preferred embodiment, the invention provides a purified and isolated polynucleotide comprising a nucleotide sequence encoding a nGPCR-x receptor allelic variant identified according to the methods described above; and an oligonucleotide that comprises the sequences that differentiate the allelic variant from the nGPCR-x sequences set forth in SEQ ID NO: 1 to SEQ ID NO:58. The invention also provides a vector comprising the polynucleotide (preferably an expression vector); and a host cell transformed or transfected with the polynucleotide or vector. The invention also provides an isolated cell line that is expressing the allelic variant nGPCR-x polypeptide; purified cell membranes from such cells; purified polypeptide; and synthetic peptides that embody the allelic variation amino acid sequence. In one particular embodiment, the invention provides a purified polynucleotide comprising a nucleotide sequence encoding a nGPCR-x seven transmembrane receptor protein of a human that is affected with a mental disorder; wherein said polynucleotide hybridizes to the complement of a sequence selected from the group consisting of SEQ ID NO: 1 to SEQ ID NO:58 under the following hybridization conditions: (a) hybridization for 16 hours at 42° C. in a hybridization solution comprising 50% formamide, 1% SDS, 1 M NaCl, 10% dextran sulfate and (b) washing 2 times for 30 minutes at 60° C. in a wash solution comprising 0.1 × SSC and 1% SDS; and wherein the polynucleotide encodes a nGPCR-x amino acid sequence that differs from a sequence selected from the group consisting of SEQ ID NO:59 to SEQ ID NO: 116, by at least one residue.

An exemplary assay for using the allelic variants is a method for identifying a modulator of nGPCR-x biological activity, comprising the steps of: (a) contacting a cell expressing the allelic variant in the presence and in the absence of a putative modulator compound; (b) measuring nGPCR-x biological activity in the cell; and (c) identifying a putative modulator compound in view of decreased or increased nGPCR-x biological activity in the presence versus absence of the putative modulator.

Additional features of the invention will be apparent from the following Examples. Examples 1 and 2 are actual while the remaining Examples are prophetic. Additional features and variations of the invention will be apparent to those skilled in the art from the entirety of this application, including the detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

### EXAMPLES

### Example 1

### IDENTIFICATION OF nGPCR-X

A. Database search

The Celera database was searched using known GPCR receptors as query sequences to find patterns suggestive of novel G protein-coupled receptors. Positive hits were further analyzed with the GCG program BLAST to determine which ones were the most likely candidates to encode G protein-coupled receptors, using the standard (default) alignment produced by BLAST as a guide.

Briefly, the BLAST algorithm, which stands for Basic Local Alignment Search Tool is suitable for determining sequence similarity (Altschul et al., J. Mol. Biol., 1990, 215, 403-410, which is incorporated herein by reference in its entirety). Software for performing BLAST analyses is publicly available through the National Center for Biotechnology Information. This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands.

The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a nucleic acid is considered similar to a GPCR gene or cDNA if the smallest sum probability in comparison of the test nucleic acid to a GPCR nucleic acid is less than about 1, preferably less than about 0.1, more preferably less than about 0.01, and most preferably less than about 0.001.

Homology searches are performed with the program BLAST version 2.08. A collection of 340 query amino acid sequences derived from GPCRs was used to search the genomic DNA sequence using TBLASTN and alignments with an E-value lower than 0.01 were collected from each BLAST search. The amino acid sequences have been edited to remove regions in the sequence that produce non-significant alignments with proteins that are not related to GPCRs.

Multiple query sequences may have a significant alignment to the same genomic region, although each alignment may not cover exactly the same DNA region. A procedure is used to determine the region of maximum common overlap between the alignments from several query sequences. This region is called the consensus DNA region. The procedure for determining this consensus involves the automatic parsing of the BLAST output files using the program MSPcrunch to produce a tabular report. From this tabular report the start and end of each alignment in the genomic DNA is extracted. This information is used by a PERL script to derive the maximum common overlap. These regions are reported in the form of a unique sequence identifier, a start and the end position in the sequence. The sequences defined by these regions were extracted from the original genomic sequence file using the program fetchdb.

The consensus regions are assembled into a non-redundant set by using the program phrap. After assembly with phrap a set of contigs and singletons were defined as candidate DNA regions coding for nGPCRs. These sequences were then submitted for further sequence analysis.

Further sequence analysis involves the removal of sequences previously isolated and removal of sequences that are related to olfactory GPCR's.

nGPRCR-x cDNAs were sequenced directly using an ABI377 fluorescence-based sequencer (Perkin-Elmer/Applied Biosystems Division, PE/ABD, Foster City, Calif.) and the ABI PRISM™ Ready Dye-Deoxy Terminator kit with Taq FS™ polymerase. Each ABI cycle sequencing reaction contained about 0.5 fig of plasmid DNA. Cycle-sequencing was performed using an initial denaturation at 98° C. for 1 minute, followed by 50 cycles using the following parameters: 98° C. for 30 seconds, annealing at 50° C. for 30 seconds, and extension at 60° C. for 4 minutes. Temperature cycles and times were controlled by a Perkin-Elmer 9600 thermocycler. Extension products were purified using Centriflexm gel filtration cartridges (Advanced Genetic Technologies Corp., Gaithersburg, Md.). Each reaction product was loaded by pipette onto the column, which is then centrifuged in a swinging bucket centrifuge (Sorvall model RT6000B tabletop centrifuge) at 1500 ×g for 4 minutes at room temperature. Column-purified samples were dried under vacuum for about 40 minutes and then dissolved in 5F1 of a DNA loading solution (83% deionized formamide, 8.3mM EDTA, and 1.6 mg/ml Blue Dextran). The samples were then heated to 90° C. for three minutes and loaded into the gel sample wells for sequence analysis using the ABI377 sequencer. Sequence analysis was performed by importing ABI377 files into the Sequencer program (Gene Codes, Ann Arbor, MI). Generally, sequence reads of 700 bp were obtained. Potential sequencing errors were minimized by obtaining sequence information from both DNA strands and by re-sequencing difficult areas using primers annealing at different locations until all sequencing ambiguities were removed.

The following Table 5 contains the sequences of the polynucleotides and polypeptides of the invention. The transmembrane domains within the polypeptide sequence are identified by underlining.

TABLE 5
The following DNA sequence Seq-86 <SEQ ID NO. 1> was identified in H.
sapiens:
ACACAGTGTGCACACACGTGCAGGGACATACCCCCTTCCCCAACTGCCTGGCCTGCACACTTGGCATTTCC
AGTATTTCTAGGAAGTGATGGCTCTGTGCATCCTGAGCCAATCCAGCTCCGAGCCTCCAAGGCATCCTGGT
GATGGGCAGCTGGAAGCTCTGCCTCTGAGGCCTTCACACACCCACCTTCGGTCAAACTTGCTTCTGCTGAG
GAACTTGGTGTGTCTTCCTTCTGGGCAGGAGGTCACATTTGAGAGCACAGGAGCAGTGCCTGCCCCCCGGG
AATGTGGCTCTGGGTAGAATTGCAGGCTCAGGGGTTTTGGGCAGGAGAGCACCAACCGTGCCACACCCACA
CAGACACGGTCACTGGCGCCCTGCAGCAGGGACGACCGCACTTCCCAAAGGGCTGGGAAGCCATGTCCAGA
GGAGGCCATGCTCTAGCTCCCTTGGGCAGGGCTGGCTGCAAGGAGGGTGAAGTTGGGCATCTTGAACCCAG
AGAAGTAGAGGACTCAGCACCAGCACAACCAGCTCGGCGCATTAATACACATTCCTCTCCCACTTCTCCCC
AAGCCTGAAAAAACCTCAAACCAGCCTCTTTGCAGCTCCCTGAGGTCATGACTCACGAACCATGCTCGGGG
CAGGGGAAAAGAAAAGCATCCG
The following amino acid sequence <SEQ ID NO. 59> is the predicted
amino acid sequence derived from the DNA sequence of SEQ ID NO. 1:
DAFLFPCPEHGSVMTSGSCKEAGLRFFQAWGEVGEECVLMRRAGCAGAESSTSLGSRCPTSPSLQPALPKG
ARAWPPLDMASQPFGKCGRPCCPAPVTVSVWVWHGWCSPAQNPACNSTQSHIPGGQALLLCSQMPPAQKED
TPSSSAEASLTEGGCVKASEAELPAAHHQDALEARSWIGSGCTEPSLPRNTGNAKCAGQAVGEGGMSLHVC
AHC
The following DNA sequence Seq-87 SEQ ID NO. 2> was identified in H.
sapiens:
CTAAAGGAGGAATAGATGTCTTTAAGAAGAAATGAAAAAATAAAGTAAATGTGAAAATTTCCCTTACTTAT
TTCCAAACAAGTGCTCCTCCAAAAAAATGCAAATAATTAAGTTTCTGAAATGGTGAACATATCAGATTAGT
AGACATATGGCAGGAGCAGCAAATGAGCAGATCAAGTTGAAGTCCTAGTATTACCAATCTGTTAATGTTGA
CAGGAAGACTCATTTTGACTGTTCCTTTTATATCAATAAATGAGTGGATTTCAACTACTCTAAATAGGAAT
GCTAAAAGCAGCACTGCTAAAAGTGCATATCAAACCAATAATTTTCTGATGCTGTTTTGGTATATCCTACA
AACATTTGTAGGACAACAACTCAGAAGGGAAAAAAATATCTTATGCCTTTGAGGTCTGTACTGAATGCTAA
TGCATTTGTATATGATGGGTTTAATACAGAACTGAGAATAAATTACTTTCAGCAGCTCCACTCTAGACCTA
TAAATCGCTCTGAGTACTACAAAATCCATACAAAGGAAGAACAGCTGGATAATTTACACCACCAGTATTTG
TCAAAAAAAAAAAAAAAAAGCTGAAAATACAGAACCTGATTTTGTCCCTTTTTCGAGTA
The following amino acid sequence <SEQ ID NO. 60> is the predicted
amino acid sequence derived from the DNA sequence of SEQ ID NO. 2:
LEKGTKSGSVFSAFFFFFQILVVIIQLFFLCMDFVVLRAIYRSRVQLLKVIYSQFCIKPIIYKCISIQYRP
QRHKIFFSLLSCCPTNVCRIYQNSIRKLLVYALLAVLLLAFLFRVVEIHSFTDIKGTVKMSLPVNINRLVI
LGLQLDLLICCSCHMSTNLICSPFQKLNYLHFFGGALVWKVREIFTFTLFFHFFLKTSIPPL
The following DNA sequence Seq-88 <SEQ ID NO. 3> was identified in H.
sapiens:
AGGGGCCCTCCAGCACTGGTCTTGAAGGGGTGACAGGCTCTGGGGTCTGACTCCCACCTCCACCACTTCCC
ACCTGAGGGCCCTGGAATGAATCCTTTCCTGGATCTGAGCTGCCACATCATCAGTGAAAATGACACCTATA
TGGGACTTCAGTGAGAACACAAATGCAACGTTCCTGCCACGGAACAACCCATGTACTCACTGGGAGCATTG
AGAGTAGATCCACACTGATTGACACAGGGACTCCAGGCCTGACCCATGATATGTACTGGATACATGGCCAT
GAGTGCTCCACAG
The following amino acid sequence <SEQ ID NO. 61> is the predicted
amino acid sequence derived from the DNA sequence of SEQ ID NO.3:
VEHSWPCIQYISWVRPGVPVSISVDLLSNLPVSTWVVPWQERCICVLTEVPYRCHFHCGSSDPGKDSFQGP
QVGSGGGGSQTPDPVTPSRPVLEGP
The following DNA sequence Seq-89 <SEQ ID NO. 4> was identified in H.
sapiens:
ACACCAAATACTGCTTTGCTGCCTTAGGCTTCAGCACATTAGCATGGCTTCCTCCCTTGGCATGGTAACTG
TAGCTGAACTTGGAGGGTTTGTATTACCCATTATAATTATTACTTATTTCACATGGAAAACAAGAAAATCT
TTATGGGAATTCCAAGTTCCCCCTAGGAATACCAAAGAGAGGAAAAAGGCTTTGAGGATGGTCCTGATGTG
TGAAGTGGTGTTCATTGTGTGTTTCACTCCTTACCACCTCAACTTCCCATTCTTTATGATGGTGAAGGAAC
ATGTCTTTTTGAACTGCTCTTTTATAAAGATCATTCTCTGTTTCCACATTATTTCCCTGTGTCTTGCAAAT
CTGAATTGTTGTCTTGATCCAGTTGTATATTATTTTATGACCTCAAAATTTCATGATCAATTTTCAGATCA
TGGCAGCTTGGTTCTTCAGTCATGTATGAGATGTAATAACAGTACCTTAGAAATTCATCAGAGGAAGGGAG
GATCTTCAAACTATCTCTCTTGAATGTTTGAAAGATTCCAAGACAATATAATCAAATAATTAACTAGAAAA
ATCGATATGCTCTATTAGTGTATCTATGTCACTTTGAAGATTTTTCTTTTTTTTTTTTCTTTTTTTTTATT
ATACTTTAAG
The following amino acid sequence <SEQ ID NO. 62> is the predicted
amino acid sequence derived from the DNA sequence of SEQ ID NO. 4:
HQILLCCLRLQHISMASSLGMVTVAELGGFVLPIIIITYFTWKTRKSLWEFQVPPRNTKERKKALRMVLMC
EVVFIVCFTPYHLNFPFFMMVKEHVFLNCSFIKIILCFHIISLCLANLNCCLDPVVYYFMTSKFHDQFSDH
GSLVLQSCMRCNNSTLEIHQRKGGSSNYLSMFERFQDNIIKLTRKIDMLYCYVTLKIFLFFFSFFLLYFK
The following DNA sequence Seq-90 <SEQ ID NO. 5> was identified in H.
sapiens:
AATGCTACTGCTCCTGCATATAATAGCTGCTTTGAAGTGTTTGTCTGCTATGTCCAAAACATAGACTCTTT
CAAAAGCACTTTCTGTTGTCTCCTTTTTCTCTTGCATAGGAGTCACATTTTTCTGTCTCTTCACATATTTC
ATATTTATTTTTGTTGAAAACCAGACATTTTAGATAATGTGTTGTAGCAGTCCAGATACTGATTCTCTCCC
CCAGGAGCTGTTGTCTTTCTTACTTGTATATGTGTTTAGTGACTTGGCTGGACTATTTTAATAATGTGTAT
TTCCCTGTAGTATATACCATCTTTTATACTAATGTTACTTTTCCGATAGTGCAGCCTTGGGCATGGACAGA
GTTATCCTGGGATGACAGTAACTTTTAATAGCGCTCTCTATGACTATCTCTTTCCCTGATGTCCCTGTTAA
GCTATCTGCATCTCTTGGTATCACACCTAGCCTTTGACTTCCACTAATTGTTTGATCATTGCCTCACTGTT
TTTGGCAGTGCCCTAAGCCATAAAGTGTTCCACAGTCTGATATAATTAAATTCAGATTCTTACAAGAGTGG
TCTTTGAGGCCAGTCCTTGAGGTTTGTGCTGACTCTGGGAGGGCTCAAATGTTTCCCT
The following amino acid sequence <SEQ ID NO. 63> is the predicted
amino acid sequence derived from the DNA sequence of SEQ ID NO. 5:
CYCSCILLSVCLLCPKHRLFQKHFLLSPFSLAESHFSVSSHISYLFLLKTRHFRCVVAVQILILSPRSCCL
SYLYMCLVTWLDYFNNVYFPVVYTIFYTNVTFPIVQPWAWTELSWDDSNFGSLLSLSLMSLLSYLHLLVSH
LAFDFHLFDHCLTVFGSALRHKVFHSLILNSDSYKSGLGQSLRFVLTLGGLKCFP
The following DNA sequence Seq-91 <SEQ ID NO. 6> was identified in H.
sapiens:
CCTCACATCCCCTTCCCCTCAAACCCTGGCAACCCCAAACTGTTCTTGACAGCCTCCTTTGGCATTTCCTC
ATTTTGGTGTCAGATCTCACAGCAGAATTTCTTACCTATTATATACCAGTGCCTCAGTGTGAAGTTCCGGT
TTAACTTCTTGTTACCACGAGCCCACTATCTTGCCCCAATAATACCCTCCCCCAATTCACAAACACACAAG
CATTCCCTCCTACAGCTTTGGGCCTCCTATCTGAGTCCTTCAGGAAAGAAGTGCTGTGTAACTCCCTTCGC
AGTGAGTGTAGACTTGGTCCAAGGAAGATGAGCACCAGTCAGGGCAGCTGGGCCCTCTTCTCTCCCTGGCC
ATCAGCAAATCAGCACTGCCCATCGATGCCCAGGCAATGGGAGCG
The following amino acid sequence <SEQ ID NO. 64> is the predicted
amino acid sequence derived from the DNA sequence of SEQ ID NO. 6:
PHIPFPSNPGNPKLFLTASFGISSFWCQISQQNFLPIIYQCLSVKFRFNFLLPRAHYLAPIIPSPNSQTHK
HSLLQLWASYLSPSGKKCCVTPLAVSVDLVQGRAPVRAAGPSSLPGHQQISTAHRCPGNGS
The following DNA sequence Seq-92 <SEQ ID NO. 7> was identified in H.
sapiens:
ATTACTATTTTTCAACCTCTTTTACTCCAGGGACTTCTATGCACCCTCTCCCTCAACTCCCCCTCAATTTG
TTCTCATAATCCCCATGACCCCCAGTTTTATAACACCACTGTCAGGAGCCCAAAGCTGCCATTCATTCACT
TCCATTAGCATGACTCTTCATGTACTTTGGGGTCTTCAGTCTCTCCCCTTCTCCTAATTTCCAGGCTTCCA
TTCTGCTTCTGCTGGCTTCCCTACAAAGCCTGCAACATCATAAGCCATTTCAGGAAAGAGCTTGATCATCT
TTTGATGAACCCTGCATTCATGACTCACTGCCTTACCTGTCTTTGGCTCTGCATGTCCCCCAGTTTCCGTT
TCTTTCTCTGGAAAGAGAGATTGCCCAAGAGTCCTGCACATCAGCATTACTAGAAATGCATGCAGACCAGC
TTCAGCTGCTTGCCAACTCTTTAAAAAATGAGTAAACAATTTTCTAAAGGGGAAAAAATCTCTTCACCTCC
TCACACCAACTATTTGCATAATTCAGTGACCTTTTATAAACCGTGCCATTGTATAAGCA
The following amino acid sequence <SEQ ID NO. 65> is the predicted
amino acid sequence derived from the DNA sequence of SEQ ID NO. 7:
ITIFQPLLLQGLLCTLSLNSPSICSHNPHDPQFYNTTVRSPKLPFIHFHITIFQPLLLQGLLCTLSLNSHD
SSCTLGSSVSPLLLISRVPFCFCWLPYKACNIISHFRKELDHLLMNPAFMTHCLTCLWLCMSPSFRFFLWK
ERLPKSPAHQHYKCMQTSFSCLPTLKMSKQFSKGEKISSPPHTNYLHNSVTFYKPCHCIS
The following DNA sequence Seq-93 <SEQ ID NO. 8> was identified in H.
sapiens:
CACCGTCCTCATCATGATCCTCTTCGTCATCTGCTGCTGGGGGCCCTACTGCTTCCTGGTGCTGCTGGCCG
CCGCCCGGCAGGCCCAGACCATGCAGGCCCCCTCGCTCCTCAGCGTGGTGGCCGTCTGGCTGACCTGGGCC
AATGGGGCCATCAACCCTGTCATCTACGCCATCCCCAATCCCAACATTTCGATGCTCCTAGGGCGCAACCG
CGAGGAGGGCTACCGGACTAGGAATGTGGACGCTTTCCTGCCCAGCCAGGGCCCGGGTCTGCAAGCCAGAA
GCCGCAGTCGCCTTCGAAACCGCTATGCCAACCGGCTGGGGGCCTGCAACAGGATGTCCTCTTCCAACCCG
GCCAGCGGAGTGGCAGGGGACGTGGCCATGTGGGCCCGCAAAAATCCAGTTGTACTTTTCTGCCGAGAGGG
ACCACCAGAGCCGGTGACGGCAGTGACCAAACAGCCTAAATCCGAAGCTGGGGATACCAGCCTCTAAGACG
GTTGGAATCGCCAGCTTATGAAGGCAAATTTCCACTCGCATTATTTAATGATGGAAGATTCTGGGGGAGAG
TTGTGGATTTCATAAAGCCAAACATTTAAAGCTAGAGACGGGGGAGGCTTACCACTTTCCCCAAACAACAT
AAAAGACAATGTCCCTTCTTTCAAAAAGTGC
The following amino acid sequence <SEQ ID NO. 66> is the predicted
amino acid sequence derived from the DNA sequence of SEQ ID NO. 8:
TVLIMIVFVICCWGPYCFLVLLAAARQAQTMQAPSLLSVVAVWLTWANGAINPVIYAIRNPNISMLLGRNR
EEGYRTRNVDAFLPSQGPGLQARSRSRLRNRYANRLGACNRMSSSNPASGVAGDVAMWARKNPVVLFCREC
PPEPVTAVTKQPKSEAGDTSLDGWNGQLMKANFHSHYLMNEDSGGELWISSQTFKARDGGGLPLSPNNIKD
NVPSFKKC
The following DNA sequence nGPCR-93 <SEQ ID NO. 9> was identified in H.
sapiens:
CGCTCCTGCGTAAACACGCGGTTCCCTCGGCAACGCTGGAACCCACGTCAAAGGCTCCGCCAGGTCCCCAG
CGACCGCCACCCCTCCGGCCGAGCCCAGCTCCCCGCGGCGGCCGCTAGCCCCCGGCCCCGAGCCACCACTC
CGACCTAGCGGCCGCCGCCCCCGGTGCGGGATGAGGAGATCCGCGGCCGCCACTGGCCCCCATGGAGGAGC
CGCAGCCGCCCCGCCCACCAGCGAGCATGGCCTTACTGCGCAGCCAGCACTCCGGCGCCCCCTCCCCGGCC
GGCCCACCTGGCGCGACTTCCTCCGCGGCCACGGCGGCCGTGCTCTCCTTCAGCACCGTGGCGACCGCGGC
GCTGGGGAACCTGAGCGACGCAAGCGGAGGCGGCACAGCTGCCGCTCCCGGTGGCGGCGGCCTTGGCGGGT
CCGGGGCAGCGCCGGAGGCGCGGGCGGCGGTGACGCGGCCGCTAGCGACGGAGGCGGCGCCGCTGCTGTCG
CACGGAGCTGCAGTGGCGGCCCAGGCGCTCGTCCTCCTGCTCATCTTCCTGCTGTCTAGCCTTGGCAACTG
CGCGGTGATGGGGGTGATTGTGAAGCACCGGCAGCTCCGCACCGTCACCAACGCCTTCATCCTGTCGCTGT
CCCTATCGGATCTGCTCACGGCGCTGCTCTGCCTGCCCGCCGCCTTCCTGGACCTCTTCACTCCGCCCGGG
GGTTCGGCGCCTGCCGCCGCCGCGGGGCCCTGGCGCGCCTTCTGCGCCGCCAGCCGCTTCTTCAGCTCGTG
CTTCGGCATCGTGTCCACCCTCAGCGTGGCGCTCATCTCGTTGGACCGTTACTGCGCTATCGTGCGGCCGC
CGCGGGAGAAGATCGGCCGCCGCCGCGCGCTGCAGCTGCTGGCGGGCGCCTGGCTGACGGCCCTGGGCTTC
TCCTTGCCCTGGCAGCTGCTCGGGGCGCCCCGGGAACTCGCGGCGGCGCAGAGCTTCCACGGCTGCCTCTA
CCGGACCTCCCCGGACCCCGCGCAGCTGGGCGCGGCCTTCAGCGTGGGGCTGGTGGTGGCCTGCTACCTGC
TGCCCTTCCTGCTCATGTGCTTCTGCCACTACCACATCTGCAAGACGGTGCGCCTGTCGGACGTGCGCGTG
CGGCCGGTGAACACCTACGCGCGCGTGCTGCGCTTCTTCAGCGAGGTGCGCACGGCCACCACCGTCCTCAT
CATGATCGTCTTCGTCATCTGCTGCTGGGGGCCCTACTGCTTCCTGGTGCTGCTGGCCGCCGCCCGGCAGG
CCCAGACCATGCAGGCCCCCTCGCTCCTCAGCGTGGTGGCCGTCTGGCTGACCTGGGCCAATGGGGCCATC
AACCCTGTCATCTACGCCATCCGCAATCCCAACATTTCGATGCTCCTAGGGCGCAACCGCGAGGAGGGCTA
CCGGACTAGGAATGTGGACGCTTTCCTGCCCAGCCAGGGCCCGGGTCTGCAAGCCAGAAGCCGCAGTCGCC
TTCGAAACCGCTATGCCAACCGGCTGGGGGCCTGCAACAGGATGTCCTCTTCCAACCCGGCCAGCGGAGTG
GCAGGGGACGTGGCCATGTGGGCCCGCAAAAATCCAGTTGTACTTTTCTGCCGAGAGGGACCACCAGAGCC
GGTGACGGCAGTGACCAAACAGCCTAAATCCGAAGCTGGGGATACCAGCCTCTAAGACGGTTGGAATGGCC
AGCTTATGAAGGCAAATTTCCACTCGCATTATTTAATGATGGAAGATTCTGGGGGAGAGTTGTGGATTTCA
TAAAGCCAAACATTTAAAGCTAGAGACGGGGGAGGCTTACCACTTTCCCCAAACAACATAAAAGACAATGT
CCCTTCTTCAAAAG
The following amino acid sequence <SEQ ID NO. 67> is the predicted
amino acid sequence derived from the DNA sequence of SEQ ID NO. 9:
LEPTSKAPPGPQRPPPLRPSPAPRGGRPPAPSHHSDLAAAAPGAGGDPRPPLGPMEEPQPPRPPASMALLG
SQHSGAPSAAGPPGGTSSAATAAVLSFSTVATAALGNLSDASGGGTAAAPGGGGLGGSGAAREAGAAVRRP
LATEAAPLLSHGAAVAAQALVLLLIFLLSSLGNCAVMGVIVKHRQLRTVTNAFILSLSLSDLLTALLCLPA
AFLDLFTPPGGSAPAAAAGPWRGFCAASRFFSSCGIVSTLSVALISLDRYCAIVRPPREKIGRRRALQLLA
GAWLTALGFSLPWELLGAPRELAAAQSFHGCLYRTSPDPAQLGAAFSVGLVVACYLLPFLLHCFCHYHICK
TVRLSDVRVRPVNTYARVLRFFSEVRTATTVLIMIVFVICCWGPYCFLVLLAAARQAQTMQAPSLLSVVAV
WLTWANGAINPVIYAIRNPNISMLLGRNREEGYRTRNVDAFLPSQGPGLQARSRSRLRNRYANRLGACNRM
SSSNPASGVAGDVAMWARKNPVVLFCREGPPEPVTAVTKQPKSEAGDTSLDGWNGQLMKANFHSHYLMMED
SGGELWISSQTFKARDGGGLPLSPNNI
The following DNA sequence Seq-2588 <SEQ ID NO. 10> was identified in
H. sapiens:
TCTCAAAAAATAAATAAAAACCACTGTACATCAACAAGGCCCTTGGGGGACAGCTGGGGCATAAGTAGGTG
TCAGCCATACATCAGAGCAGTGTGCCTGCCCTGAGCTGCTTGGGGTTGACCAGCCTGGTGTCCAGAAATGC
CTGCTGGAGGGAGTCGTGGTACAGGAAACCTTGTGCTCTTAGAAGGTCTCCTGAGAGGCCCTGCAAAGCCA
GAGTCCCTCTTAGCAGCTCAGATCAGTGCTATCAAAGTATAGCTCGGGGATTGCTGCCAGCATACAAACTT
TTACTGGTCTGCAGCGAGATAAGTACAGAAATTGAAAGTAAGCATTTAGAAACTTTTATAACAATTTTACA
AGGTCTTGTCAAATGTTATTAAAACAAAGCTGAGGCTGGAATTTCACCTTTTTCATTTCGTTTTTTTCAAT
TTAAACAAATTGTAGTAAAATATAGGTAATATAAATGTACCATTTTAGCCATTTTTGAGCGTACAATTTAG
TAGCAGTAAGTGCTTTCACAATATTGTGTAACCACTAGTATTATATAGTATATATTTTTAAAATTTTACAG
AAGTATTAAGTTAGCAGCAGATTAAACATTTTTTTCTTAAATTGAGCTTGAGAAGCGCTGGC
The following amino acid sequence <SEQ ID NO. 68> is the predicted
amino acid sequence derived from the DNA sequence of SEQ ID NO. 10:
ASASQAQFKKKMFNLLLTYFCKILKIYTIYWLHNIVKALTATKLYAQKWLKWYIYITYILLQFVIEKNEMK
KVKFQPQLCFNNIQDLVKLLKFLNAYFQFLYLSRCRPVKVCMLAAIPELYFDSTDLSCEGLWLCRASQETF
EHKVSCTTTPSSRNFWTPGWSTPSSSGQAHCSDVWLTPTYAPAVPQGPCCTVVFIYFLR
The following DNA sequence Seq-2589 SEQ ID NO. 11> was identified in H.
sapiens:
AGAGAGCAGATTGCCCTGTGTAGGTCAGGTCTGGCTTCTTTCTAGTCCAGAGTAGGGAAGAAACAGGAAAG
AGGGCTGGTGTTGAAGGACCTTCAGCCACGAGAAGGGCTGTGTACCATGTAGCCCTCTGGGGAGGCACAAA
AAGGCTCACCATTTTCTGAAAATGACTAGACTGCAGGATCCACGTGAGTGTGACTATTGCATTCATGACCT
TATCCACAGGGCCTCACAAGGTGCCTGACATGCAGTAGGCTCCAGATGCATATTTATTATAAAGTGAATAG
TCCTTAAGCTGCAGGGTCCCTTCTATTTGCATTCTAAGAAATAGTCACTTTTATGCCTAATTTTGTATTTG
CAGTTTTATAAGTTTTATAAGAGGGTCTCCCAAATAGTATAAACTTCAAGCCCCACAAAACCTATGTTTGC
CTCCCATAGGCATGCAATAAATGTTCGTGGATCTAATGAGTAACAAGAAAAAGAAGGAACAAAACCCTAAC
CCCTCCCCTACCCAAACCAGTGGCAACCGGGGAGGATCAAATTCAACCTTGATCAGTCAGAGGCAGCATTC
CTAAATTATTCCCAAGCAGCAATAGACAATGATTTACCTCAATTAATTCAGCCAGTTAAAAGCTTAGTTCT
TACTTGCCAACCGAAGGCTTGAAGGCAAAATGTGTTTAAGCCTC
The following amino acid sequence <SEQ ID NO. 69> is the predicted
amino acid sequence derived from the DNA sequence of SEQ ID NO.11:
RLKHILPSSLRLASKNAFNWLNLRIIVYCCLGIIEGCLLIKVEFDPPRLPLVWVGEGLGFCSFFFLLLIRS
TNIYCMPMGGKHRFCGASLYYLGDPLIKLIKLQIQNAKLFLEMQIEGTLQLKDYSLYNKYAS
GAYCNSGTLGPVDKVNNAIVTLTWILQSSHFQKNVSLFVPPQRATWYTALLVAEGPSTPALPPVSSLLWTR
KNPDLTYTGQSAL
The following DNA sequence Seq-2591 <SEQ ID NO. 12> was identified in
H. sapiens:
TTCAGGCAGATGTCACTTAAAAACTTACCTCTGCACACTGCAAAAACTGTATAGCCCTGAACAGATACTTT
TCTTGAGCATAGTTCCTTTGTCTCTAAAGCAGGCATAATTGCCAATGTGGGGATGATATTTAGAAATCTGA
ACTGATGTTTATTCTCTAGGGGTCTTCTCATTTGACCTGGGATTGGAGATGTCTAGTGTCTCAGAGCAGCA
ATAAGAAAACAGAAACCTCTTCCAGCTTCTGACATCCAAATGTCAAGCTCTTAGGAGAAGAATGGAAAGTC
CTCAAGAAATGCAAATAGCTTTGGCAGAATAGCTGATGAAGACCACCTCTCCCCCCTCCAGAAAGGCATTG
GTTCCCCATTCATGGAAAAGGGAATGTAGAGAGAGATTAGATAATAGTACATCCATAAGGTTCCTGGAATC
TGCATCTGAGGAAGAGGGGCGTCAGAGACCCCAGCTGTTATCTATAATCCCTCCT
The following amino acid sequence <SEQ ID NO. 70> is the predicted
amino acid sequence derived from the DNA sequence of SEQ ID NO. 12:
EGLITAGVSDAPLPQMQIPGTLWMYYYLISLYIPFSMNGEPNPFWRGERWSSSAILPKLFAFLEDFPFFSE
LDIWMSEAGRGFCFLIAALRHTSPIPAQMRRPLENKHQFRFLNIIPTLAIMPALETKELCSRKVSVQGYTV
FAVCRGKFLTDICL
The following DNA sequence Seq-2592 <SEQ ID NO. 13> was identified in
H. sapiens:
GCACTAGGGCAAAGTCAAGACATACGGGTGTCCAGTTCTAGCTTTGCACCTAACTCGTTATATATTTTTAA
GTTACAGTCACTCTGCGCCAGTTTCCTCATTTTTAATAGAGTGGGTTAGAACTAGATAAATACTTTCATTT
TGTCAAGCTCTAAATTCTGACTTCAGGAAAAAACCATAAGGCACTGGAGGTTTATTCATAGGTTTTTCTGC
TGACCCCGTCCCTCTCTGTTTCTTCAACCACCACAAGACAATCAACTTCCCTGATTGGAGATTGGAACAGG
TGTGTTCTAATTCTAAATGCATCACTTAACTATTAGTTCCAACTCTCTGGGGCTTCCTTCAAATAGGGGAA
TTAGACTGGTCTCCAATCTCTTTGTACAGATGACTAACTTTATTTACCCAAAGATTTAGTATTAACAGTCG
GGAGCAGGAGGGAGAATACTTATGAGACAACAGCCATTTCCACAGTGGAGAGGAATGGTTTGTTCCCAATA
GAAGTTACCAGATTTCAGTCCCATTGCCAAATAGATATTATGAGCAAGGAAGAAATCTATAGTAGTAACTT
AAGACCACCAGAAAGATCAAAGCCCAGAGGGTGAGGGTATGGCAATAAACATTAGACATATCTCTAACCCT
CTTTTGTTTGAAATACTCATTACCCTGTGGTACTGGGAATACCTGTGCCTACAA
The following amino acid sequence <SEQ ID NO. 71> is the predicted
amino acid sequence derived from the DNA sequence of SEQ ID NO. 13:
LAQVFPVPQGNEYFKQKRVRDMSNVYCHTLTLWALIFLVVLSYYYRFLPCSYLFGNGTEIWLLLGTNHSSP
LWKWLLSHKYSPSCSRLLILNLWVNKVTHLYKEIGDQSNSPIRKPQRVGTNSVMHLELEHTCSNLQSGKLI
VLWWLKKQRGTGSAEKPMNKPPVPYGFFLKSEFRAQNESIYLVLTHSIKNEETGAELLKNIPVSCKARTGH
PYVLTLPC
The following DNA sequence Seq-2593 <SEQ ID NO. 14> was identified in
H. sapiens:
TTCTGCCATCGCAAGGGGAGGGAAGAGCACCTAAAGGGCTTATGAGAGGTTTGACTGACCAAGGGAGGGAA
CAGAACACATTCCTTTCCATTGGCTAGGACTCGGTCACATGGCTTTCCCTCATTATTAGTGAGGCTTGGAG
AATTCATCTATTTGTGAGCCCAGGAAGAAGAGAAAACAAATTGTGGTAAACATTTAQCAGTCTCTATGACA
ATAGTCTGTATGTTGACTGCAAAGGTGGATGAACAAAACCAAGCCTCCTTTAAAGCAATACAATCTGGCAG
AGTCCCTGGGTTATCATTCTGAACATAGATGCTTATTGTTCAAGAGTTAAGAAAATTAGCATGACTGCATT
CCAGTTCTATAAATTTAATCTTTATTCAGCATATTGTCATCCACATGTCTTAAAAAATAAAATAAAAAACA
AAAAACCTAGTAACTACGTTTTATATAGCAAGGAACACTCATATATATCACTTCATTGTATCCTTACAACA
ATCCTGTGCAGTATATCTTTTACTCCCTTTCTTCTATGTTTTGTATATAAAGAAATGAGCCCCAGGGAGTT
GAATGCCTTGCCCCAACTAGTGAAGCTAAAACTCCAATCCAGGTCTTTTTATTTCCAAATCCATAATCTAC
AACCATCTGTAGAGAGTTATAATTAAGAGATATGAATGGTCAGGGGCCTTTCCATTTCAGTGCAAGTCTGC
CCAGCTCCAACTACCAGCATCTG
The following amino acid sequence <SEQ ID NO. 72> is the predicted
amino acid sequence derived from the DNA sequence of SEQ ID NO. 14:
LPSQGEGRAPKGLMRGLTDQCREQNTFLSIGDSVTWLSLIISEAWRIHLFVSPGRRENKLWTFSSLYDNSL
YVDCKCGTKPSLLSNTIWQSPWVIILNIDAYCSRVKKISMTAFQFYKFNLYSAYCHPHVLKNKIKNKKPSN
YVLYSKEHSYISLHCILTTILCSICFTPFLLCFVYKEmSPRELNGLPQLVKLKLQSRSFYFQIHNLQPSVE
SYNEImVRGLSISVQVCPAPTTSI
The following DNA sequence Seq-2594 <SEQ ID NO. 15> was identified in
H. sapiens:
AATCCTGCATTTCCCATGCTCTGGGGTGAGAACGAATTAGCTGGGAGCCAATTAGCAATCTTGTCAGAAGC
AAATGAATTTTGAATAAACTGGATACTTAAACTGAAATGAGACCATTGAAACCAGAAGAGCCTGAGATCCA
TCAGTTAGAGGAAATAAAAGAAGTGGCATTTTCCTTGCCATCTGGGTGCAGTGTGAGTGATTTTTATAATC
CTACCACATTTTTATCCCTGCTTCCCTTAAACTGTAGGACCCAAGGAACCTGGCTGTTTTGTTCAACATCA
TGTGACCCCATACCTAACCAGGCCAGGCACAAAATTGGCCTCCAATAAGTAGTGGATCAAAGTATGAATGG
ATAAACTGAATGAATGAAGCCAAACTTGAATTTCTCCATACCTTATCCAAATGGGAATGGTAAAAATCATA
AGCTTTTGAGAAGAGAACTTATTAAGAAGCCCTACATCAGTCATGACTGGCATCATTGGTTAGTTTACCCA
ATTTTCTCCTTCCCTTCATCTTCCTAATGCAACTCTGGTTTGGGCTGCAGTATACCCAGTTAGAATACGCC
CTCCCCAGAGTCTCATCCAGCTGGAGATGATCAAGTCACCAGTTCTAGCTAATAAGTTGCCAGCCAAAGTA
TTCAGGATGAGACTTCCAGAAAAAGCATTGTTTTCCTGATAACAATGGACAGACC
The following amino acid sequence <SEQ ID NO. 73> is the predicted
amino acid sequence derived from the DNA sequence of SEQ ID NO. 15:
SVHCYQENNAFSGSLILNTLAGNLLARTGDLIISSWMRLWGGRILTGYTAAQTRVALGRREGENWVNPMMP
VMTDVGLLNKFSSQKLMIFTIPIWISYGEIQVWLHSFSLSIHTLIHYLLEANFVPGLVRYGVTSCTKQPGS
LGPTVGKQGKCGRIIKITHTAPRWQGKCHFFYFLLMDLRLFWFQWSHFSLSIQFIQNSFASDKIANWLPAN
SFSPQSMGNAG
The following DNA sequence Seq-2595 <SEQ ID NO. 16> was identified in
H. sapiens:
GACTGTTATTACTGAAAGTCATTGCTTTTAGACTCTTCCAACTACAGAGCAAGGAAGTTTATGTGTATATA
GTAATCTGTGAATATACACATACATACACATATTTCTATATGTAATCATCCATATTTAAATTAAGTAGAAT
ATGAGTTCATACTGATATCTCCAATCCTAATCAGTTACCACAGGGATTATTCCGGCCTTTTTCCCTTGGAA
GTTTGCAACTCCTGCTTCAACAGTTAGAAATCTGGCTTCCATATTCATTTGCTTAATTGTTCAATTCCAGT
ACACATAAATGGTGGCTTCAGAATTAATAACTTATACCTCCATGGGAAATAACTTTATTAACTAAAGTACA
GCACTTATGTATAGTACTTTTTGAATTTTTAGACTTAGAGATTCCTCTTCTTTTCCAAAGTTACTTAGGTC
AGAACCATTTTCCATTCTTCAGTGAAGTTGTCTTATGTATTTGTAATACAGTTAGATTGTTCTGTCATATG
GTGCATTCCATCCTGGGATTTCCTATCTCTTTTTTTAACATTTGCATATATTAAGTTTCATTCTTTTGTGC
TGTATCATTCTATGGGTTTCAATTAATGCATAGTGTCATGAATCTGCCACCATAGGAGCATCATACAGAGT
AGTTTCACCAACTTAAAAAATTCCCTATGTTTTAC
The following amino acid sequence <SEQ ID NO. 74> is the predicted
amino acid sequence derived from the DNA sequence of SEQ ID NO. 16:
LLLLKVIAFRLFQLQSKEVYVYIVICEYTHTYTYFYNSSIFKLSRIVHTDISNPNQLPQGLFRPFSLGSLQ
LLLQQLEIWLPYSFALFNSSTHKWWLQNLIPPWEITLLTKVQHLCIVLFEFLDLEIPLLFQSYLGQNHFPF
FSEVVLCICNTVRLFCHMVHSILGFPISFFNICIYVSFFCAVSFYGFQLMHSVMNLPPEHHTEFHQLKKFP
MFY
The following DNA sequence Seq-2596 <SEQ ID NO. 17> was identified in
H. sapiens:
CGTTCCTTCCTCTGTGTCATAATGGACATGATGATAGTTGGCTCACTCAGTAAACATTCTGTGTCTGGAAG
GATTTGATTTGTCCTTTTCTTGAGGCAACAATTTTGAGGTGATTTGAAAAATCTTTCTTGAAAAATTAAAA
AATTTTTCTAATTAAAAATAATGCAAGCTCATTAGAAAAAAATTGAAAAATAAATAAAAGCACAATTTTTC
TTAACCACTTAAAGATGACCATTGTTAGTTTTTTTTTTTTTTTGGTGCTTTTTTCCGTTTCCAATCTCTTT
TCTATTAAAACTTCTGAAATGTGATTGTAGCAATGACGCATAAGGGGCCCTTGACACATTGAGAAATTTAT
AAATACGCTGGCTTCTTGTCTTGCTTTTGTCCCCAGCTTAACTGGGAACTCTTTTTCTATATCTTTGAAAC
TCCAAATCCTAGATAATTCTTCAAGGTCAAGCTCCAATGTCTTGCTGGATTCTTCTCAGCAGGAATTGATC
TATTTTCTCTGTATTTTTCTGCCACAGGATCTATGACTCTCTTATGGCAACTACCACCTTCTGCCTTATAT
TACGATTTTTGAATCTTCCAACAAAGTCTAATTTTTTTTTTTTCAAATGAAGTCTCGCTATATTGCCCAAG
CTGGAG
The following amino acid sequence <SEQ ID NO. 75> is the predicted
amino acid sequence derived from the DNA sequence of SEQ ID NO. 17:
FLPLCHNGHDDSWLTQTFCVWKDLICPFLEATILRFEKSFLKNKIFLIKNNASSLEKNKINKSTIFLNH5LK
MTIVSFFFFLVLFSVSNLFSIKTSEMLQRIRGPHIEKFINTLASCLAFVPSLTGNSFSISLKLQILDNSSR
SSSNVLLDSSQQELIYFLCIFVPQDLLSYGNYHLLPYITIFESSNKVFFFFQMKSRYIAQAG
The following DNA sequence Seq-2598 <SEQ ID NO. 18> was identified in
H. sapiens:
ATGTCATGGGAAATGCAAGAATATGTGTCCAGCATGGAAGGGAATCAGTATGGAAGTCTTTTGATAAATTG
TGGCATTTATCACTAACATTGCCTCAAAACTTTAGACTACCTGCCATATACAAATTAGAGGTGAAAATTAC
TTCCATGTAATATACAAGCCAACACAAAGAATCCTATCCCAGTTTCTTGGATGGATAGGCAAGAATCTGGG
TAAGGTTTATTGTGCAATAATCCTCTTCTCTCTTCTATAGGCCAGGATTTAAGTTTACCTCAAAAATGGAA
AATTTTGGCTGGGAAAATTACATGTGGGAAGACATCTTCAGTGGAGATTTTAGTAATTACAGTTTCAGCTA
TGACCCTACCCCTTTTCTACTAGATTCTGCCCCATGTTGGCCAGAATCCCTAGAAATCAATTATGTTTTGA
TCATCATCTATGCCCTGATGTTTCTACTGAACGTGATGTGAAACTCCCTGCCGATGCTGGTCATCTTATTC
AGCTGAGTCAGCCACTGTCACCGATGTCTACCTGCTGACCCTGGCCTTGGCCGACCTGTTCTTTTCCCTGA
CATTGCCCATCTTGGCTGCCTCCAAGAATGAATGGCTGGGATTTTTGGCACAATCTGTGCCAGGTGGTCTA
GCTCCTGAAGGAAGTCAACTTCTACGGGGGGTATTCTACTACTGGCCTGCCGCAGCATGGGACTGTTA
The following amino acid sequence <SEQ ID NO. 76> is the predicted
amino acid sequence derived from the DNA sequence of SEQ ID NO. 18:
VMGNARICVQHGRESVWKSFDKLWHLSLTLPQNFRLPAIYKLEVKITSMYTSQHKESYPSFLDGARIWVRF
IVQSSSLFYRPGFKFTSKMENFGWENYMWEDIFSGDFSNYSFSYDPTPFLLDSAPCWPESLEINYVLIIIY
ALMFLLNVMNSLPMLVILFSVSHCHRCLPADPGLGRPVLFPDIAHLGCLQEMAGIFGTICARWSSSRKSTS
TGGILLLACRSMGLL
The following DNA sequence Seq-2600 <SEQ ID NO. 19> was identified in
H. sapiens:
TATGATATTTCAGCCATGGTGCTGAACATTTCCAAACAGCATAAATGCACCATGTGTGTATGTTTTCCTTT
GGGATGCTGTGCTTAGAGGGTAGCAGACAGGGTGCAAAGTGAGAAGGACCTGGCTCTGCACCCAACACTGC
CAGTATTGAATCCTGACTCCATCATCTGGGAGCTGTGCAACCTACGCAAGGTACTTGGCCTCAGTTTCCTC
ATCATCCCCATGGCATTTTTGTGAGAATTAAATGAGCTGAAACCTTGAAACCCCTTCAAACAGCAGCTGGC
ACAGAGGAAGCACACAATCAATGTCAGCTGTACTCTTCCTGGCACTGTGGAGATCCCAGCTCTGCCCCTAG
CTAGTCACTTCTCTTCTTGGAATCTCAGTTCCTTCATCTGGGAAATGGGAGCAGATGTGAAAAGGGGCAGG
GTGAGAATACATATGAAAGTGCTGGCTCCTGGTGCATAGCAGGCACTTAATAATGATACACTTTTCCATCT
TCTGCCTTCCCCAGGGATGCATTGTGCCATGTAAGAGAGAGAGCCTCCAGGGTTGGCGAGAGTTTTTGATC
CAGGCTTTTTCAGGTGTCAAAGATGAGCTGGGTGATTCTCCATAGATTTTCCTTTCTAACAGGTGACAGTT
CTGTTTCAGAAATACTGTGGATGTTCAGGTTTACAGCACAT
The following amino acid sequence <SEQ ID NO. 77> is the predicted
amino acid sequence derived from the DNA sequence of SEQ ID NO. 19:
VLTTSTVFLKQNCHLLERKIYGESPSSSLTPEKAWIKNSRQPWRLSLLHGTMHPWGRQKMEKCIIIKCLLC
TRSQHFHMYSHPAPFHICSHFPDEGTEIPRREVTSGQSWDLHTARKSTADIDCVLPLCQLLFEGVSRFQLI
FWQKCHGDDEETEAKYLAVAQLPDDGVRIQYWQCWVQSQVLLTLHPVCYPLSTASQRKTYTHGAFMLFGNV
QHHGNII
The following DNA sequence Seq-2601 <SEQ ID NO. 20> was identified in
H. sapiens:
TTTATGCTCATGTAGTTCTTTCCAAGAAGAGAAATTACAGAGTCAAATTGTAGAAATATTTAAAAATCTTT
GGCACACATAAACAGTATCCATATAATTTATACCATCTTTTAGATGAGTTTTAACACCAAATGATAGAAAT
CTCAGTTTCATACAGATTTGGTGGGCTGGAACCAAATACTTGCCTGATAGGCTGTCCCCTCGTCTTTCCTA
GCTGTTCTGGGAAAGGCAGTTCCTGGTAAGAACTCTCCCTACGGCCCCTTTCATCTCACTGTTCCTCAGGG
CATAGATAAGTGGGTTGAGCAGTGGGGTTCCCAATGTGTACACCAGTGAGATGAACTGATCTTGCTTGGGG
TTGTAGCTGGAGCTGGGGCACAGGTACATGAAGGCACAGCAGCCATACTGCAGCAGCACCACAGTGAGGTG
GGAAGAGCAGGTGGAGAAAGCCCGGTGGCGGCCAGCAGCCGAGTGGATCTTGA
The following amino acid sequence <SEQ ID NO. 78> is the predicted
amino acid sequence derived from the DNA sequence of SEQ ID NO. 20:
KIHSAAGRHRAFSTCSSHLTVVLLQYGCCAFMYLCPSSSYNPKQDQFISLVYTLGTPLLNPLIYALRNSEM
KGAVGRVLTRNCLSQNSERRGDSLSGKYLVPAHQICMKLRFLSFGVKTHLKDGINYMDTVYVCQRFLNIST
ILCNFSSWKELHEHK
The following DNA sequence Seq-2602 SEQ ID NO. 21> was identified in H.
sapiens:
TTTAAGCCACCCAGTCTGTGGTGTTCTGTTATGGCAGCCCAAGCCAGCTACTACAGGGTGGGACGAGGGGA
GGAGCATGGCCTCTGCTGGAAGTGCAGGCAAATGATCCCCCAGGAACAATGATGGGAGCTTCTGATTGCTC
TCATTATCTCTGCAAAGTAGGAAGAAAGATTCATCAGCTGAGCATGAGGATGGTAGAAAACATCTTTGGGA
AATTTCAGAAGTGAAGGAAGGCATAAAATAGTCATCTAAAAAAAGCAGGAAAGGGAAAAGACAGAGAAATC
CAGTATGAGTCCCAGGACTCCAGGAAGCATCAGGACCCACTTGAAATTGCCAATGCTGAATTTAAAATGAG
GCCAGTCTGTACAGAAGCACTTCTGGAATTTGCTAACAGCTAAATAGAGTAGAATCAATACTTTAGAGAAT
ACGAGTAACCAAAGGAATAAAATTAACTGATCAACTTTTGTGGTTTTTACTATTAATATTTTCTTCAGTGT
AAATCATAGCTGCCTGAATTCCTGAACCCCTCTTATATAAATCTAAAAAGCTCTGGTTTATCATGGTTGAA
AATTCATGGCTAACTTATCAGGCAAACTGTCCCTAAAGCATTTTTTGAATAGCTTTAGTATCAAGATGGTA
CTGAGTGTACATTTCATTTCCCTGCTTAAAGGAAGGCTTAGTTATTTTAAACCAAGTCTTATTTTTATAG
The following amino acid sequence <SEQ ID NO. 79> is the predicted
amino acid sequence derived from the DNA sequence of SEQ ID NO. 21:
IKIRLGLKLSLPLSREMKCTLSTILILKLFKKCFRDSLPDKLAMNFQPTRAFIYIRGVQEFRQLFTLKKIL
IVKTTKVDQLILFLWLLVFSKVLILLYLAVSKFQKCFCTDWPHFKFSIGNFKWVLMLPGVLGLILDFSVFS
LSCFFMTILCLPSLLKFPKDVFYHPHAQLMNLSSYFAEIMRAIRSSHHCSWGIICLHFQQRPCSSPRPTLL
AWAAITEHHRLGGL
The following DNA sequence Seq-2603 <SEQ ID NO. 22> was identified in
H. sapiens:
ATTTCTGGATTTATGCCTCCCCTGACCCATTCCAGGATTTACCCCAAACCTTCCACACTCTCTTCTAACAG
GGAAAGTTCTGTTATGACACAATAGTACTTATTAAGACAGATTTACCTTCTAAGTCTCAGGACAGCATTTC
ACAACCAGAAATAACTGGTCACATGAAGAACCAGGAGTCTGGTAGTAGTGAAATTCATTTTCCTTCTTGAA
AAAGTGGATCAAAGGATTCAAACAGCAAGTGGTGAATCAATGAAAAGTGGTAAAATGGTGAGGAAAAAATG
TTACTAAAAGATGACCTCAAGATTACTGGTGCATATGAATTGCTTTTTTATATAGGAAAATACTGGATAAT
TTCTTATTGTCATAGTATAATTAGAAGCAATTTCATGTGTTCATTTTGCCACATGAGTTTAAATGGAATAG
ATTTGGTTCCCTCTCTAACATGAGTTCAGTGTCTGAACTTGGGCAAATTTCTAAACAATTCTGAGCTTCAC
TACCTCTGCTTGAAAGTGAG
The following amino acid sequence <SEQ ID NO. 80> is the predicted
amino acid sequence derived from the DNA sequence of SEQ ID NO. 22:
SLSSRGSEAQNCLEICPSSDTELMLEREPNLFHLNSCGKMNTNCFLYYDNKKLSSIFLYKKAIHMHQSGHL
LVTFFPHHFTTFHFTTCCLNPLIHFFKKENEFHYYQTPGSSCDQLFLVVKCCPETKVNLSVLLCHNRTFPV
RRECGRFGVNPGMGQGRHKSRN
The following DNA sequence Seq-2604 <SEQ ID NO. 23> was identified in
H. sapiens:
CTTTGGAATTTTATTCTAAGCATCAATCAAGAGGTATAGTACGAGAAAGGTAGAACATGTAATTATAAATT
CAGGATTCAGGAAGTTTATTTTTCTCTTCTTTTTAATTCTCTCAAAATGATCTTGATTCCTGCAAAGTGTT
AGTATATCTGGTAAGTAAGAGTCTATTTCTTTTAAACTTCATCTGTATTAACCAGCTTTATATGACCAAAA
TGTCCCCCAAATTTAAATCTTTGCACAGTAAGGCCTTATATGTACACCTGGCCTCATTTCAAAAGACTAAA
GCAGTTGTTCTCAAATTCAGCTGCACATTAATATAAACTGGAAAACTGTTTAAGCTCCTGATGACAAAGCC
ACATGTGAGACTAATTTATGCTGAATCACTGGGCCAAGGACCCAGGTATCAGCATTTTTTAAAACTATAGA
GGAATAACCAGGGTTGAGAACCACTGCACAAAATGGTAAATGCAACTTTTATTTAAGTTATTTTTTTTAAA
TAAATAATGGTTGAATTGATACTGATCTTAGTACCAAGTCATGGCAATTTTTTCAGACTTAGAGAATTCAT
CCTGGCATTGAGATTATTAAAGAACCTAGAAATCCAAGTGTTTTTGTTTATATTTTTCCTGTAAATATTAG
AGTATGCTAGTGCTCATCCTTATTTGATAATTTTGGAAAAATATATTAAAACATTT
The following amino acid sequence <SEQ ID NO. 81> is the predicted
amino acid sequence derived from the DNA sequence of SEQ ID NO. 23:
LEFYSKHQSRGIVRERNMLIQDSGSLFFSSFFSQNDLDSCKVLVYLVSKSLFLLNFICINQLYMTKMSPKF
KSLHSKALYVHLASFQKTKAVVLKFSCTLITGKLFKLLMTKPHVRLIYAESLGQGPRYQHFLKLRNNQGEP
LHKMVNATFIVIFFKIMVELILILVPSHGNFFRLREFILALRLLKNLEIQVFLFIFFLILEYASAHPYLII
LEKYIKTF
The following DNA sequence Seq-2606 <SEQ ID NO. 24> was identified in
H. sapiens:
ATTATCATTTGAATGTTGATATTACATCATATACAAATTGATTGCAACATAGTTATTTAATGTAACATTCT
ATTTTAAAAGATAAATTTATCAGAATCATACATTGCTACAGTAGTCTCCCTTATCCACAGGTTCATTTTCT
ATGGTTTCAGTTACCTACTGTCAACAAGGATCCAACAATATTACATGGGAAAATCACAGAAATAAACAGTT
TGTAAGTTTTAATTTGTGCGCTGTTCTGGGCAACGTGATAAAATCTCATGCTGTTCCTCTCTATCTTGCCT
GAACATGAATTATCCTTTGTCCAGTATATCCACACTACATATGCTACCTTCCCATTCATCATTTAGTAGCT
GTTTTGATTATCTGATAGAAAAAACACACAGTATATATAGAGTTTTTTATGGGGCAAGGGAAAACTTTCTC
TTTGTCCTCTGAAGATTTCACTGAAAACTCAACTCACAAGGGCAGACTAATAGGAATGAAGGTAAAAAAAA
AATATATTAACATCAATGGAGATAACTACAGAGTGATTATTCCATTGCCATCAATGGACTACAGTGGCTTA
AATATCGTTTTGAGGTTACAAAAAGAGTGGAAGTCTTGGGATCTTGGCAAAACAGGTTATGGGAAGAAGAG
AAGAGAAACCCTGGTTAGCAAAGGTCATCTTGTGATGC
The following amino acid sequence <SEQ ID NO. 82> is the predicted
amino acid sequence derived from the DNA sequence of SEQ ID NO. 24:
IIIMLILHHIQIDCNIVICNILFKINLSESYIATVVSLIHRFIFYGFSYLLSTRIQQYYMGKSQKTVCKFF
VRCSGQRDKISCCSSLSCLNMNYPLSSISTLHMLPSHSSFSSCFDYLIEKTHSIYRVFYGARENFLFVLRF
TENSTHKGRLIGMKVKKKIYHQWRLQSDYSIAINGLQWLKYRFEVTKRVEVLGSWQNRLWEEEKRNPGQRS
SCD
The following DNA sequence Seq-2607 <SEQ ID NO. 25> was identified in
H. sapiens:
TTTTTTCCCCCTGAGTGTTTCTCTCATGCTTTCCTCCAAATGGAGATGGAGAGGTTTCACCTCACTTTTCT
CTAACTCTCCCTAGTTTTTTGGTTTCTTTTCCTCCACATCTAAAAGTGTGCAGAATGTCCCTTTAGCACAT
AGAAAATCTTTTCTTGACCCTGCCACCTACTTAACTAAAATCCCACACTTTTCTTCTTCTTTTAAGATTTC
CTTTATAATGGTGTGTGTCAATGGCCACATCCACCTTATCCATTCCTTCTTAAAGTTCCAGAAAAACGGTT
TTGTTTCCTGTTACTTTAATGGAATTATTTTTCCAAAGATCAACAGGACTTTCCCTCAAGCCCAATCCAGT
CGGTAG
The following amino acid sequence <SEQ ID NO. 83> is the predicted
amino acid sequence derived from the DNA sequence of SEQ ID NO. 25:
FFPLSVSLMLSSKWRWRGFTSLFSNSPFFGFFSSTSKSVQNVPLAHRKSFLDPATYLTKIPHFSSSFKISF
IMVCVNGHIHLIHSFLKFQKNGFVSCYFNGIIFPKINRTFPQAQSSR
The following DNA sequence Seq-2608 <SEQ ID NO. 26> was identified in
H. sapiens:
ATACATGATAAGGTACATGGATCCAGGGGAAGGATGAAGGGCAGTGTGGGATTGCTTTTGAATTTCTCCAA
ACTCGCCCATAAAAGCAGACAGGACAAACTAAGATAACTAAACAAAAAAACCCACAGACAAAACTATTACA
AACCCCAAAAGAAGTGTGGTGGGAACAAACATCTGATAGAATCAGACACATTACTGGTGACCGGACATAAG
CCCTGTTAATGAGAAGCTTACATTTAGGAGAGTCAATTAAGTACACGCTATACACAACCTAAAGTGGTAAA
TGCTACCTTGGTTATTCAACTTCACTGTTACATGCCTTGAAGTGTGGGGTGCACTGGCCTGAACCATTCTG
GTTGTGTTTGATTCCTTAGGATGCCACCAACAAATAACATTGAGAAATACCCAGCTACTTTTCATTGTTCT
CCAATGGCAGCAAAGTACAAATGATCTCTATGA
The following amino acid sequence <SEQ ID NO. 84> is the predicted
amino acid sequence derived from the DNA sequence of SEQ ID NO. 26:
IEIICTLLPLENNEKLGISQCYLLVASGIKHNQNGSGQCTPHFKACNSEVEPRHLPLVVYSVYLIDSPKCK
LLINRAYVRSPVMCLILSDVCSHHTSFGVCNSFVCGFFCLVILVCPVCFYGRVWRNSKAIPHCPSSFPWIH
VPYHV
The following DNA sequence Seq-2609 <SEQ ID NO. 27> was identified in
H. sapiens:
TCCAGGCAGTATTCTCCATGACAATGAGGAAGGTAAGTCTGCAACAGAAGAACAATGGCAGAAATTTTAAG
AAAAGTTTACCGCCTGGGACTATGACTCACCTTTTGGGAGAAAATGTGACTAACCCTTTGTAAGAGCTTGT
TGAGAGCTCACTTTCCTGGGAGGAGTCGGGAGAAGGGGAGCATCAGCTGACGAAGAGGTGAAGGAGGTACC
CAACAAGAAAAGCGTAGAAGGACCAGGGATTTGGGGTCGGGTCTTCCTCCTGATTCCAAGGGATGGCATAA
GATATTGCCAAGTGAAGGAAGCGAAGTAGAGCCAGCAAAGGAAGGTGAACTGTTGTTTCATTAGAAATAAT
ATGTTGTGATAATTATACAAAGTACTAATTAGTAAATTTCTTTCCAACCTCGACACTCCAAAAATCCCTGT
ACTTATATCCCGAAGGCCTCTTCTTCCCCAAGCTGGAAGACACGGTCACTCATTAGTCACCCACTGTCACA
GGAGTAACAGAGACTACAAATATTGGACAGGACATAAGTGAGGGTCAAGCATCTGGATGCAGATGCATGAC
AGGATGCAAGTCTTCCCAGCTCTCATGGACTTTGCGACAGATGCACAGAGTGAGGTA
The following amino acid sequence <SEQ ID NO. 85> is the predicted
amino acid sequence derived from the DNA sequence of SEQ ID NO. 27:
TSLCASVAKSMPAGKTCILSCICIQMLDPHLCPVQYLSLLLLQWVTNEPCLPAWGRRGLRDISTGIFGVSR
LERNLLISTLYNYHNILFLMKQQFTFLCWLYFASFTWQYLMPSLGIRRKTRPQIPGPSTLFLLGTSFTSSS
ADAPLLPTPPRKVSSQQALTKGSHFLPKGESSQAVNFSNFCHCSSVADLPSSLSWRILPG
The following DNA sequence Seq-2610 <SEQ ID NO. 28> was identified in
H. sapiens:
ATTAAATGCCTGAACTCCCTTCAGCTCTGAAACTCTGTGGTGTATTCTCTGAGGACATTACCTCTCTAAGG
GACCCAAATTAAACAGCTCACACCATCCATCATTTTTCTGTCTGAGGTTTTATTTCCCTAATCAGATTTGG
GTAAATTTTCAGCCTCTCTCTGTCTCTTACTTCCAATCCAATAAAACCTGTATGGATTTGTTTTGTATTTC
ATCTAATGTCATTATTCATTCTAAGAGTCACTGCCTGACCATTTCCCTGCCTATAGCATAATTAGCTATTA
AAAAGCTACACTGGCATGGTTTTCAAACTTGCATCCTCTTTTTCTGAGGTGGATTGATTCTAAACTGATTA
AAATATCTCAGAATTTCCAATACAATTTTTAAAATGCAACAGATTTTCAAGACTGCCTCATGACTCTGCCA
AGCCAAGGGAGTTAGCTGCCAACTCTCTCTGACTGCCAAGGAAGCCAAATAAATAATCCTGATGGTGGTTT
TAAAATGAGAGGCAAGTGCCCATTTCTTAGGTTGACAGTGCCACCCTACACATTGACTTCTCCAGGGTTTG
TAAGACACCAAGGGTGATGTTTCAGATTTTCCC
The following amino acid sequence <SEQ ID NO. 86> is the predicted
amino acid sequence derived from the DNA sequence of SEQ ID NO. 28:
LNATPFSSETLWCILGHYLSKGPKLNSSHHPSFFCLRFYFPNQIWVNFQPLSVSYFQSNKTCMDLFCISSN
VIIHSKSHCLTISLPIALAIKKLHWHGFQTCILFFGGLILNLKYLRISNTIFKMQQIFKTASLCQAKGVSC
QLSLTAKEAKIILMVVLKEASAHFLGQCHPTHLLQGLDTKGDVSDFP
The following DNA sequence Seq-2611 <SEQ ID NO. 29> was identified in
H. sapiens:
TCCACATTCTTTCTAAAGTTCTGAGCTTTTCCATGGGCTTCCATGGTAGGGAAAGCACATGGCCTGGGTGT
GGGTAGAGCAGGTGCGGCCATTTATATGTATGGTTCTTTGCAAGTCTGGCATTTGTAAAATGGGTGATGCT
TGTATTGTGTTTATTTATTCAATCATGTAATAGAAGATGCACATAAGATTATTTTGAAAAGTATGCCTTCC
ATTTTCATGCTGAGAATAATGCAGGAAGTTCAGTGTAATGCAGTTATAATAAAATAGTAGCAAAACAATAT
TTTGCTTTAAATCATGGAATTAGCAAGTAAAGACTAATTGGAAGCCAATCTTTTGCAAATTTTTTAAATGT
AAGTTTATTTGGAGGATATGACTTGTTGGCCCAGAGTACATATAAAGAACAAAAGAGTATAATTAACAACA
GTTTCAAATATGGACTTACCAGGCATCTTGATAAAATCAGTATTGACATGTATGTGAATGCCAACATTGTG
TTTTTCCAATTCAATACTATGTTATGCCATAAAACTGGTAGCAGTTATGAAAATTAGAATTGGTTAAAAAC
TGTTGAAATCTTTAAATTTTTCCTGTTTA
The following amino acid sequence <SEQ ID NO. 87> is the predicted
amino acid sequence derived from the DNA sequence of SEQ ID NO. 29:
NRKNLKISTVFNQFFSLLPVLWHNIVLNWKNTMLAFTYMSILILSRCLVSPYLKLLLIILFCSLYVLWANK
SYPPNKLTFKKFAKDWLPISLYLLIPFKAKYCFATILLLHYTELPALFSAKWKAYFSKSYVHLLLHDINKH
NTSITHFTNARLAKNHTYKWPHLLYPHPGHVLSLPWKPMEKLRTLERMW
The following DNA sequence Seq-2613 <SEQ ID NO. 30> was identified in
H. sapiens:
TACGTGGGTTTCCCTATCGTCCTCTTCATGAGTTCTTTGTGAAAACAGAAAGACTGAGTCTGCCAATAACC
AGCAAGAGAACAAGATAAAATAAATAAAATTAACCATAAGACTTTAACATATGACAAACAACTGGTAAGGA
ATCCATATTTTAGATATAAAAGTCTCTTGCACAGGCCAGAAAATGAAACTTTAATTTAAGCAATAAAATTC
ATCCATATTTTAGATATAAAAGTCTCTTGCACAGGCCAGAAAATGAAACTTTAATTTAAGCAATAAAATTC
CCCTTTGTAGACTGCAAATGGAGAACATGCTATCTAGCTTCATTTTTCTTCAACTTACATAAAAATGAAAC
AATGGTTAATGTTCTGGCGGCATCTCTAAACATATTCAGTGAAACAAAATTTCCTTACAAATGTCAACAGC
TTACAACAAATAACATTTTATCCTGTTTAATTATTTAGAAACAAAATCAGTTATGCTGAGATATGTTTGCA
TGGGATTTATATACTCTGATCATAGAAACAAATTATTGACATCTGAATCTGAAAGCTGCAAAACATGATAA
AAGACATAATAAAATCACAGATTTGTTATTCTCTCAGGAACTTTTTCTAG
The following amino acid sequence <SEQ ID NO. 88> is the predicted
amino acid sequence derived from the DNA sequence of SEQ ID NO. 30:
KKFLREQICDFIMSFIMFCSFQIQMSIICFYDQSIIPCKHISALILFLNNTGNVICCKLLTRFVRKCFTEY
VRCRQNINHCFIFMVEEKSIACSPPAVYKGEFYCLNSFIFWPVQETFISKIWMYVFHILEFIVWKNTIKVD
QKILKILTSCLSYVKVLWLILFILSCSLAGYWQTQSFCFHKELMKRTIGKPT
The following DNA sequence Seq-2614 SEQ ID NO. 31> was identified in H.
sapiens:
GCTGCCCATGCTTGGTGGTAGGATGTATGAAGCTCCTTGCTCCTCCAGCTGGGCATCCTGCCACTTGCTGA
GCCAAATAAGGAATGTGGGGAAGCAGCAGGCCACCAGCCAGATGAGGGCCACTGCCTTCCAGGCAGCCCCA
TGGGACATGAAGGAGAGGTAGCGCAGTGGATGGATGACTGCCAGGTAGGTGTGCAGCACAATGGcGGTGAA
GGACAGGATGGTGCTGGTGCAGGCGGCGAAGACAGCATCAGTGAGAATGCCACAGGCCATGCGGCCCAGCT
CCCAGCCACCCAGGCTGCTGGAGGAGATGAGCATGTGGAGGAGAATGTAGGCCAGGTCTGAGAGCAGGATG
TTAGCCGGGAGCAGGTAGTGGGGCTCCTGTCGCAGCCGTTGGTTCCGCAGGATGGTCACCAGCAGCAGGGG
GCTGACAGCCAGTGTGGCTGCAGCCAGCAGGCTTGAGGGAAGGAAAAGCCAGTACAGCATGGAGCTGGGCA
CCCTGAGGTCCCCCAGGCCCAAGGAAGTGTTGCTGGCTGCTTGGGGCATGCAGGGTGTCTTGCTGATGAGC
TGGATCAGGGCCGGCCAAGCTGTAGTGCCCACAGGGCAAGGTGCCAGCTCATCCCCCATGCTTCCTGGCAG
GGATCGCTGGCTTTGT
The following amino acid sequence <SEQ ID NO. 89> is the predicted
amino acid sequence derived from the DNA sequence of SEQ ID NO. 31:
QSQPSLPGSMGDELAPCPVGTTAWPALIQLISKTPCMPQAASNTSLGLGDLRVPSSMLYWLFLPSSLLAAA
TLAVSPLLLVTILRNQRLRQEPHYLLPANILLSDLAYILLHMLISSSSLGGWELGRMACGILTDAVFAACTP
STILSFTAIVLHTYLAVIHPLRYLSFMSHGAAWKAVALIWLVACCFPTFLIWLSKWQDAQLEEQGASYILP
PSMGT
The following DNA sequence Seq-2615 <SEQ ID NO. 32> was identified in
H. sapiens:
AACACTGACTTCTCTGAAGCAGTTGTCTAAAAGAACCTACACCATTTTTATTTAGCAAAAAGGCTTTTGTT
AAAAGCAGGGGATAGCAGAAAGAGCTTTGTAAAAAATATGTCATGGATTTTAGGAGTTTCTAAGAGCAAGA
AAACGTTTCTTAAATAGAGGAATGAAGCAATTAGAGTTCCATAAAAATCACCTAATGGGCCTTCCAAAAGG
CAAATGCTAAAGCCCCAGAAATCATCACTGAGGAAGTCTGAAGTAGGAAGAGACCTTGTTCTAGAAAGCCG
ACAAGGTAGAAATTAAAATGGAACAGGCCCAACTTGAAATTCCGAGACCAAAAGAGGAGCTGATGACATTG
GTGGGAGACACGTGTGGGAATAAAGAATGTTGGTAGATTCTAGAGACATTCCAGCGATAACACAGACACGA
CTTTGTGACTGACTGTATGGGGCAGCTGCAGGGGTAGGAGAGGAGGAACGATTAAGACATGATGAACTGGG
CTATGAGTTGGCAGCTCCATTTACTCCAGAGAACACAGGAGGTGAAAATCATGGGAGACTTGATGAAAACA
CTTTGAGAGGCACCATGGGGATAAAAGCCAGAAATAAGGTGGGAAATGGTGGAAGCTATTCATTCTAGAAA
AGAGGGTGGGAGGATGAGCATAAGTTAACAGGAAACAAGTTAATTTTTTAAAAGTGCT
The following amino acid sequence <SEQ ID NO. 90> is the predicted
amino acid sequence derived from the DNA sequence of SEQ ID NO. 32:
HFKINLFPVNLCSSSHPLFNELPPFPTLFLAFIPMVPLKVFSSSLPFSPPVFSGVNGAANSPSSSCLNRSS
SPTPAAAPYSQSQSPVCVIAGMSLESTNILYSHTCLPPMSSAPLLVSEFQVGPVPFFLPCRLSRTRSLPTS
DFLSDDFWGFSICLLEGPLGDFYGTLIASFLYLRNVFLLLETPKIHDIFFTKLFLLSPAFNKSLFAKKWCR
FFTTASEKSV
The following DNA sequence Seq-2616 <SEQ ID NO. 33> was identified in
H. sapiens:
TTTCCCAGATAAATTGTATGCACAGTAACTGGTGTTGCAGTATACCATAGCATATATACATCCATTTGGCA
CACTGCAGGTGCCAGTGGGACAACATACCAGAGTGTAAGTCTTCCTGATCATTTTCATGATGTCCTCAGTT
ATTTACCTTGTAATAAGCTTGTAAACGTCTATGATTGTTTTTGAGTCATCCCAATGCAGTCATGTAATAAC
AACATGTATTTTAAATGAAACTTGGGGATTTTCCTCCATACCTGAATCTCTAGTATTCACATAAATGAAAA
ATCAAAATTAGGATAAGTTAGTGTCAAACATTAATGGATTTTTACAATGCTAATTGGTGTTCCTTTTTAAA
TTATTGCTGCCTACAGACACATAGCTATAGTTCCATGCACTTTCAACCACCAATGCTGCCAGGCTAGTAAA
GCAGTTAATGTATATTTGGGGTTAATTATCAGAATCACCAGAAACAATTTTTTTAATTTTTAAAATATTTT
ATTTTTCCACAGATTATTGGGGTACAGATGCTGTTTGATTACATAAGTTCTTTACTGGTGATTTGAGAGAT
TTGGGTGCACCCAACATCCGAGCAGTATACATTATTCCCTATG
The following amino acid sequence <SEQ ID NO. 91> is the predicted
amino acid sequence derived from the DNA sequence of SEQ ID NO. 33:
FPRIVCTVTGVAVYHSIYTSIWHTAGASGTTYQSVSLPDHFHDVLSYLPCNKLVNVYDCFVIPMQSCNNNM
YFKNLGIFLHTISSIHINEKSKLGVSVKHWIFTMLIGVPFIIAAYRHIAIVPCTFNHQCCQASKAVNVYLG
LIIRITRNNFFNFNILFFHRLLGYRCCLITVLYWFERFGCTQHPSSIHYSL
The following DNA sequence Seq-2617 <SEQ ID NO. 34> was identified in
H. sapiens:
CGGGTTATTTAGAGAACCACTTGAAATACCACCTCCTTGGTAACACCAGCTCCCTCCACCCCCTGAGCTCA
CGGTCTCTTCCCTGTGAGATGCAGCACCAGGTAAGGTCATTAACAACCAGGTTTAGAGTAAACAGTGCTGG
GCTGTATTTCTGATCCTGCCTTTCCCTAACTCGGTGCTCTTTCGCAAGTTATTAAGTTACTTCATCTGTAC
AATGGGTTACACTTATGCCTTTTACATATGGTTGTTGCGAAGATTGAGTGATATGCATACCAAAAATGCTG
AGCAGAACACCTTGTCCATATCTTTCCTCTCTGTTATTAAATGGAGGCCTTTAAGGTTAAGTAATTTGTTA
TTGTTGTGGTTAATTTTAGTCCTCTGAATTTTAATCTAGTACAAATTGTGCTGCATTTGGCACATGGTACA
TGTTCATGAATATTGAGTGTTGTATAAAGGAATGAAAAATCAATTACATGAAAAGAAATTCCAAATCTTAC
ATTTTACAAACACAGACACAAAGAATACTAAGATTTAACTCAGGGGCAAAAGTTAAGATTTGGCCACCAGC
ACGTGGTCAGCTTCCTTGAAAGTTTGTTTCTGGC
The following amino acid sequence <SEQ ID NO. 92> is the predicted
amino acid sequence derived from the DNA sequence of SEQ ID NO. 34:
GLFREPLEIPPPWHQLPPPPELTVSSLDAAPGKVINNQVSKQCWAVFLILPFPNWVLFGKLLSYFICTMGY
TYAFYIWLLRRLSDMHTKNAEQNTLSISFLSVIKWRPLRLSNLLLLWLILVLILIYKLCCIWHMVHVHEYV
LYKGMKNQLHEKKFQILHFTNTDTKNTKILRGKSDLATSTWASLKVCFW
The following DNA sequence Seq-2618 <SEQ ID NO. 35> was identified in
H. sapiens:
ATTCTAATGCCATTAATCCATAGCATTATCTCCATCTCTGTTTTAAATATCATGCATCCTCATTCTATGAT
TCAATTTAAAGGAATCCTTCAAAAGGATCAATCTAAATAAATAACAAGTTAGCTTTCAGGCAAACAAATAA
ATTTGCTTTGTTTTATATTCACCATAAATATTTCACTTAATTACTGAGGTACCTTGTTCAGGAAACACAAA
ACAACATTATAAATTAATTAGCACTGTCCCTGCTGACGTTTTAGTCCTGTGGAATGCAAAAGCTAAAAGTA
AAAACAGGCCATGAAGCCCAACCAGAGCACACATCGTATGCAAATGATAAAGCCCACAAACATCATGGGAT
CATTCCTGGGACATTCTGAATCACCAAAATTTTGTCTTTAATCAAGTATTGCCCTATTTATTTTCAAATTC
AA
The following amino acid sequence <SEQ ID NO. 93> is the predicted
amino acid sequence derived from the DNA sequence of SEQ ID NO. 35:
LNLKINRAILDRQNFGDSECPRNDPMMFVGFIICIRCVLWLGFMACFYFLLHSTGLKRQQGQCLIYNVVLC
FLNKVPQLSEIFMVNIKQSKFICLPESLVIYLDSFRIPLNIIEGCMIFKTEMEIMLWINAIR
The following DNA sequence Seq-2619 <SEQ ID NO. 36> was identified in
H. sapiens:
TTTGGGTCTAGAATCCCCTTGGTTTGGGAAGCATCCAGAAGGAGCTTCCATCCCCATCCATTTCTTGCTCA
CTTCCTCCTCTCTAGCTTTGTTTACATGTCTCTCGATACCTAGACAGAGGCAGAGGCATGGACTTCTGGTT
CTAATCATTCAAGCCTTACACGTCCTTCAAGGCTCCATTCAGAATTATCTTTCCTCGGGGAGTCTGCTTCT
CCTATTTCAGGATTTACTGACTATTCTCTTATCTCTTGTAACATTTAATATCCCACTCCTTAGCATTAACT
TTTAAACTTGCTTTCTAATCCTGAGGTTTGTGTTCCTTTGCCTTGTAAAATTCTTTGTAAATGGCCAGCCC
AGTACGTAGCCCAGTCCCAAGCACCACGTAGGCAATTGAAGGAGCTGGGACAAAAAGAGTTCTTTGTTTGA
ATTTCTTTTACTGCTCTGAGTTTACTCTGTATTTGCACATGAGTTTTAATGTTTTGGGGCCATTGAACTAT
TTGAGAATCTAGAAGATAATACACCTCTTTTCAGAAAAACACATATGAATACACACACACACATGCCACCT
ACACACACAATTTTGCATGTAATTTTAAGGATTCATTAACCTTAGCTTACCAGACTGTAAGTTCCTTTGCA
TATT
The following amino acid sequence <SEQ ID NO. 94> is the predicted
amino acid sequence derived from the DNA sequence of SEQ ID NO. 36:
YAKELTVWAKVNESLKLHAKLCVVACVCVYSYVFFKEVYYLLDSQIVQWPQNIKTHVQIQSKLRAVKEIQT
KNSFCPSSFNCLRGAWDWATYWAGHLQRILQGKGTQTSGLESKFKSCGVGYMLQEIRESVNPEIGEADSPR
KDNSEWSLEGRVRLELEPEVHASASVVSRDMTKLERRKARNGWGWKLLLDASQTKGILDP
The following DNA sequence Seq-2621 <SEQ ID NO. 37> was identified in
H. sapiens:
GCAAGTTATCTGTATTTATCCCCCTACAAACACACACTCCTAACATACAGTGGTGAGAGAGGAACAACATA
ACTGCAGAGGAAGTAAGTGAGAGACACAAAGCAGTCATTGGTTCATTGCTATAATGAAATTCTCCTAGACA
AATGCTGCCAGGATCTCTTCCCTGGGGATAAGGTCTAGTTATCTTCCTGGAAGTGGTTTCCAGCTCACTAT
TCTCTACTGTATAATTACAGTGACTCCCTCATCCATCCTCTTGTCTTCTCAGATCTTAACTTTATCCTCTA
GACTCCAGGCTCCTCCTCTGAGATGTTCTCACTTTTCTGCAACAAAAGCTGAGTCTATTTCTCAATCTGTT
TGCTGTCCATAGAAAATGGAAGGTTCAGAGGCTTTTATTCAATTTTCTCAGTCTCTTTATTGCAAGCTGGG
TCCCATTTACTTATATAACTCTTTTAAAAAGTTTTTGTGGGCTTTCTATGTATCAGATAATAGACCACTTC
ATTTGATAAAAAGCCACATTCTTTGTTTTCCAGACAAGCTTTCTATATTTTGGACAAGTAAGGCCACTTA
The following amino acid sequence <SEQ ID NO. 95> is the predicted
amino acid sequence derived from the DNA sequence of SEQ ID NO. 37:
KLSVFIPLQTHTPNIQWERNNITAEEVSERHKAVIGSLLNSPRQMLPGSLPWGGLVIFLEVVSSSLFSTVL
QLPHPSSCLLRSLYPLDSRLLLDVLTFLQQKLSLFLNLFAVHRKWKVQRLLFNFLSLFIASWVPFTYITLL
KSFCGLSMYQIIDHFIKATFFVFQTSFLYFGQVRPL
The following DNA sequence Seq-2624 <SEQ ID NO. 38> was identified in
H. sapiens:
TTATGGTGTTTGTAAGATCTTATTGCCCAAAGAGTCTGTTCTGTCCATCTTATGATATCTGTTTTAACATT
AATGATGCTCAGTTGTGTCTAGACCCTAAAAGAAGAAGTTTGTATGACTTTCCATGCTGTTATGGTCAGGA
ATTTAGTTTTAAGCTTTTTTGGGGCCTCTAAGCCACAAGGGGATCTGTTCAGTCAGTTCAGTAGAGGGCTT
AGGATTTATCATCTTTAATTCACATTCCCCCATTTTGGTCAAAATATGCCAAAAGTAGCATCAATAGCCAA
GCTCTTATTTCATTCCATATTATTACCAGGTGGTGTGGCTATCTATCTCAGATATATTCTGTTCTTCAATG
GGACCCATATAGCCAAGGGACTTATAGCCAAAAGACTTACAGCCAATTAAACATTCTAGGACAAAAGGGAA
TGGAGGTGGGAAGGCATTCATTATTCCTTAAAAACCTTTTGAGCAATATAAGAGCCACAAACCAAAAGCCA
AAAAGTAAGCTTACAAAACCGATTTATCTATAAGTTCTATGTGTTGGGCCATCGGCTCTTAGGCATCTGTG
AGCCCATCTTTTTTGGAGGATCTGAA
The following amino acid sequence <SEQ ID NO. 96> is the predicted
amino acid sequence derived from the DNA sequence of SEQ ID NO. 38:
MVFVRSYCPKSLFCPSYDICFNINDAQLCLDPKRRSLYDFPCCYGQEFSFKLFWGLATRGSVQSVQRADLS
SLIHIPPFWSKYAKSSINSQALISFHIITRWCGYLSQIYSVLQWDPYSQGTYSQKTYSQLNILGQKGMEVG
RHSLFLKNLLSNIRATNQKPKSKLTKPIYLVLCVGPSALRHLAHLFWRI
The following DNA sequence Seq-2625 <SEQ ID NO. 39> was identified in
H. sapiens:
AAGGCAGAGGGGGCCAGCAGGGCGGGTTACAGAACCATGATGTGTTTTTAACTGGACTCACTTCTGCCAGT
ATCTGCCTGACTCTTCAGCCCATGTCTCTTTTCCTTGTTGTAATACTAATGGGGGCATTAAGCAGCCAGAG
AAGGGGCCTCCGACGCCACTGCTTGTACCTCTGGAGTTACATTTAGCGGCATTTATATTTTGTCATGTGAA
ATTCGAAATCCTCATCCAAAATGCAACTGTGGGGGAACTCTCATAGGAATTTCAGCCAATTCTGGCTCC
The following amino acid sequence <SEQ ID NO. 97> is the predicted
amino acid sequence derived from the DNA sequence of SEQ ID NO. 39:
GRGGQQGGLQNHDVFLTGLTSASICLTLQPMSLFLVVILMGALRSQRRGLRRHCLYWSYIRHLYFVMNSK
SSSKMQLWGNSHRNFSQFWL
The following DNA sequence Seq-2626 <SEQ ID NO. 40> was identified in
H. sapiens:
CCCTTCCTCCCCAGCCATACCGTGACCCACCCATAAGCTGGCCCCCTTAGCTCTGGCTCACCTGGCTCAGA
CTTAGAGGTCGCAGCATTCCTGCTGCTCAGGAAATAAGGACTGCTCTTGAGCTCCTCACAGGCCCCAGGAA
TCCCAACAAAAGCCAACCAAGGCTACCTTCAGGCCTTCCAGAAGGGGGTGGTAGTGTCCTCATCAGGTTCC
CCAAGTTTAGGGAGAGGGCAGCTGGGCCCAGGGCCCTTCTCCTTGTGGCTCAGGATTTAGCCCCACTTACC
ATGGTGCAGCCCCAGCCTTCCAGCCAACCCAGCATTAGAGGCAGTGGCTCCTCTTAATGCCAGGCCCTAGT
TGGCTCAGGCATAATCCAGCCAGGAAACCTCTCACCTTTCCACAGCAATGGCCACCAGTGTGAAAACGGAA
GCCGACACAGACATGCCCTGCACCAAGCCGCTCATCTTGCATGTGGCATTGTCGAAGGGCCACCCTGCAAT
GACAGAGGCCCCCACAGAGTGAGAGATGCCCACGCATCAAGAGCCAGAGACTGAAAGCCCTCCAAGCCAGG
TCCCCTCTGAGCTTGGATCTTTCCTCCATGACCTGCTAGGTGTTATCTGGTCTCTGCT
The following amino acid sequence <SEQ ID NO. 98> is the predicted
amino acid sequence derived from the DNA sequence of SEQ ID NO. 40:
SRDQITPSRSWRKDPSSEGTWLGGLSVSGSCVGISHSVGASVIAGWPFDNATCKMSGLVQGMSVSASVFTL
VAIAVEREVSWLDYAANGLALRGATASNAGLAGRLGLHHGKWGILSHKEKGPGPSCPLPKLGEPDEDTTTP
FWKARPWLAFVGIPGACEELKSSPYFLSSRNPATSKSEPGEPELRGPAYGWVTVWLGRK
The following DNA sequence Seq-2627 SEQ ID NO. 41> was identified in H.
sapiens:
AAACTCCCAAACGATAGTAACTTAAATAACTTAGGTCTTTAATACTCTCTTCAGTAAAAGAATTCTAGTAG
TTGGAGAGTCCACCATCCCTAGGAATGTAGTTCTTGTCCTCATGGTTCAATATAGCTGCTGATGCTCCAGC
CATTACAGCCACATTCCAGACACCAAAATATGGAAAGAGAATGAAGAGAAGAAGAGCGTGCCTAGGAGTCC
CATGTATTATTTCCATATATATTTGGGCAGAACCTAGTCACAGGGCCACTCCATACGTATCTGTTAGCTAT
TGCTACATAGCAACCACAAAATTTCCATGTCATACAACACATATCTGCAGGTTGGCTAGGGTTCAGTTCCT
CCATGCTGGTCTCAGACAGGCAGTTCTGCTTCCGGTTACAGTGGCTGAGCTGATTCCATTTCTCACTGCAG
GTCTGTGTTTCAGTTGAGTGACTGTCCCATGTGCCTTTCATCTTCCTTGGGTTGATGAAAGGAAGCCACAT
CTTTCAACAGGGCTAGCCACATCTGTTCCTCATGGCCCAAAGAGACACCAAAGAGCAGATAGAAATAGGTG
AGACCTCTTAAGGTCTAGACTCAAAACTGGCACACTGCCACGTCTGTTCACAAGCTATTAGCCAAAGCATA
GATGCATTACCAAGCCCCAAGTCAAGGCCAAGAAGTACAATCCACC
The following amino acid sequence <SEQ ID NO. 99> is the predicted
amino acid sequence derived from the DNA sequence of SEQ ID NO. 41:
TPKRLKLRSLILSSVKEFLESPPSLGMFLSSWFNIAADAPAITATFQTAKYGKRMKRRRACLGVPCIISIY
IWAEPSHRATPYVSVSYCYIATTKFPCHTTHICRLARVQFLHAGLRQAVLLRVTVAELIPFLTAGLCFSVT
VPCAFHLPWVDERKPHLSTGLATSVPHGPKRHQRADRNRDLLRSRLKTGTLPRLFTSYPKHRCITKPQVKG
KKYNP
The following DNA sequence Seq-2628 <SEQ ID NO. 42> was identified in
H. sapiens:
ACAATAATTTGTTGTATATTCCAAAATAGCTAGTAGTGTAATGTTTCCAATACAAAGAAAAGATAAATGTT
TGTGGTGATGCATATTTCAAGTACCCTGATCTGATAATTGCACATTGTATACATCTATCAAAATATCAGCA
GTACCTCCAAAATATGCTCAATTATTGTATAAGTACAAAAAAATTTAAACAATTATAATGTATTATTTATT
TCTAAATGGTTTATTAGATTTAAAATTTTCTTGGTGTTTAATTTTTTCATATATTACCTTATACCCTTTAA
CTTCCTAAAATATATTAGGTCTTCATATTTTAGAGTAAAATTCTGAAAATCCTTTGAGTATCTGATTTTAC
AATCTTTTCTTCCACTGATTTTCCCTTAGCAATGGCCTGTTTAAAGTGTTGTGATGATGTTACTGAGAAAT
GGGCTGGCTACCTGATGCACATAGAAGCCAATACTATGGCAGTGGTTTTCTAGAAAAGAAAAGGCTTTACT
GTGAGTCTACTGGCAAGGAGACAGGTGGCAACACTCAAATCTGTCTCCCTGAACTGAGGATGGTGGGGT
The following amino acid sequence <SEQ ID NO. 100> is the predicted
amino acid sequence derived from the DNA sequence of SEQ ID NO. 42:
TIICCIFQNSCNVSNTKKRMFVVMHISSTLILHIVYIYQNISSTSKICSIIVVQKNLNNYNVLFISKWFIR
FKIFLVFNFFIYYLIPFNFLKYIRSSYFRVKFKSFEYLILQSFLPLIFPQWPVSVVMMLLRNGLATCTKPI
LWQWFSRKEKALLVYWQGDRWQHSNLSPTEDGG
The following DNA sequence Seq-2629 <SEQ ID NO. 43> was identified in
H. sapiens:
CCTCTTACTTGGGCCCCGTTCACTAGTCCTTCAGCCAAACTGCCTCACATGCTATTCCCAGTATGAAAATC
TTGCCATTCCCTTTATCTTTTTTCTCTTCTCTCATTTACAGCCCTGTGCTAGTTTCTTCATTCCCTTCAAG
TTCTGGCCAAACTTTATTTACCTCTTGACTGACCACTCCATCTAAAATAGTACTCATCACTGTGTATCCCC
TCAACACACTTTATAGGTCATGGCCATCACCTGATAATGTGTTATGTATTTTTTGGTTTACTTGTTGTGTT
AGTTCATTCTTGCATTGCTGTAAAGAAATTCCTGAGACTGGGTAATTTATAAAGAAAAGAGGTTTAATTGA
CTCACAGTTCTGCAGGCTGTATGGGAAGCATGTTGCTGGCATCTGCTTGGCTTCTGGGGAGGACTCAGGAA
ACTTACAATCATGGGGAAGGTGACGGGGGAGCAGGCACATCTGACATAGCAGGAGCAGCAAGTGAGCAAAG
GGCGACGTGCCACACACTTCTAAGTAACCAGACCTCATGAGAACTCACTATCATGAGAACAGTACCAGGGG
ATGGTGCTAG
The following amino acid sequence <SEQ ID NO. 101> is the predicted
amino acid sequence derived from the DNA sequence of SEQ ID NO. 43:
SYLGPVHSFSQTASHAIPSMKILPFPLSFFSSLIYSPVLVSSFPSSSGQTLFTSLTTPSKIVLITVYPLNT
LYRSWPSPDNVLCIFWFTCCVSSFLHCCKEIPETGFIKKRGLIDSQFCRLYGKHVAGICLASGEDSGNLQS
WGRRGSRHIHSRSSKAKGDVPHTSKPDLMRTHYHENSTRGWC
The following DNA sequence Seq-2630 <SEQ ID NO. 44> was identified in
H. sapiens:
AGTATAACAATTCACTGCTTTACATCTCTATATTTTGCTTATCTCAAGTATCCACTTTGTCTGGTATAGTG
TGCTCATTCCACAGTTTTTGGCTGTCCTGGGAACAACAATCTAGTGCAACTCCAGCAATGTGAGTTATAGT
GCAAATGTCAAACCAGAGCAGCATCACCATCTAGAGGTCAAAATGATAACTGCAAACTTTCTCACCTTTAT
GAGCCTTCCGTATTCTGTATACATAGCAGTTTATGTGAATGTACAGAAAATAATGTTTGCTATTGTTTTCT
CTCCAGTTGGGTTTCCAGAAAGAGATCATGGCATAAAGCAGGAACCACCTGTATTTACAGATGGCATAGGG
AAGCATACATCGCAGAGCCATATATCAGCAGCACTACAGCATGTTTCAACCAAAGATGAGCCTCCCACATG
TCAGACAAACCACCTACATTGGGACCACAGCAGTGACAGTGTTTTTTAGCACATTCCTGATAATGAAATCT
ATGTTGAACTCAACATGAATGGCTTTTCCTTTCTCTTGGCAGTCAACAGCCTACACCATTCTGCATTTGAC
TGTTTAGTTTATTCTCCCCTCTGGAAAGGCATGACTATGGAAACAGAGTAGAGGATATTTTGGGGATTTAT
GAAACTATTAATATAATTTACTCTCATTGCTGTGCTTTCTACAAA
The following amino acid sequence <SEQ ID NO. 102> is the predicted
amino acid sequence derived from the DNA sequence of SEQ ID NO.44:
YNNSLLYISIFCLSQVSTLSGIVCSFHSFWLSWEQQSSATPANVIVQMSNQSSITIRSKLQTFSPLAFRIL
YTQFMMYRKCLLLFSLQLGFQKEIMASRNHLYLQMAGSIHRRAIYQQHYSMFQPKMSLPHVRQTTYIGTTA
VTVFFSTFLIMKSMLNSTMAFPFSWQSTAYTILHLTVFILPSGKALWKQSRGYFGDLNYYNLLSLLCFLQ
The following DNA sequence Seq-2631 <SEQ ID NO. 45> was identified in
H. sapiens:
TCAGCTTATCTGGTCAATAGCTTTTCGCTCTGTTGCATACCTTGAGCATATGCATCAGCTACAATGTTTAT
AGGTAGCTGTATGGTGTTTGACACAGCACATGGCGTACCTTTAAAACAATTATAGCACTGGGATTTGGATC
TGAATTTATGTTGCCTTGTCAAAGTTTCCTCTTTGTAACATGGTAGCCTTTTAAATATTAGGCAGCTACCT
GCAACACTGGGCATTCAGACTAACCCATCAGGCTTATGGCATCTTGCTCTTCTCGTTCCCTCTCTGTGTGT
TGGTACATCATGTTAGGTTTATGCAGTAGACGTAGATAGGAAGCAAGCCAATTGGCTACAGGGTATTGAAA
GTCAATTGCTGAGAATGATAAAAGACAAGGATACCCTTCTCTGCAAAGAAGTGCTAAGAAGATTCTAAACG
TATACAAGGATCTCAAGAGAAACAGTCCCAGATAGCAACACTATTCAGTCTTAGACTATGCCTGATACTAT
ACACTTCTCCAGCTCCTCTGCTCCTCAGAGCAGAAAACAGAAGATTTTGAAATGAGCACCACCCCAGCTCC
TGAATACAATGGTACCTTTCATCTATTTCTGGTGACTTTTATTTTCTTTTGTTGCTGGATCCCCTACATAA
TTGTAAGCATATCGCAGGCAAGCACAATGGTAAACAGTGGGTGGACGCTTCCTC
The following amino acid sequence <SEQ ID NO. 103> is the predicted
amino acid sequence derived from the DNA sequence of SEQ ID NO. 45:
SLSGQLFALLHTLSICISYNVYRLYCVHSTWRTFKTIIALGFGSEFMLPCQSFLFVTWPFKYAATCNTGHS
DPIRLMASCSSRSLSVCWYINLGLCSRRREASQLATGYKSIAENDKRQGPSLQRSAKKILNVYKDLKRNSP
RQHYSVLDYGYYTLLQLLCSSEQKTEDFEMSTTPAPEYNGTFHLFLVTFIFFCCWIPYIIVSISQASTMVN
SGWTLP
The following DNA sequence Seq-2632 <SEQ ID NO. 46> was identified in
H. sapiens:
ATAGAACTTGATTATATTGGTATTTTTATTTCAAATTTTCAATTTTTGGAATGGCAGAATGTTGCTATTGA
AAAGTGTCTTAAAGGTCACCACTGTAACCCCTTCATTGTGCTTGAGACCTGCTCAGCTCCTAAATTTAACA
GGGGACGGATCTGAGAAACTGACTCCAAGTTGTAACCTCTTGCTTAGTTTTCTTTCTAGGGAGATATCCGT
CTCTCCAAACCTGTCGAAATCTAAATTTATTACCTCTTACCTAATACTTCGTCCCCTGTGGACTTCACTTC
ACTGTTTGTGCTAATAGCCTTTTCATCACCATCTTGACTTTGGATTCTAGAGCATCACCTACTTCCCCATT
TTCTGTGACCCTTACATTCCTCCTGTCAGTCACTATGTCTGATTTATTGTTCTCCCCTATCTTTTGCCCTT
TGCAAATCCTCAAGCCCTCATTCTGGTTCAGACCTTTAAAAGGCTGAGTTACTGGACTATAGTGTTACCCA
AAGTGAGTTGTTCCATAAAAAATTAGTAAGTTGGAAAAAAAAACAAAAAACAAAAAAATACCCTACCCATA
AAGTTGGTAAATGTTCCTGTAAAAAGGGTTCCTTGGCCAGGTACATGTTAGAATAGCTGGTTAAGTTTCTT
TGCAGAAAGACTTCTCCTGGCCTTCATTTGTGACTGTG
The following amino acid sequence <SEQ ID NO. 104> is the predicted
amino acid sequence derived from the DNA sequence of SEQ ID NO. 46:
RTLYWYFYFKFSIFGMAECCYKVSRSPLPLHCADLLSSIQGTDLRNLQVVTSCLVFFLGRYPSLQTCRNLN
LLPLTYLVPCGLHFTVCANSLFITILTLDSRASPTSPFSVTLTFLLSVTMSDLLFSPIFCPLQILKPSFWF
RPLKGVTGVCYPKVVPKISKLEKKTKNKKIPYPSWMFLKGFLGQVHVRIAGVSLQKDFSWPSFVTV
The following DNA sequence Seq-2633 <SEQ ID NO. 47> was identified in
H. sapiens:
GCAATTAAGTTTTGTACTGTATGGACAGTGTGAAAAACATTATGGAAAAACAACTTGAAAGAAAATGTGAC
AGAATTTCTCCTAACAATGTCATTGCTTCAACCAGCTACAAATTTCCAACCTAGTTTCTTTCTTTTGCTGT
TTCTTCTTTTGTCTTTGATACAATCATACAGCCTCTCTTCCTTGAAGAGATAATAAAAGACTAACAGTTAA
AAGATCTGGAAGACTCATATTCTTTTCTTTTTCACTGGCTACGGTTTTGAAAAGAGGCTGTTGGCTTTTGA
TTTTTTCTTTTGGGTTCTTTACATCGCCCAATTCAAACAGGTCTGCTCTCAAAGAAAACAAATCAAAATTG
TCAAGACCTGTGAAGCATGAAAAATAAATTGCTTTTTCCAACTCCAAAAAGCACCAGAAAAGCATTAATTT
TGATCTTTTATAAACCTCTATCCCCTATCCTCTAATCTATAGATTTCACAGAATGTTTATATATTCTTCTG
TATAATACAGGAGATCAAACCTTATTATGAATAAATTGAATTGAACCTGTAATACAACTAATATTTAAACT
AGTGTTATTTTGGAGTTCAACTAGACACATATAAAACATTTCAAGTGAGATGACACAAATTCCTGGGGCTG
CCAGTATAAAATAAACAGTCCAGTAAGCTGCATCTACCATGCCGTTAAGGGACTCTGTCCTTTTAGCTGGT
GGGAGCACAGGCTTCATAA
The following amino acid sequence <SEQ ID NO. 105> is the predicted
amino acid sequence derived from the DNA sequence of SEQ ID NO. 47:
MKPVLPPAKRTESLNGMVDAAYWTVYFILAAPGICVISLEMFYMCLVELQNNTSLNISCITGSIQFIHNKV
SPVLYRRIYKHSVKSIDRIGDRGLKIKINAFLVLFGVGKSNLFFMLHRSQFFVFFESRPVIGRCKEPKRKN
QKPTASFQNRSQKRKEYESSRSFNCSFIISSRKRGCMIVSKTKEETAKERNVGNLLVEAMTLLGEILSHFL
SSCFSIMFFTLSIQYKTL
The following DNA sequence Seq-2634 <SEQ ID NO. 48> was identified in
H. sapiens:
TCCTGAGAAGACCTGCAGCACAGGGTAAAATATGCAAGGGAGGGCCATATAACTTTTATCTTTACTTAATT
TATTTTAATTTACTAATTTTTAAGTATTAACCTATTTTGTTTTTATTAAATCTCTGTGGTTGCACAGAATT
CAAATTGCAGCAAAAATCATTCAGGGCTAAACACTGGAAAAATCTCTTAATTCTAAGGTACATGACACAAT
GGACTCAAAAACAGTTGCTGAGTCCCTTTCACTGGAGAAATTTAAAGAAAGGGTATAGAAAAGTTTTGACC
AATTCCACCCAATCCTGCATCCCCAATTCCAATCTCAAGGACCAGTTTCCATCTGATCTCTCTCCACCTAC
AGATGGTGGTCCTGAATCTCCAAATCAACAAACCAAAAACTGAATCCATCATCTTCTCACACCTGGTTTTT
CCTTCCAACTCCCTCATTTCTGTGACCTGCCCCATAACCTTACCAGGAATCCAGCCCCCAAAGCAGGGTGG
ACTCCTCCCTCTGCAATGGACACCAGGGATTCAGGTCCTGTTGCTGGCTCCAAAATGCCCACAATGCCCTG
TTCTCCCAAATCAGCACATTCAACAGT
The following amino acid sequence <SEQ ID NO. 106> is the predicted
amino acid sequence derived from the DNA sequence of SEQ ID NO. 48:
SEDLQHRVKYAREGHITFIFTFILIYFLSINLFCFYISVVAQNSNCSKNHSGLNTGKISFGTHNGLKNSCV
PFTGEIRKGIEKFPIPPNPASPIPISRTSFHLISLHLQMVVLNLQINKPKTESIIFSHLVFPSNSLISVTC
PITLPGIQPPKQGGLLPLQWTPGIQVLLLAPKCPQCPVLPNQHIQQ
The following DNA sequence Seq-2635 <SEQ ID NO. 49> was identified in
H. sapiens:
TTTGACTTAACCCTTTGTAGCCCAGGTAATAAATCCAAACTCAGCAAGTATGGGCTGGACCCCAGTAGCTC
TGTGGTTGCCACTTTTTGGCCCATATTGAACCGACGTCCCCTTGGCATCTACCAGGGACTCCTCAGGGAGA
GTGTGGGAATGATGGGGGAAGACTCGTCACTCTTTTGTAGAGCGTGGGGCAGATGATAGCAGAGACCTTCC
AGGGCCCAGGGCTGGGGTCTTGTCTTCCTTGGATGTGGTCTAGCGTTGCTCCAGATGGTGGGTTTGTGGCA
GGTGGGGCAGAAGCAGATGATGCAGTTGAGGCGGGTCTCTGGTAGAGAGTGATGTCAAAGATGAGCACTCC
TTTTATCCCCTGACTCTTCTGAGGATGGCTGCCTCCTTGGTGAGCCACTTGGAGGTCTCAGGCCGATCATG
CGGGATGGTGGCCCAGATGAGGAAGGGGATCCAAGGCGGTGGCCTTCCCAGATGCACTGGGCCCCAGCCCT
TCTTCCTAGCTTCGGCTGATTACTGTCGGCTTCAGCAACCAGGGCCTACCTGTAGGTCTCCACATTTGTAA
GCACCACAGAACCCAGTGCATCTTTGTGCACTCAAATCGCCCTTGATCCAGGGGTATTTTCTCATTCAGAA
CACACTTTGAAAGGCGGCCATTCCCCTTCTGGAGAAAGCCTGGAGAATCTACAGTGCCCTTAATTACAGTG
The following amino acid sequence <SEQ ID NO. 107> is the predicted
amino acid sequence derived from the DNA sequence of SEQ ID NO. 49:
HCNGHCRFSRLSPEGEWPPFKVCSEENTPGSRAIVHKDALGSVVLTNVETYRALVAEAHSNQPKLGRRAGA
QCIWEGHRLGSPSSSGPPSRNIGLRPPSGSPRRQPSSEESGDKRSAHLHHSLPETRLNCIICFCPTCHKPT
IWSNARPHPRKTRPQPWALEGLCYHLPHALQKSDESSPIIPTLSLRSPWMPRGRRFNNGQKVATTELLGSS
PYLLSLDLLPGLQRVKS
The following DNA sequence Seq-2636 <SEQ ID NO. 50> was identified in
H. sapiens:
AGATGCCCAGACACCTTCACTTCAGCAGACAAGGGGCAGAGTCCTGGAAAATCTAGGCAGGGAAGACTTGC
GCCTCTAAGAGTAAAAGGCCTCCCAGAGAGGACATCGATGAAAGGAGGACCACCTTCCAATGCCACTCTCC
AAAGCAGGAAACATCCAAATAAAGGATGTTGATTTTCAGGACCCCATCCCTTCATGAGTGCTTACACAACT
GGTATATCCTCTCCCGTCTCTTCCTCTGGTAGCCAAGACCTTATAGCAGTTTGAGTATCCTTTATCCAAAA
TGCTTGGGGTCAGAAGTGTTTTGAATTTCAGATATTTTTAAATTTTGGAATATTTATATCATACCTCTTGG
TTGAACCTTCCAGATACAAAAATCTGGAGTCCAGTGAGTATTTCCTTTGAGTGTCATGTCAGTGCTCAAAA
AGTTTTAGATTTTGGAGCGTTTCAGATTTCAGGTTTTTGAAATTGGAATACTCAACCTGTACTCTCTGTCC
TTGTTCTACCTCTACCAGACCCTCCCCCACAGGAATGATTTAGATCTGAAAA
The following amino acid sequence <SEQ ID NO. 108> is the predicted
amino acid sequence derived from the DNA sequence of SEQ ID NO. 50:
FRSKFIPVGECLVEVEQGQRVQVEYSNFKNLKSETLQNLKLFEHHDTQRKYSLDSRFLYLEGSTKRYDINI
PKFKNINSKHFPQAFWIKDTQTGIRSWLPEEETGEDIPVVALMKGWGPENQHPLFGCFLLWRVALEGGPPF
IHVLSGRPFTLRGASLPCLDFPGLCPLSAEVKVSGH
The following DNA sequence Seq-2637 SEQ ID NO. 51> was identified in H.
sapiens:
TCATCCTCCGCTGTCTATTTTGAGCTGTGAGTTTATCCACAAAGGAACAGAGCTGAAATGAAACAATTTCA
CCACAGTAACTTGTTAATCGGGCATCCTTTAAGTATGCTGGATTTAACACTGGAAGTTCTTTTGAAGACTC
TGAAAGTTTTCTTTAATCGTCATGAGATTTTTCCAAACTAAGTTCATGATATGGATTTTTTTCACTGTATC
TAGCTTAAGTCACATTTCAATTCAAATCTAAACCTAAACTGATGGAGCTGGAGCTAGTGACTTCAGGCAAT
TGGCATCTTTTCGCTGAATACAAACATCCTATTTAAAAGACCAAACACATGACTCCATTCAAAAATTAAAA
CAGTCATGTCTAGTGAAACAGCAAGAACACGGTCTGAGAAACGTGTCCTTGCACACACAGCGTGAATGCAC
TCACGCAAGCCTAGACGGTGCGGCTGCCGCACACCAGGCCCTGTGGTACAGCCTGTCAATTCCAGGCCCCA
AGCCTGCATACCATGTTGCTGTGCGGGACGCTGCCGGCGGCTGTAGCACAATGCTAAGTATCTGTGTATCT
CAACACAGAAGAGGTAGAGTAAAGTACAGTATTATGATCGTACGGGACGCGTGTTGTACACACAGTCTATC
ATTGATGGAAGCATCGTTATATGGCACATTACTGCACTGTAAAAAGACACCAAACTTCGGCCGGCGCAGTG
GCTCATGCCTGTAATCCCAGCACTTTGGGACGCTGAG
The following amino acid sequence <SEQ ID NO. 109> is the predicted
amino acid sequence derived from the DNA sequence of SEQ ID NO. 51:
SASQSAGITGMSHCAGRSLVSFYSAVMCKITMLPSMIDCVYNTRPVRSYCTLLYLFCVEIHRYLALCYSRR
QRPAQQHGMQAWGLELTGCTTGPGVRQPHRLGLRECIHAVCARTRFSDRVLAVSLHMTVLIFEWSHVFGLL
NRMFVFSEKNPIASHLQLHQFRFRFELKCDLSIQKKSISTFGKTSRLKKTFRVFKRTSSVKSSILKGCPIN
KLLWNCFISALFLCGTHSSKTAED
The following DNA sequence Seq-2639 <SEQ ID NO. 52> was identified in
H. sapiens:
TTCTTCCTTTTCCTTTCATTATCATTTTCTTTTTGTCTCAAAATAATGAAAAATGCATAAGGGTCTGTAGA
GAGAAGAAAATGTCCTTGCCCATGAACTTCTTGCAGGTATTTATCTTGCTTCTTTATCTTACTAAAAATAG
AATTGAAAGTTTTTCATTTTTTGTTTTTCAATTTTAGAGGATACAATGGAGATTCAGGAACGAATAGAAAA
TAGTTTTAAGTCTTTACTAGACCAGTTAAAAGGTAAGTTTTCCTACTGTAGATTCCTGTATTTGTATCTGG
TTGTATGGCAATAGCTTCGAAGTTCTTTCCCCTATTCCCAAGCCCAATCACCCAGAGATAAGTAAGTAGTT
TTAACACTTTGGAGTCAATACTCCTAGATGCCACCTAAACACATATGTGTGTGAATCAAAATACAGATAAA
AAGTAATCTTTAAACATAGGAAATGGTGTAATCCATGCTTTTTTGACTTTAATTTTTTTGTTATTTTGGAT
ACCTTTCCATGTCAGTTATATATACCCCATTTATTTTCAAGACTGCGTAATATTCTATAGTATTGTATTAA
CATTTTTTATGTTATCGCAATTGGTGACATATTATGTATATGAGTTATTTCTTCTACTGATGCTGAAATGA
ATATCTTGGGACAAATTGTTAGGGGTATTATTTGAGTCCTTCCTTGGGATTAAATT
The following amino acid sequence <SEQ ID NO. 110> is the predicted
amino acid sequence derived from the DNA sequence of SEQ ID NO. 52:
FFLFLSLSFSFCLKIMKNAGSVERRKCPCPTSCRYLSCFFILLKIELKVFHFLFFNFRGYNGDSGTNRKFV
FTRPVKRVFLLIPVFVSGCMAIASKFFPLFPSPITQRVSSFNTLESILLDATTHMCVNENTDKKSLNIGNG
VIHAFLTLIFLLFWIPFHVSYIYPIYFQDCVIFYSIVLTFFMLSQLVTYYVYELFLLLMLKISWDKLLGVL
FESFLGIK
The following DNA sequence Seq 2640 <SEQ ID NO. 53> was identified in
H. sapiens:
CTTTTGAGGATTAAAAATTCCTGCTTACTGTCGTTATAACACGGGGATTAATAAGCACCTTACTGGAATCT
CTCACCTACCATAATTTTAGTATGCTATGTGAGGGAATGAACAGTCTCACACATTTAATAATGACTACTCA
TATAATGCTTTTAATTGGTAATGACCTATATGAAACATGATATAGAAAACACATTACAGCTTCTCAAATGA
CCCCTATAAGTTAACCAATTGCTTAGGTTTCTGACAAATTTGAATCTGGCCCCATGCACCTTTGCTGGGCC
CCACAAAACAAGGAGGTAGATTATTTATGAAGGTCAACCACTCTGGCAATATCACCATTAAATATCAAGCT
CATCTGCCCCATAGCTCCTCCATCTTCAGGTCCAGGACTCTGGATTGGAATGACCTACCTCCACATTCAGT
TCTGTAAGTCATTAGGCATCATCCAAGATGGTAGATGATGAATAAATGGACAATGACTTAAGCTTTTTTTA
CTCTCTCATCCATTCCAATGCTTTCTTCCCTGGTCTTTGCTCATTATTTCCATGTTATTTAATATATATTT
GGAAGAATTCATGGCAGTGATAACAATAATGGCTACAATTTTTTATTACCTATGTATGCCAGGCATTGTGC
TAAGTGCTTCAGGTATAAGATCTTGTAAGGGATTGGTTACATTTTACAGATGGTAAGACTGGGATTCAGAT
GTTAGTTGCCTGTTTAAGTCAATAA
The following amino acid sequence <SEQ ID NO. 111> is the predicted
amino acid sequence derived from the DNA sequence of SEQ ID NO. 53:
FEDKFLLTVVITRGLISTLLESLTYHNFSMLCEGMNSLTHLIMTTHIMLLIGNDLYETYRKHITASQMTPI
SPIAVSDKFESGPMHLCWAPQNKEVDYLRSTTLAISPLNIKLICPIAPPSSGPGLWIGMTYLNIQFCKSLG
IIQDGRINGQLKLFLLSHPFQCFLPWSLLIISMLFNIYLEEFMAVITIMATIFYYLCMPGIVLSASGIRSC
KGLVTFYRWDWDSDVSCLFKSI
The following DNA sequence Seq-264l <SEQ ID NO. 54> was identified in
H. sapiens:
CTCTTCTCCCTAGGTGGTTTGCTGGCAATCTTTGGCATTCCTTAGCTTGTGGAAGTATCACTCCATCTCTG
TCCTGATTTCTACATGGTGTTCTTCCTGTGTGCATGTCTGTCTCCAAATTTCCCCATTTTATAAGGACACA
GTCATACTGGATTCGGGCTCATTCTAAAGACCTCATTTAATTTAATTCCATAAAGACCCTATCTCCAAATA
ATGTCACATTCTGTGGTACTGGGGGTTATGACTTAAACATATAAATTTTAGGGAGACAAATTTGAACCTCT
AACAGTACTGAACATCCAGGATGGAAGAACATGGTATTAGGTTGAGCCAAACAGAGTTGCTTACGTTTTGG
TTTTCCTCACCAGGACAAGAAACCCCCAGTGCAGGAAAATTGGAGACATGGAAAACAGGGCTTAAGTAAAC
A
The following amino acid sequence <SEQ ID NO. 112> is the predicted
amino acid sequence derived from the DNA sequence of SEQ ID NO. 54:
SSPVVCWQSLAFLSJLWKYHSISVLISTWCSSCVHVCLQISPFYKDTVILDSGSFRPHLIFHKDPISKCHIL
WYWGLLKHTNFRETNLNLQYTSPMEEHGIRLSQTQLLTFWFSSPGQETPSAGKLJETWKTGLKT
The following DNA sequence Seq-2642 <SEQ ID NO. 55> was identified in
H. sapiens:
TTATTATACTCAACACTGCTAGGAAAGAATCAGTGATGTTGAAGATATATATATATATATTTGCTTGTGTA
TTTGTGTGTGAGAGACACACATAGAAAAAAAGAGAGAGAGAAATATATTGGTTGACACTGGCTTCTTTGAA
AAAAGGCAGTTTAGTAACAATGGCCTTTACTAGACAGACATGTTAGAAGGCAGCAGGAGAAAGGGAATGTG
GTATCAGATATTTTCTGTAAAAGGTTTGTTATTAATATTCATGTGGCAAATTGTAGCTGATGTCAAAGTAG
TTATAAAGCAAGGGGAACACAATTCTTTTACAGCAATGTTGAGGTCTAAGAAACATAAAACAAATACCTGG
TAAGTACCATGCATATATACATACATAAACAATCAATAACTCACAAAACATTCACATATTTGCAACACTGC
TTTTCAGTTTATGCAGTTTATTTTTTGTTCTTTTTAAGCTTTTTATTATAGTGAATGTCTTATATTTCATT
AAAAGTTTTGATATTATATGTGAAACAACAGTTCTGATAAAGCAATATCTAGATAAAGGCTATTACTTACC
TTTCTCAAATTGATAGATTTTCTCCTTGTAACAAGCTCTGATATAAAATATGATAATTTGTTGAAAACTTT
TACACATTCAAAACTAAATTATCATATATTTAATGAGACTTTGGGTGTGTATGTGTGAGTGTGTGTCTGTG
TGT
The following amino acid sequence <SEQ ID NO. 113> is the predicted
amino acid sequence derived from the DNA sequence of SEQ ID NO. 55:
HTDTHSHIHTQSLIKYMIIFMCKSFQQIIIFYIRACYKEKIYQFEKGKPLSRYCFIRTVVSHIISKLLMKY
KTFTIIKSLKRTKNKLHKLKSSVANMMFCELLIVYVCIYAWYLPGICFMFLRPQHCCKRIVFPLLYNYFDI
SYNLPHEYQTFYRKYLIPHSLSPAAFHVCLVKAIVTKLPFFKEASVNQYISLSLFFYVCLSHTNTQANIYI
YIFNITDSFLAVLSII
The following DNA sequence Seq-2643 <SEQ ID NO. 56> was identified in
H. sapiens:
AAAACTTGGTTTTTTAAAGCAAACACAGAAACAATGTAATATAGGTCTTATTACATATGTAGGAAATAAAA
ATAATATGTATGACGACAACAGTAGTCTAAAATTCAGGAGACAGAGAATGGAAGTACATTGTTGCAAGGTT
TTCTAATACACATGTACAAAGTGGTATAATGTTACTTGAAAGATAACTGTGATAAGTTAAAGACGTAATCA
ATGACACTATATCAACCACTAAAATAATACAACAAAGGATATACGAAATATTTTTAAAAGTATAATTAACC
CAAAAGAAAGCATAGAGGAAAAAGGGAACAAAGAATAATAGATGGAATAAACAGAAAAAACTAGCCAGCTG
GTAAATTTAAAACCGATCATATACATATTCACATTAAATACAAAAAGTTTAAACACTTCAAAGTCAAGTCA
GAGGTGTCATATTGGATAAAAAGAAAGACTCAACTATATGTTACCTATAAGGAATGCACTTTAAATATACA
AACATATTAAAATAAAAAGATGAAAAGTTATATACAATGTTAATACTCATCAAAATAAAGCTAATGAGGCT
ATATTCATATTAAAAAGTAGGTTTTAAAGCAAAGATTAC
The following amino acid sequence <SEQ ID NO. 114> is the predicted
amino acid sequence derived from the DNA sequence of SEQ ID NO. 56:
SLLNLLFNMNIASLALFVLTLYITFHLFILICLYISAFLIGNILSLSFYPIHLLDFEVFKLFVFNVNMYMI
GFKFTSWLVFSVYSIYYSLFPFSSMLSFGLIILLKIFRISFVVLFWLICHLRLLITVIFQVTLYHFVHVYK
TLQQCTSILCLLNFRLLLSSYILFLFPTYVIRPILHCFCVCFKKPSF
The following DNA sequence Seq-2644 <SEQ ID NO. 57> was identified in
H. sapiens:
TCAAGTCCATGCTTTTACGGAAAGACCCCAGTTCCTGCCTCTTCTATATATTTATCTACCTTGTGGTGAAG
AGCATGTGTGTGCAACACCTTTGCCTGAAATGGTATGGTTTGGCATTAATGAATTGTGGGTCCATTGAAAA
GAAATCTCCTCTTGTTTCTCGTGTTATGGACAGTTCAAGGTTTGCCTTAGAACTAACTTCAAGGAAAAGTA
GCAGAATCGTAGGAAGGGACAATCTTGCCTTCAGTCCCACCCTCTGTTCCGGGCAGGTCTGGGTGGCTATC
TTCTTTCGGGGGCTTTTCCTTGCAGAAGAACTTCTTCAGCATGTCCTGGATTTCCTTCTTAATGGTCTTGT
GCATGTAGCCATAGACATAGGGGTGGATGCAGCACTGCAGGAAGAAAAGCCAGATGATTATGGTGATCACC
CACTGGGGTACCTGGGTTTCGACATCCACCCACACGGCCAGGACTGCTAAAAAGCAGTAGGGCCCCAGGGA
TAGCACATAGGAGAAAATGATGATGAAGATCACTTTAGCAGCTTTGCACTGGTAGCACCTGGGCAGAGGAG
GGTTGCTGTTGCTGTTACGACGACTGGGTGGGAGGCTCTCCGGGATGTTCACTGCCTCGACGTCATCCTCA
CTGAAATTGATGTCGTCTTCACCAAACTCCATGTCATCTTCACCCAAGTCAATGCTGCACTGGTTGACCTC
TGTGCGACCCTTGTCTGCCTTCATGCTGTTCTCCTC
The following amino acid sequence <SEQ ID NO. 115> is the predicted
amino acid sequence derived from the DNA sequence of SEQ ID NO. 57:
EENSMKADKGRTEVNQCSIDLGEDDMEFGEDDINFSEDDVEAVNIPESLPPSRRNSNSNPPLPRCYQCKAA
KVIFIIIFSYVLSLGPYCFLAVLAVWVDVETQVPQWVITIIIWLFFLQCCIHPYVYGYMHKTIKKEIQDML
KKFFCKEKPPKEDSHPDLPGTEGGTEGKIVPSYDSATFPSFGKPTVHNTRNKRRFLFNGPTIHCQTIPFQA
KVLHTHALHHKVDKYIEEAGTGVFPKHGL
The following DNA sequence Seq-2645 <SEQ ID NO. 58> was identified in
H. sapiens:
AGTGGAAAGACCACACCTAGGAACCGACTCTAGCTCTTACCACCCTGTAAGCCTGAGGCTCAGTTGCTGTC
CCTGGAGAACAGAAAACATAATCATGGCTATTCTGAGGGTCAGGGGCAAGTGCTTTGCAAGTGGGATTGTG
GTGGGCAGTGGGAGGGATTCTGGGGTTCACTGTCATGCTAGTTGTGTAACTGGGCAATGCAACCGTGTAAG
TGTCAGGAAACCCTCAATAAGACTGAGCCAGAGGCCAATAAGAAGCCAGCATTTACATGATGTTCTTTTCC
TTTTTGTAACTAGGAAATTTCGATTTGCACACTGATTTGGCCCACCATTCCTGGAGAGATCTCGTGGGATG
TCTCTTTTGTTACTTTGAACTTCTTGGTGCCAGGACTGGTCATTGTGATCAGTTACTCCAAAATTTTACAG
GTATGTTTTCTGCAAGTGCTGCCACTGAACTTCACCCAGGCTTGGGGTTATTTCTGCTAGAATCTTAGAAT
TTGGGGTCGGAGAACACCTAAGAGTTCACGCCAGCTCAATCTTGATTCACTGCCCAGGTCTACAACACTGA
GGAAGGAGAGGATTTTTTTAGAAGTTATATCTTTGTGATTATGTTTTTTGCTCATCACTAAAGTAATACT
The following amino acid sequence <SEQ ID NO. 116> is the predicted
amino acid sequence derived from the DNA sequence of SEQ ID NO. 58:
SGKTTPRNRLLLPPCKPEAQLLSLENRKHNHGYSEGQGQVLCKWDCGGQWEGFWGSLSCLCNWAMQPCKCQ
ETLNKTEPEANKKPAFTCSFPFCNEISICTLIWPTIPGEISWDVSFVTLNFLVPGLVIVISYSKILQVCFL
QVLPLNFTQAWGYFCNLRIWGRRTPKSSRQLNLDSLPRSTTLRKERIFLEVISLLCFLLITKVI

### Example 2

### CLONING OF nGPCR-x

cDNAs may be sequenced directly using an AB 1377 or ABI373A fluorescence-based sequencer (Perkin Elmer/Applied Biosystems Division, PE/ABD, Foster City, Calif.) and the ABI PRISM Ready Dye-Deoxy Terminator kit with Taq FS polymerase. Each ABI cycle sequencing reaction contains about 0.5 kg of plasmid DNA. Cycle-sequencing is performed using an initial denaturation at 98° C. for 1 min, followed by 50 cycles: 98° C. for 30 sec, annealing at 50° C. for 30 sec, and extension at 60° C. for 4 min. Temperature cycles and times are controlled by a Perkin-Elmer 9600 thermocycler. Extension products are purified using Centriflex gel filtration (Advanced Genetic Technologies Corp., Gaithersburg, Md.). Each reaction product is loaded by pipette onto the column, which is then centrifuged in a swinging bucket centrifuge (Sorvall model RT6000B table top centrifuge) at 1500 ×g for 4 min at room temperature. Column-purified samples are dried under vacuum for about 40 min and then dissolved in 5[l of a DNA loading solution (83% deionized formamide, 8.3 mM EDTA, and 1.6 mg/ml Blue Dextran). The samples are then heated to 90° C. for three min and loaded into the gel sample wells for sequence analysis by the ABI377 sequencer. Sequence analysis is performed by importing ABI373A files into the Sequencher program (Gene Codes, Ann Arbor, MI). Generally, sequence reads of 700 bp are obtained. Potential sequencing errors are minimized by obtaining sequence information from both DNA strands and by re-sequencing difficult areas using primers at different locations until all sequencing ambiguities are removed.

To isolate a cDNA clone encoding full length nGPCR, a DNA fragment corresponding to a nucleotide sequence selected from the group consisting of SEQ ID NO: 1 to SEQ ID NO:58, or a portion thereof, can be used as a probe for hybridization screening of a phage cDNA library. The DNA fragment is amplified by the polymerase chain reaction (PCR) method. The PCR reaction mixture of 50[t contains polymerase mixture (0.2mM dNTPs, lx PCR Buffer and 0.75 μl Expand High Fidelity Polymerase (Roche Biochemicals)), 1 μg of 3206491 plasmid, and 50 pmoles of forward primer and 50 pmoles of reverse primer. The primers are preferably 10 to 25 nucleotides in length and are determined by procedures well known to those skilled in the art. Amplification is performed in an Applied Biosystems PE2400 thermocycler, using the following program: 95° C. for 15 seconds, 52° C. for 30 seconds and 72° C. for 90 seconds; repeated for 25 cycles. The amplified product is separated from the plasmid by agarose gel electrophoresis, and purified by Qiaquick gel extraction kit (Qiagen).

A lambda phage library containing cDNAs cloned into lambda ZAPII phage-vector is plated with E. coli XL-1 blue host, on 15 cm LB-agar plates at a density of 50, 000 pfu per plate, and grown overnight at 37° C.; (plated as described by Sambrook et al., supra). Phage plaques are transferred to nylon membranes (Amersham Hybond N.J.), denatured for 2 minutes in denaturation solution (0.5 M NaOH, 1.5 M NaCl), renatured for 5 minutes in renaturation solution (1 M Tris pH 7.5, 1.5 M NaCl), and washed briefly in 2×SSC (20× SSC: 3 M NaCl, 0.3 M Na-citrate). Filter membranes are dried and incubated at 80° C. for 120 minutes to cross-link the phage DNA to the membranes.

The membranes are hybridized with a DNA probe prepared as described above. A DNA fragment (25 ng) is labeled with α-³² P-dCTP (NEN) using Rediprime random priming (Amersham Pharmacia Biotech), according to the manufacturer's instructions. Labeled DNA is separated from unincorporated nucleotides by S200 spin columns (Amersham Pharmacia Biotech), denatured at 95° C. for 5 minutes and kept on ice. The DNA-containing membranes (above) are pre-hybridized in 50ml ExpressHyb (Clontech) solution at 68° C. for 90 minutes. Subsequently, the labeled DNA probe is added to the hybridization solution, and the probe is left to hybridize to the membranes at 68° C. for 70 minutes. The membranes are washed five times in 2× SSC, 0.1% SDS at 42° C. for 5 minutes each, and finally washed 30 minutes in 0.1× SSC, 0.2% SDS. Filters are exposed to Kodak XAR film (Eastman Kodak Company, Rochester, N.Y., USA) with an intensifying screen at −80° C. for 16 hours. One positive colony is isolated from the plates, and re-plated with about 1000 pfu on a 15 cm LB plate. Plating, plaque lift to filters and hybridization are performed as described above. About four positive phage plaques are isolated form this secondary screening.

cDNA containing plasmids (pBluescript SK-) are rescued from the isolated phages by in vivo excision by culturing XL-1 blue cells co-infected with the isolated phages and with the Excision helper phage, as described by the manufacturer (Stratagene). XL-blue cells containing the plasmids are plated on LB plates and grown at 37° C. for 16 hours. Colonies (18) from each plate are replated on LB plates and grown. One colony from each plate is stricken onto a nylon filter in an ordered array, and the filter is placed on a LB plate to raise the colonies. The filter is then hybridized with a labeled probe as described above. About three positive colonies are selected and grown up in LB medium. Plasmid DNA is isolated from the three clones by Qiagen Midi Kit (Qiagen) according to the manufacturer's instructions. The size of the insert is determined by digesting the plasmid with the restriction enzymes NotI and SalI, which establishes an insert size. The sequence of the entire insert is determined by automated sequencing on both strands of the plasmids.

### Example 3

### SUBCLONING OF THE CODING REGION OF nGPCR-X VIA PCR

Additional experiments may be conducted to subclone the coding region of nGPCR and place the isolated coding region into a useful vector. Two additional PCR primers are designed based on the coding region of nGPCR, corresponding to either end. To protect against exonucleolytic attack during subsequent exposure to enzymes, e.g., Taq polymerase, primers are routinely synthesized with a protective run of nucleotides at the 5′ end that were not necessarily complementary to the desired target.

is performed in a 50μl reaction containing 34μl H₂O, 5 gl 10×TT buffer (140 mM ammonium sulfate, 0.1% gelatin, 0.6 M Tris-tricine, pH 8.4), 5p115mM MgSO₄, 2 μ dNTP mixture (dGTP, dATP, dTTP, and dCTP, each at 10 mM), 3μl genomic phage DNA (0.25 μg/μl), 0.3μl Primer 1 (1 μg/μl), 0.31 μl Primer 2 (1 μg/μl), 0.4 μl High Fidelity Taq polymerase (Boehringer Mannheim). The PCR reaction was started with 1 cycle of 94° C. for 2 minutes;

followed by 25 cycles at 94° C. for 30 seconds, 55° C. for 30 seconds, and 72° C. for 1.3 minutes.

02461 The contents from the PCR reaction are loaded onto a 2% agarose gel and fractionated.

The DNA band of expected size is excised from the gel, placed in a GenElute Agarose spin column (Supelco) and spun for 10 minutes at maximum speed in a microfuge. The eluted DNA is precipitated with ethanol and resuspended in 6 μl H₂O for ligation.

The PCR-amplified DNA fragment containing the coding region is cloned into pCR2. 1 using a protocol standard in the art. In particular, the ligation reaction consists of 6 μl of GPCR DNA, 1 μl 10× ligation buffer, 2 μl pCR2.1 (25 ng/μl, Invitrogen), and 1 μl T4 DNA ligase (Invitrogen). The reaction mixture is incubated overnight at 14° C. and the reaction is then stopped by heating at 65° C. for 10 minutes. Two microliters of the ligation reaction are transformed into One Shot cells (Invitrogen) and plated onto ampicillin plates. A single colony containing a recombinant pCR2.1 bearing an insert is used to inoculate a 5ml culture of LB medium. Plasmid DNA is purified using the Concert Rapid Plasmid Miniprep System (GibcoBRL) and sequenced. Following confirmation of the sequence, a 50 ml culture of LB medium is inoculated with the transformed One Shot cells, cultured, and processed using a Qiagen Plasmid Midi Kit to yield purified pCR-GPCR.

### Example 4

### HYBRIDIZATION ANALYSIS TO DEMONSTRATE nGPCR-X EXPRESSION IN BRAIN

The expression of nGPCR-x in mammals, such as the rat, may be investigated by in situ hybridization histochemistry. To investigate expression in the brain, for example, coronal and sagittal rat brain cryosections (20pm thick) are prepared using a Reichert-Jung cryostat. Individual sections are thaw-mounted onto silanized, nuclease-free slides (CEL Associates, Inc., Houston, Tex.), and stored at −80° C.. Sections are processed starting with post-fixation in cold 4% paraformaldehyde, rinsed in cold phosphate-buffered saline (PBS), acetylated using acetic anhydride in triethanolamine buffer, and dehydrated through a series of alcohol washes in 70%, 95%, and 100% alcohol at room temperature. Subsequently, sections are delipidated in chloroform, followed by rehydration through successive exposure to 100% and 95% alcohol at room temperature. Microscope slides containing processed cryosections are allowed to air dry prior to hybridization. Other tissues may be assayed in a similar fashion.

A nGPCR-x-specific probe is generated using PCR. Following PCR amplification, the fragment is digested with restriction enzymes and cloned into pBluescript II cleaved with the same enzymes. For production of a probe specific for the sense strand of nGPCR-x, the nGPCR-x clone in pBluescript II is linearized with a suitable restriction enzyme, which provides a substrate for labeled run-off transcripts (i.e., cRNA riboprobes) using the vector-borne T7 promoter and commercially available T7 RNA polymerase. A probe specific for the antisense strand of nGPCR-x is also readily prepared using the nGPCR-x clone in pBluescript II by cleaving the recombinant plasmid with a suitable restriction enzyme to generate a linearized substrate for the production of labeled run-off cRNA transcripts using the T3 promoter and cognate polymerase. The riboprobes are labeled with [35S]-UTP to yield a specific activity of about 0.40×10⁶ cpm/pmol for antisense riboprobes and about 0.65×106 cpM/pmol for sense-strand riboprobes. Each riboprobe is subsequently denatured and added (2 pmol/ml) to hybridization buffer which contained 50% formamide, 10% dextran, 0.3 M NaCl, 10 mM Tris (pH 8.0), 1 mM EDTA, 1× Denhardt's Solution, and 10 mM dithiothreitol. Microscope slides containing sequential brain cryosections are independently exposed to 45 μl of hybridization solution per slide and silanized cover slips are placed over the sections being exposed to hybridization solution. Sections are incubated overnight (15-18 hours) at 52° C. to allow hybridization to occur. Equivalent series of cryosections are exposed to sense or antisense nGPCR-x-specific cRNA riboprobes.

Following the hybridization period, coverslips are washed off the slides in 1× SSC, followed by RNase A treatment involving the exposure of slides to 20 μg/ml RNase A in a buffer containing lOmnM Tris-HCl (pH 7.4), 0.5M EDTA, and 0.5M NaCl for 45 minutes at 37° C.. The cryosections are then subjected to three high-stringency washes in 0.1 × SSC at 52° C. for 20 minutes each. Following the series of washes, cryosections are dehydrated by consecutive exposure to 70%, 95%, and 100% ammonium acetate in alcohol, followed by air drying and exposure to Kodak BioMax™ MR-1 film. After 13 days of exposure, the film is developed. Based on these results, slides containing tissue that hybridized, as shown by film autoradiograms, are coated with Kodak NTB-2 nuclear track emulsion and the slides are stored in the dark for 32 days. The slides are then developed and counterstained with hematoxylin. Emulsion-coated sections are analyzed microscopically to determine the specificity of labeling. The signal is determined to be specific if autoradiographic grains (generated by antisense probe hybridization) are clearly associated with cresyl violate-stained cell bodies. Autoradiographic grains found between cell bodies indicates non-specific binding of the probe.

As discussed above, it is well known that GPCRs are expressed in many different tissues and regions, including in the brain. Expression of nGPCR-x in the brain provides an indication that modulators of nGPCR-x activity have utility for treating neurological disorders, including but not limited to, mental disorder, affective disorders, ADHD/ADD (i.e., Attention Deficit-Hyperactivity Disorder/Attention Deficit Disorder), and neural disorders such as Alzheimer's disease, Parkinson's disease, migraine, and senile dementia. Some other diseases for which modulators of nGPCR-x may have utility include depression, anxiety, bipolar disease, epilepsy, neuritis, neurasthenia, neuropathy, neuroses, and the like. Use of nGPCR-x modulators, including nGPCR-x ligands and anti-nGPCR-x antibodies, to treat individuals having such disease states is intended as an aspect of the invention.

### Example 5

### TISSUE EXPRESSION PROFILING

A PCR-based system (RapidScan™ Gene Expression Panel, OriGene Technologies, Rockville, Md.) may be used to generate a comprehensive expression profile of the putative nGPCR-x in human tissue, and in human brain regions. The RapidScan Expression Panel is comprised of first-strand cDNAs from various human tissues and brain regions that are serially diluted over a 4-log range and arrayed into a multi-well PCR plate. Human tissues in the array may include: brain, heart, kidney, spleen, liver, colon, lung, small intestine, muscle, stomach, testis, placenta, salivary gland, thyroid, adrenal gland, pancreas, ovary, uterus, prostate, skin, PBL, bone marrow, fetal brain, and fetal liver.

Expression of nGPCR-x in various tissues is detected using PCR primers designed based on the available sequence of the receptor that will prime the synthesis of a predetermined size fragment in the presence of the appropriate cDNA.

PCR is performed in a 50μl reaction containing 34 μl H₂O, 5 μl 10× TT buffer (140 mM ammonium sulfate, 0.1% gelatin, 0.6 M Tris-tricine, pH 8.4), 5 μl 15mM MgSO₄, 2 μl dNTP mixture (dGTP, dATP, dTTP, and dCTP, each at 10 mM), 0.3 μl forward primer (1 μg/μl), 0.3 μl reverse primer (1 μg/μl), 0.4μl High Fidelity Taq polymerase (Boehringer Mannheim). The PCR reaction mixture is added to each well of the PCR plate. The plate is placed in a MJ Research PTC100 thermocycler, and is then exposed to the following cycling parameters: Pre-soak 94° C. for 3 min; denaturation at 94° C. for 30 seconds; annealing at primer 57° C. for 45 seconds; extension 72° C. for 2 minutes; for 35 cycles. PCR productions are then separated and analyzed by electrophoresis on a 1.2% agarose gel stained with ethidium bromide.

The 4-log dilution range of cDNA deposited on the plate ensures that the amplification reaction is within the linear range and, hence, facilitates semi-quantitative determination of relative mRNA accumulation in the various tissues or brain regions examined.

### Example 6

### NORTHERN BLOT ANALYSIS

Northern blots are performed to examine the expression of nGPCR-x mRNA. The sense orientation oligonucleotide and the antisense-orientation oligonucleotide, described above, are used as primers to amplify a portion of the GPCR-x cDNA sequence selected from the group consisting of SEQ ID NO:l to SEQ ID NO:58.

Multiple human tissue northern blots from Clontech (Human II # 7767-1) are hybridized with the probe. Pre-hybridization is carried out at 42 C for 4 hours in 5×SSC, 1× Denhardt's reagent, 0.1% SDS, 50% formamide, 250 mg/ml salmon sperm DNA. Hybridization is performed overnight at 42° C. in the same mixture with the addition of about 1.5×10⁶ cpm/ml of labeled probe.

The probe is labeled with α-³²P-dCTP by RediprimeTm DNA labeling system (Amersham Pharmacia), purified on Nick Column™ (Amersham Pharmacia) and added to the hybridization solution. The filters are washed several times at 42° C. in 0.2× SSC, 0.1% SDS. Filters are exposed to Kodak XAR film (Eastman Kodak Company, Rochester, N.Y., USA) with intensifying screen at −80° C..

### Example 7

### RECOMBINANT EXPRESSION OF nGPCR-X IN EUKARYOTIC HOST CELLS

A. Expression of nGPCR-x in Mammalian Cells

To produce nGPCR-x protein, a nGPCR-x-encoding polynucleotide is expressed in a suitable host cell using a suitable expression vector and standard genetic engineering techniques. For example, the nGPCR-x-encoding sequence described in Example 1 is subcloned into the commercial expression vector pzeoSV2 (Invitrogen, San Diego, Calif.) and transfected into Chinese Hamster Ovary (CHO) cells using the transfection reagent FuGENE6™ (Boehringer-Mannheim) and the transfection protocol provided in the product insert. Other eukaryotic cell lines, including human embryonic kidney (HEK-293) and COS cells, are suitable as well. Cells stably expressing nGPCR-x are selected by growth in the presence of I 00tg/ml zeocin (Stratagene, LaJolla, Calif.). Optionally, nGPCR-x may be purified from the cells using standard chromatographic techniques. To facilitate purification, antisera is raised against one or more synthetic peptide sequences that correspond to portions of the nGPCR-x amino acid sequence, and the antisera is used to affinity purify nGPCR-x. The nGPCR-x also may be expressed in-frame with a tag sequence (e.g., polyhistidine, hemagluttinin, FLAG) to facilitate purification. Moreover, it will be appreciated that many of the uses for nGPCR-x polypeptides, such as assays described below, do not require purification of nGPCR-x from the host cell.

B. Expression of nGPCR-x in HEK-293 cells

For expression of nGPCR-x in mammalian cells HEK293 (transformed human, primary embryonic kidney cells), a plasmid bearing the relevant nGPCR-x coding sequence is prepared, using vector pSecTag2A (Invitrogen). Vector pSecTag2A contains the murine IgK chain leader sequence for secretion, the c-myc epitope for detection of the recombinant protein with the anti-myc antibody, a C-term-inal polyhistidine for purification with nickel chelate chromatography, and a Zeocin resistant gene for selection of stable transfectants. The forward primer for amplification of this GPCR cDNA is determined by routine procedures and preferably contains a 5′ extension of nucleotides to introduce the HindlII cloning site and nucleotides matching the GPCR sequence. The reverse primer is also determined by routine procedures and preferably contains a 5′ extension of nucleotides to introduce an XhoI restriction site for cloning and nucleotides corresponding to the reverse complement of the nGPCR-x sequence. The PCR conditions are 55° C. as the annealing temperature. The PCR product is gel purified and cloned into the HindIII-XhoI sites of the vector.

The DNA is purified using Qiagen chromatography columns and transfected into HEK-293 cells using DOTAP™ transfection media (Boehringer Mannheim, Indianapolis, Id.). Transiently transfected cells are tested for expression after 24 hours of transfection, using western blots probed with anti-His and anti-nGPCR-x peptide antibodies. Permanently transfected cells are selected with Zeocin and propagated. Production of the recombinant protein is detected from both cells and media by western blots probed with anti-His, anti-Myc or anti-GPCR peptide antibodies.

C. Expression of nGPCR-x in COS cells

For expression of the nGPCR-x in COS7 cells, a polynucleotide molecule having a sequence selected from the group consisting of SEQ ID NO: 1 to SEQ ID NO:58 can be cloned into vector p3-CI. This vector is a pUC18-derived plasmid that contains the HCMV (human cytomegalovirus) promoter-intron located upstream from the bGH (bovine growth hormone) polyadenylation sequence and a multiple cloning site. In addition, the plasmid contains the dhrf (dihydrofolate reductase) gene which provides selection in the presence of the drug methotrexane (MTX) for selection of stable transformants.

The forward primer is determined by routine procedures and preferably contains a 5′ extension which introduces an XbaI restriction site for cloning, followed by nucleotides which correspond to a sequence selected from the group consisting of SEQ ID NO: I to SEQ ID NO:58. The reverse primer is also determined by routine procedures and preferably contains 5′-extension of nucleotides which introduces a Sail cloning site followed by nucleotides which correspond to the reverse complement of a sequence selected from the group consisting of SEQ ID NO: 1 to SEQ ID NO:58. The PCR consists of an initial denaturation step of 5 min at 95° C. 30 cycles of 30 sec denaturation at 95° C., 30 sec annealing at 58° C. and 30 sec extension at 72° C., followed by 5 min extension at 72° C.. The PCR product is gel purified and ligated into the XbaI and Sall sites of vector p3-Cl. This construct is transformed into E. coli cells for amplification and DNA purification. The DNA is purified with Qiagen chromatography columns and transfected into COS 7 cells using Lipofectamine™ reagent from BRL, following the manufacturer's protocols. Forty-eight and 72 hours after transfection, the media and the cells are tested for recombinant protein expression.

nGPCR-x expressed from a COS cell culture can be purified by concentrating the cell-growth media to about 10 mg of protein/ml, and purifying the protein by, for example, chromatography. Purified nGPCR-x is concentrated to 0.5 mg/ml in an Amicon concentrator fitted with a YM-10 membrane and stored at −80° C..

D. Expression of nGPCR-x in Insect Cells

For expression of nGPCR-x in a baculovirus system, a polynucleotide molecule having a sequence selected from the group consisting of SEQ ID NO:1 to SEQ ID NO:58 can be amplified by PCR. The forward primer is determined by routine procedures and preferably contains a 5′ extension which adds the NdeI cloning site, followed by nucleotides which correspond to a sequence selected from the group consisting of SEQ ID NO: 1 to SEQ ID NO:58. The reverse primer is also determined by routine procedures and preferably contains a 5′ extension which introduces the KpnI cloning site, followed by nucleotides which correspond to the reverse complement of a sequence selected from the group consisting of SEQ ID NO: 1 to SEQ ID NO:58.

The PCR product is gel purified, digested with NdeI and Kpnl, and cloned into the corresponding sites of vector pACHTL-A (Pharmingen, San Diego, Calif.). The pAcHTL expression vector contains the strong polyhedrin promoter of the Autographa califomnica nuclear polyhedrosis virus (AcMlPV), and a 6×His tag upstream from the multiple cloning site. A protein kinase site for phosphorylation and a thrombin site for excision of the recombinant protein precede the multiple cloning site is also present. Of course, many other baculovirus vectors could be used in place of pAcHTL-A, such as pAc373, pVL941 and pAcIMl. Other suitable vectors for the expression of GPCR polypeptides can be used, provided that the vector construct includes appropriately located signals for transcription, translation, and trafficking, such as an in-frame AUG and a signal peptide, as required. Such vectors are described in Luckow et al., Virology 170:31-39, among others.

The virus is grown and isolated using standard baculovirus expression methods, such as those described in Summers et al. (A Manual of Methods for Baculovirus Vectors and Insect Cell Culture Procedures, Texas Agricultural Experimental Station Bulletin No. 1555 (1987)).

In a preferred embodiment, pAcBLT-A containing nGPCR-x gene is introduced into baculovirus using the “BaculoGold” transfection kit (Pharmingen, San Diego, Calif.) using methods established by the manufacturer. Individual virus isolates are analyzed for protein production by radiolabeling infected cells with ³⁵S-methionine at 24 hours post infection. Infected cells are harvested at 48 hours post infection, and the labeled proteins are visualized by SDS-PAGE. Viruses exhibiting high expression levels can be isolated and used for scaled up expression.

For expression of a nGPCR-x polypeptide in a Sf9 cells, a polynucleotide molecule having a sequence selected from the group consisting of SEQ ID NO: 1 to SEQ ID NO:58 can be amplified by PCR using the primers and methods described above for baculovirus expression. The nGPCR-x cDNA is cloned into vector pAcHLT-A (Pharmingen) for expression in Sf9 insect. The insert is cloned into the NdeI and KpnI sites, after elimination of an internal NdeI site (using the same primers described above for expression in baculovirus). DNA is purified with Qiagen chromatography columns and expressed in Sf9 cells. Preliminary Western blot experiments from non-purified plaques are tested for the presence of the recombinant protein of the expected size which reacted with the GPCR-specific antibody. These results are confirmed after further purification and expression optimization in HiG5 cells.

### Example 8

### INTERACTION TRAP/TWO-HYBRID SYSTEM

In order to assay for nGPCR-x-interacting proteins, the interaction trap/two-hybrid library screening method can be used. This assay was first described in Fields et al., Nature, 1989, 340, 245, which is incorporated herein by reference in its entirety. A protocol is published in Current Protocols in Molecular Biology 1999, John Wiley & Sons, N.Y., and Ausubel, F. M. et al. 1992, Short protocols in molecular biology, Fourth edition, Greene and Wiley-interscience, N.Y., each of which is incorporated herein by reference in its entirety. Kits are available from Clontech, Palo Alto, CA (Matchmaker Two-Hybrid System 3).

A fusion of the nucleotide sequences encoding all or partial nGPCR-x and the yeast transcription factor GAL4 DNA-binding domain (DNA-BD) is constructed in an appropriate plasmid (i.e., pGBKT7) using standard subcloning techniques. Similarly, a GAIA active domain (AD) fusion library is constructed in a second plasmid (i.e., pGADT7) from cDNA of potential GPCR-binding proteins (for protocols on forming cDNA libraries, see Sambrook et al. 1989, Molecular cloning: a laboratory manual, second edition, Cold Spring Harbor Press, Cold Spring Harbor, N.Y.), which is incorporated herein by reference in its entirety. The DNA-BD/nGPCR-x fusion construct is verified by sequencing, and tested for autonomous reporter gene activation and cell toxicity, both of which would prevent a successful two-hybrid analysis. Similar controls are performed with the AD/library fusion construct to ensure expression in host cells and lack of transcriptional activity. Yeast cells are transformed (ca. 105 transformants/mg DNA) with both the nGPCR-x and library fusion plasmids according to standard procedures (Ausubel et al., 1992, Short protocols in molecular biology, fourth edition, Greene and Wiley-interscience, NY, which is incorporated herein by reference in its entirety). In vivo binding of DNA-BD/nGPCR-x with AD/library proteins results in transcription of specific yeast plasmid reporter genes (i.e., lacZ, HIS3, ADE2, LEU2). Yeast cells are plated on nutrient-deficient media to screen for expression of reporter genes. Colonies are dually assayed for β-galactosidase activity upon growth in Xgal (5-bromo-4-chloro-3-indolyl-β-D-galactoside) supplemented media (filter assay for β-galactosidase activity is described in Breeden et al., Cold Spring Harb. Symp. Quant. Biol., 1985, 50, 643, which is incorporated herein by reference in its entirety). Positive AD-library plasmids are rescued from transformants and reintroduced into the original yeast strain as well as other strains containing unrelated DNA-BD fusion proteins to confirm specific nGPCR-x/library protein interactions. Insert DNA is sequenced to verify the presence of an open reading frame fused to GAL4 AD and to determine the identity of the nGPCR-x-binding protein.

### Example 9

### MOBILITY SHIFT DNA-BINDING ASSAY USING GEL ELECTROPHORESIS

A gel electrophoresis mobility shift assay can rapidly detect specific protein-DNA interactions. Protocols are widely available in such manuals as Sambrook et al. 1989, Molecular cloning: a laboratory manual, second edition, Cold Spring Harbor Press, Cold Spring Harbor, N.Y. and Ausubel, F. M. et al., 1992, Short Protocols in Molecular Biology, fourth edition, Greene and Wiley-interscience, NY, each of which is incorporated herein by reference in its entirety.

Probe DNA (<300 bp) is obtained from synthetic oligonucleotides, restriction endonuclease fragments, or PCR fragments and end-labeled with ³²p. An aliquot of purified nGPCR-x (ca. 15 μg) or crude nGPCR-x extract (ca. 15 ng) is incubated at constant temperature (in the range 22-37C.) for at least 30 minutes in 10-15 μl of buffer (i.e. TAE or TBE, pH 8.0-8.5) containing radiolabeled probe DNA, nonspecific carrier DNA (ca. 1 , ug), BSA (300 tg/ml), and 10% (v/v) glycerol. The reaction mixture is then loaded onto a polyacrylamide gel and run at 30-35 mA until good separation of free probe DNA from protein-DNA complexes occurs. The gel is then dried and bands corresponding to free DNA and protein-DNA complexes are detected by autoradiography.

### Example 10

### ANTIBODIES TO nGPCR-X

Standard techniques are employed to generate polyclonal or monoclonal antibodies to the nGPCR-x receptor, and to generate useful antigen-binding fragments thereof or variants thereof, including “humanized” variants. Such protocols can be found, for example, in Sambrook et al. (1989) and Harlow et al. (Eds.), Antibodies A Laboratory Manual; Cold Spring Harbor Laboratory; Cold Spring Harbor, N.Y. (1988). In one embodiment, recombinant nGPCR-x polypeptides (or cells or cell membranes containing such polypeptides) are used as antigen to generate the antibodies. In another embodiment, one or more peptides having amino acid sequences corresponding to an immunogenic portion of nGPCR-x (e.g., 6, 7, 8, 9, 10, 11, 12, 13, 14, 15, 16, 17, 18, 19, 20, or more amino acids) are used as antigen. Peptides corresponding to extracellular portions of nGPCR-x, especially hydrophilic extracellular portions, are preferred. The antigen may be mixed with an adjuvant or linked to a hapten to increase antibody production.

A. Polyclonal or Monoclonal antibodies

As one exemplary protocol, recombinant nGPCR-x or a synthetic fragment thereof is used to immunize a mouse for generation of monoclonal antibodies (or larger mammal, such as a rabbit, for polyclonal antibodies). To increase antigenicity, peptides are conjugated to Keyhole Lympet Hemocyanin (Pierce), according to the manufacturer's recommendations. For an initial injection, the antigen is emulsified with Freund's Complete Adjuvant and injected subcutaneously. At intervals of two to three weeks, additional aliquots of nGPCR-x antigen are emulsified with Freund's Incomplete Adjuvant and injected subcutaneously. Prior to the final booster injection, a serum sample is taken from the immunized mice and assayed by western blot to confirm the presence of antibodies that immunoreact with nGPCR-x. Serum from the immunized animals may be used as polyclonal antisera or used to isolate polyclonal antibodies that recognize nGPCR-x. Alternatively, the mice are sacrificed and their spleen removed for generation of monoclonal antibodies.

To generate monoclonal antibodies, the spleens are placed in 10 ml serum-free RPMI 1640, and single cell suspensions are formed by grinding the spleens in serum-free RPMI 1640, supplemented with 2 mM L-glutamine, 1 mM sodium pyruvate, 100 units/ml penicillin, and 100 gg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a Feeder Layer. NS-1 myeloma cells, kept in log phase in RPMI with 10% fetal bovine serum (FBS) (Hyclone Laboratories, Inc., Logan, Utah) for three days prior to fusion, are centrifuged and washed as well.

To produce hybridoma fusions, spleen cells from the immunized mice are combined with NS-1 cells and centrifuged, and the supernatant is aspirated. The cell pellet is dislodged by tapping the tube, and 2 ml of 37° C. PEG 1500 (50% in 75 mM HEPES, pH 8.0) (Boehringer-Mannheim) is stirred into the pellet, followed by the addition of serum-free RPMI. Thereafter, the cells are centrifuged, resuspended in RPMI containing 15% FBS, 100 FM sodium hypoxanthine, 0.4 μM aminopterin, 16 μM thymidine (HAT) (Gibco), 25 units/ml IL-6 (Boehringer-Mannheim) and 1.5×10⁶ thymocytes/ml, and plated into 10° C.orning flat-bottom 96-well tissue culture plates (Corning, Corning New York).

0278] On days 2, 4, and 6 after the fusion, 1001 μl of medium is removed from the wells of the fusion plates and replaced with fresh medium. On day 8, the fusions are screened by ELISA, testing for the presence of mouse IgG that binds to nGPCR-x. Selected fusion wells are further cloned by dilution until monoclonal cultures producing anti-nGPCR-x antibodies are obtained. DOCKET NO.: 00196 US1 PATENT

B. Humanization of anti-nGPCR-x monoclonal antibodies

0279] The expression pattern of nGPCR-x as reported herein and the proven track record of GPCRs as targets for therapeutic intervention suggest therapeutic indications for nGPCR-x inhibitors (antagonists). nGPCR-x-neutralizing antibodies comprise one class of therapeutics useful as nGPCR-x antagonists. Following are protocols to improve the utility of anti-nGPCR-x monoclonal antibodies as therapeutics in humans by “humanizing” the monoclonal antibodies to improve their serum half-life and render them less immunogenic in human hosts (i.e., to prevent human antibody response to non-human anti-nGPCR-x antibodies).

The principles of humanization have been described in the literature and are facilitated by the modular arrangement of antibody proteins. To minimize the possibility of binding complement, a humanized antibody of the IgG4 isotype is preferred.

For example, a level of humanization is achieved by generating chimeric antibodies comprising the variable domains of non-human antibody proteins of interest with the constant domains of human antibody molecules. (See, e.g., Morrison et al., Adv. Immunol., 44:65-92 (1989)). The variable domains of nGPCR-x-neutralizing anti-nGPCR-x antibodies are cloned from the genomic DNA of a B-cell hybridoma or from cDNA generated from mRNA isolated from the hybridoma of interest. The V region gene fragments are linked to exons encoding human antibody constant domains, and the resultant construct is expressed in suitable mammalian host cells (e.g., myeloma or CHO cells).

To achieve an even greater level of humanization, only those portions of the variable region gene fragments that encode antigen-binding complementarity determining regions (“CDR”) of the non-human monoclonal antibody genes are cloned into human antibody sequences. (See, e.g., Jones et al., Nature 321:522-525 (1986); Riechmann et al., Nature 332:323-327 (1988); Verhoeyen et al., Science 239:1534-36 (1988); and Tempest et al., Bio/Technology 9: 266-71 (1991)). If necessary, the P-sheet framework of the human antibody surrounding the CDR3 regions also is modified to more closely mirror the three dimensional structure of the antigen-binding domain of the original monoclonal antibody. (See Kettleborough et al., Protein Engin., 4:773-783 (1991); and Foote et al., J. Mol. Biol., 224:487-499 (1992)).

In an alternative approach, the surface of a non-human monoclonal antibody of interest is humanized by altering selected surface residues of the non-human antibody, e.g., by site-directed mutagenesis, while retaining all of the interior and contacting residues of the non-human antibody. See Padlan, Molecular Imnnunol., 28(4/5):489-98 (1991).

The foregoing approaches are employed using nGPCR-x-neutralizing anti-nGPCR-x monoclonal antibodies and the hybridomas that produce them to generate humanized nGPCR-x-neutralizing antibodies useful as therapeutics to treat or palliate conditions wherein nGPCR-x expression or ligand-mediated nGPCR-x signaling is detrimental.

C. Human nGPCR-x-Neutralizing Antibodies from Phage Display

Human nGPCR-x-neutralizing antibodies are generated by phage display techniques such as those described in Aujame et al., Human Antibodies 8(4):155-168 (1997); Hoogenboom, TIBTECH 15:62-70 (1997); and Rader et al., Curr. Opin. Biotechnol. 8:503-508 (1997), all of which are incorporated by reference. For example, antibody variable regions in the form of Fab fragments or linked single chain Fv fragments are fused to the amino terminus of filamentous phage minor coat protein plll. Expression of the fusion protein and incorporation thereof into the mature phage coat results in phage particles that present an antibody on their surface and contain the genetic material encoding the antibody. A phage library comprising such constructs is expressed in bacteria, and the library is screened for nGPCR-x-specific phage-antibodies using labeled or immobilized nGPCR-x as antigen-probe.

Human nGPCR-x-neutralizing antibodies from transgenic mice

Human nGPCR-x-neutralizing antibodies are generated in transgenic mice essentially as described in Bruggemann et al., Immunol. Today 17(8):391-97 (1996) and Bruggemann et al., Curr. Opin. Biotechnol. 8:455-58 (1997). Transgenic mice carrying human V-gene segments in germline configuration and that express these transgenes in their lymphoid tissue are immunized with a nGPCR-x composition using conventional immunization protocols. Hybridomas are generated using B cells from the immunized mice using conventional protocols and screened to identify hybridomas secreting anti-nGPCR-x human antibodies (e.g., as described above).

### Example 11

### ASSAYS TO IDENTIFY MODULATORS OF nGPCR-X ACTIVITY

Set forth below are several nonlimiting assays for identifying modulators (agonists and antagonists) of nGPCR-x activity. Among the modulators that can be identified by these assays are natural ligand compounds of the receptor; synthetic analogs and derivatives of natural ligands; antibodies, antibody fragments, and/or antibody-like compounds derived from natural antibodies or from antibody-like combinatorial libraries; and/or synthetic compounds identified by high-throughput screening of libraries; and the like. All modulators that bind nGPCR-x are useful for identifying nGPCR-x in tissue samples (e.g., for diagnostic purposes, pathological purposes, and the like). Agonist and antagonist modulators are useful for up-regulating and down-regulating nGPCR-x activity, respectively, to treat disease states characterized by abnormal levels of nGPCR-x activity. The assays may be performed using single putative modulators, and/or may be performed using a known agonist in combination with candidate antagonists (or visa versa).

A. cAMP Assays

In one type of assay, levels of cyclic adenosine monophosphate (cAMP) are measured in nGPCR-x-transfected cells that have been exposed to candidate modulator compounds. Protocols for cAMP assays have been described in the literature. (See, e.g., Sutherland et al., Circulation 37: 279 (1968); Frandsen et al., Life Sciences 18: 529-541 (1976); Dooley et al., Journal of Pharmacology and Experimental Therapeutics 283 (2): 735-41 (1997); and George et al., Journal of Biomolecular Screening 2 (4): 235-40 (1997)). An exemplary protocol for such an assay, using an Adenylyl Cyclase Activation FlashPlate® Assay from NEN™ Life Science Products, is set forth below.

Briefly, the nGPCR-x coding sequence (e.g., a cDNA or intronless genomic DNA) is subcloned into a commercial expression vector, such as pzeoSV2 (Invitrogen), and transiently transfected into Chinese Hamster Ovary (CHO) cells using known methods, such as the transfection protocol provided by Boehringer-Mannheim when supplying the FuGENE 6 transfection reagent. Transfected CHO cells are seeded into 96-well microplates from the FlashPlate® assay kit, which are coated with solid scintillant to which antisera to cAMP has been bound. For a control, some wells are seeded with wild type (untransfected) CHO cells. Other wells in the plate receive various amounts of a cAMP standard solution for use in creating a standard curve.

One or more test compounds (i.e., candidate modulators) are added to the cells in each well, with water and/or compound-free medium/diluent serving as a control or controls. After treatment, cAMP is allowed to accumulate in the cells for exactly 15 minutes at room temperature. The assay is terminated by the addition of lysis buffer containing [¹²⁵I]-labeled cAMP, and the plate is counted using a Packard Topcount™ 96-well microplate scintillation counter. Unlabeled cAMP from the lysed cells (or from standards) and fixed amounts of [125I]-cAMP compete for antibody bound to the plate. A standard curve is constructed, and cAMP values for the unknowns are obtained by interpolation. Changes in intracellular cAMP levels of cells in response to exposure to a test compound are indicative of nGPCR-x modulating activity. Modulators that act as agonists of receptors which couple to the Gs subtype of G proteins will stimulate production of cAMP, leading to a measurable 3-10 fold increase in cAMP levels. Agonists of receptors which couple to the Gi/ subtype of G proteins will inhibit forskolin-stimulated cAMP production, leading to a measurable decrease in cAMP levels of 50-100%. Modulators that act as inverse agonists will reverse these effects at receptors that are either constitutively active or activated by known agonists.

B. Aequorin Assays

In another assay, cells (e.g., CHO cells) are transiently co-transfected with both a nGPCR-x expression construct and a construct that encodes the photoprotein apoaquorin. In the presence of the cofactor coelenterazine, apoaquorin will emit a measurable luminescence that is proportional to the amount of intracellular (cytoplasmic) free calcium. (See generally, Cobbold, et al. “Aequorin measurements of cytoplasmic free calcium, ” In: McCormack J.G. and Cobbold P. H., eds., Cellular Calcium: A Practical Approach. Oxford:IRL Press (1991); Stables et al., Analytical Biochemistry 252: 115-26 (1997); and Haugland, Handbook of Fluorescent Probes and Research Chemicals. Sixth edition. Eugene OR: Molecular Probes (1996).)

In one exemplary assay, nGPCR-x is subcloned into the commercial expression vector pzeoSV2 (Invitrogen) and transiently co-transfected along with a construct that encodes the photoprotein apoaquorin (Molecular Probes, Eugene, OR) into CHO cells using the transfection reagent FuGENE 6 (Boehringer-Mannheim) and the transfection protocol provided in the product insert.

The cells are cultured for 24 hours at 37° C. in MEM (Gibco/BRL, Gaithersburg, Md.) supplemented with 10% fetal bovine serum, 2 mM glutamine, 10 U/ml penicillin and 10 fig/ml streptomycin, at which time the medium is changed to serum-free MEM containing 5 μm coelenterazine (Molecular Probes, Eugene, OR). Culturing is then continued for two additional hours at 37° C.. Subsequently, cells are detached from the plate using VERSEN (Gibco/BRL), washed, and resuspended at 200, 000 cells/ml in serum-free MEM.

Dilutions of candidate nGPCR-x modulator compounds are prepared in serum-free MEM and dispensed into wells of an opaque 96-well assay plate at 50 μl/well. Plates are then loaded onto an MLX microtiter plate luminometer (Dynex Technologies, Inc., Chantilly, Va.). The instrument is programmed to dispense 50μl cell suspensions into each well, one well at a time, and immediately read luminescence for 15 seconds. Dose-response curves for the candidate modulators are constructed using the area under the curve for each light signal peak. Data are analyzed with SlideWrite, using the equation for a one-site ligand, and EC₅₀ values are obtained. Changes in luminescence caused by the compounds are considered indicative of modulatory activity. Modulators that act as agonists at receptors which couple to the Gq subtype of G proteins give an increase in luminescence of up to 100 fold. Modulators that act as inverse agonists will reverse this effect at receptors that are either constitutively active or activated by known agonists.

C. Luciferase Reporter Gene Assay

The photoprotein luciferase provides another useful tool for assaying for modulators of nGPCR-x activity. Cells (e.g., CHO cells or COS 7 cells) are transiently co-transfected with both a nGPCR-x expression construct (e.g., nGPCR-x in pzeoSV2) and a reporter construct which includes a gene for the luciferase protein downstream from a transcription factor binding site, such as the cAMP-response element (CRE), AP-1, or NF-kappa B. Agonist binding to receptors coupled to the G, subtype of G proteins leads to increases in cAMP, thereby activating the CRE transcription factor and resulting in expression of the luciferase gene. Agonist binding to receptors coupled to the Gq subtype of G protein leads to production of diacylglycerol that activates protein kinase C, which activates the AP-1 or NF-kappa B transcription factors, in turn resulting in expression of the luciferase gene. Expression levels of luciferase reflect the activation status of the signaling events. (See generally, George et al., Journal of Biomolecular Screening 2(4): 235-240 (1997); and Stratowa et al., Current Opinion in Biotechnology 6: 574-581 (1995)). Luciferase activity may be quantitatively measured using, e.g., luciferase assay reagents that are commercially available from Promega (Madison, WI).

In one exemplary assay, CHO cells are plated in 24-well culture dishes at a density of 100, 000 cells/well one day prior to transfection and cultured at 37° C. in MEM (Gibco/BRL) supplemented with 10% fetal bovine serum, 2 mM glutamine, 10 U/ml penicillin and 10 μg/ml streptomycin. Cells are transiently co-transfected with both a nGPCR-x expression construct and a reporter construct containing the luciferase gene. The reporter plasmids CRE-luciferase, AP-1-luciferase and NF-kappaB-luciferase may be purchased from Stratagene (LaJolla, Calif.). Transfections are performed using the FuGENE 6 transfection reagent (Boehringer-Mannheim) according to the supplier's instructions. Cells transfected with the reporter construct alone are used as a control. Twenty-four hours after transfection, cells are washed once with PBS pre-warmed to 37° C.. Serum-free MEM is then added to the cells either alone (control) or with one or more candidate modulators and the cells are incubated at 37° C. for five hours. Thereafter, cells are washed once with ice-cold PBS and lysed by the addition of 100 μll of lysis buffer per well from the luciferase assay kit supplied by Promega. After incubation for 15 minutes at room temperature, 15μl of the lysate is mixed with 50 , ul of substrate solution (Promega) in an opaque-white, 96-well plate, and the luminescence is read immediately on a Wallace model 1450 MicroBeta scintillation and luminescence counter (Wallace Instruments, Gaithersburg, Md.).

Differences in luminescence in the presence versus the absence of a candidate modulator compound are indicative of modulatory activity. Receptors that are either constitutively active or activated by agonists typically give a 3 to 20-fold stimulation of luminescence compared to cells transfected with the reporter gene alone. Modulators that act as inverse agonists will reverse this effect.

D. Intracellular calcium measurement using FLIPR

Changes in intracellular calcium levels are another recognized indicator of G protein-coupled receptor activity, and such assays can be employed to screen for modulators of nGPCR-x activity. For example, CHO cells stably transfected with a nGPCR-x expression vector are plated at a density of 4 ×10⁴ cells/well in Packard black-walled, 96-well plates specially designed to discriminate fluorescence signals emanating from the various wells on the plate. The cells are incubated for 60 minutes at 37° C. in modified Dulbecco's PBS (D-PBS) containing 36 mg/L pyruvate and 1 g/L glucose with the addition of 1% fetal bovine serum and one of four calcium indicator dyes (Fluo-3™ AM, Fluo-4™ AM, Calcium Green™ 1 AM, or Oregon Green™ 488 BAPTA-1 AM), each at a concentration of 4 aμm. Plates are washed once with modified D-PBS without 1% fetal bovine serum and incubated for 10 minutes at 37° C. to remove residual dye from the cellular membrane. In addition, a series of washes with modified D-PBS without 1% fetal bovine serum is performed immediately prior to activation of the calcium response.

A calcium response is initiated by the addition of one or more candidate receptor agonist compounds, calcium ionophore A23187 (10 μm; positive control), or ATP (4 μm; positive control). Fluorescence is measured by Molecular Device's FLIPR with an argon laser (excitation at 488 nm). (See, e.g., Kuntzweiler et al., Drug Development Research, 44(1): 14-20 (1998)). The F-stop for the detector camera was set at 2.5 and the length of exposure was 0.4 milliseconds. Basal fluorescence of cells was measured for 20 seconds prior to addition of candidate agonist, ATP, or A23187, and the basal fluorescence level was subtracted from the response signal. The calcium signal is measured for approximately 200 seconds, taking readings every two seconds. Calcium ionophore A23187 and ATP increase the calcium signal 200% above baseline levels. In general, activated GPCRs increase the calcium signal approximately 10-15% above baseline signal.

E. Mitogenesis Assay

In a mitogenesis assay, the ability of candidate modulators to induce or inhibit nGPCR-x-mediated cell division is determined. (See, e.g., Lajiness et al., Journal of Pharmacology and Experimental Therapeutics 267(3): 1573-1581 (1993)). For example, CHO cells stably expressing nGPCR-x are seeded into 96-well plates at a density of 5000 cells/well and grown at 37° C. in MEM with 10% fetal calf serum for 48 hours, at which time the cells are rinsed twice with serum-free MEM. After rinsing, 80tt of fresh MEM, or MEM containing a known mitogen, is added along with 2041 MEM containing varying concentrations of one or more candidate modulators or test compounds diluted in serum-free medium. As controls, some wells on each plate receive serum-free medium alone, and some receive medium containing 10% fetal bovine serum. Untransfected cells or cells transfected with vector alone also may serve as controls.

After culture for 16-18 hours, 1μ Ci of [³H]-thymidine (2° C.i/mmol) is added to the wells and cells are incubated for an additional 2 hours at 37° C.. The cells are trypsinized and collected on filter mats with a cell harvester (Tomtec); the filters are then counted in a Betaplate counter. The incorporation of [³H]-thymidine in serum-free test wells is compared to the results achieved in cells stimulated with serum (positive control). Use of multiple concentrations of test compounds permits creation and analysis of dose-response curves using the non-linear, least squares fit equation: A=B×[C/(D+C)]+G where A is the percent of serum stimulation; B is the maximal effect minus baseline; C is the EC₅₀; D is the concentration of the compound; and G is the maximal effect. Parameters B, C and G are determined by Simplex optimization.

Agonists that bind to the receptor are expected to increase [³ H]-thymidine incorporation into cells, showing up to 80% of the response to serum. Antagonists that bind to the receptor will inhibit the stimulation seen with a known agonist by up to 100%.

F. [³⁵]SIGTPγS Binding Assay

Because G protein-coupled receptors signal through intracellular G proteins whose activity involves GTP binding and hydrolysis to yield bound GDP, measurement of binding of the non-hydrolyzable GTP analog [³⁵S]GTPγS in the presence and absence of candidate asmodulators provides another assay for modulator activity. (See, e.g., Kowal et al., Neuropharmacology 37:179-187 (1998).)

In one exemplary assay, cells stably transfected with a nGPCR-x expression vector are grown in 10 cm tissue culture dishes to subconfluence, rinsed once with 5 ml of ice-cold Ca2+/Mg²⁺-free phosphate-buffered saline, and scraped into 5 ml of the same buffer. Cells are pelleted by centrifugation (500 ×g, 5 minutes), resuspended in TEE buffer (25 mM Tris, pH 7.5, 5 mM EDTA, 5 mM EGTA), and frozen in liquid nitrogen. After thawing, the cells are homogenized using a Dounce homogenizer (one ml TEE per plate of cells), and centrifuged at 1,000 ×g for 5 minutes to remove nuclei and unbroken cells.

The homogenate supernatant is centrifuged at 20,000 ×g for 20 minutes to isolate the membrane fraction, and the membrane pellet is washed once with TEE and resuspended in binding buffer (20 mM HEPES, pH 7.5, 150 mM NaCl, 10 mM MgCl₂, 1 mM EDTA). The resuspended membranes can be frozen in liquid nitrogen and stored at −70° C. until use.

Aliquots of cell membranes prepared as described above and stored at −70° C. are thawed, homogenized, and diluted into buffer containing 20 mM HEPES, 10 mM MgCl₂, 1 mM EDTA, 120 mM NaCl, 10μm GDP, and 0.2 mM ascorbate, at a concentration of 10-50 μg/ml. In a final volume of 90μl, homogenates are incubated with varying concentrations of candidate modulator compounds or 100 μM GTP for 30 minutes at 30° C. and then placed on ice. To each sample, 10 Atl guanosine 5′-O-(3[³⁵S]thio) triphosphate (NEN, 1200° C.i/mmol; [³⁵S]-GTPγS), was added to a final concentration of 100-200 pM. Samples are incubated at 30° C. for an additional 30 minutes, 1 ml of 10 mM HEPES, pH 7.4, 10 mM MgCl₂, at 4° C. is added and the reaction is stopped by filtration.

Samples are filtered over Whatman GF/B filters and the filters are washed with 20 ml ice-cold 10 mM HEPES, pH 7.4, 10 mM MgCl₂. Filters are counted by liquid scintillation spectroscopy. Nonspecific binding of [³⁵S]-GTPγS is measured in the presence of 100 Iμm GTP and subtracted from the total. Compounds are selected that modulate the amount of [355]-GTPγS binding in the cells, compared to untransfected control cells. Activation of receptors by agonists gives up to a five-fold increase in [³⁵S]GTPγS binding. This response is blocked by antagonists.

G. MAP Kinase Activity Assay

Evaluation of MAP kinase activity in cells expressing a GPCR provides another assay to identify modulators of GPCR activity. (See, e.g., Lajiness et al., Journal of Pharmacology and Experimental Therapeutics 267(3):1573-1581 (1993) and Boulton et al., Cell 65:663-675 (1991).)

In one embodiment, CHO cells stably transfected with nGPCR-x are seeded into 6-well plates at a density of 70,000 cells/well 48 hours prior to the assay. During this 48-hour period, the cells are cultured at 37° C. in MEM medium supplemented with 10% fetal bovine serum, 2mM glutamine, 10 U/ml penicillin and 10Rg/ml streptomycin. The cells are serum-starved for 1-2 hours prior to the addition of stimulants.

For the assay, the cells are treated with medium alone or medium containing either a candidate agonist or 200 nM Phorbol ester- myristoyl acetate (i.e., PMA, a positive control), and the cells are incubated at 37° C. for varying times. To stop the reaction, the plates are placed on ice, the medium is aspirated, and the cells are rinsed with 1 ml of ice-cold PBS containing InM EDTA. Thereafter, 200 μl of cell lysis buffer (12.5 mM MOPS, pH 7.3, 12.5 mM glycerophosphate, 7.5 mM MgCl₂, 0.5 mM EGTA, 0.5 mM sodium vanadate, 1 mM benzamidine, ImM dithiothreitol, 10 μg/ml leupeptin, 10 μg/ml aprotinin, 2, g/ml pepstatin A, and 1 μlM okadaic acid) is added to the cells. The cells are scraped from the plates and homogenized by 10 passages through a 23¾ G needle, and the cytosol fraction is prepared by centrifugation at 20,000 ×g for 15 minutes.

Aliquots (5-10 μl containing 1-5 μg protein) of cytosol are mixed with 1 mM MAPK Substrate Peptide (APRTPGGRR (SEQ ID NO: 117), Upstate Biotechnology, Inc., N.Y.) and 50 μM [γ-³²P]ATP (NEN, 3000 Ci/mmol), diluted to a final specific activity of ˜2000 cpm/pmol, in a total volume of 25 μl. The samples are incubated for 5 minutes at 30° C., and reactions are stopped by spotting 20 μl on 2 cm² squares of Whatman P81 phosphocellulose paper. The filter squares are washed in 4 changes of 1% H₃PO₄, and the squares are subjected to liquid scintillation spectroscopy to quantitate bound label. Equivalent cytosolic extracts are incubated without MAPK substrate peptide, and the bound label from these samples are subtracted from the matched samples with the substrate peptide. The cytosolic extract from each well is used as a separate point. Protein concentrations are determined by a dye binding protein assay (Bio-Rad Laboratories). Agonist activation of the receptor is expected to result in up to a five-fold increase in MAPK enzyme activity. This increase is blocked by antagonists.

H. [³H]Arachidonic Acid Release

The activation of GPCRs also has been observed to potentiate arachidonic acid release in cells, providing yet another useful assay for modulators of GPCR activity. (See, e.g., Kanterman et al., Molecular Pharmacology 39:364-369 (1991).) For example, CHO cells that are stably transfected with a nGPCR-x expression vector are plated in 24-well plates at a density of 15,000 cells/well and grown in MEM medium supplemented with 10% fetal bovine serum, 2 mM glutamine, 10 U/ml penicillin and 10 jg/ml streptomycin for 48 hours at 37° C. before use. Cells of each well are labeled by incubation with [³H]-arachidonic acid (Amersham Corp., 210 Ci/mmol) at 0.5 μCi/ml in 1 ml MEM supplemented with 10 mM HEPES, pH 7.5, and 0.5% fatty-acid-free bovine serum albumin for 2 hours at 37° C. The cells are then washed twice with 1 ml of the same buffer.

Candidate modulator compounds are added in 1 ml of the same buffer, either alone or with 10μM ATP and the cells are incubated at 37° C. for 30 minutes. Buffer alone and mock-transfected cells are used as controls. Samples (0.5 ml) from each well are counted by liquid scintillation spectroscopy. Agonists which activate the receptor will lead to potentiation of the ATP-stimulated release of [³H]-arachidonic acid. This potentiation is blocked by antagonists.

I. Extracellular Acidification Rate

In yet another assay, the effects of candidate modulators of nGPCR-x activity are assayed by monitoring extracellular changes in pH induced by the test compounds. (See, e.g., Dunlop et al., Journal of Pharmacological and Toxicological Methods 40(1):47-55 (1998).) In one embodiment, CHO cells transfected with a nGPCR-x expression vector are seeded into 12 mm capsule cups (Molecular Devices Corp.) at 4 ×10⁵ cells/cup in MEM supplemented with 10% fetal bovine serum, 2 mM L-glutamine, 10 U/ml penicillin, and 10 μg/ml streptomycin. The cells are incubated in this medium at 37° C. in 5% CO₂ for 24 hours.

Extracellular acidification rates are measured using a Cytosensor microphysiometer (Molecular Devices Corp.). The capsule cups are loaded into the sensor chambers of the microphysiometer and the chambers are perfused with running buffer (bicarbonate-free MEM supplemented with 4 mM L-glutamine, 10 units/ml penicillin, 10 ttg/ml streptomycin, 26 mM NaCl) at a flow rate of 100 μl/minute. Candidate agonists or other agents are diluted into the running buffer and perfused through a second fluid path. During each 60-second pump cycle, the pump is run for 38 seconds and is off for the remaining 22 seconds. The pH of the running buffer in the sensor chamber is recorded during the cycle from 43-58 seconds, and the pump is re-started at 60 seconds to start the next cycle. The rate of acidification of the running buffer during the recording time is calculated by the Cytosoft program. Changes in the rate of acidification are calculated by subtracting the baseline value (the average of 4 rate measurements immediately before addition of a modulator candidate) from the highest rate measurement obtained after addition of a modulator candidate. The selected instrument detects 61 mV/pH unit. Modulators that act as agonists of the receptor result in an increase in the rate of extracellular acidification compared to the rate in the absence of agonist. This response is blocked by modulators which act as antagonists of the receptor.

### Example 12

### Using nGPCR-x proteins to isolate neurotransmitters

Isolated nGPCR-x proteins of the present invention can be used to isolate novel or known neurotransmitters (Saito et al., Nature 400: 265-269, 1999). The cDNAs that encode the isolated nGPCR-x can be cloned into mammalian expression vectors and used to stably or transiently transfect mammalian cells including CHO, Cos or HEK293 cells. Receptor expression can be determined by Northern blot analysis of transfected cells and identification of an appropriately sized mRNA band (predicted size from the cDNA). Brain regions shown by mRNA analysis to express each of the nGPCR-x proteins could be processed for peptide extraction using any of several protocols ((Reinsheidk R.K. et al., Science 270: 243-247, 1996; Sakurai, T., et al., Cell 92; 573-585, 1998; Hinuma, S., et al., Nature 393: 272-276, 1998). Chromotographic fractions of brain extracts could be tested for ability to activate nGPCR-x proteins by measuring second messenger production such as changes in cAMP production in the presence or absence of forskolin, changes in inositol 3-phosphate levels, changes in intracellular calcium levels or by indirect measures of receptor activation including receptor stimulated mitogenesis, receptor mediated changes in extracellular acidification or receptor mediated changes in reporter gene activation in response to cAMP or calcium (these methods should all be referenced in other sections of the patent). Receptor activation could also be monitored by co-transfecting cells with a chimeric Glq/i₃ to force receptor coupling to a calcium stimulating pathway (Conklin et al., Nature 363; 274-276, 1993). Neurotransmitter mediated activation of receptors could also be monitored by measuring changes in [³⁵ S]-GTPKS binding in membrane fractions prepared from transfected mammalian cells. This assay could also be performed using baculoviruses containing nGPCR-x proteins infected into SF9 insect cells.

The neurotransmitter which activates nGPCR-x proteins can be purified to homogeneity through successive rounds of purification using nGPCR-x proteins activation as a measurement of neurotransmitter activity. The composition of the neurotransmitter can be determined by mass spectrometry and Edman degradation if peptidergic. Neurotransmitters isolated in this manner will be bioactive materials which will alter neurotransmission in the central nervous system and will produce behavioral and biochemical changes.

### Example 13

### Using nGPCR-x proteins to isolate and purify G proteins

cDNAs encoding nGPCR-x proteins are epitope-tagged at the amino terminuus end of the cDNA with the cleavable influenza-hemagglutinin signal sequence followed by the FLAG epitope (IBI, New Haven, CT). Additionally, these sequences are tagged at the carboxyl terminus with DNA encoding six histidine residues. (Amino and Carboxyl Terminal Modifications to Facilitate the Production and Purification of a G Protein-Coupled Receptor, B. K. Kobilka, Analytical Biochemistry, Vol. 231, No. 1, 0ct 1995, pp. 269-271). The resulting sequences are cloned into a baculovirus expression vector such as pVL1392 (Invitrogen). The baculovirus expression vectors are used to infect SF-9 insect cells as described (Guan, X. M., Kobilka, T. S., and Kobilka, B. K. (1992) J. Biol. Chem. 267, 21995-21998). Infected SF-9 cells could be grown in 1000-ml cultures in SF900 II medium (Life Technologies, Inc.) containing 5% fetal calf serum (Gemini, Calabasas, Calif.) and 0.1 mg/ml gentamicin (Life Technologies, Inc.) for 48 hours at which time the cells could be harvested. Cell membrane preparations could be separated from soluble proteins following cell lysis. nGPCR-x protein purification is carried out as described for purification of the θ2 receptor (Kobilka, Anal.

Biochem., 231 (1): 269-271, 1995) including solubilization of the membranes in 0.8-1.0% n-dodecyl-D-maltoside (DM) (CalBiochem, La Jolla, Calif.) in buffer containing protease inhibitors followed by Ni-column chromatography using chelating Sepharose™ (Pharmacia, Uppsala, Sweden). The eluate from the Ni-column is further purified on an Ml anti-FLAG antibody column (IBI). Receptor containing fractions are monitored by using receptor specific antibodies following western blot analysis or by SDS-PAGE analysis to look for an appropriate sized protein band (appropriate size would be the predicted molecular weight of the protein).

This method of purifying G protein is particularly useful to isolate G proteins that bind to the nGPCR-x proteins in the absence of an activating ligand.

Some of the preferred embodiments of the invention described above are outlined below and include, but are not limited to, the following embodiments. As those skilled in the art will appreciate, numerous changes and modifications may be made to the preferred embodiments of the invention without departing from the spirit of the invention. It is intended that all such variations fall within the scope of the invention.

The entire disclosure of each publication cited herein is hereby incorporated by reference.

What is claimed is:

1. An isolated nucleic acid molecule comprising a nucleotide sequence that encodes a polypeptide comprising an amino acid sequence homologous to sequences selected from the group consisting of: SEQ ID NO:59 to SEQ ID NO: 116; said nucleic acid molecule encoding at least a portion of nGPCR-x.

2. The isolated nucleic acid molecule of claim 1 comprising a sequence that encodes a polypeptide comprising a sequence selected from the group consisting of SEQ ID NO:59 to SEQ ID NO:116.

3. The isolated nucleic acid molecule of claim 1 comprising a sequence homologous to a sequence selected from the group consisting of SEQ ID NO: 1 to SEQ ID NO:58.

4. The isolated nucleic acid molecule of claim 1 comprising a sequence selected from the group of sequences consisting of SEQ ID NO: 1 to SEQ ID NO:58.

5. The isolated nucleic acid molecule of claim 1 wherein said nucleic acid molecule is DNA.

6. The isolated nucleic acid molecule of claim 1 wherein said nucleic acid molecule is RNA.

7. An expression vector comprising a nucleic acid molecule of any one of claims 1 to 4.

8. The expr ession vector of claim 7 wherein said nucleic acid molecule compr ises a sequence selected from the group of sequences consisting of SEQ ID NO: 1 to SEQ ID NO:58.

9. The expression vector of claim 7 wherein said vector is a plasmid.

10. The expression vector of claim 7 wherein said vector is a viral particle.

11. The expression vector of claim 10 wherein said vector is selected from the group consisting of adenoviruses, baculoviruses, parvoviruses, herpesviruses, poxviruses, adeno-associated viruses, Semliki Forest viruses, vaccinia viruses, and retroviruses.

12. The expression vector of claim 7 wherein said nucleic acid molecule is operably connected to a promoter selected from the group consisting of simian virus 40, mouse mammary tumor virus, long terminal repeat of human immunodeficiency virus, maloney virus, cytomegalovirus immediate early promoter, Epstein Barr virus, rous sarcoma virus, human actin, human myosin, human hemoglobin, human muscle creatine, and human metalothionein.

13. A host cell transformed with an expression vector of claim 7.

14. The transformed host cell of claim 13 wherein said cell is a bacterial cell.

15. The transformed host cell of claim 14 wherein said bacterial cell is E. coli.

16. The transformed host cell of claim 13 wherein said cell is yeast.

17. The transformed host cell of claim 16 wherein said yeast is S. cerevisiae.

18. The transformed host cell of claim 13 wherein said cell is an insect cell.

19. The transformed host cell of claim 18 wherein said insect cell is S. frugiperda.

20. The transformed host cell of claim 13 wherein said cell is a mammalian cell.

21. The transformed host cell of claim 20 wherein mammalian cell is selected from the group consisting of chinese hamster ovary cells, HeLa cells, African green monkey kidney cells, human BEK-293 cells, and murine 3T3 fibroblasts.

22. An isolated nucleic acid molecule comprising at least 10 nucleotides, said nucleic acid molecule comprising a nucleotide sequence complementary to at least a portion of a sequence selected from the group of sequences consisting of SEQ ID NO: 1 to SEQ ID NO:58.

23. The nucleic acid molecule of claim 22 wherein said molecule is an antisense oligonucleotide directed to a region of a sequence selected from the group of sequences consisting of SEQ ID NO:1 to SEQ ID NO:58.

24. The nucleic acid molecule of claim 23 wherein said oligonucleotide is directed to a regulatory region of a sequence selected from the group of sequences consisting of SEQ ID NO: 1 to SEQ ID NO:58.

25. A composition comprising a nucleic acid molecule of any one of claims 1 to 4 or 22 and an acceptable carrier or diluent.

26. A composition comprising a recombinant expression vector of claim 7 and an acceptable carrier or diluent.

27. A method of producing a polypeptide that comprises a sequence selected from the group of sequences consisting SEQ ID NO:59 to SEQ ID NO: 116, and homologs thereof, said method comprising the steps of:
a) introducing a recombinant expression vector of claim 8 into a compatible host cell;
b) growing said host cell under conditions for expression of said polypeptide; and
c) recovering said polypeptide.

28. The method of claim 27 wherein said host cell is lysed and said polypeptide is recovered from the lysate of said host cell.

29. The method of claim 27 wherein said polypeptide is recovered by purifying the culture medium without lysing said host cell.

30. An isolated polypeptide encoded by a nucleic acid molecule of claim 1.

31. The polypeptide of claim 30 wherein said polypeptide comprises a sequence selected from the group of sequences consisting of SEQ ID NO:59 to SEQ ID NO: 116.

32. The polypeptide of claim 30 wherein said polypeptide comprises an amino acid sequence homologous to a sequence selected from the group of sequences consisting of SEQ ID NO:59 to SEQ ID NO:116.

33. The polypeptide of claim 30 wherein said sequence homologous to a sequence selected from the group of sequences consisting of SEQ ID NO:59 to SEQ ID NO:116 comprises at least one conservative amino acid substitution compared to the sequences in the group of sequences consisting of SEQ ID NO:59 to SEQ ID NO: 116.

34. The polypeptide of claim 30 wherein said polypeptide comprises an allelic variant of a polypeptide with a sequence selected from the group of sequences consisting of SEQ ID NO:59 to SEQ ID NO:116.

35. A composition comprising a polypeptide of claim 34 and an acceptable carrier or diluent.

36. An isolated antibody which binds to an epitope on a polypeptide of claim 30.

37. The antibody of claim 36 wherein said antibody is a monoclonal antibody.

38. A composition comprising an antibody of claim 36 and an acceptable carrier or diluent.

39. A method of inducing an immune response in a mammal against a polypeptide of claim 30 comprising administering to said mammal an amount of said polypeptide sufficient to induce said immune response.

40. A method for identifying a compound which binds nGPCR-x comprising the steps of:
a) contacting nGPCR-x with a compound; and
b) determining whether said compound binds nGPCR-x.

41. The method of claim 40 wherein the nGPCR-x comprises an amino acid sequence selected from the group consisting of SEQ ID NO:59 to SEQ ID NO: 116.

42. The method of claim 40 wherein binding of said compound to nGPCR-x is determined by a protein binding assay.

43. The method of claim 40 wherein said protein binding assay is selected from the group consisting of a gel-shift assay, Western blot, radiolabeled competition assay, phage-based expression cloning, co-fractionation by chromatography, co-precipitation, cross linking, interaction trap/two-hybrid analysis, southwestern analysis, and ELISA.

44. A compound identified by the method of claim 40.

45. A method for identifying a compound which binds a nucleic acid molecule encoding nGPCR-x comprising the steps of:
a) contacting said nucleic acid molecule encoding nGPCR-x with a compound; and
b) determining whether said compound binds said nucleic acid molecule.

46. The method of claim 45 wherein binding is determined by a gel-shift assay.

47. A compound identified by the method of claim 45.

48. A method for identifying a compound which modulates the activity of nGPCR-x comprising the steps of:
a) contacting nGPCR-x with a compound; and
b) determining whether nGPCR-x activity has been modulated.

49. The method of claim 48 wherein the nGPCR-x comprises an amino acid sequence selected from the group consisting of SEQ ID NO:59 to SEQ ID NO: 116.

50. The method of claim 48 wherein said activity is neuropeptide binding.

51. The method of claim 48 wherein said activity is neuropeptide signaling.

52. A compound identified by the method of claim 48.

53. A method of identifying an animal homolog of nGPCR-x comprising the steps:
a) comparing the nucleic acid sequences of the animal with a sequence selected from the group of sequence consisting of SEQ ID NO: 1 to SEQ ID NO:58, and portions thereof, said portions being at least 10 nucleotides; and
b) identifying nucleic acid sequences of the animal that are homologous to said sequence selected from the group sequence consisting of SEQ ID NO: 1 to SEQ ID NO:58, and portions thereof, said portions comprising at least 10 nucleotides.

54. The method of calim 53 wherein comparing the nucleic acis sequences of the animal with a sequence selected from the group of sequences consisting of SEQ ID NO: 1 to SEQ ID NO: 58, and portions thereof, said portions being at least 10 nucleotides, is performed by DNA hybridization.

55. The method of claim 53 wherein comparing the nucleic acid sequences of the animal with a sequence selected from the group of sequences consisting of DEQ ID NO: 1 to SEQ ID NO: 58, and portions thereof, said portions being at least 10 nucleotides, is performed by computer homology search.

56. A method of screening a human subject to diagnose a disorder affecting the brain or genetic predisposition therefor, comprising the steps of:
(a) assaying nucleic acid of a human subjecto to dtermine a presence or an absence of a mutation altering an amino acid sequence, expression, or biological activity of at least one nGPCR-x that is expressed in the brain, wherein the nGPCR-x comprises an amino acid sequence selected from the group consisting of SEQ ID NO: 59 to SEQ ID NO: 116, and allelic variants thereof, and wherein the nuvbleic acid coorisponds to a gene encoding the nGPCR-x and;
(b) diagnosing the disorder or predisposition from the presence or absence of said mutation, wherein the presence of a mutation altering the amino acid sequence, expression, or biological activity of the nGPCR-x in the nucleic acid correlates with an increased risk of developing the disorder.

57. A method according to claim 56, wherein the disease is mental disorder.

58. A method according to claim 56, wherein the assaying step comprises at least one procedure selected from the group consisting of:
a) comparing nucleotide sequences from the human subject and reference sequences and dtermining a difference of at lease a nucleotide of at least one codon between the nucleotide sequences from the human subject that encodes a nGPCR-x reference seuence;
b) performing a hybridization assay to dtermine whether nucleic acid from the human subject has a nucleotide sequence identical to or different from one or more reference sequences;
(c) performing a polynucleotide migration assay to dtermine whether nucleic acid from the human subject has a nucleotide sequence identical to or different from one or more reference sequences; and
(d) performing a restriction endonuclease digestion to dtermine whether nucleic acid from the human subject has a nucleotide sequence identical to or different from one or more reference dequences.

59. A method according to claim 58 wherein the assaying step comprises: performing a polymerase chain reaction assay to amplify nucleic acid comprising nGPCR-x coding sequence, and determining nucleotide sequence of the amplified nucleic acid.

60. A method of screening for an nGPCR-x hereditary mental disorder genotype in a human patient, comprising the steps of:
(a) providing a biological sample comprising nucleic acid from said patient, said nucleic acid including sequences corresponding to alleles on nGPCR-;x and
(b) detecting the presence of one or more mutations in the nGPCR-x allele;
wherein the presence of a mutation in a nGPCR-x allele is indicative of a hereditary mental disorder genotype.

61. The method according to claim 60 wherein said biological sample is a cell sample.

62. The method according to claim 60 wherein said detecting the presence of a mutation comprises sequencing at least a portion of said nucleic acid, said portion comprising at least one codon of said n-GPCR-x allele, said portion comprising at least 10 nucleotides.

63. The method according to claim 60 wherein said nucleic acid is DNA.

64. The method according to claim 60 wherein said nucleic acid is RNA.

65. A kit for screening a human subject to diagnose a mental disorder or a genetic predisposition therefor, comprising, in association:
(a) an oligonucleotide useful as a probe for identifying polymorphisms in a human nGPCR-x gene, the oligonucleotide comprising 6-50 nucleotides in a sequenct that is identical or complementary to a sequence of a wild-type human nGPCR-x gene sequence or nGPCR-x coding sequence, except for one sequence difference selected from the gruop consisting of a nucleotide addition, a nucleotide deletion, or nucleotide substitution; and
(b) a media packaged with the oligonucleotide, said media containing information for identifying polumorphisms that correlate with mental disorder or genetic predisposition therefor, the polymorphisms being identifiable using the oligonucleitide as a probe.

66. A method of identifying a nGPCR-x allelic variant that correlates with a mental disorder, comprising the steps of:
(a) providing a biological sample comprising nucleic acid from a human patient diagnosed with a mental disorder, or from the patient's genetic progenitors or progeny;
(b) detecting in the nucleic acid the presence of one or more mutations in an nGPCR-x that is expressed in the brain, wherein the nGPCR-x comprises an amino acid sequence selected from the group consiting of SED ID NO:59 to SEQ ID NO:116, and allelic variants thereof, and wherein the nucleic acid includes sequence corresponding to the gene or genes encoding nGPCR-x;
wherein the one or more mutations detected indicates an allelic variant that correlates with a mental disorder.

67. A purified and isolated polynucleotide comprising a nucleotide sequence encoding a nGPCR-x allelic variant identified according to claim 66.

68. A host cell transformed or transfected with a polynucleotide according to claim 67 or with a vector comprising the polynucleotide.

69. A puirified polynucleotide comprising a nucleotide sequence encoding nGPCR-x of a human with a mental disorder;
wherein said polynucleotide hybridizes to the complement of a sequence selected from the group consisting of SEQ ID NO:1 to SEQ ID NO:58 under the following hybridization conditions:
(a) hybridization for 16 hours at 42° C. in a hybridization solution comprising 50% formamide, 1% SDS, 1 M NaCl, 10% dexatran sulfate and
(b) washing 2 times for 30 minutes at 60° C. in a wash solution comprising 0.1× SSC and 1% SDS; and
wherein the polynucleotide that encodes nGPCR-x amino acid sequence of the human diffes from the sequence selected from the group consisting of SEQ ID NO:1 to SEQ ID NO:58 by at least one residue.

70. A vector comprising a polynucleotide according to claim 69.

71. A host cell that has been transformed or transfected with a polynucleotide according to claim 69 and that expresses the nGPCR-x protein encoded by the polynucletide.

72. A host cell accordint to claim 71 that has been co-transfected with a polynucleotide encoding the nGPCR-x amino acid sequence set forth in a sequence selected from the group consisting of SEQ ID NO:1 to SEQ ID NO:58 and that expresses the nGCPR-x protein having the amino acid sequence set forth in SEQ ID NO:59 to SEQ ID NO:116.

73. A method for identifying a modilatior of a biological activity of nGPCR-x comprising the steps of:
a) contacting a cell according to calim 72 in the presence and in the absence of a putative modulator compound;
b) measuring nGPCR-x biological activity in the cell;
wherein decreased or increased nGPCR-x biological activity in the presence versus absence of the putative modulator is indicative of a modulator of biological activity.

74. A method to identify compounds useful for the treatment of a mental disorder, said method comprising the steps of:
(a) contacting a composition comprising nGPCR-x with a compound suspected of binding nGPCR-x;
(b) detecting binding between nGPCR-x and the compound suspected of binding nGPCR-x;
wherein compound identified as binding nGPCR-x are candidate compounds useful for the treatment of a mental disorder.

75. A method for identifying a compound uselful as a modulator of binding between nGPCR-x and a binding partner of nGPCR-x comprising the steps of:
(a) contacting the binding partner and a composition comprising nGPCR-x in the presence and in the absence of a putative modulator compound;
(b) detecting binding between the binding partner and nGPCR-x;
wherein decreased or increased binding between the binding partner and nGPCR-x in the presence of the putative modulator, as compared to binding in the absence of the putative modulator is indicative a modulator compound useful for the treatment of a mental disorder.

76. A method according to claim 74 or 75 wherein the composition comprises a cell expressing nGPCR-x on its surface.

77. A method according to claim 76 wherein the composition comprises a cell transformed or transfected with a polynucleotide that encodes nGPCR-x.

78. A method of purifying a G protein from a sample containing said G protein comprising the steps of:
a) contacting said sample with a polypeptide of claim 1 for a time sufficient to allow said G protein to form a complex with said polypeptide;
b) isolating said complex from remaining components of said sample;
c) maintaining said complex under conditions which result in dissociation of said G protein from said polypeptide; and
d) isolating said G protein from said polypeptide.

79. The method of claim 78 wherein said sample comprises an amino acid sequence selected from the group of sequences consisting of SEQ ID NO:59 to SEQ ID NO:116.

80. The method of claim 78 wherein said polypeptide comprises an amino acid sequence homologous to a sequence selected from the group of sequences consisting of SEQ ID NO:59 to SEQ ID NO:116.

81. The method of claim 78 wherein said polypeptide comprises an amino acid sequence selected from the group consisting of: SEQ ID NO:59 to SEQ ID NO: 116.

82. The isolated nucleic acid molecule of claim 1 comprising a sequence that encodes a polypeptide comprising a sequence of SEQ ID NO:67.

83. The isolated nucleic acid molecule of claim 1 comprising a sequence homologous to a sequence of SEQ ID NO:9.

84. The isolated nucleic acid molecule of claim 1 comprising a sequence of SEQ ID NO: 9.

85. An expression vector comprising a nucleic acid molecule of any one of claims 82 to 84.

86. An isolated nucleic acid molecule comprising at least 10 nucleotides, said nucleic acid molecule comprising a nucleotide sequence complementary to at least a portion of a sequence of SEQ ID NO:9.

87. The nucleic acid molecule of claim 86 wherein said molecule is an antisense oligonucleotide directed to a region of a sequence of SEQ ID NO:9.

88. The nucleic acid molecule of claim 87 wherein said oligonucleotide is directed to a regulatory region of a sequence of SEQ ID NO:9.

89. A method of producing a polypeptide that comprises a sequence of SEQ ID NO:67, and homologs thereof, said method comprising the steps of:
a) introducing a recombinant expression vector of claim 85 into a compatible host cell;
b) growing said host cell under conditions for expression of said polypeptide; and
c) recovering said polypeptide.

90. An isolated polypeptide encoded by a nucleic acid molecule of claim 82.

91. The polypeptide of claim 90 wherein said polypeptide comprises a sequence of SEQ ID NO:67.

92. The polypeptide of claim 90 wherein said polypeptide comprises an amino acid sequence homologous to a sequence of SEQ ID NO:67.

93. An isolated antibody which binds to an epitope on a polypeptide of claim 90.

94. The antibody of claim 93 wherein said antibody is a monoclonal antibody.

95. A method of inducing an immune response in a mammal against a polypeptide of claim 90 comprising administering to said mammal an amount of said polypeptide sufficient to induce said immune response.

96. A method for identifying a compound which binds nGPCR-93 comprising the steps of:
a) contacting nGPCR-93 with a compound; and
c) determining whether said compound binds nGPCR-93.

97. The method of claim 96 wherein nGPCR-93 comprises an amino acid sequence of SEQ ID NO:67.

98. The method of claim 96 wherein binding of said compound to nGPCR-x is determined by a protein binding assay.

99. A compound identified by the method of claim 96.

100. A method for identifying a compound which binds a nucleic acid molecule encoding nGPCR-93 comprising the steps of:
a) contacting said nucleic acid molecule encoding nGPCR-93 with a compound; and
b) determining whether said compound binds said nucleic acid molecule.

101. The method of claim 100 wherein binding is determined by a gel-shift assay.

102. A compound identified by the method of claim 100.

103. A method for identifying a compound which modulates the activity of nGPCR-93 comprising the steps of:
a) contacting nGPCR-93 with a compound; and
b) determining whether nGPCR-93 activity has been modulated.

104. The method of claim 103 wherein the nGPCR-x comprises an amino acid sequence of SEQ ID NO:67.

105. The method of claim 103 wherein said activity is neuropeptide binding.

106. The method of claim 103 wherein said activity is neuropeptide signaling.

107. A compound identified by the method of claim 103.

108. A method of identifying an animal homolog of nGPCR-93 comprising the steps:
a) comparing the nucleic acid sequences of the animal with a sequence of SEQ ID NO:9, and portions thereof, said portions being at least 10 nucleotides; and
b) identifying nucleic acid sequences of the animal that are homologous to said sequence of SEQ ID NO:9, and portions thereof, said portions comprising at least 10 nucleotides.

109. The method of claim 108 wherein comparing the nucleic acid sequences of the animal with a sequence of SEQ ID NO:9, and portions thereof, said portions being at least 10 nucleotides, is performed by DNA hybridization.

110. The method of claim 108 wherein comparing the nucleic acid sequences of the animal with a sequence of SEQ ID NO:9, and portions thereof, said portions being at least 10 nucleotides, is performed by computer homology search.

111. A method of screening a human subject to diagnose a disorder affecting the brain or genetic predisposition therefor, comprising the steps of:
(a) assaying nucleic acid of a human subject to determine a presence or an absence of a mutation altering an amino acid sequence, expression, or biological activity of at least one nGPCR-x that is expressed in the brain, wherein the nGPCR-x comprises an amino acid sequence of SEQ ID NO:67, and allelic variants thereof, and wherein the nucleic acid corresponds to a gene encoding the nGPCR-x; and
(b) diagnosing the disorder or predisposition from the presence or absence of said mutation, wherein the presence of a mutation altering the amino acid sequence, expression, or biological activity of the nGPCR-x in the nucleic acid correlates with an increased risk of developing the disorder.

112. A method according to claim 11, wherein the disease is a mental disorder.

113. A purified polynucleotide comprising a nucleotide sequence encoding nGPCR-x of a human with a mental disorder;
wherein said polynucleotide hybridizes to the complement of a sequence of SEQ ID NO:9 under the following hybridization conditions:
(a) hybridization for 16 hours at 42° C. in a hybridization solution comprising 50% formamide, 1% SDS, 1 M NaCl, 10% dextran sulfate and (b) washing 2 times for 30 minutes at 60° C. in a wash solution comprising 0.1x SSC and 1% SDS; and
wherein the polynucleotide that encodes nGPCR-x amino acid sequence of the human differs from the sequence of SEQ ID NO:9 by at least one residue.

114. A vector comprising a polynucleotide according to claim 113.

115. A host cell that has been transformed or transfected with a polynucleotide according to claim 113 and that expresses the nGPCR-x protein encoded by the polynucleotide.

116. A host cell according to claim 115 that has been co-transfected with a polynucleotide encoding the nGPCR-x amino acid sequence set forth in a sequence of SEQ ID NO:9 and that expresses the nGPCR-x protein having the amino acid sequence set forth in SEQ ID NO:67.

117. A method for identifying a modulator of biological activity of nGPCR-93 comprising the steps of:
a) contacting a cell according to claim 11-in the presence and in the absence of a putative modulator compound;
b) measuring nGPCR-93 biological activity in the cell;
wherein decreased or increased nGPCR-93 biological activity in the presence versus absence of the putative modulator is indicative of a modulator of biological activity.

118. A method for identifying a compound useful as a modulator of binding between nGPCR-93 and a binding partner of nGPCR-93 comprising the steps of:
(a) contacting the binding partner and a composition comprising nGPCR-93 in the presence and in the absence of a putative modulator compound;
(b) detecting binding between the binding partner and nGPCR-93;
wherein decreased or increased binding between the binding partner and nGPCR-93 in the presence of the putative modulator, as compared to binding in the absence of the putative modulator is indicative a modulator compound useful for the treatment of a mental disorder.

119. A method according to claim 117 or 118 wherein the composition comprises a cell expressing nGPCR-93 on its surface.

120. A method according to claim 119 wherein the composition comprises a cell transformed or transfected with a polynucleotide that encodes nGPCR-93.

121. A method of purifying a G protein from a sample containing said G protein comprising the steps of:
a) contacting said sample with a polypeptide of claim 90 for a time sufficient to allow said G protein to form a complex with said polypeptide;
b) isolating said complex from remaining components of said sample;
c) maintaining said complex under conditions which result in dissociation of said G protein from said polypeptide; and
d) isolating said G protein from said polypeptide.

122. The method of claim 121 wherein said sample comprises an amino acid sequence of SEQ ID NO:67.

123. The method of claim 121 wherein said polypeptide comprises an amino acid sequence homologous to a sequence of SEQ ID NO:67.

124. The method of claim 121 wherein said polypeptide comprises an amino acid sequence of SEQ ID NO:67.
